(19) United States
(12) Patent Application Publication
(10) Pub. No.: US 2006/0211603 A1
(43) Pub. Date: Sep. 21, 2006

(54) Ramoplanin derivatives possessing antibacterial activity

(75) Inventors: Bore G. Raju, Fremont, CA (US); Romeo Ciabatti, Novate Milanese (IT); Sonia Ilaria Maffioli, Milano (IT); Upinder Singh, Fremont, CA (US); Gabriella Romano, Legnano (IT); Elena Michelucci, Legnano (IT); Paolo Simone Tiseni, Milano (IT); Gianpaolo Candiani, Gorgonzola (IT); Bum Kim, Union City, CA (US); Hardwin O'Dowd, Hayward, CA (US)

Correspondence Address: O'MELVENY & MYERS LLP, 610 NEWPORT CENTER DRIVE, 17TH FLOOR, NEWPORT BEACH, CA, 92660, US

(73) Assignee: VICURON PHARMACEUTICALS INC., King of Prussia, PA (US)

(21) Appl. No.: 11/198,763

(22) Filed: Aug. 4, 2005

Related U.S. Application Data
(60) Provisional application No. 60/602,780, filed on Aug. 18, 2004.

(51) Int. Cl. A61K 38/14; C07K 9/00; C07K 7/64; A61K 38/12

(52) U.S. Cl. 514/8; 514/9; 530/317

(57) ABSTRACT

Novel ramoplanin derivatives are disclosed. These ramoplanin derivatives exhibit antibacterial activity. As the compounds of the subject invention exhibit potent activities against gram positive bacteria, they are useful antimicrobial agents. Methods of synthesis and of use of the compounds are also disclosed.

### BACKGROUND OF THE INVENTION

1. Field of the Invention

This invention relates to ramoplanin derivatives that exhibit antibacterial activity.

2. State of the Art

Ramoplanin is a biosynthetic product that adversely affects growth of various microorganisms, in particular gram positive bacteria.

Ramoplanin is a known member of the cyclic peptide antibiotics more precisely known as glycolipodepsipeptides which have been been described in U.S. Pat. Nos. 4,303,646 and 4,328,316. It is a complex substance whose separate factors A₁, A₂ and A₃ have been described in U.S. Pat. No. 4,427,656. Ramoplanin factors A′₁, A′₂ and A′₃ have been described in EP-B-318680. The aglycones of the above factors have been described in EP-B-0337203. A method for selectively increasing the ratio of single major components A₂ and A₃ is described in EP-B-0259780.

The structure of ramoplanin and its factors and derivatives have been described in several articles and publications, see R. Ciabatti et al., J. Antib. 1989, 42, 254-267, J. K. Kettenring et al., J. Antib. 1989, 42, 268-275, R. Ciabatti and B. Cavalleri, Bioactive Metabolites from Microorganisms, Elsevier Science Publishers, 1989, 205-219 and M. Kurz and W. Guba, Biochemistry 1996, 35, 12570-12575.

N. J. Skelton et al. in J. Am. Chem. Soc. 1991, 113, 7522-7530 describe another member of this family, termed ramoplanose.

In addition to the natural compounds disclosed in the above publications, other semisynthetic derivatives related thereto have been described in U.S. Pat. No. 5,708,988, EP-B-0337203, WO 03/076460, Jiang et al., J. Am. Chem. Soc. 2002, 124: 5288-5290; Jiang et al., 3. Am. Chem. Soc. 2003, 124: 5288-5290; Helm et al., J. Am. Chem. Soc. 2002, 124: 13970-13971; Wanner et al., Bioorg. Med. Chem. Lett. 2003, 13: 1169-1173; Hu et al. J. Am. Chem. Soc. 2003, 125: 8736-8737; Skelton et al., J. Am. Chem. Soc. 1991, 113: 7522-7530; and Maplestone et al., FEBS Lett. 1993, 326: 95-100.

Ramoplanin derivatives remain attractive targets for antibacterial drug discovery. Accordingly, ramoplanin derivatives that possess antimicrobial activity are desired as potential antibacterial agents.

All references cited herein are incorporated by reference in their entirety.

### SUMMARY OF THE INVENTION

The present invention provides ramoplanin derivatives that possess antibacterial activity. In one of its composition aspects, this invention is directed to a compound of Formula (I): 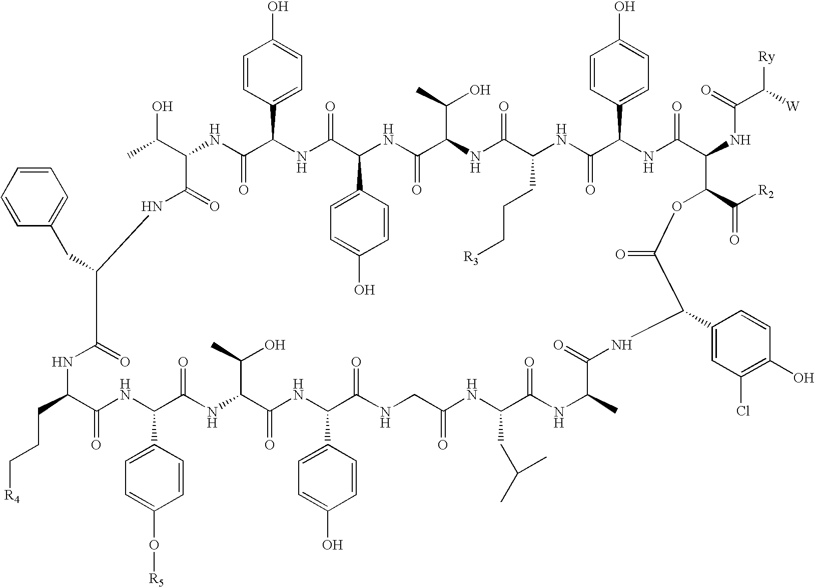
wherein:
   R² is selected from the group consisting of: —NH₂, —NHR¹⁸, —OH, and —OR¹⁶, wherein R¹⁸ is alkyl, aminoalkyl, alkylaminoalkyl, or alkoxycarbonylaminoalkyl, and wherein R¹⁶ is alkyl or aminoalkyl;
   R³ and R⁴ are independently selected from the group consisting of: —NH₂, —NHCO(CH₂)ₙNH₂, —NHCO(CH₂)ₙCO₂H, —NH(CH₂)ₙCO₂H, —NHCOC(R¹⁷)NH₂, —NH—C(═NH)—NH₂, —NH-alkyl, —NH(CH₂)ₙNH₂, —N(CH₃)₂, —NHCO—C₆H₄-p-CH₂NH₂, and 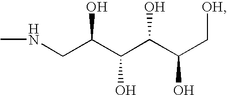
   wherein n is 1-5, and wherein R¹⁷ is a natural or synthetic amino acid side chain;
   R⁵ is selected from the group consisting of: H, α-D-mannopyranosyl, and 2-O-α-D-mannopyranosyl-α-D-mannopyranosyl;
   R^{y} is selected from the group consisting of: —H, —CH₂CONH₂, —CH₂CONHR¹⁸, —CH₂CO₂H, and —CH₂CO₂R¹⁶, wherein R¹⁸ is alkyl, aminoalkyl, alkylaminoalkyl, or alkoxycarbonylaminoalkyl, and wherein R¹⁶ is alkyl or aminoalkyl;
   W is selected from the group consisting of: —NH—C(O)—R^{x}, —NH—C(S)—NH—R^{z}, —NH—C(O)—NH—R^{z}, —NH—C(O)O—R^{z}, —NH—R′, —NH—S(O₂)—R″, —N(CH₃)—S(O₂)—R″, —NH—C(O)—CH═N—NH—R²⁰, and substituted aryl;
   R^{x} is selected from the group consisting of: alkyl, substituted alkyl, alkenyl, substituted cycloalkyl, cycloalkenyl, aryl, substituted aryl, heteroaryl, substituted heteroaryl, heterocyclic, substituted heterocyclic, substituted carbonyl-amino acid residue (—C(O)—CH(R¹¹)—NH—C(O)—R¹²), wherein R¹¹ is a natural or synthetic amino acid side chain, and R¹² is alkyl or substituted alkyl;
   R^{z} is selected from the group consisting of: alkyl, substituted alkyl, cycloalkyl, substituted cycloalkyl, alkenyl, alkynyl, aryl, substituted aryl,
   R′ is -alkylene-R¹⁰, wherein R¹⁰ is selected from the group consisting of: H, aryl, substituted aryl, cycloalkyl, substituted cycloalkyl;
   R″ is selected from the group consisting of: aryl and haloaryl;
   R²⁰ is selected from the group consisting of:
      carbonyl substituted with aryl, substituted aryl, arylalkyl, heteroaryl, heterocyclic, and substituted amino, and
      thiocarbonyl substituted with substituted amino;
   and prodrugs, tautomers or pharmaceutically acceptable salts thereof;
   with the following provisos:
   (1) when R^{y} is —CH₂CONH₂, R² is —NH₂, R³ and R⁴ are —NH₂ or —NH(protecting group), R⁵ is H, α-D-mannopyranosyl, or 2-O-α-mannopyranosyl-α-D-mannopyranosyl, and W is —NH—C(O)—R^{x}, then R^{x} is not alkyl, alkenyl, —R²¹, -(C1-C4 alkylene)-R²¹, or -(C2-C4 alkenylene)-R²¹;
   wherein R²¹ is alkoxy, alkenyloxy, alkoxy substituted with halo; alkenyloxy substituted with halo; phenyl; phenyl substituted with R²², halo, cyano, nitro, alkyl, haloalkyl, alkoxy, haloalkoxy, alkylsulfanyl, and/or haloalkylsulfanyl; napthyl; napthyl substituted with halo, alkyl, haloalkyl, alkoxy, and/or haloalkoxy, phenoxy; phenoxy substituted with halo, cyano, nitro, alkyl, haloalkyl, alkoxy, haloalkoxy, alkylsulfanyl, and/or haloalkylsulfanyl; napthoxy, napthoxy substituted with halo, alkyl, haloalkyl, alkoxy, and/or haloalkoxy;
   wherein R²² is phenyl, phenylalkyl, phenoxy, or phenoxyalkyl, and wherein the phenyl portion of said phenyl, phenylalkyl, phenoxy, and phenoxyalkyl may be substituted with halo, cyano, nitro, alkyl, haloalkyl, alkoxy, haloalkoxy, alkylsulfanyl, and/or haloalkylsulfanyl; and
   (2) when R^{y} is —CH₂CONH₂, R² is —NH₂, R³ and R⁴ are —NH₂ or —NHCOCH(CH₃)NH₂, R⁵ is 2-O-α-D-mannopyranosyl-α-D-mannopyranosyl, and W is —NH—C(O)—R^{x}, then R^{x} is not alkyl or alkenyl.

In one embodiment, the compound of Formula I has a minimum inhibition conoentration of 128 μg/mL or less against at least one of the organisms selected from the group consisting of Actinomyces spp, Bacillus spp, Bacillus anthracis, Bacillus cereus, Clostridium spp, Clostridium difficile, Clostridium perfringens, Clostridium botulinum, Clostridium tetani, Clostridium ramosum, Clostridium, Corynebacterium spp, Corynebacterium dihpteriae, Enterococcus spp, Enterococcus faecalis, Enterococcus faecium, Enterococcus gallinarum, Enterococcus casseliflavus, Enterococcus avium, Enterococcus durans, Enterococcus raffinosus, Entrerococcus hirae, Enterococcus pseudoavium, Enterococcus malodoratus, Enterococcus mundtii, Erysipelothrix rhusiopathiae, Eubacterium, Gemella haemolysans, Gemella morbillorum, Lactobacillus spp, Lactobacillus rhamnosus, Lactobacillus paracasei, Leuconostoc spp, Leuconostoc mesenteroides, Listeria monocytogenes, Peptostreptococcus magnus, Peptostreptococcus asaccharolyticus, Peptostreptococcus anaerobius, Peptostreptococcus prevotii, Peptostreptococcus micros, Peptostreptococcus hydrogenalis, Propionibacterium acne, Staphylococcus spp, Staphylococcus aureus, Staphylococcus epidermidis, Staphylococcus hominis, Staphylococcus haemolyticus, Staphylococcus saprophyticus, Streptococcus spp, Streptococcus pneumoniae, Streptococcus pyogenes, Streptococcus agalactiae, Streptococcus mutans, Streptococcus sanguis, Streptococcus mitis, Streptococcus bovis, Streptococcus salivarius, Steptococcus anginosus, Streptococcus constellatus, and Streptococcus intermedius.

In one embodiment, R² is selected from the group consisting of: —NH₂, —OH, —OCH₃, —NH—CH₂CH(CH₃)₂, —NH—CH₂CH₂NHBoc, —NH—CH₂CH₂NH₂, —NHCH₂CH₂CH₂NH₂, —NHCH₂CH₂CH₂CH₂NH₂, —NHCH₂CH₂NHCH₃, —NHCH₂CH₂N(CH₃)₂, and —OCH₂CH₂NH₂. In another embodiment, R² is —NH—CH₂CH₂NH₂. In another embodiment, R² is —NH₂.

In one embodiment, R³ and R⁴ are independently selected from the group consisting of: —NH₂, —N-(aminomethyl-carbonyl)-amino, —N-(2-amino-ethyl-carbonyl)amino, —N-3-amino-propyl-carbonyl)amino, —N-(4-amino-butyl-carbonyl)amino, —N-(5-amino-pentyl-carbonyl)amino, —N-(1,5-diamino-pentyl-carbonyl)amino, —NHCOCH₂CH₂COOH, —NHCH₂CH₂CH₃, —N(CH₃)₂, —NHCH₂COOH, —NH—C(═NH)NH₂, 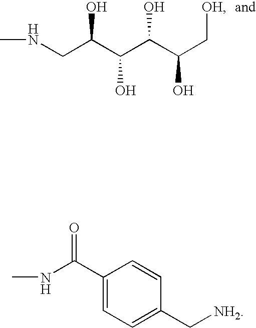
In another embodiment, R³ and R⁴ are independently selected from the group consisting of: —N-(1,5-diamino-pentyl-carbonyl)amino and 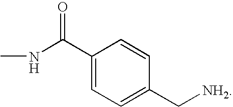
In another embodiment, R³ and R⁴ are —NH₂.

In one embodiment, R⁵ is 2-O-α-D-mannopyranosyl-α-D-mannopyranosyl. In another embodiment, R⁵ is H.

In one embodiment, R^{y} is selected from the group consisting of: —H, —CH₂COOH, —CH₂CONH₂, —CH₂COOCH₃, —CH₂CONHCH₂CH(CH₃)₂, —CH₂CONHCH₂CH₂NHBoc, and —CH₂CONHCH₂CH₂NH₂. In another embodiment, R^{y} is selected from the group consisting of: —CH₂COOH, —CH₂CONH₂, —CH₂COOCH₃, —CH₂CONHCH₂CH(CH₃)₂, —CH₂CONHCH₂CH₂NHBoc, and —CH₂CONHCH₂CH₂NH₂. In another embodiment, R^{y} is —CH₂CONHCH₂CH₂NH₂. In another embodiment, R_{y} is —CH₂CONH₂.

In one embodiment, W is —NH—C(O)—R^{x}.

In one embodiment, R^{x} is selected from the group consisting of: thiophen-2-yl-methyl; 3-methyl-benzo[b]thiophen-2-yl-methyl; benzo[b]thiophen-3-yl-methyl; 5-chloro-benzo[b]thiophen-3-yl-methyl; thiophen-3-yl-methyl; benzo[1,3]dioxol-5-yl-methyl; (±)-2,3-dihydro-benzo[1,4]dioxin-2-yl; 2-benzyloxy-benzyl; 2-phenylsulfanyl-benzyl; 4-thiophen-2-yl-phenyl; benzo[d]isoxazol-3-yl-methyl; benzothiazol-5-yl; 5-phenyl-thiophen-2-yl; 3-methyl-thiophen-2-yl-methyl; 2-E-(3-methyl-thiophen-2-yl)ethenyl; 2-(3-methyl-thiophen-2-yl)-ethyl; 3-phenyl-isoxazol-5-yl; 5-methyl-isoxazol-3-yl; 5-methyl-2-phenyl-2H-[1,2,3]-triazol-4-yl; 5-tert-butyl-2-methyl-2H-pyrazol-3-yl; 3-pyridin-2-yl-isoxazol-5-yl; 3-ethyl-isoxazol-5-yl; 3-propyl-isoxazol-5-yl; 3-isopropyl-isoxazol-5-yl; 3-isobutyl-isoxazol-5-yl; 3-butyl-isoxazol-5-yl; 3-tert-butyl-isoxazol-5-yl; 3-(1-methylpropyl)-isoxazol-5-yl; indol-1-yl-methyl; 2-E-(5-methyl-thiophen-2-yl)-ethenyl; 2-(5-methyl-thiophen-2-yl)ethyl; methyl-sulfonyl-N-phenyl-amino-methyl; phenyl-sulfonyl-N-phenyl-amino-methyl; 5-methyl-thiophen-2-yl; 4-methyl-thiophen-2-yl; 3-methyl-thiophen-2-yl; 5-methyl-thiophen-2-yl-methyl; 4-methyl-thiophen-2-yl-methyl; 2-E-(4-methyl-thiophen-2-yl) ethenyl; 2-(4-methyl-thiophen-2-yl)-ethyl; 5-phenyl-isoxazol-3-yl; 3-phenyl-isoxazol-5-yl-methyl; 3-isobutyl-isoxazol-5-yl-methyl; (5-phenylimidazol-1-yl)methyl; (benzimidazol-1-yl)methyl; (2-phenylimidazol-1-yl)methyl; biphenyl-2-yl-oxy-methyl; biphenyl-3-yl-oxy-methyl; biphenyl-4-yl-oxy-methyl; 3-methyl-isoxazol-5-yl-methyl; benzofuran-2-yl; 1H-indol-3-yl-methyl; 1H-indol-2-yl; 5-ethyl-8-oxo-5,8-dihydro-[1,3]dioxolo-[4,5-g]quinolin-7-yl; 7-chloro-1-cyclopropyl-6-fluoro-4-oxo-1,4-dihydro-quinolin-3-yl; 8-Fluoro-3-methyl-9-(4-methyl-piperazin-1-yl)-2,3-dihydro-1-oxa-3a-aza-phenalen-6-one-5-yl; I-Ethyl-7-methyl-4-oxo-1,4-dihydro-[1,8]naphthyridin-3-yl; quinolin-4-yl; quinolin-8-yl; quinolin-6-yl; 2,2-difluoro-benzo[1,3]dioxol-5-yl; 2,2-Difluoro-benzo[1,3]dioxol-4-yl; quinolin-2-yl; quinolin-5-yl; quinolin-3-yl; (1-oxo-1,3-dihydroisoindol-2-yl)methyl; (2-oxo-2,3-dihydroindol-1-yl)methyl; (2-oxo-benzoxazol-3-yl)methyl; (benzotriazol-1-yl)methyl; (indazol-1-yl)methyl; 2,2-difluoro-benzo[1,3]dioxol-4-yl-methyl; 1-methyl-1H-indol-3-yl-methyl; 5-phenyl-isoxazol-3-yl-methyl; 3-isopropyl-isoxazol-5-yl-methyl; benzo[1,3]dioxol-4-yl; 2,2-difluoro-benzo[1,3]dioxol-5-yl-methyl; (3-methyl-2-oxo-2,3-dihydrobenzimidazol-1-yl)methyl; (2-oxo-2,3-dihydrobenzimidazol-1-yl)methyl; (3-ethyl-2-oxo-2,3-dihydro-benzimidazol-1-yl)methyl; (4-methyl-2-oxo-benzooxazol-3-yl)methyl; (5-methyl-2-oxo-benzooxazol-3-yl)methyl; (6-methyl-2-oxo-benzooxazol-3-yl)methyl; 4-(4-methoxy-phenyl)-thiophen-2-yl; 2-phenyl-thiazolyl-methyl; 2-phenyl-thiazol-4-yl; 2-phenyl-oxazol-4-yl-methyl; 1-methyl-1H-indol-2-yl; 2-phenyl-oxazol-4-yl; 2-methyl-thiazol-4-yl-methyl; 2-methyl-oxazol-4-yl-methyl; (5-methyl-2-phenyl-2H-[1,2,3]triazol-4-yl)methyl; (5-phenyltetrazol-1-yl)methyl; (4R,5S)-(+)-4-methyl-5-phenyl-oxazolidin-2-one-3-yl-methyl; (4S,5R)-(−)-4-methyl-5-phenyl-oxazolidin-2-one-3-yl-methyl; pyrrolidin-2-one-1-yl-methyl; 2-cyclohexyl-oxazolyl-methyl; (4R)-4-phenyl-oxazolidin-2-one-3-yl-methyl; (4S)-4-phenyl-oxazolidin-2-one-3-yl-methyl; (2-cyclohexylthiazol-4-yl)methyl; 5-(4-methyl-phenyl)-tetrazol-1-yl-methyl; 5-(4-methoxy-phenyl)-tetrazol-1-yl-methyl; 2-ethenyl-benzyl; 4-difluoromethoxy-phenyl; 4-trifluoromethoxy-phenyl; 2-ethynyl-benzyl; 1-aceto-piperidin-4-yl; 1-(4-chloro-benzyl)-pyrrolidin-2-one-4-yl; bicyclo[4.2.0]octa-1 (6),2,4-trien-7-yl; 5-methyl-1-phenyl-1H-pyrazol-4-yl; 1-methyl-5-phenyl-1H-pyrazol-3-yl-methyl; (2-methyl-5-phenyl-2H-pyrazol-3-yl)methyl; 1-ethyl-5-phenyl-1H-pyrazol-3-yl-methyl; (2-ethyl-5-phenyl-2H-pyrazol-3-yl)methyl; (2,5-diphenyl-2H-pyrazol-3-yl)methyl; (2-tert-butyl-5-phenyl-2H-pyrazol-3-yl)methyl; (2-cyclohexyl-5-phenyl-2H-pyrazol-3-yl)methyl; (5-methyl-2-phenyl-2H-pyrazol-3-yl)methyl; 2-methyl-5-phenyl-2H-pyrazol-3-yl; 1-methyl-5-phenyl-1H-pyrazol-3-yl; (5-phenyl-1-propyl-1H-pyrazol-3-yl)methyl; 1-butyl-5-phenyl-1H-pyrazol-3-yl-methyl; 1-isobutyl-5-phenyl-1H-pyrazol-3-yl-methyl; (5-phenyl-pyrazol-1-yl)methyl; (3-methyl-5-phenyl-pyrazol-1-yl)methyl; (5-methyl-3-phenylpyrazol-1-yl)methyl; (3-phenylpyrazol-1-yl)methyl; 2-phenyl-2H-pyrazol-3-yl; 2-(bis-methylsulfonylamino)-benzyl; L-phenyl-sulfonyl-amino-phenylmethyl; L-phenyl-sulfonyl-N-methyl-amino-phenylmethyl; phenyl-sulfonyl-amino-methyl; phenyl-sulfonyl-N-methyl-amino-methyl; phenyl-sulfonyl-N-ethyl-amino-methyl; phenyl-sulfonyl-N-isopropyl-amino-methyl; phenyl-sulfonyl-N-propyl-amino-methyl; phenyl-sulfonyl-N-benzyl-amino-methyl; benzyl-sulfonyl-amino-methyl; benzyl-sulfonyl-N-methyl-amino-methyl; benzyl-sulfonyl-N-propyl-amino-methyl; benzyl-sulfonyl-N-benzyl-amino-methyl; benzyl-sulfonyl-N-ethyl-amino-methyl; benzyl-sulfonyl-N-isopropyl-amino-methyl; (4-phenyl-[1,2,3]triazol-1-yl)methyl; (5-phenyl-[1,2,3]triazol-1-yl)methyl; (5-phenyltetrazol-2-yl)methyl; 5-phenyl-oxazol-4-yl; 5-phenyl-oxazol-4-yl-methyl; N-(n-butyl-carbonyl)amino-methyl; N-(n-butyl-carbonyl)amino-benzylmethyl; N-(1-ethyl-n-pentyl-carbonyl)amino-methyl; N-(2-methyl-benzyl-carbonyl)amino-benzylmethyl; 1-N-(n-butyl-carbonyl)amino-ethyl; 1-N-(2-methyl-benzyl-carbonyl)amino-ethyl; N-2-methyl-benzyl-carbonyl)amino-methyl; 1-N-(1-ethyl-n-pentyl-carbonyl)amino-ethyl; N-(1-ethyl-n-pentyl-carbonyl)amino-benzylmethyl; 1-N-(2-methyl-benzyl-carbonyl)amino-2-methyl-butyl; cyclopentyl; cyclopentyl-methyl; 2-cyclopentyl-ethyl; 1-phenyl-cyclopentyl; bicyclo[2.2.1]heptylmethyl; cyclohexylmethyl; 4-methyl-cyclohexyl-methyl; 2-methyl-cyclohexyl-methyl; 4-pentyl-cyclohexyl-methyl; cycloheptyl; cyclopropyl; 2-methylcyclopropyl; 1-methylcyclopropyl; 2,2,3,3-tetramethyl-cyclopropyl; 2-(2-methyl-prop-1-enyl)-3,3-dimethyl-cyclopropyl; 2-phenyl-cyclopropyl; 1-phenyl-cyclopropyl; cyclobutyl; cyclohexen-3-yl; and 2-methyl-benzyl.

In one embodiment, R^{x} is selected from the group consisting of: thiophen-2-yl-methyl; 3-methyl-benzo[b]thiophen-2-yl-methyl; benzo[b]thiophen-3-yl-methyl; 5-chloro-benzo[b]thiophen-3-yl-methyl; thiophen-3-yl-methyl; benzo[1,3]dioxol-5-yl-methyl; (±)-2,3-dihydro-benzo[1,4]dioxin-2-yl; 2-benzyloxy-benzyl; 2-phenylsulfanyl-benzyl; 4-thiophen-2-yl-phenyl; benzo[d]isoxazol-3-yl-methyl; benzothiazol-5-yl; 5-phenyl-thiophen-2-yl; 3-methyl-thiophen-2-yl-methyl; 2-E-(3-methyl-thiophen-2-yl)-ethenyl; 2-(3-methyl-thiophen-2-yl)-ethyl; 3-phenyl-isoxazol-5-yl; 5-methyl-isoxazol-3-yl; 5-methyl-2-phenyl-2H-[1,2,3]-triazol-4-yl; 5-tert-butyl-2-methyl-2H-pyrazol-3-yl; 3-pyridin-2-yl-isoxazol-5-yl; 3-ethyl-isoxazol-5-yl; 3-propyl-isoxazol-5-yl; 3-isopropyl-isoxazol-5-yl; 3-isobutyl-isoxazol-5-yl; 3-butyl-isoxazol-5-yl; 3-tert-butyl-isoxazol-5-yl; 3-(1-methylpropyl)-isoxazol-5-yl; indol-1-yl-methyl; 2-E-(5-methyl-thiophen-2-yl)-ethenyl; 2-(5-methyl-thiophen-2-yl)-ethyl; methyl-sulfonyl-N-phenyl-amino-methyl; phenyl-sulfonyl-N-phenyl-amino-methyl; 5-methyl-thiophen-2-yl; 4-methyl-thiophen-2-yl; 3-methyl-thiophen-2-yl; 5-methyl-thiophen-2-yl-methyl; 4-methyl-thiophen-2-yl-methyl; 2-E-(4-methyl-thiophen-2-yl)-ethenyl; 2-(4-methyl-thiophen-2-yl)-ethyl; 5-phenyl-isoxazol-3-yl; 3-phenyl-isoxazol-5-yl-methyl; 3-isobutyl-isoxazol-5-yl-methyl; (5-phenylimidazol-1-yl)methyl; (benzimidazol-1-yl)methyl; (2-phenylimidazol-1-yl)methyl; biphenyl-2-yl-oxy-methyl; biphenyl-3-yl-oxy-methyl; biphenyl-4-yl-oxy-methyl; 3-methyl-isoxazol-5-yl-methyl; benzofuran-2-yl; 1H-indol-3-yl-methyl; 1H-indol-2-yl; 5-ethyl-8-oxo-5,8-dihydro-[1,3]dioxolo-[4,5-g]quinolin-7-yl; 7-chloro-1-cyclopropyl-6-fluoro-4-oxo-1,4-dihydro-quinolin-3-yl; 8-Fluoro-3-methyl-9-(4-methyl-piperazin-1-yl)-2,3-dihydro-1-oxa-3a-aza-phenalen-6-one-5-yl; 1-Ethyl-7-methyl-4-oxo-1,4-dihydro-[1,8]naphthyridin-3-yl; quinolin-4-yl; quinolin-8-yl; quinolin-6-yl; 2,2-difluoro-benzo[1,3]dioxol-5-yl; 2,2-Difluoro-benzo[1,3]dioxol-4-yl; quinolin-2-yl; quinolin-5-yl; quinolin-3-yl; (1-oxo-1,3-dihydroisoindol-2-yl)methyl; (2-oxo-2,3-dihydroindol-1-yl)methyl; (2-oxo-benzoxazol-3-yl)methyl; (benzotriazol-1-yl)methyl; (indazol-1-yl)methyl; 2,2-difluoro-benzo[1,3]dioxol-4-yl-methyl; 1-methyl-1H-indol-3-yl-methyl; 5-phenyl-isoxazol-3-yl-methyl; 3-isopropyl-isoxazol-5-yl-methyl; benzo[1,3]dioxol-4-yl; 2,2-difluoro-benzo[1,3]dioxol-5-yl-methyl; (3-methyl-2-oxo-2,3-dihydrobenzimidazol-1-yl)methyl; (2-oxo-2,3-dihydrobenzimidazol-1-yl)methyl; (3-ethyl-2-oxo-2,3-dihydro-benzimidazol-1-yl)methyl; (4-methyl-2-oxo-benzooxazol-3-yl)methyl; (5-methyl-2-oxo-benzooxazol-3-yl)methyl; (6-methyl-2-oxo-benzooxazol-3-yl)methyl; 4-(4-methoxy-phenyl)-thiophen-2-yl; 2-phenyl-thiazol-4-yl-methyl; 2-phenyl-thiazol-4-yl; 2-phenyloxazol-4-yl-methyl; 1-methyl-1H-indol-2-yl; 2-phenyl-oxazol-4-yl; 2-methyl-thiazol-4-yl-methyl; 2-methyl-oxazol-4-yl-methyl; (5-methyl-2-phenyl-2H-[1,2,3]triazol-4-yl)methyl; (5-phenyltetrazol-1-yl)methyl; (4R,5S)-(+)-4-methyl-5-phenyl-oxazolidin-2-one-3-yl-methyl; (4S,5R)-(−)-4-methyl-5-phenyl-oxazolidin-2-one-3-yl-methyl; pyrrolidin-2-one-1-yl-methyl; 2-cyclohexyl-oxazol-4-yl-methyl; (4R)-4-phenyl-oxazolidin-2-one-3-yl-methyl; (4S)-4-phenyl-oxazolidin-2-one-3-yl-methyl; (2-cyclohexylthiazol-4-yl)methyl; 5-(4-methyl-phenyl)-tetrazol-1-yl-methyl; 5-(4-methoxy-phenyl)-tetrazol-1-yl-methyl; 2-ethenyl-benzyl; 4-difluoromethoxy-phenyl; 4-trifluoromethoxy-phenyl; 2-ethynyl-benzyl; 1-aceto-piperidin-4-yl; 1-(4-chloro-benzyl)-pyrrolidin-2-one-4-yl; bicyclo[4.2.0]octa-1(6),2,4-trien-7-yl; 5-methyl-1-phenyl-1H-pyrazol-4-yl; 1-methyl-5-phenyl-1H-pyrazol-3-yl-methyl; (2-methyl-5-phenyl-2H-pyrazol-3-yl)methyl; 1-ethyl-5-phenyl-1H-pyrazol-3-yl-methyl; (2-ethyl-5-phenyl-2H-pyrazol-3-yl)methyl; (2,5-diphenyl-2H-pyrazol-3-yl)methyl; (2-tert-butyl-5-phenyl-2H-pyrazol-3-yl)methyl; (2-cyclohexyl-5-phenyl-2H-pyrazol-3-yl)methyl; (5-methyl-2-phenyl-2H-pyrazol-3-yl)methyl; 2-methyl-5-phenyl-2H-pyrazol-3-yl; 1-methyl-5-phenyl-1H-pyrazol-3-yl; (5-phenyl-1-propyl-1H-pyrazol-3-yl)methyl; 1-butyl-5-phenyl-1H-pyrazol-3-yl-methyl; 1-isobutyl-5-phenyl-1H-pyrazol-3-yl-methyl; (5-phenyl-pyrazol-1-yl)methyl; (3-methyl-5-phenyl-pyrazol-1-yl)methyl; (5-methyl-3-phenylpyrazol-1-yl)methyl; (3-phenylpyrazol-1-yl)methyl; 2-phenyl-2H-pyrazol-3-yl; 2-(bis-methylsulfonylamino)-benzyl; L-phenyl-sulfonyl-amino-phenylmethyl; L-phenyl-sulfonyl-N-methyl-amino-phenylmethyl; phenyl-sulfonyl-amino-methyl; phenyl-sulfonyl-N-methyl-amino-methyl; phenyl-sulfonyl-N-ethyl-amino-methyl; phenyl-sulfonyl-N-isopropyl-amino-methyl; phenyl-sulfonyl-N-propyl-amino-methyl; phenyl-sulfonyl-N-benzyl-amino-methyl; benzyl-sulfonyl-amino-methyl; benzyl-sulfonyl-N-methyl-amino-methyl; benzyl-sulfonyl-N-propyl-amino-methyl; benzyl-sulfonyl-N-benzyl-amino-methyl; benzyl-sulfonyl-N-ethyl-amino-methyl; benzyl-sulfonyl-N-isopropyl-amino-methyl; (4-phenyl-[1,2,3]triazol-1-yl)methyl; (5-phenyl-[1,2,3]triazol-1-yl)methyl; (5-phenyltetrazol-2-yl)methyl; 5-phenyl-oxazol-4-yl; 5-phenyl-oxazol-4-yl-methyl; N-(n-butyl-carbonyl)amino-methyl; N-(n-butyl-carbonyl)amino-benzylmethyl; N-(1-ethyl-n-pentyl-carbonyl)amino-methyl; N-(2-methyl-benzyl-carbonyl)amino-benzylmethyl; 1-N-n-butyl-carbonyl)amino-ethyl; 1-N-(2-methyl-benzylcarbonyl)amino-ethyl; N-(2-methyl-benzyl-carbonyl)amino-methyl; 1-N−1-ethyl-n-pentyl-carbonyl)amino-ethyl; N-(1-ethyl-n-pentyl-carbonyl)amino-benzylmethyl; 1-N-(2-methyl-benzyl-carbonyl)amino-2-methyl-butyl; cyclopentyl; cyclopentyl-methyl; 2-cyclopentyl-ethyl; 1-phenyl-cyclopentyl; bicyclo[2.2.1]heptylmethyl; cyclohexylmethyl; 4-methyl-cyclohexyl-methyl; 2-methyl-cyclohexyl-methyl; 4-pentyl-cyclohexyl-methyl; cycloheptyl; cyclopropyl; 2-methylcyclopropyl; 1-methylcyclopropyl; 2,2,3,3-tetramethyl-cyclopropyl; 2-(2-methyl-prop-1-enyl)-3,3-dimethyl-cyclopropyl; 2-phenyl-cyclopropyl; 1-phenyl-cyclopropyl; cyclobutyl; and cyclohexen-3-yl.

In one embodiment, R^{x} is selected from the group consisting of: benzo[d]isoxazol-3-yl-methyl, 3-methyl-thiophen-2-yl-methyl, 1-methyl-5-phenyl-1H-pyrazol-3-yl-methyl, (2-methyl-S-phenyl-2H-pyrazol-3-yl)methyl, (indazol-1-yl)methyl, (2-oxo-benzoxazol-3-yl)methyl, and (5-phenyltetrazol-1-yl)methyl.

In one embodiment, R^{x} is —H═CH—CH═CH—CH₂—CH(CH₃)₂, —(CH₂)₅CH(CH₃)₂, or 2-methyl-benzyl.

In one embodiment, R^{x} is —CH═CH—CH═CH—CH₂—CH(CH₃)₂.

In one embodiment, R^{x} is phenyl, a 5-membered heteroaryl ring, a 6-membered heteroaryl ring, a 5-membered heterocyclic ring, or a 6-membered heterocyclic ring, wherein the phenyl, 5-membered heteroaryl ring, 6-membered heteroaryl ring, 5-membered heterocyclic ring, or 6-membered heterocyclic ring has a single substituent at the ortho position.

In one embodiment, R^{x} is —CH₂—R²³, wherein R²³ is phenyl, a 6-membered heterocyclic ring, or a 6-membered heteroaryl ring, wherein the phenyl, 6-membered heterocyclic ring, or 6-membered heteroaryl ring has a single substituent at the ortho or meta position.

In one embodiment, R^{x} is —CH₂—R²⁴, wherein R²⁴ is a 5-membered heteroaryl or 5-membered heterocyclic ring, wherein the 5-membered heteroaryl or heterocyclic ring has a single substituent at the ortho position.

In one embodiment, R^{x} is not N-benzyl-aminomethyl, N-benzyl-N-(2,4-dinitrophenyl)-aminomethyl, N-benzyl-N-(2,4-diaminophenyl)-aminomethyl, 5-(5-isopropyl-[1,2,3]trioxolan-4-yl)-[1,2,3]trioxolan-4-yl, 5-(5-isobutyl-[1,2,4]trioxolan-3-yl)-[1,2,4]trioxolan-3-yl, N-benzylamino-hydroxymethyl, or N-benzyliminomethyl.

In one embodiment, W is —NH—C(S)—NH—R^{z}.

In one embodiment, R^{z} is selected from the group consisting of: 2-methyl-phenyl; 3-methyl-phenyl; 4-methyl-phenyl; 2-fluoro-phenyl; 3-fluoro-phenyl; 4-fluoro-phenyl; 2,6-difluoro-phenyl; benzyl; 2-phenyl-ethyl; napth-1-yl; cyclohexyl; 4′-propyl-4-cyclohexyl-phenyl; and phenyl. In another embodiment, R^{z} is selected from the group consisting of: 2-fluoro-phenyl; 3-fluoro-phenyl; and 4-fluoro-phenyl.

In one embodiment, W is —NH—C(O)—NH—R^{z}.

In one embodiment, R^{z} is selected from the group consisting of: n-butyl; n-octyl; cyclohexyl; benzyl; phenyl; 2-trifluoromethyl-phenyl; 3-trifluoromethyl-phenyl; 4-trifluoromethyl-phenyl; 2-methoxy-phenyl; 2,6-dimethyl-phenyl; napth-1-yl; 1-napth-1-yl-ethyl; and 2-methyl-phenyl. In another embodiment, R^{z} is selected from the group consisting of: benzyl; phenyl; and 2-methyl-phenyl. In another embodiment, R^{z} is selected from the group consisting of: n-butyl; n-octyl; cyclohexyl; benzyl; phenyl; 2-trifluoromethyl-phenyl; 3-trifluoromethyl-phenyl; 4-trifluoromethyl-phenyl; 2-methoxy-phenyl; 2,6-dimethyl-phenyl; napth-1-yl; and 1-napth-1-yl-ethyl.

In one embodiment, W is —NH—C(O)O—R^{z}.

In one embodiment, R^{z} is selected from the group consisting of: propyl; butyl; hexyl; octyl; decyl; isopropyl; isobutyl; 2,2-dimethyl-propyl; 2-ethyl-hexyl; (1S,2R,5S)-2-isopropyl-5-methylcyclohex-1-yl; (1R,2S,5R)-2-isopropyl-5-methyl-cyclohex-1-yl; ethenyl; prop-2-enyl; but-3-enyl; 1-methyl-ethenyl; but-3-ynyl; but-2-ynyl; 4-fluorophenyl; 4-bromophenyl; 4-nitrophenyl; 4-methoxycarbonyl-phenyl; 2-chloro-phenyl; 4-chloro-phenyl; 2-methoxy-phenyl; 4-methoxy-phenyl; 4-methyl-phenyl; 2-nitro-phenyl; 3-trifluoromethyl-phenyl; 2-nitro-3,4-dimethoxy-phenyl; benzyl; 2-chloro-phenylmethyl; (2-trifluoromethyl-phenyl)-chloro-methyl; and (4-nitro-phenyl)-methyl. In another embodiment, R^{z} is selected from the group consisting of: hexyl; 4-methyl-phenyl; and 4-nitrophenyl.

In one embodiment, W is —NH—R′.

In one embodiment, R′ is selected from the group consisting of: 3,6-difluoro-benzyl; 3,6-dimethyl-benzyl; 2,3-dihydro-benzo[1,4]dioxin-6-yl-methyl; 2-phenyl-ethyl; cyclohexyl-methyl; n-nonyl; n-heptyl; 2-phenyl-propyl; 4-bromo-benzyl; napth-2-yl-methyl; and 4-phenoxy-benzyl. In another embodiment, R′ is selected from the group consisting of: 4-bromo-benzyl and napth-2-yl-methyl.

In one embodiment, R′ is not benzyl. In another embodiment, when R^{y} is H, then R′ is not benzyl.

In one embodiment, W is —NH—S(O₂)—R″.

In one embodiment, R″ is selected from the group consisting of: 4-fluoro-phenyl, napth-2-yl, and phenyl. In another embodiment, R″ is napth-2-yl.

In one embodiment, W is —N(CH₃)S(O₂)—R″.

In one embodiment, R″ is phenyl-sulfonyl-N-methyl-amino.

In one embodiment, W is —NH—C(O)—CH═N—NH—R²⁰.

In one embodiment, R²⁰ is selected from the group consisting of: phenylaminothiocarbonyl; N-ethylaminothiocarbonyl; N-prop-2-enylamino-thiocarbonyl; phenylaminocarbonyl; phenylcarbonyl; 3-methoxy-phenylcarbonyl; pyridine-4-yl-carbonyl; thiophen-2-ylcarbonyl; and benzylcarbonyl. In another embodiment, R²⁰ is selected from the group consisting of: phenylaminothiocarbonyl and benzylcarbonyl.

In one embodiment, W is substituted aryl. In one embodiment, W is 2-methyl-phenyl.

In another one of its composition aspects, this invention is direction to a compound of Formula (I), with the proviso: when R^{y} is —CH₂CONH₂, R² is —NH₂, R³ and R⁴ are —NH₂ or —NH(protecting group), R⁵ is H, α-D-mannopyranosyl, or 2-O-α-D-mannopyranosyl-α-D-mannopyranosyl, and W is substituted carbonyl, then W is not —CO-alkyl, —CO-alkenyl, —CO—R²¹, —CO-(C1-C4 alkylene)-R²¹, or —CO-(C2-C4 alkenylene)-R²¹ wherein R²¹ is alkoxy; substituted alkoxy; alkenyloxy; substituted alkenyloxy; phenyl; substituted phenyl; napthyl, substituted napthyl; phenoxy; substituted phenoxy; napthoxy; or substituted napthoxy.

In one embodiment, the compound is selected from the group consisting of compounds 1-297 as shown in Tables I-VIII, and prodrugs, tautomers and pharmaceutically acceptable salts thereof.

In another embodiment, the compound is selected from the group consisting of: compounds 11, 14, 29, 37, 38, 42, 44, 68, 70, 77, 88, 91, 92, 105, 108, 110, 111, 112, 113, 118, 119, 123, 124, 126, 144, and 147 and prodrugs, tautomers and pharmaceutically acceptable salts thereof. In another embodiment, the compound is selected from the group consisting of: compounds 92, 123 and 147, and prodrugs, tautomers and pharmaceutically acceptable salts thereof. In a preferred embodiment, the compound is compound 92. In another preferred embodiment, the compound is compound 123. In another preferred embodiment, the compound is compound 147. In another preferred embodiment, the compound is compound 271.

In another aspect, this invention is directed to a pharmaceutical composition comprising a pharmaceutically acceptable carrier and a therapeutically effective amount of a compound of the invention.

In another aspect, this invention is directed to a method for the treatment of a microbial infection in a mammal comprising administering to the mammal a therapeutically effective amount of a compound of the invention. In one embodiment, the compound is administered to the mammal orally, parenterally, transdermally, topically, rectally, or intranasally in a pharmaceutical composition. In another embodiment, the compound is administered in an amount of from about 0.1 to about 100 mg/kg of body weight/day.

Ramoplanin derivatives within the scope of this invention include those set forth in Tables I-VIII as follows:

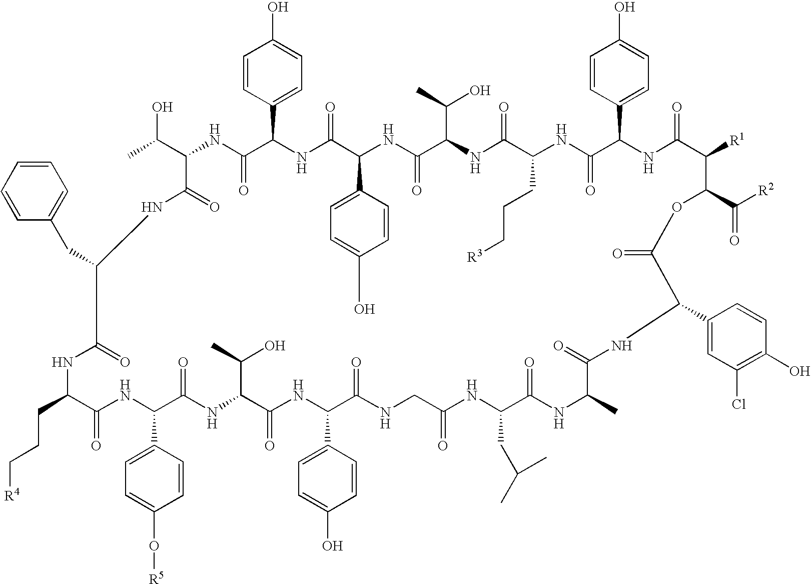

TABLE I

| | |
|---|---|
| | Formula (IA) |
| 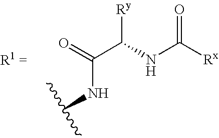 | |

| Ex | | | | | | |
|---|---|---|---|---|---|---|
| No. | R^{x} | R^{y} | R² | R³ | R⁴ | R⁵ |
| 1 | Thiophen-2-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 2 | 3-methyl-benzo[b]thiophen-2-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 3 | benz[b]thiophen-3-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 4 | 5-chloro-benzo[b]thiophen-3-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 5 | Thiophen-3-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 6 | Benzo[1,3]dioxol-5-yl-methyl | —CH₂CONH₂ (L-Asn) | —NH₂ | —NH₂ | —NH₂ | 2-O-α-D-mannopyranasyl-α-D- |
| | | | | | | mannopyranosyl |
| 7 | (±)-2,3-dihydro-benzo[1,4]dioxin-2-yl | —CH₂CONH₂ (L-Asn) | —NH₂ | —NH₂ | —NH₂ | 2-O-α-D-mannopyranosyl-α-D- |
| | | | | | | mannopyranosyl |
| 8 | 2-benzyloxy-benzyl | —CH₂CONH₂ (L-Asn) | —NH₂ | —NH₂ | —NH₂ | 2-O-α-D-mannopyranosyl-α-D- |
| | | | | | | mannopyranosyl |
| 9 | 2-phenylsulfanyl-benzyl | —CH₂CONH₂ (L-Asn) | —NH₂ | —NH₂ | —NH₂ | 2-O-α-D-mannopyranosyl-α-D- |
| | | | | | | mannopyranosyl |
| 10 | 4-thiophen-2-yl-phenyl | —CH₂CONH₂ (L-Asn) | —NH₂ | —NH₂ | —NH₂ | 2-O-α-D-mannopyranosyl-α-D- |
| | | | | | | mannopyranosyl |
| 11 | Benzo[d]isoxazol-3-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 12 | Benzothiazol-5-yl | —CH₂CONH₂ (L-Asn) | —NH₂ | —NH₂ | —NH₂ | 2-O-α-D-mannopyranosyl-α-D- |
| | | | | | | mannopyranosyl |
| 13 | 5-phenyl-thiophen-2-yl | —CH₂CONH₂ (L-Asn) | —NH₂ | —NH₂ | —NH₂ | 2-O-α-D-mannopyranosyl-α-D- |
| | | | | | | mannopyranosyl |
| 14 | 3-methyl-thiophen-2-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 15 | 2-E-(3-methyl-thiophen-2-yl)-ethenyl | —CH₂CONH₂ (L-Asn) | —NH₂ | —NH₂ | —NH₂ | 2-O-α-D-mannopyranosyl-α-D- |
| | | | | | | mannopyranosyl |
| 16 | 2-(3-methyl-thiophen-2-yl)-ethyl | —CH₂CONH₂ (L-Asn) | —NH₂ | —NH₂ | —NH₂ | 2-O-α-D-mannopyranosyl-α-D- |
| | | | | | | mannopyranosyl |
| 17 | 3-phenyl-isoxazol-5-yl | —CH₂CONH₂ (L-Asn) | —NH₂ | —NH₂ | —NH₂ | 2-O-α-D-mannopyranosyl-α-D- |
| | | | | | | mannopyranosyl |
| 18 | 5-methyl-isoxazol-3-yl | —CH₂CONH₂ (L-Asn) | —NH₂ | —NH₂ | —NH₂ | 2-O-α-D-mannopyranosyl-α-D- |
| | | | | | | mannopyranosyl |
| 19 | 5-methyl-2-phenyl-2H-[1,2,3]-triazol-4-yl | —CH₂CONH₂ (L-Asn) | —NH₂ | —NH₂ | —NH₂ | 2-O-α-D-mannopyranosyl-α-D- |
| | | | | | | mannopyranosyl |
| 20 | 5-tert-butyl-2-methyl-2H-pyrazol-3-yl | —CH₂CONH₂ (L-Asn) | —NH₂ | —NH₂ | —NH₂ | 2-O-α-D-mannopyranosyl-α-D- |
| | | | | | | mannopyranosyl |
| 21 | 3-pyridin-2-yl-isoxazol-5-yl | —CH₂CONH₂ (L-Asn) | —NH₂ | —NH₂ | —NH₂ | 2-O-α-D-mannopyranosyl-α-D- |
| | | | | | | mannopyranosyl |
| 22 | 3-ethyl-isoxazol-5-yl | —CH₂CONH₂ (L-Asn) | —NH₂ | —NH₂ | —NH₂ | 2-O-α-D-mannopyranosyl-α-D- |
| | | | | | | mannopyranosyl |
| 23 | 3 -propyl-isoxazol-5-yl | —CH₂CONH₂ (L-Asn) | —NH₂ | —NH₂ | —NH₂ | 2-O-α-D-rnannopyranosyl-α-D- |
| | | | | | | mannopyranosyl |
| 24 | 3-isopropyl-isoxazol-5-yl | —CH₂CONH₂ (L-Asn) | —NH₂ | —NH₂ | —NH₂ | 2-O-α-D-mannopyranosyl-α-D- |
| | | | | | | mannopyranosyl |
| 25 | 3-isobutyl-isoxazol-5-yl | —CH₂CONH₂ (L-Asn) | —NH₂ | —NH₂ | —NH₂ | 2-O-α-D-mannopyranosyl-α-D- |
| | | | | | | mannopyranosyl |
| 26 | 3-butyl-isoxazol-5-yl | —CH₂CONH₂ (L-Asn) | —NH₂ | —NH₂ | —NH₂ | 2-O-α-D-mannopyranosyl-α-D- |
| | | | | | | mannopyranosyl |
| 27 | 3-tert-butyl-isoxazol-5-yl | —CH₂CONH₂ (L-Asn) | —NH₂ | —NH₂ | —NH₂ | 2-O-α-D-mannopyranosyl-α-D- |
| | | | | | | mannopyranosyl |
| 28 | 3-(1-methylpropyl)-isoxazol-5-yl | —CH₂CONH₂ (L-Asn) | —NH₂ | —NH₂ | —NH₂ | 2-O-α-D-mannopyranosyl-α-D- |
| | | | | | | mannopyranosyl |
| 29 | indol-1-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 30 | 2-E-(5-methyl-thiophen-2-yl)-ethenyl | —CH₂CONH₂ (L-Asn) | —NH₂ | —NH₂ | —NH₂ | 2-O-α-D-mannopyranosyl-α-D- |
| | | | | | | mannopyranosyl |
| 31 | 2-(5-methyl-thiophen-2-yl)-ethyl | —CH₂CONH₂ (L-Asn) | —NH₂ | —NH₂ | —NH₂ | 2-O-α-D-mannopyranosyl-α-D- |
| | | | | | | mannopyranosyl |
| 32 | Methyl-sulfonyl-N-phenyl-amino-methyl | —CH₂CONH₂ (L-Asn) | —NH₂ | —NH₂ | —NH₂ | 2-O-α-D-mannopyranosyl-α-D- |
| | | | | | | mannopyranosyl |
| 33 | phenyl-sulfonyl-N-phenyl-amino-methyl | —CH₂CONH₂ (L-Asn) | —NH₂ | —NH₂ | —NH₂ | 2-O-α-D-mannopyranosyl-α-D- |
| | | | | | | mannopyranosyl |
| 34 | 5-methyl-thiophen-2-yl | —CH₂CONH₂ (L-Asn) | —NH₂ | —NH₂ | —NH₂ | 2-O-α-D-mannopyranosyl-α-D- |
| | | | | | | mannopyranosyl |
| 35 | 4-methyl-thiophen-2-yl | —CH₂CONH₂ (L-Asn) | —NH₂ | —NH₂ | —NH₂ | 2-O-α-D-mannopyranosyl-α-D- |
| | | | | | | mannopyranosyl |
| 36 | 3-methyl-thiophen-2-yl | —CH₂CONH₂ (L-Asn) | —NH₂ | —NH₂ | —NH₂ | 2-O-α-D-mannopyranosyl-α-D- |
| | | | | | | mannopyranosyl |
| 37 | 5-methyl-thiophen-2-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 38 | 4-methyl-thiophen-2-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 39 | 2-E-(4-methyl-thiophen-2-yl)-ethenyl | —CH₂CONH₂ (L-Asn) | —NH₂ | —NH₂ | —NH₂ | 2-O-α-D-mannopyranosyl-α-D- |
| | | | | | | mannopyranosyl |
| 40 | 2-(4-methyl-thiophen-2-yl)-ethyl | —CH₂CONH₂ (L-Asn) | —NH₂ | —NH₂ | —NH₂ | 2-O-α-D-mannopyranosyl-α-D- |
| | | | | | | mannopyranosyl |
| 41 | 5-phenyl-isoxazol-3-yl | —CH₂CONH₂ (L-Asn) | —NH₂ | —NH₂ | —NH₂ | 2-O-α-D-mannopyranosyl-α-D- |
| | | | | | | mannopyranosyl |
| 42 | 3-phenyl-isoxazol-5-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 43 | 3-isobutyl-isoxazol-5-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 44 | (5-phenylimidazol-1-yl)methyl | —CH₂CONH₂ (L-Asn) | —NH₂ | —NH₂ | —NH₂ | 2-O-α-D-marmopyranosyl-α-D- |
| | | | | | | mannopyranosyl |
| 45 | (benzimidazol-1-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 46 | (2-phenylimidazol-1-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 47 | Biphenyl-2-yl-oxy-methyl | —CH₂CONH₂ (L-Asn) | —NH₂ | —NH₂ | —NH₂ | 2-O-α-D-mannopyranosyl-α-D- |
| | | | | | | mannopyranosyl |
| 48 | Biphenyl-3-yl-oxy-methyl | —CH₂CONH₂ (L-Asn) | —NH₂ | —NH₂ | —NH₂ | 2-O-α-D-mannopyranosyl-α-D- |
| | | | | | | mannopyranosyl |
| 49 | Biphenyl-4-yl-oxy-methyl | —CH₂CONH₂ (L-Asn) | —NH₂ | —NH₂ | —NH₂ | 2-O-α-D-mannopyranosyl-α-D- |
| | | | | | | mannopyranOsyl |
| 50 | 3-methyl-isoxazol-5-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 51 | Benzofuran-2-yl | —CH₂CONH₂ (L-Asn) | —NH₂ | —NH₂ | —NH₂ | 2-O-α-D-mannopyranosyl-α-D- |
| | | | | | | mannopyranosyl |
| 52 | 1H-indol-3-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 53 | 1H-indol-2-yl | —CH₂CONH₂ (L-Asn) | —NH₂ | —NH₂ | —NH₂ | 2-O-α-D-mannopyranosyl-α-D- |
| | | | | | | mannopyranosyl |
| 54 | 5-ethyl-8-oxo-5,8-dihydro-[1,3]dioxolo- | —CH₂CONH₂ (L-Asn) | —NH₂ | —NH₂ | —NH₂ | 2-O-α-D-mannopyranosyl-α-D- |
| | [4,5-g]quinolin-7-yl | | | | | mannopyranosyl |
| 55 | 7-chloro-1-cyclopropyl-6-fluoro-4-oxo- | —CH₂CONH₂ (L-Asn) | —NH₂ | —NH₂ | —NH₂ | 2-O-α-D-mannopyranosyl-α-D- |
| | 1,4-dihydro-quinolin-3-yl | | | | | mannopyranosyl |
| 56 | 8-Fluoro-3-methyl-9-(4-methyl-piperazin- | —CH₂CONH₂ (L-Asn) | —NH₂ | —NH₂ | —NH₂ | 2-O-α-D-mannopyranosyl-α-D- |
| | 1-yl)-2,3-dihydro-1-oxa-3a-aza-phenalen- | | | | | mannopyranosyl |
| | 6-one-5-yl | | | | | |
| 57 | 1-Ethyl-7-methyl-4-oxo-1,4-dihydro- | —CH₂CONH₂ (L-Asn) | —NH₂ | —NH₂ | —NH₂ | 2-O-α-D-mannopyranosyl-α-D- |
| | [1,8]naphthyridin-3-yl | | | | | mannopyranosyl |
| 58 | Quinolin-4-yl | —CH₂CONH₂ (L-Asn) | —NH₂ | —NH₂ | —NH₂ | 2-O-α-D-mannopyranosyl-α-D- |
| | | | | | | mannopyranosyl |
| 59 | Quinolin-8-yl | —CH₂CONH₂ (L-Asn) | —NH₂ | —NH₂ | —NH₂ | 2-O-α-D-mannopyranosyl-α-D- |
| | | | | | | mannopyranosyl |
| 60 | Quinolin-6-yl | —CH₂CONH₂ (L-Asn) | —NH₂ | —NH₂ | —NH₂ | 2-O-α-D-mannopyranosyl-α-D- |
| | | | | | | mannopyranosyl |
| 61 | 2,2-difluoro-benzo[1,3]dioxol-5-yl | —CH₂CONH₂ (L-Asn) | —NH₂ | —NH₂ | —NH₂ | 2-O-α-D-mannopyranosyl-α-D- |
| | | | | | | mannopyranosyl |
| 62 | 2,2-Difluoro-benzo[1,3]dioxol-4-yl | —CH₂CONH₂ (L-Asn) | —NH₂ | —NH₂ | —NH₂ | 2-O-α-D-mannopyranosyl-α-D- |
| | | | | | | mannopyranosyl |
| 63 | Quinolin-2-yl | —CH₂CONH₂ (L-Asn) | —NH₂ | —NH₂ | —NH₂ | 2-O-α-D-mannopyranosyl-α-D- |
| | | | | | | mannopyranosyl |
| 64 | Quinolin-5-yl | —CH₂CONH₂ (L-Asn) | —NH₂ | —NH₂ | —NH₂ | 2-O-α-D-mannopyranosyl-α-D- |
| | | | | | | mannopyranosyl |
| 65 | quinolin-3-yl | —CH₂CONH₂ (L-Asn) | —NH₂ | —NH₂ | —NH₂ | 2-O-α-D-mannopyranosyl-α-D- |
| | | | | | | mamiopyranosyl |
| 66 | (1-oxo-1,3-dihydroisoindol-2-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 67 | (2-oxo-2,3-dihydroindol-1-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 68 | (2-oxo-benzoxazol-3-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 69 | (benzotriazol-1-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 70 | (indazol-1-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 71 | 2,2-difluoro-benzo[1,3]dioxol-4-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 72 | 1-methyl-1H-indol-3-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 73 | 5-phenyl-isoxazol-3-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 74 | 3-isopropyl-isoxazol-5-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 75 | Benzo[1,3]dioxol-4-yl | —CH₂CONH₂ (L-Asn) | —NH₂ | —NH₂ | —NH₂ | 2-O-α-D-mannopyranosyl-α-D- |
| | | | | | | mannopyranosyl |
| 76 | 2,2-difluoro-benzo[1,3]dioxol-5-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 77 | (3-methyl-2-oxo-2,3-dihydrobenzimidazol- | —CH₂CONH₂ (L-Asn) | —NH₂ | —NH₂ | —NH₂ | 2-O-α-D-mannopyranosyl-α-D- |
| | 1-yl)methyl | | | | | mannopyranosyl |
| 78 | (2-oxo-2,3-dihydrobenzimidazol-1- | —CH₂CONH₂ (L-Asn) | —NH₂ | —NH₂ | —NH₂ | 2-O-α-D-mannopyranosyl-α-D- |
| | yl)methyl | | | | | mannopyranosyl |
| 79 | (3-ethyl-2-oxo-2,3-dihydro-benzimidazol- | —CH₂CONH₂ (L-Asn) | —NH₂ | —NH₂ | —NH₂ | 2-O-α-D-mannopyranosyl-α-D- |
| | 1-yl)methyl | | | | | mannopyranosyl |
| 80 | (4-methyl-2-oxo-benzooxazol-3-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 81 | (5-methyl-2-oxo-benzooxazol-3-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 82 | (6-methyl-2-oxo-benzooxazol-3-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 83 | 4-(4-methoxy-phenyl)-thiophen-2-yl | —CH₂CONH₂ (L-Asn) | —NH₂ | —NH₂ | —NH₂ | 2-O-α-D-mannopyranosyl-α-D- |
| | | | | | | mannopyranosyl |
| 84 | 2-phenyl-thiazol-4-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 85 | 2-phenyl-thiazol-4-yl | —CH₂CONH₂ (L-Asn) | —NH₂ | —NH₂ | —NH₂ | 2-O-α-D-mannopyranosyl-α-D- |
| | | | | | | mannopyranosyl |
| 86 | 2-phenyl-oxazol-4-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 87 | 1-methyl-1H-indol-2-yl | —CH₂CONH₂ (L-Asn) | —NH₂ | —NH₂ | —NH₂ | 2-O-α-D-mannopyranosyl-α-D- |
| | | | | | | mannopyranosyl |
| 88 | 2-phenyl-oxazol-4-yl | —CH₂CONH₂ (L-Asn) | —NH₂ | —NH₂ | —NH₂ | 2-O-α-D-mannopyranosyl-α-D- |
| | | | | | | mannopyranosyl |
| 89 | 2-methyl-thiazol-4-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 90 | 2-methyl-oxazol-4-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 91 | (5-methyl-2-phenyl-2H-[1,2,3]triazol-4- | —CH₂CONH₂ (L-Asn) | —NH₂ | —NH₂ | —NH₂ | 2-O-α-D-mannopyranosyl-α-D- |
| | yl)methyl | | | | | mannopyranosyl |
| 92 | (5-phenyltetrazol-1-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 93 | (4R,5S)-(+)-4-methyl-5-phenyl-oxazolidin- | —CH₂CONH₂ (L-Asn) | —NH₂ | —NH₂ | —NH₂ | 2-O-α-D-mannopyranosyl-α-D- |
| | 2-one-3-yl-methyl | | | | | mannopyranosyl |
| 94 | (4S,5R)-(−)-4-methyl-5-phenyl-oxazolidin- | —CH₂CONH₂ (L-Asn) | —NH₂ | —NH₂ | —NH₂ | 2-O-α-D-mannopyranosyl-α-D- |
| | 2-one-3-yl-methyl | | | | | mannopyranosyl |
| 95 | Pyrrolidin-2-one-1-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 96 | 2-cyclohexyl-oxazol-4-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 97 | (4R)-4-phenyl-oxazolidin-2-one-3-yl- | —CH₂CONH₂ (L-Asn) | —NH₂ | —NH₂ | —NH₂ | 2-O-α-D-mannopyranosyl-α-D- |
| | methyl | | | | | mannopyranosyl |
| 98 | (4S)-4-phenyl-oxazolidin-2-one-3-yl- | —CH₂CONH₂ (L-Asn) | —NH₂ | —NH₂ | —NH₂ | 2-O-α-D-mannopyranosyl-α-D- |
| | methyl | | | | | mannopyranosyl |
| 99 | (2-cyclohexylthiazol-4-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 100 | 5-(4-methyl-phenyl)-tetrazol-1-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 101 | 5-(4-methoxy-phenyl)-tetrazol-1-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 102 | 2-ethenyl-benzyl | —CH₂CONH₂ (L-Asn) | —NH₂ | —NH₂ | —NH₂ | 2-O-α-D-mannopyranosyl-α-D- |
| | | | | | | mannopyranosyl |
| 103 | 4-difluoromethoxy-phenyl | —CH₂CONH₂ (L-Asn) | —NH₂ | —NH₂ | —NH₂ | 2-O-α-D-mannopyranosyl-α-D- |
| | | | | | | mannopyranosyl |
| 104 | 4-trifluoromethoxy-phenyl | —CH₂CONH₂ (L-Asn) | —NH₂ | —NH₂ | —NH₂ | 2-O-α-D-mannopyranosyl-α-D- |
| | | | | | | mannopyranosyl |
| 105 | 2-ethynyl-benzyl | —CH₂CONH₂ (L-Asn) | —NH₂ | —NH₂ | —NH₂ | 2-O-α-D-mannopyranosyl-α-D- |
| | | | | | | mannopyranosyl |
| 106 | 1-aceto-piperidin-4-yl | —CH₂CONH₂ (L-Asn) | —NH₂ | —NH₂ | —NH₂ | 2-O-α-D-mannopyranosyl-α-D- |
| | | | | | | mannopyranosyl |
| 107 | 1-(4-chloro-benzyl)-pyrrolidin-2-one-4-yl | —CH₂CONH₂ (L-Asn) | —NH₂ | —NH₂ | —NH₂ | 2-O-α-D-mannopyranosyl-α-D- |
| | | | | | | mannopyranosyl |
| 108 | bicyclo[4.2.0]octa-1(6),2,4-trien-7-yl | —CH₂CONH₂ (L-Asn) | —NH₂ | —NH₂ | —NH₂ | 2-O-α-D-mannopyranosyl-α-D- |
| | | | | | | mannopyranosyl |
| 109 | 5-methyl-1-phenyl-1H-pyrazol-4-yl | —CH₂CONH₂ (L-Asn) | —NH₂ | —NH₂ | —NH₂ | 2-O-α-D-mannopyranosyl-α-D- |
| | | | | | | mannopyranosyl |
| 110 | 1-methyl-5-phenyl-1H-pyrazol-3-yl- | —CH₂CONH₂ (L-Asn) | —NH₂ | —NH₂ | —NH₂ | 2-O-α-D-mannopyranosyl-α-D- |
| | methyl | | | | | mannopyranosyl |
| 111 | (2-methyl-5-phenyl-2H-pyrazol-3- | —CH₂CONH₂ (L-Asn) | —NH₂ | —NH₂ | —NH₂ | 2-O-α-D-mannopyranosyl-α-D- |
| | yl)methyl | | | | | mannopyranosyl |
| 112 | 1-ethyl-5-phenyl-1H-pyrazol-3-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 113 | (2-ethyl-5-phenyl-2H-pyrazol-3-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 114 | (2,5-diphenyl-2H-pyrazol-3-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 115 | (2-tert-butyl-5-phenyl-2H-pyrazol-3- | —CH₂CONH₂ (L-Asn) | —NH₂ | —NH₂ | —NH₂ | 2-O-α-D-mannopyranosyl-α-D- |
| | yl)methyl | | | | | mannopyranosyl |
| 116 | (2-cyclohexyl-5-phenyl-2H-pyrazol-3- | —CH₂CONH₂ (L-Asn) | —NH₂ | —NH₂ | —NH₂ | 2-O-α-D-mannopyranosyl-α-D- |
| | yl)methyl | | | | | mannopyranosyl |
| 117 | (5-methyl-2-phenyl-2H-pyrazol-3- | —CH₂CONH₂ (L-Asn) | —NH₂ | —NH₂ | —NH₂ | 2-O-α-D-mannopyranosyl-α-D- |
| | yl)methyl | | | | | mannopyranosyl |
| 118 | 2-methyl-5-phenyl-2H-pyrazol-3-yl | —CH₂CONH₂ (L-Asn) | —NH₂ | —NH₂ | —NH₂ | 2-O-α-D-mannopyranosyl-α-D- |
| | | | | | | mannopyranosyl |
| 119 | 1-methyl-5-phenyl-1H-pyrazol-3-yl | —CH₂CONH₂ (L-Asn) | —NH₂ | —NH₂ | —NH₂ | 2-O-α-D-mannopyranosyl-α-D- |
| | | | | | | mannopyranosyl |
| 120 | (5-phenyl-1-propyl-1H-pyrazol-3- | —CH₂CONH₂ (L-Asn) | —NH₂ | —NH₂ | —NH₂ | 2-O-α-D-mannopyranosyl-α-D- |
| | yl)methyl | | | | | mannopyranosyl |
| 121 | 1-butyl-5-phenyl-1H-pyrazol-3-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 122 | 1-isobutyl-5-phenyl-1H-pyrazol-3-yl- | —CH₂CONH₂ (L-Asn) | —NH₂ | —NH₂ | —NH₂ | 2-O-α-D-mannopyranosyl-α-D- |
| | methyl | | | | | mannopyranosyl |
| 123 | (5-phenyl-pyrazol-1-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 124 | (3-methyl-5-phenyl-pyrazol-1-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 125 | (5-methyl-3-phenylpyrazol-1-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 126 | (3-phenylpyrazol-1-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 127 | 2-phenyl-2H-pyrazol-3-yl | —CH₂CONH₂ (L-Asn) | —NH₂ | —NH₂ | —NH₂ | 2-O-α-D-mannopyranosyl-α-D- |
| | | | | | | mannopyranosyl |
| 128 | 2-(bis-methylsulfonylamino)-benzyl | —CH₂CONH₂ (L-Asn) | —NH₂ | —NH₂ | —NH₂ | 2-O-α-D-mannopyranosyl-α-D- |
| | | | | | | mannopyranosyl |
| 129 | L-Phenyl-sulfonyl-amino-phenylmethyl | —CH₂CONH₂ (L-Asn) | —NH₂ | —NH₂ | —NH₂ | 2-O-α-D-mannopyranosyl-α-D- |
| | | | | | | mannopyranosyl |
| 130 | L-Phenyl-sulfonyl-N-methyl-amino- | —CH₂CONH₂ (L-Asn) | —NH₂ | —NH₂ | —NH₂ | 2-O-α-D-mannopyranosyl-α-D- |
| | phenylmethyl | | | | | mannopyranosyl |
| 131 | Phenyl-sulfonyl-amino-methyl | —CH₂CONH₂ (L-Asn) | —NH₂ | —NH₂ | —NH₂ | 2-O-α-D-mannopyranosyl-α-D- |
| | | | | | | mannopyranosyl |
| 132 | Phenyl-sulfonyl-N-methyl-amino-methyl | —CH₂CONH₂ (L-Asn) | —NH₂ | —NH₂ | —NH₂ | 2-O-α-D-mannopyranosyl-α-D- |
| | | | | | | mannopyranosyl |
| 133 | Phenyl-sulfonyl-N-ethyl-amino-methyl | —CH₂CONH₂ (L-Asn) | —NH₂ | —NH₂ | —NH₂ | 2-O-α-D-mannopyranosyl-α-D- |
| | | | | | | mannopyranosyl |
| 134 | Phenyl-sulfonyl-N-isopropyl-amino- | —CH₂CONH₂ (L-Asn) | —NH₂ | —NH₂ | —NH₂ | 2-O-α-D-mannopyranosyl-α-D- |
| | methyl | | | | | mannopyranosyl |
| 135 | Phenyl-sulfonyl-N-propyl-amino-methyl | —CH₂CONH₂ (L-Asn) | —NH₂ | —NH₂ | —NH₂ | 2-O-α-D-mannopyranosyl-α-D- |
| | | | | | | mannopyranosyl |
| 136 | Phenyl-sulfonyl-N-benzyl-amino-methyl | —CH₂CONH₂ (L-Asn) | —NH₂ | —NH₂ | —NH₂ | 2-O-α-D-mannopyranosyl-α-D- |
| | | | | | | mannopyranosyl |
| 137 | Benzyl-sulfonyl-amino-methyl | —CH₂CONH₂ (L-Asn) | —NH₂ | —NH₂ | —NH₂ | 2-O-α-D-mannopyranosyl-α-D- |
| | | | | | | mannopyranosyl |
| 138 | benzyl-sulfonyl-N-methyl-amino-methyl | —CH₂CONH₂ (L-Asn) | —NH₂ | —NH₂ | —NH₂ | 2-O-α-D-mannopyranosyl-α-D- |
| | | | | | | mannopyranosyl |
| 139 | benzyl-sulfonyl-N-propyl-amino-methyl | —CH₂CONH₂ (L-Asn) | —NH₂ | —NH₂ | —NH₂ | 2-O-α-D-mannopyranosyl-α-D- |
| | | | | | | mannopyranosyl |
| 140 | benzyl-sulfonyl-N-benzyl-amino-methyl | —CH₂CONH₂ (L-Asn) | —NH₂ | —NH₂ | —NH₂ | 2-O-α-D-mannopyranosyl-α-D- |
| | | | | | | mannopyranosyl |
| 141 | benzyl-sulfonyl-N-ethyl-amino-methyl | —CH₂CONH₂ (L-Asn) | —NH₂ | —NH₂ | —NH₂ | 2-O-α-D-mannopyranosyl-α-D- |
| | | | | | | mannopyranosyl |
| 142 | benzyl-sulfonyl-N-isopropyl-amino-methyl | —CH₂CONH₂ (L-Asn) | —NH₂ | —NH₂ | —NH₂ | 2-O-α-D-mannopyranosyl-α-D- |
| | | | | | | mannopyranosyl |
| 143 | (4-phenyl-[1,2,3]triazol-1-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 144 | (5-phenyl-[1,2,3]triazol-1-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 145 | (5-phenyltetrazol-2-yl)methyl | —CH₂CONH₂ (L-Asn) | —NH₂ | —NH₂ | —NH₂ | 2-O-α-D-mannopyranosyl-α-D- |
| | | | | | | mannopyranosyl |
| 146 | 5-phenyl-oxazol-4-yl | —CH₂CONH₂ (L-Asn) | —NH₂ | —NH₂ | .—NH₂ | 2-O-α-D-mannopyranosyl-α-D- |
| | | | | | | mannopyranosyl |
| 147 | 5-phenyl-oxazol-4-yl-methyl | —CH₂CONH₂ (L-Asn) | —NH₂ | —NH₂ | —NH₂ | 2-O-α-D-mannopyranosyl-α-D- |
| | | | | | | mannopyranosyl |
| 148 | N-(n-butyl-carbonyl)amino-methyl | —CH₂CONH₂ (L-Asn) | —NH₂ | —NH₂ | —NH₂ | 2-O-α-D-mannopyranosyl-α-D- |
| | | | | | | mannopyranosyl |
| 149 | N-(n-butyl-carbonyl)amino-benzylmethyl | —CH₂CONH₂ (L-Asn) | —NH₂ | —NH₂ | —NH₂ | 2-O-α-D-mannopyranosyl-α-D- |
| | | | | | | mannopyranosyl |
| 150 | N-(1-ethyl-n-pentyl-carbonyl)amino- | —CH₂CONH₂ (L-Asn) | —NH₂ | —NH₂ | —NH₂ | 2-O-α-D-mannopyranosyl-α-D- |
| | methyl | | | | | mannopyranosyl |
| 151 | N-(2-methyl-benzyl-carbonyl)amino- | —CH₂CONH₂ (L-Asn) | —NH₂ | —NH₂ | —NH₂ | 2-O-α-D-mannopyranosyl-α-D- |
| | benzylmethyl | | | | | mannopyranosyl |
| 152 | 1-N-(n-butyl-carbonyl)amino-ethyl | —CH₂CONH₂ (L-Asn) | —NH₂ | —NH₂ | —NH₂ | 2-O-α-D-mannopyranosyl-α-D- |
| | | | | | | mannopyranosyl |
| 153 | 1-N-(2-methyl-benzyl-carbonyl)amino- | —CH₂CONH₂ (L-Asn) | —NH₂ | —NH₂ | —NH₂ | 2-O-α-D-mannopyranosyl-α-D- |
| | ethyl | | | | | mannopyranosyl |
| 154 | N-(2-methyl-benzyl-carbonyl)amino- | —CH₂CONH₂ (L-Asn) | —NH₂ | —NH₂ | —NH₂ | 2-O-α-D-mannopyranosyl-α-D- |
| | methyl | | | | | mannopyranosyl |
| 155 | 1-N-(1-ethyl-n-pentyl-carbonyl)amino- | —CH₂CONH₂ (L-Asn) | —NH₂ | —NH₂ | —NH₂ | 2-O-α-D-mannopymnosyl-α-D- |
| | ethyl | | | | | mannopyranosyl |
| 156 | N-(1-ethyl-n-pentyl-carbonyl)amino- | —CH₂CONH₂ (L-Asn) | —NH₂ | —NH₂ | —NH₂ | 2-O-α-D-mannopyranosyl-α-D- |
| | benzylmethyl | | | | | mannopyranosyl |
| 157 | 1-N-(2-methyl-benzyl-carbonyl)amino-2- | —CH₂CONH₂ (L-Asn) | —NH₂ | —NH₂ | —NH₂ | 2-O-α-D-mannopyranosyl-α-D- |
| | methyl-butyl | | | | | mannopyranosyl |
| 158 | Cyclopentyl | —CH₂CONH₂ (L-Asn) | —NH₂ | —NH₂ | —NH₂ | 2-O-α-D-mannopyranosyl-α-D- |
| | | | | | | mannopyranosyl |
| 159 | Cyclopentyl-methyl | —CH₂CONH₂ (L-Asn) | —NH₂ | —NH₂ | —NH₂ | 2-O-α-D-mannopyranosyl-α-D- |
| | | | | | | mannopyranosyl |
| 160 | 2-cyclopentyl-ethyl | —CH₂CONH₂ (L-Asn) | —NH₂ | —NH₂ | —NH₂ | 2-O-α-D-mannopyranosyl-α-D- |
| | | | | | | mannopyranosyl |
| 161 | 1-phenyl-cyclopentyl | —CH₂CONH₂ (L-Asn) | —NH₂ | —NH₂ | —NH₂ | 2-O-α-D-mannopyranosyl-α-D- |
| | | | | | | mannopyranosyl |
| 162 | Bicyclo[2.2.1]heptylmethyl | —CH₂CONH₂ (L-Asn) | —NH₂ | —NH₂ | —NH₂ | 2-O-α-D-mannopyranosyl-α-D- |
| | | | | | | mannopyranosyl |
| 163 | Cyclohexylmethyl | —CH₂CONH₂ (L-Asn) | —NH₂ | —NH₂ | —NH₂ | 2-O-α-D-mannopyranosyl-α-D- |
| | | | | | | mannopyranosyl |
| 164 | 4-methyl-cyclohexyl-methyl | —CH₂CONH₂ (L-Asn) | —NH₂ | —NH₂ | —NH₂ | 2-O-α-D-mannopyranosyl-α-D- |
| | | | | | | mannopyranosyl |
| 165 | 2-methyl-cyclohexyl-methyl | —CH₂CONH₂ (L-Asn) | —NH₂ | —NH₂ | —NH₂ | 2-O-α-D-mannopyranosyl-α-D- |
| | | | | | | mannopyranosyl |
| 166 | 4-pentyl-cyclohexyl-methyl | —CH₂CONH₂ (L-Asn) | —NH₂ | —NH₂ | —NH₂ | 2-O-α-D-mannopyranosyl-α-D- |
| | | | | | | mannopyranosyl |
| 167 | Cycloheptyl | —CH₂CONH₂ (L-Asn) | —NH₂ | —NH₂ | —NH₂ | 2-O-α-D-mannopyranosyl-α-D- |
| | | | | | | mannopyranosyl |
| 168 | Cyclopropyl | —CH₂CONH₂ (L-Asn) | —NH₂ | —NH₂ | —NH₂ | 2-O-α-D-mannopyranosyl-α-D- |
| | | | | | | mannopyranosyl |
| 169 | 2-methylcyclopropyl | —CH₂CONH₂ (L-Asn) | —NH₂ | —NH₂ | —NH₂ | 2-O-α-D-mannopyranosyl-α-D- |
| | | | | | | mannopyranosyl |
| 170 | 1-methylcyclopropyl | —CH₂CONH₂ (L-Asn) | —NH₂ | —NH₂ | —NH₂ | 2-O-α-D-mannopyranosyl-α-D- |
| | | | | | | mannopyranosyl |
| 171 | 2,2,3,3-tetramethyl-cyclopropyl | —CH₂CONH₂ (L-Asn) | —NH₂ | —NH₂ | —NH₂ | 2-O-α-D-mannopyranosyl-α-D- |
| | | | | | | mannopyranosyl |
| 172 | 2-(2-methyl-prop-1-enyl)-3,3-dimethyl- | —CH₂CONH₂ (L-Asn) | —NH₂ | —NH₂ | —NH₂ | 2-O-α-D-mannopyranosyl-α-D- |
| | cyclopropyl | | | | | mannopyranosyl |
| 173 | 2-phenyl-cyclopropyl | —CH₂CONH₂ (L-Asn) | —NH₂ | —NH₂ | —NH₂ | 2-O-α-D-mannopyranosyl-α-D- |
| | | | | | | mannopyranosyl |
| 174 | 1-phenyl-cyclopropyl | —CH₂CONH₂ (L-Asn) | —NH₂ | —NH₂ | —NH₂ | 2-O-α-D-mannopyranosyl-α-D- |
| | | | | | | mannopyranosyl |
| 175 | Cyclobutyl | —CH₂CONH₂ (L-Asn) | —NH₂ | —NH₂ | —NH₂ | 2-O-α-D-mannopyranosyl-x-D- |
| | | | | | | mannopyranosyl |
| 176 | Cyclohexen-3-yl | —CH₂CONH₂ (L-Asn) | —NH₂ | —NH₂ | —NH₂ | 2-O-α-D-mannopyranosyl-α-D- |
| | | | | | | mannopyranosyl |

TABLE II

| | |
|---|---|
| | Formula (IB) |
| 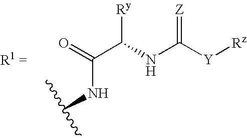 | |

| Ex. | | | | | | | | |
|---|---|---|---|---|---|---|---|---|
| No. | Y | Z | R^{z} | R^{y} | R² | R³ | R⁴ | R⁵ |
| 148 | NH | S | 2-methyl-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 149 | NH | S | 3-methyl-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 150 | NH | S | 4-methyl-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 151 | NH | S | 2-fluoro-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 152 | NH | S | 3-fluoro-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 153 | NH | S | 4-fluoro-phenyl | —CH₂CONH₂ (L-Asn) | —NH₂ | —NH₂ | —NH₂ | 2-O-α-D-mannopyranosyl-cv-D- |
| | | | | | | | | mannopyranosyl |
| 154 | NH | S | 2,6-difluoro-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 155 | NH | S | Benzyl | —CH₂CONH₂ (L-Asn) | —NH₂ | —NH₂ | —NH₂ | 2-O-α-D-mannopyranosyl-α-D- |
| | | | | | | | | mannopyranosyl |
| 156 | NH | S | 2-phenyl-ethyl | —CH₂CONH₂ (L-Asn) | —NH₂ | —NH₂ | —NH₂ | 2-O-α-D-mannopyranosyl-α-D- |
| | | | | | | | | mannopyranosyl |
| 157 | NH | S | Napth-1-yl | —CH₂CONH₂ (L-Asn) | —NH₂ | —NH₂ | —NH₂ | 2-O-α-D-mannopyranosyl-α-D- |
| | | | | | | | | mannopyranosyl |
| 158 | NH | S | Cyclohexyl | —CH₂CONH₂ (L-Asn) | —NH₂ | —NH₂ | —NH₂ | 2-O-α-D-mannopyranosyl-α-D- |
| | | | | | | | | mannopyranosyl |
| 159 | NH | S | 4′-propyl-4-cyclohexyl-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 160 | NH | S | Phenyl | —CH₂CONH₂ (L-Asn) | —NH₂ | —NH₂ | —NH₂ | 2-O-~-D-mannopyranosyl-α-D- |
| | | | | | | | | mannopyranosyl |
| 161 | NH | O | n-butyl | —CH₂CONH₂ (L-Asn) | —NH₂ | —NH₂ | —NH₂ | 2-O-α-D-mannopyranosyl-α-D- |
| | | | | | | | | mannopyranosyl |
| 162 | NH | O | n-Octyl | —CH₂CONH₂ (L-Asn) | —NH₂ | —NH₂ | —NH₂ | 2-O-α-D-mannopyranosyl-α-D- |
| | | | | | | | | mannopyranosyl |
| 163 | NH | O | Cyclohexyl | —CH₂CONH₂ (L-Asn) | —NH₂ | —NH₂ | —NH₂ | 2-O-α-D-mannopyranosyl-α-D- |
| | | | | | | | | mannopyranosyl |
| 164 | NH | O | Benzyl | —CH₂CONH₂ (L-Asn) | —NH₂ | —NH₂ | —NH₂ | 2-O-α-D-mannopyranosyl-α-D- |
| | | | | | | | | mannopyranosyl |
| 165 | NH | O | Phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 166 | NH | O | 2-trifluoromethyl-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 167 | NH | O | 3-trifluoromethyl-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 168 | NH | O | 4-trifluoromethyl-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 169 | NH | O | 2-methoxy-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 170 | NH | O | 2,6-dimethyl-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 171 | NH | O | Napth-1-yl | —CH₂CONH₂ (L-Asn) | —NH₂ | —NH₂ | —NH₂ | 2-O-α-D-mannopyranosyl-α-D- |
| | | | | | | | | mannopyranosyl |
| 172 | NH | O | 1-Napth-1-yl-ethyl | —CH₂CONH₂ (L-Asn) | —NH₂ | —NH₂ | —NH₂ | 2-O-α-D-mannopyranosyl-α-D- |
| | | | | | | | | mannopyranosyl |
| 173 | NH | O | 2-methyl-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 174 | O | O | Propyl | —CH₂CONH₂ (L-Asn) | —NH₂ | —NH₂ | —NH₂ | 2-O-α-D-mannopyranosyl-~-D- |
| | | | | | | | | mannopyranosyl |
| 175 | O | O | Butyl | —CH₂CONH₂ (L-Asn) | —NH₂ | —NH₂ | —NH₂ | 2-O-α-D-mannopyranosyl-α-D- |
| | | | | | | | | mannopyranosyl |
| 176 | O | O | Hexyl | —CH₂CONH₂ (L-Asn) | —NH₂ | —NH₂ | —NH₂ | 2-O-α-D-mannopyranosyl-α-D- |
| | | | | | | | | mannopyranosyl |
| 177 | O | O | Octyl | —CH₂CONH₂ (L-Asn) | —NH₂ | —NH₂ | —NH₂ | 2-O-~-D-mannopyranosyl-α-D- |
| | | | | | | | | mannopyranosyl |
| 178 | O | O | Decyl | —CH₂CONH₂ (L-Asn) | —NH₂ | —NH₂ | —NH₂ | 2-O-α-D-mannopyranosyl-α-D- |
| | | | | | | | | mannopyranosyl |
| 179 | O | O | isopropyl | —CH₂CONH₂ (L-Asn) | —NH₂ | —NH₂ | —NH₂ | 2-O-α-D-mannopyranosyl-α-D- |
| | | | | | | | | mannopyranosyl |
| 180 | O | O | isobutyl | —CH₂CONH₂ (L-Asn) | —NH₂ | —NH₂ | —NH₂ | 2-O-α-D-mannopyranosyl-α-D- |
| | | | | | | | | mannopyranosyl |
| 181 | O | O | 2,2-dimethyl-propyl | —CH₂CONH₂ (L-Asn) | —NH₂ | —NH₂ | —NH₂ | 2-O-α-D-mannopyranosyl-α-D- |
| | | | | | | | | mannopyranosyl |
| 182 | O | O | 2-ethyl-hexyl | —CH₂CONH₂ (L-Asn) | —NH₂ | —NH₂ | —NH₂ | 2-O-α-D-mannopyranosyl-α-D- |
| | | | | | | | | mannopyranosyl |
| 183 | O | O | (1S,2R,5S)-2-isopropyl-5-methyl- | —CH₂CONH₂ (L-Asn) | —NH₂ | —NH₂ | —NH₂ | 2-O-α-D-mannopyranosyl-α-D- |
| | | | cyclohex-1-yl | | | | | mannopyranosyl |
| 184 | O | O | (1R,2S,5R)-2-isopropyl-5-methyl- | —CH₂CONH₂ (L-Asn) | —NH₂ | —NH₂ | —NH₂ | 2-O-α-D-mannopyranosyl-α-D- |
| | | | cyclohex-1-yl | | | | | mannopyranosyl |
| 185 | O | O | Ethenyl | —CH₂CONH₂ (L-Asn) | —NH₂ | —NH₂ | —NH₂ | 2-O-α-D-mannopyranosyl-α-D- |
| | | | | | | | | mannopyranosyl |
| 186 | O | O | prop-2-enyl | —CH₂CONH₂ (L-Asn) | —NH₂ | —NH₂ | —NH₂ | 2-O-α-D-mannopyranosyl-α-D- |
| | | | | | | | | mannopyranosyl |
| 187 | O | O | But-3-enyl | —CH₂CONH₂ (L-Asn) | —NH₂ | —NH₂ | —NH₂ | 2-O-α-D-mannopyranosyl-α-D- |
| | | | | | | | | mannopyranosyl |
| 188 | O | O | 1-methyl-ethenyl | —CH₂CONH₂ (L-Asn) | —NH₂ | —NH₂ | —NH₂ | 2-O-α-D-mannopyranosyl-α-D- |
| | | | | | | | | mannopyranosyl |
| 189 | O | O | But-3-ynyl | —CH₂CONH₂ (L-Asn) | —NH₂ | —NH₂ | —NH₂ | 2-O-α-D-mannopyranosyl-α-D- |
| | | | | | | | | mnannopyranosyl |
| 190 | O | O | But-2-ynyl | —CH₂CONH₂ (L-Asn) | —NH₂ | —NH₂ | —NH₂ | 2-O-α-D-mannopyranosyl-α-D- |
| | | | | | | | | mannopyranosyl |
| 191 | O | O | 4-fluorophenyl | —CH₂CONH₂ (L-Asn) | —NH₂ | —NH₂ | —NH₂ | 2-O-α-D-mannopyranosyl-α-D- |
| | | | | | | | | mannopyranosyl |
| 192 | O | O | 4-bromophenyl | —CH₂CONH₂ (L-Asn) | —NH₂ | —NH₂ | —NH₂ | 2-O-α-D-mannopyranosyl-α-D- |
| | | | | | | | | mannopyranosyl |
| 193 | O | O | 4-nitrophenyl | —CH₂CONH₂ (L-Asn) | —NH₂ | —NH₂ | —NH₂ | 2-O-α-D-mannopyranosyl-α-D- |
| | | | | | | | | mannopyranosyl |
| 194 | O | O | 4-methoxycarbonyl-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 195 | O | O | 2-chloro-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 196 | O | O | 4-chloro-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 197 | O | O | 2-methoxy-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 198 | O | O | 4-methoxy-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 199 | O | O | 4-methyl-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 200 | O | O | 2-nitro-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 201 | O | O | 3-trifluoromethyl-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 202 | O | O | 2-nitro-3,4-dimethoxy-phenyl | —CH₂CONH₂ (L-Asn) | —NH₂ | —NH₂ | —NH₂ | 2-O-α-D-mannopyranosyl-α-D- |
| | | | | | | | | mannopyranosyl |
| 203 | O | O | Benzyl | —CH₂CONH₂ (L-Asn) | —NH₂ | —NH₂ | —NH₂ | 2-O-α-D-mannopyranosyl-α-D- |
| | | | | | | | | mannopyranosyl |
| 204 | O | O | 2-chloro-phenylmethyl | —CH₂CONH₂ (L-Asn) | —NH₂ | —NH₂ | —NH₂ | 2-O-α-D-mannopyranosyl-α-D- |
| | | | | | | | | mannopyranosyl |
| 205 | O | O | (2-trifluoromethyl-phenyl)-chloro- | —CH₂CONH₂ (L-Asn) | —NH₂ | —NH₂ | —NH₂ | 2-O-α-D-mannopyranosyl-α-D- |
| | | | methyl | | | | | mannopyranosyl |
| 206 | O | O | (4-nitro-phenyl)-methyl | —CH₂CONH₂ (L-Asn) | —NH₂ | —NH₂ | —NH₂ | 2-O-α-D-mannopyranosyl-α-D- |
| | | | | | | | | mannopyranosyl |

TABLE III

| | |
|---|---|
| | Formula (IC) |
| 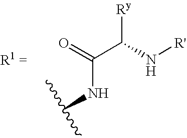 | |

| Ex | | | | | | |
|---|---|---|---|---|---|---|
| No. | R′ | R^{y} | R² | R³ | R⁴ | R⁵ |
| 207 | 4-n-butoxy-benzyl | —CH₂CONH₂ (L-Asn) | —NH₂ | —NH₂ | —NH₂ | 2-O-α-D-mannopyranosyl-α-D-mannopyranosyl |
| 208 | 3,6-difluoro-benzyl | —CH₂CONH₂ (L-Asn) | —NH₂ | —NH₂ | —NH₂ | 2-O-α-D-mannopyranosyl-α-D-mannopyranosyl |
| 209 | 3,6-dimethyl-benzyl | —CH₂CONH₂ (L-Asn) | —NH₂ | —NH₂ | —NH₂ | 2-O-α-D-mannopyranosyl-α-D-mannopyranosyl |
| 210 | 2,3-dihydro-benzo[1,4]dioxin-6- | —CH₂CONH₂ (L-Asn) | —NH₂ | —NH₂ | —NH₂ | 2-O-α-D-mannopyranosyl-α-D-mannopyranosyl |
| | yl-methyl | | | | | |
| 211 | 2-phenyl-ethyl | —CH₂CONH₂ (L-Asn) | —NH₂ | —NH₂ | —NH₂ | 2-O-α-D-mannopyranosyl-α-D-mannopyranosyl |
| 212 | cyclohexyl-methyl | —CH₂CONH₂ (L-Asn) | —NH₂ | —NH₂ | —NH₂ | 2-O-α-D-mannopyranosyl-α-D-mannopyranosyl |
| 213 | n-nonyl | —CH₂CONH₂ (L-Asn) | —NH₂ | —NH₂ | —NH₂ | 2-O-α-D-mannopyranosyl-α-D-mannopyranosyl |
| 214 | n-heptyl | —CH₂CONH₂ (L-Asn) | —NH₂ | —NH₂ | —NH₂ | 2-O-α-D-mannopyranosyl-α-D-mannopyranosyl |
| 215 | 2-phenyl-propyl | —CH₂CONH₂ (L-Asn) | —NH₂ | —NH₂ | —NH₂ | 2-O-α-D-mannopyranosyl-α-D-mannopyranosyl |
| 216 | 4-bromo-benzyl | —CH₂CONH₂ (L-Asn) | —NH₂ | —NH₂ | —NH₂ | 2-O-α-D-mannopyranosyl-α-D-mannopyranosyl |
| 217 | napth-2-yl-methyl | —CH₂CONH₂ (L-Asn) | —NH₂ | —NH₂ | —NH₂ | 2-O-α-D-mannopyranosyl-α-D-mannopyranosyl |
| 218 | 4-phenoxy-benzyl | —CH₂CONH₂ (L-Asn) | —NH₂ | —NH₂ | —NH₂ | 2-O-α-D-mannopyranosyl-α-D-mannopyranosyl |

TABLE IV

| | |
|---|---|
| | Formula (ID) |
| 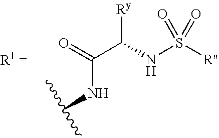 | |

| Ex. | | | | | | |
|---|---|---|---|---|---|---|
| No. | R″ | R^{y} | R² | R³ | R⁴ | R⁵ |
| 219 | 4-fluoro-phenyl | —CH₂CONH₂ (L-Asn) | —NH₂ | —NH₂ | —NH₂ | 2-O-α-D-mannopyranosyl-α-D-mannopyranosyl |
| 220 | Napth-2-yl | —CH₂CONH₂ (L-Asn) | —NH₂ | —NH₂ | —NH₂ | 2-O-α-D-mannopyranosyl-α-D-mannopyranosyl |

TABLE V

| | |
|---|---|
| | Formula (IE) |
| 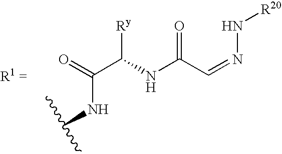 | |

| Ex. | | | | | | |
|---|---|---|---|---|---|---|
| No. | R²⁰ | R^{y} | R² | R³ | R⁴ | R⁵ |
| 221 | phenylaminothiocarbonyl | —CH₂CONH₂ (L-Asn) | —NH₂ | —NH₂ | —NH₂ | 2-O-α-D-mannopyranosyl-α-D-mannopyranosyl |
| 222 | N-ethylaminothiocarbonyl | —CH₂CONH₂ (L-Asn) | —NH₂ | —NH₂ | —NH₂ | 2-O-α-D-mannopyranosyl-α-D-mannopyranosyl |
| 223 | N-prop-2-enylamino- | —CH₂CONH₂ (L-Asn) | —NH₂ | —NH₂ | —NH₂ | 2-O-α-D-mannopyranosyl-α-D-mannopyranosyl |
| | thiocarbonyl | | | | | |
| 224 | Phenylaminocarbonyl | —CH₂CONH₂ (L-Asn) | —NH₂ | —NH₂ | —NH₂ | 2-O-α-D-mannopyranosyl-α-D-mannopyranosyl |
| 225 | Phenylcarbonyl | —CH₂CONH₂ (L-Asn) | —NH₂ | —NH₂ | —NH₂ | 2-O-α-D-mannopyranosyl-α-D-mannopyranosyl |
| 226 | 3-methoxy-phenylcarbonyl | —CH₂CONH₂ (L-Asn) | —NH₂ | —NH₂ | —NH₂ | 2-O-α-D-mannopyranosyl-α-D-mannopyranosyl |
| 227 | Pyridine-4-yl-carbonyl | —CH₂CONH₂ (L-Asn) | —NH₂ | —NH₂ | —NH₂ | 2-O-α-D-mannopyranosyl-α-D-mannopyranosyl |
| 228 | Thiophen-2-ylcarbonyl | —CH₂CONH₂ (L-Asn) | —NH₂ | —NH₂ | —NH₂ | 2-O-α-D-mannopyranosyl-α-D-mannopyranosyl |
| 229 | Benzylcarbonyl | —CH₂CONH₂ (L-Asn) | —NH₂ | —NH₂ | —NH₂ | 2-O-α-D-mannopyranosyl-α-D-mannopyranosyl |

TABLE VI

| | |
|---|---|
| | Formula (IF) |
| 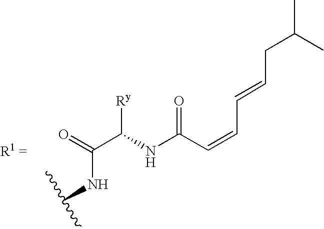 | |

| Ex. No. | R^{y} | R² | R³ | R⁴ | R⁵ |
|---|---|---|---|---|---|
| 259 | —CH₂COOH (L-Asp) | —OH | —NH₂ | —NH₂ | 2-O-α-D-mannopyranosyl-α-D- |
| | | | | | mannopyranosyl |
| 260 | —CH₂COOH (L-Asp) | —OH | —NH₂ | —NH₂ | H |
| 261 and 262 | —CH₂CONH₂ (L-Asn) | —OH | —NH₂ | —NH₂ | 2-O-α-D-mannopyranosyl-α-D- |
| (mixture) | | | | | mannopyranosyl |
| | —CH₂COOH (L-Asp) | —NH₂ | —NH₂ | —NH₂ | 2-O-α-D-mannopyranosyl-α-D- |
| | | | | | mannopyranosyl |
| 263 and 264 | —CH₂CONH₂ (L-Asn) | —OH | —NH₂ | —NH₂ | H |
| (mixture) | —CH₂COOH (L-Asp) | —NH₂ | —NH₂ | —NH₂ | H |
| 265 | —CH₂COOCH₃ | —OCH₃ | —NH₂ | —NH₂ | 2-O-α-D-mannopyranosyl-α-D- |
| | | | | | mannopyranosyl |
| 266 | —CH₂COOCH₃ | —OCH₃ | —NH₂ | —NH₂ | H |
| 267 and 268 | —CH₂CONH₂ (L-Asn) | —OCH₃ | —NH₂ | —NH₂ | 2-O-α-D-mannopyranosyl-α-D- |
| (mixture) | | | | | mannopyranosyl |
| | —CH₂COOCH₃ | —NH₂ | —NH₂ | —NH₂ | 2-O-α-D-mannopyranosyl-α-D- |
| | | | | | mannopyranosyl |
| 269 | —CH₂CONHCH₂CH(CH₃)₂ | —NH—CH₂CH(CH₃)₂ | —NH₂ | —NH₂ | 2-O-α-D-mannopyranosyl-α-D- |
| | | | | | mannopyranosyl |
| 270 | —CH₂CONHCH₂CH₂NHBoc | —NH—CH₂CH₂NHBoc | —NH₂ | —NH₂ | 2-O-α-D-mannopyranosyl-α-D- |
| | | | | | mannopyranosyl |
| 271 | —CH₂CONHCH₂CH₂NH₂ | —NH—CH₂CH₂NH₂ | —NH₂ | —NH₂ | 2-O-α-D-mannopyranosyl-α-D- |
| | | | | | mannopyranosyl |

TABLE VII

| | |
|---|---|
| | Formula (IG) |
| 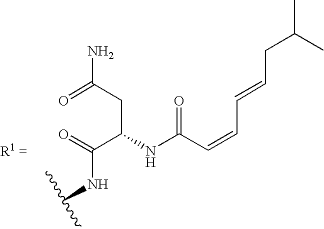 | |

| Ex | | | | |
|---|---|---|---|---|
| No. | R² | R³ | R⁴ | R⁵ |
| 272 | —NH₂ | Guanidino | guanidino | 2-O-α-D-mannopyranosyl-α-D- |
| | | | | mannopyranosyl |
| 273 | —NH₂ | NH₂ | guanidino | 2-O-α-D-mannopyranosyl-α-D- |
| | | | | mannopyranosyl |
| 274 | —NH₂ | Guanidino | NH₂ | 2-O-α-D-mannopyranosyl-α-D- |
| | | | | mannopyranosyl |
| 275 | —NH₂ | N-(1,5-diamino-pentyl- | N-(1,5-diamino-pentyl- | 2-O-α-D-mannopyranosyl-α-D- |
| | | carbonyl)amino | carbonyl)amino | mannopyranosyl |
| 276 | —NH₂ | N-(1,5-diamino-pentyl- | —NH₂ | 2-O-α-D-mannopyranosyl-α-D- |
| | | carbonyl)amino | | mannopyranosyl |
| 277 | —NH₂ | —NH₂ | N-(1,5-diamino-pentyl- | 2-O-α-D-mannopyranosyl-α-D- |
| | | | carbonyl)amino | mannopyranosyl |
| 278 | —NH₂ | N-(aminomethyl-carbonyl)-amino | N-(aminomethyl-carbonyl)-amino | 2-O-α-D-mannopyranosyl-α-D- |
| | | | | maxmopyranosyl |
| 279 | —NH₂ | N-(2-amino-ethyl-carbonyl)amino | N-(2-amino-ethyl-carbonyl)amino | 2-O-α-D-mannopyranosyl-α-D- |
| | | | | mannopyranosyl |
| 280 | —NH₂ | N-(3-amino-propyl- | N-(3-amino-propyl-carbonyl)amino | 2-O-α-D-mannopyranosyl-α-D- |
| | | carbonyl)amino | | maxmopyranosyl |
| 281 | —NH₂ | N-(4-amino-butyl-carbonyl)amino | N-(4-amino-butyl-carbonyl)amino | 2-O-α-D-mannopyranosyl-α-D- |
| | | | | mannopyranosyl |
| 282 | —NH₂ | N-(5-amino-pentyl- | N-(5-amino-pentyl-carbonyl)amino | 2-O-α-D-mannopyranosyl-α-D- |
| | | carbonyl)amino | | mannopyranosyl |
| 283 | —NH₂ | N-(aminomethyl-carbonyl)amino | —NH₂ | 2-O-α-D-mannopyranosyl-α-D- |
| | | | | mannopyranosyl |
| 284 | —NH₂ | N-(2-amino-ethyl-carbonyl)amino | —NH₂ | 2-O-α-D-mannopyranosyl-α-D- |
| | | | | mannopyranosyl |
| 285 | —NH₂ | N-(3-amino-propyl- | —NH₂ | 2-O-α-D-mannopyranosyl-α-D- |
| | | carbonyl)amino | | mannopyranosyl |
| 286 | —NH₂ | N-(4-amino-butyl-carbonyl)amino | —NH₂ | 2-O-α-D-mannopyranosyl-α-D- |
| | | | | mannopyranosyl |
| 287 | —NH₂ | N-(5-amino-pentyl- | —NH₂ | 2-O-α-D-mannopyranosyl-α-D- |
| | | carbonyl)amino | | mannopyranosyl |
| 288 | —NH₂ | 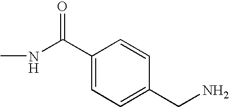 | —NH₂ | 2-O-α-D-mannopyranosyl-α-D- mannopyranosyl |
| 289 | —NH₂ | —NHCOCH₂CH₂COOH | —NH₂ | 2-O-α-D-mannopyranosyl-α-D- |
| | | | | mannopyranosyl |
| 290 | —NH₂ | —NHCH₂CH₂CH₃ | —NH₂ | 2-O-α-D-mannopyranosyl-α-D- |
| | | | | mannopyranosyl |
| 291 | —NH₂ | —N(CH₃)₂ | —NH₂ | 2-O-α-D-mannopyranosyl-α-D- |
| | | | | mannopyranosyl |
| 292 | —NH₂ | 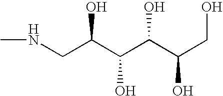 | —NH₂ | 2-O-α-D-mannopyranosyl-α-D- mannopyranosyl |
| 293 | —NH₂ | —NHCH₂COOH | —NH₂ | 2-O-α-D-mannopyranosyl-α-D- |
| | | | | mannopyranosyl |

TABLE VIII

| | |
|---|---|
| | Formula (IH) |
| 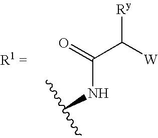 | |

| Ex. | | | | | | |
|---|---|---|---|---|---|---|
| No. | W | R^{y} | R² | R³ | R⁴ | R⁵ |
| 294 | 2-methyl-phenyl | H | —NH₂ | —NH₂ | —NH₂ | 2-O-α-D-mannopyranosyl-α-D- |
| | | | | | | mannopyranosyl |
| 295 | Phenyl-sulfonyl-amino | H | —NH₂ | —NH₂ | —NH₂ | 2-O-α-D-mannopyranosyl-α-D- |
| | | | | | | mannopyranosyl |
| 296 | Phenyl-sulfonyl-N-methyl-amino | H | —NH₂ | —NH₂ | —NH₂ | 2-O-α-D-mannopyranosyl-α-D- |
| | | | | | | mannopyranosyl |
| 297 | 2-methyl-benzyl-carbonylamino | H | —NH₂ | —NH₂ | —NH₂ | 2-O-α-D-mannopyranosyl-α-D- |
| | | | | | | mannopyranosyl |

The compounds, tautomers, prodrugs and pharmaceutically acceptable salts thereof, as defined herein, have activity against gram-positive bacteria.

In another aspect, this invention provides pharmaceutical compositions comprising a pharmaceutically acceptable carrier and a therapeutically effective amount of a compound defined herein. The pharmaceutical compositions of the present invention may further comprise one or more additional antibacterial agents.

In one of its method aspects, this invention is directed to a method for the treatment of a microbial infection in a mammal comprising administering to the mammal a therapeutically effective amount of a compound of this invention. The compound of this invention may be administered to the mammal orally, parenterally, transdermally, topically, rectally, or intranasally.

In another of its method aspects, this invention is directed to a method for the treatment of a microbial infection in a mammal comprising administering to the mammal a pharmaceutical composition comprising a therapeutically effective amount of a compound of this invention. The pharmaceutical compositions of the present invention may further comprise one or more additional antibacterial agents. The pharmaceutical composition may be administered to the mammal orally, parenterally, transdermally, topically, rectally, or intranasally.

In a preferred embodiment, the microbial infection being treated is a gram positive bacterial infection.

In yet another aspect, the present invention provides novel intermediates and processes for preparing compounds of Formula (I).

### DETAILED DESCRIPTION OF THE INVENTION

As described above, this invention relates to ramoplanin derivatives that exhibit antibacterial activity. However, prior to describing this invention in further detail, the following terms will first be defined.

Definitions

Unless otherwise stated, the following terms used in the specification and claims have the meanings given below.

“Aceto” means —C(O)CH₃.

“Acyl” means the group —C(O)R′ wherein R′ is alkyl, substituted alkyl, alkenyl, alkynyl, aryl, substituted aryl, heteroaryl, or substituted heteroaryl.

“Alkenyl” means a linear unsaturated monovalent hydrocarbon radical of two to twelve carbon atoms or a branched monovalent hydrocarbon radical of three to twelve carbon atoms containing at least one double bond, (—C═C—). An alkenyl group may contain two double bonds, or more than two double bonds. Examples of alkenyl groups include, but are not limited to, allyl, vinyl, 2-butenyl, and the like.

“Alkenylene” means a linear unsaturated divalent hydrocarbon radical of two to twelve carbon atoms or a branched divalent hydrocarbon radical of three to twelve carbon atoms.

“Alkoxy” refers to the group “alkyl-O-” wherein alkyl is as defined below, which includes, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

“Alkoxycarbonyl” means the group alkyl-O—C(O)—, where alkyl is as defined herein.

“Alkyl” means a linear saturated monovalent hydrocarbon radical of one to twelve carbon atoms or a branched saturated monovalent hydrocarbon radical of three to twelve carbon atoms. Examples of alkyl groups include, but are not limited to, groups such as methyl, ethyl, n-propyl, isopropyl, n-butyl, isobutyl, sec-butyl, t-butyl, and the like.

“Alkylene” means a linear divalent hydrocarbon radical of one to twelve carbon atoms or a branched divalent hydrocarbon group of three to twelve carbon atoms. Examples of alkylene groups include, but are not limited to, methylene, ethylene, 2-methylpropylene, and the like.

“Alkylsulfanyl” refers to the group “alkyl-S-” which includes, by way of example, methylsulfanyl, butylsulfanyl, and the like.

“Alkynyl” means a linear monovalent hydrocarbon radical of two to twelve carbon atoms or a branched monovalent hydrocarbon radical of three to twelve carbon atoms containing at least one triple bond, (—C≡C—). An alkynyl group may contain two triple bonds, or more than two triple bonds. Examples of alkynyl groups include, but are not limited to, ethynyl, propynyl, 2-butynyl, and the like.

“Aryl” means a monovalent monocyclic, bicyclic or multicyclic aromatic carbocyclic group of six to fourteen ring atoms. Examples include, but are not limited to, phenyl, naphthyl, and anthryl. Aryl groups of the present invention also include fused multicyclic rings wherein one or more of the rings within the multicyclic ring system are cycloalkyl, heterocyclic, or heteroaryl, as long as the point of attachment to the core or backbone of the structure is on the aryl ring. Representative aryl groups with fused rings include, but are not limited to, benzo[1,3]dioxole, benzofuran, benzoimidazole, benzo[d]isoxazole, benzooxazole, benzothiazole, benzo[b]thiophene, benzotriazole, and the like.

“Aryloxy” means “aryl-O-” wherein aryl is as defined above.

“Carbonyl” means the group “C(O).”

“Cycloalkyl” refers to cyclic alkyl groups of from 3 to 20 carbon atoms having a single or multiple cyclic rings including, by way of example, cyclopropyl, cyclobutyl, cyclopentyl, cyclooctyl, bicycle[2.2.1]heptyl, and the like. Cycloalkyl groups of the present invention also include fused multicyclic rings wherein one or more of the rings within the multicyclic ring system are aromatic or heterocyclic, as long as the point of attachment to the core or backbone of the structure is on the cycloalkyl ring, e.g., fluorenyl.

“Halo” or “Halogen” means fluoro, chloro, bromo, or iodo.

“Haloalkoxy” means a “alkyl-O-”, wherein alkyl is as defined above and is substituted with one or more, preferably one to 6, of the same or different halo atoms.

“Haloalkyl” means an alkyl, wherein alkyl is as defined above, substituted with one or more, preferably one to 6, of the same or different halo atoms. Examples of haloalkyl groups include, for example, trifluoromethyl, 3-fluoropropyl, 2,2-dichloroethyl, and the like.

“Heteroaryl” means a monovalent monocyclic or bicyclic aromatic radical of 5 to 10 ring atoms containing one, two, or three ring heteroatoms selected from N, O, or S, the remaining ring atoms being C. Heteroaryl groups of the present invention also include fused multicyclic ring systems wherein one or more of the rings within the multicyclic ring structure are aryl, cycloalkyl or heterocyclic, provided that the point of attachment to the core or backbone of the structure is on the heteroaryl ring.

“Heterocycle” or “heterocyclic” refers to a saturated or unsaturated group having a single ring or multiple condensed rings, from 1 to 10 carbon atoms and from 1 to 4 heteroatoms selected from the group consisting of nitrogen, sulfur, or oxygen within the ring, wherein, in fused ring systems one or more of the rings can be aryl or heteroaryl as defined herein. Heterocyclic groups of the present invention also include fused multicyclic ring systems wherein one or more of the rings within the multicyclic ring structure are aryl, cycloalkyl or heteroaryl, provided that the point of attachment to the core or backbone of the structure is on the heterocyclic ring. Examples of heterocycles and heteroaryls include, but are not limited to, benzo[1,3]dioxolyl, benzofuranyl, benzoimidazolyl, benzo[d]isoxazolyl, benzooxazolyl, benzothiazolyl, benzo[b]thiophenyl, benzotriazolyl, 2,3-dihydrobenzo[1,4]dioxinyl, 2,3-dihydrobenzoimidazolyl, 5,8-dihydro-[1,3]dioxolo-[4,5-g]quinolinyl, 2,3-dihydroindolyl, 1,3-dihydroisoindolyl, 1,4-dihydro-[1,8]naphthyridinyl, 2,3-dihydro-1-oxa-3a-aza-phenalenyl, 1,4-dihydro-quinolinyl, imidazolyl, indazolyl, indolyl, isoxazolyl, oxazolyl, oxazolidinyl, piperidinyl, piperizinyl, pyrazolyl, pyridinyl, pyrrolidinyl, quinolinyl, tetrazolyl, thiazolyl, thiophenyl, [1,2,3]triazolyl, [1,2,4]triazolyl, 1,2,3,4-tetrahydro-isoquinolinyl, 2-pyridonyl, 4,5,6,7-tetrahydrobenzo[b]thiophenyl, 4-pyridonyl, acridinyl, azetidinyl, benzothienyl, carbazolyl, carbolinyl, cinnolinyl, dihydroindolyl, furanyl, imidazolidinyl, imidazolinyl, indolinyl, indolizinyl, isoindolyl, isoquinolinyl, isothiazolyl, isoxazolyl, morpholinyl, naphthylpyridinyl, oxadiazolyl, oxazolonyl, phenanthridinyl, phenanthrolinyl, phenazinyl, phenothiazinyl, phenoxazinyl, phthalazinyl, phthalimidyl, pteridinyl, purinyl, pyrazinonyl, pyrazinyl, pyridazinonyl, pyridazinyl, pyridyl, pyrimidinonyl, pyrimidinyl, pyrrolyl, quinazolinyl, quinolizinyl, quinoxalinyl, tetrahydrofuranyl, thiadiazolyl, thiazolidinyl, thienyl, thiomorpholinyl (also referred to as thiamorpholinyl), and the like.

“Hydroxy” or “hydroxyl” means the group —OH.

“Mammal” refers to all mammals including humans, livestock, laboratory animals, and companion animals.

“Optional” or “optionally” means that the subsequently described event or circumstance may, but need not, occur, and that the description includes instances where the event or circumstance occurs and instances in which it does not. For example, “aryl group optionally mono- or di-substituted with an alkyl group” means that the alkyl may but need not be present, and the description includes situations where the aryl group is mono- or disubstituted with an alkyl group and situations where the aryl group is not substituted with the alkyl group.

“Pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. “A pharmaceutically acceptable carrier” as used in the specification and claims includes both one and more than one such carrier.

“Pharmaceutically acceptable salt” of a compound means a salt that is pharmaceutically acceptable and that possesses the desired pharmacological activity of the parent compound. Such salts include, but are not limited to,
(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-carboxylic acid, glucoheptonic acid, 4,4′methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
(2) salts formed when an acidic proton present in the parent compound either is replaced by a metal ion, e.g., an alkali metal ion, an alkaline earth metal ion, or an aluminum ion; or coordinates with an organic base such as ethanolamine, diethanolamine, triethanolamine, tromethamine, N-methylglucamine, and the like.

“Prodrugs” mean any compound which releases an active parent drug according to a compound of the subject invention in vivo when such prodrug is administered to a mammalian subject. Prodrugs of a compound of the subject invention are prepared by modifying functional groups present in a compound of the subject invention in such a way that the modifications may be cleaved in vivo to release the parent compound. Prodrugs include compounds of the subject invention wherein a hydroxy, sulfhydryl or amino group in the compound is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), carbamates (e.g., N,N-dimethylaminocarbonyl) of hydroxy functional groups, carbamates of amine functional groups in compounds of the subject invention, and the like.

“Substituted alkyl” means an alkyl group, as defined above, in which one or more of the hydrogen atoms has been replaced by a halogen (i.e., Cl, Br, F, or 1), cycloalkyl, substituted cycloalkyl, aryl, substituted aryl, heteroaryl, substituted heteroaryl, heterocyclic, substituted heterocyclic, substituted amino, alkoxy, substituted alkoxy, hydroxy, amine (primary), amine (secondary-amine substituted by alkyl above), amine (tertiary-amine substituted by alkyl as above), or —SH.

“Substituted alkenyl” means an alkenyl group where one or more of the hydrogens has been replaced by a group as defined for substituted alkyl.

“Substituted alkoxy” means substituted alkyl-O—, wherein substituted alkyl is as defined herein.

“Substituted amino” means —NR^{c}R^{d}, wherein R^{c} and R^{d} are each independently H, alkyl, alkenyl, aryl, substituted aryl, acyl, alkylsulfonyl, arylalkyl, arylsulfonyl, alkylsulfonyl, arylalkylsulfonyl.

“Substituted aryl” means an aryl ring substituted with one or more substituents, preferably one to three substituents selected from the group consisting of alkyl, haloalkyl, alkenyl, alkynyl, cycloalkyl, substituted cycloalkyl, alkoxy, haloalkoxy, alkoxycarbonyl, halo, nitro, aryl, aryloxy, heterocyclic, heteroaryl, arylalkoxy, arylsulfanyl, alkylsulfonyl, arylsulfonyl, amino, substituted amino, acyl, acyloxy, hydroxy, carboxy, cyano, alkylsulfanyl, thioalkyl, substituted heteroaryl, substituted heterocyclic. The aryl ring may be optionally fused to a 5-, 6-, or 7-membered monocyclic non-aromatic ring optionally containing 1 or 2 heteroatoms independently selected from oxygen, nitrogen, or sulfur, the remaining ring atoms being carbon where one or two carbon atoms are optionally replaced by a carbonyl.

“Substituted cycloalkyl” means a cycloalkyl substituted with 1-3 groups selected from the group consisting of alkyl, alkenyl, aryl.

“Substituted heteroaryl” means a heteroaryl ring, wherein heteroaryl is as defined above, substituted with one or more substituents, preferably one to three substituents selected from the group consisting of alkyl, substituted alkyl, halo, oxo, aryl, substituted aryl, heteroaryl, substituted heteroaryl, heterocyclic, substituted heterocyclic, cycloalkyl, substituted aryl, aceto, alkenyl, alkynyl, alkoxy, acyloxy, amino, hydroxy, carboxy, cyano, nitro, alkylsulfanyl, and thioalkyl, wherein said substituents are as defined herein.

“Substituted heterocycle” or “substituted heterocyclic” means a heterocyclic ring, wherein heterocyclic is as defined herein, substituted with one or more substituents, preferably one to three substitutents selected from the group consisting of alkyl, substituted alkyl, halo, oxo, aryl, substituted aryl, heteroaryl, substituted heteroaryl, heterocyclic, substituted heterocyclic, cycloalkyl, substituted aryl, aceto, alkenyl, alkynyl, alkoxy, acyloxy, amino, hydroxyl, carboxy, cyano, nitro, and alkylsulfanyl as these terms are defined herein.

“Therapeutically effective amount” means the amount of a compound or composition that, when administered to a mammal for treating a disease, is sufficient to effect such treatment for the disease. The “therapeutically effective amount” will vary depending on the compound or composition, the disease and its severity and the age, weight, etc., of the mammal to be treated. “Treating” or “treatment” of a disease includes:
(1) preventing the disease, i.e. causing the clinical symptoms of the disease not to develop in a mammal that may be exposed to or predisposed to the disease but does not yet experience or display symptoms of the disease,
(2) inhibiting the disease, i.e., arresting or reducing the development of the disease or its clinical symptoms, or
(3) relieving the disease, i.e., causing regression of the disease or its clinical symptoms.

“Tautomer” refers to an isomer in which migration of a hydrogen atom results in two or more structures.

Substituted groups may be substituted up to seven times, e.g., -substituted alkyl-substituted aryl-substituted amino-acyl-substituted alkyl-substituted aryl-alkyl.

The compounds of the present invention are generally named according to the IUPAC or CAS nomenclature system. Abbreviations that are well known to one of ordinary skill in the art may be used (e.g. “Ph” for phenyl, “Me” for methyl, “Et” for ethyl, “Bn” for benzyl, “h” for hour and “rt” for room temperature).

General Synthetic Schemes

Compounds of this invention can be made by the methods depicted in the reaction schemes shown below.

The starting materials and reagents used in preparing these compounds are either available from commercial suppliers such as Acros Organics (Morris Plains, N.J.), Toronto Research Chemicals (North York, ON Canada), Aldrich Chemical Co. (Milwaukee, Wis., USA), Bachem (Torrance, Calif., USA), Emka-Chemie, or Sigma (St. Louis, Mo., USA) or are prepared by methods known to those skilled in the art following procedures set forth in references such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

As it will be apparent to those skilled in the art, conventional protecting groups may be necessary to prevent certain functional groups from undergoing undesired reactions. Suitable protecting groups for various functional groups, as well as suitable conditions for protecting and deprotecting particular function groups are well known in the art. For example, numerous protecting groups are described in T. W. Greene and G. M. Wuts, Protecting Groups in Organic Synthesis, Second Edition, Wiley, New York, 1991, and references cited therein.

The starting materials and the intermediates of the reaction may be isolated and purified if desired using conventional techniques, including but not limited to filtration, distillation, crystallization, chromatography, and the like. Such materials may be characterized using conventional means, including physical constants and spectral data.

The compounds of this invention will typically contain one or more chiral centers. Accordingly, if desired, such compounds can be prepared or isolated as pure stereoisomers. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

Additional synthetic schemes for may be found in PCT application WO 03/076460 and in European Patent Application 0337203, which are herein incorporated by reference in their entirety.

Preparation of Compounds of Formula (I)

In general, to prepare the compounds of Formula (I) of the present invention, the following general synthetic schemes may be used. In Schemes 1-14, R₁, R₂, and R₃ are consistent with the ramoplanin derivatives defined above in the “Summary of the Invention.” In Schemes 14 and 15, R₁, R₂, and R₃ are as defined in said Scheme. Further synthetic methods may be found in General Methods AA-W hereinbelow. Modifications on the following schemes will be apparent to those of skill in the art.

Activated esters may be synthesized from the corresponding acid according to the following general procedure:

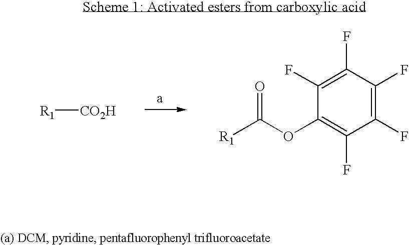

Homologation of carboxylic acids may be performed according to the following general procedure:

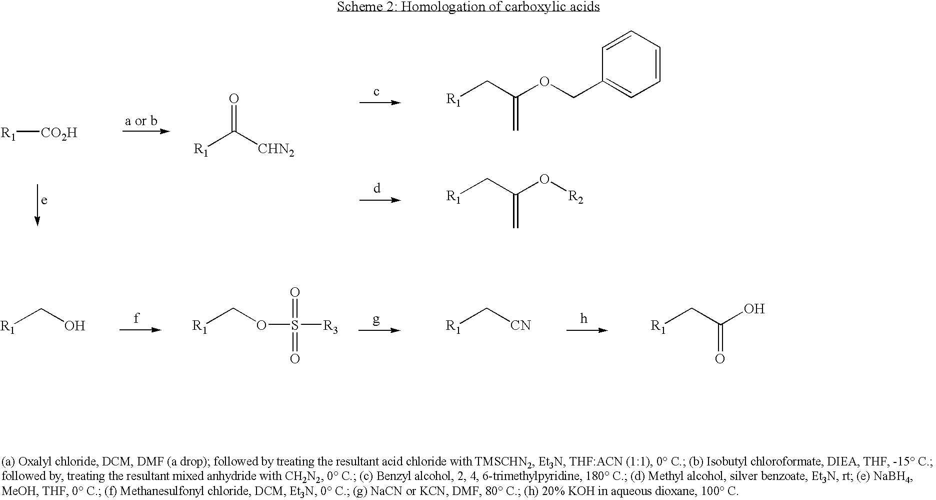

α,β-unsaturated acids and substituted propionic acids may be synthesized from the corresponding aldehyde according to the following general procedure:

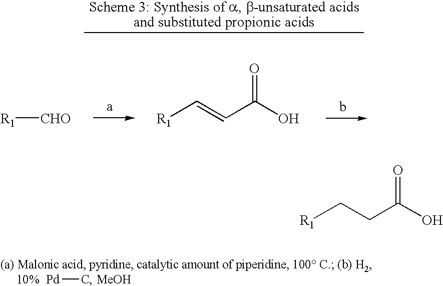

Substituted isoxazoles may be synthesized according to the following general procedure:

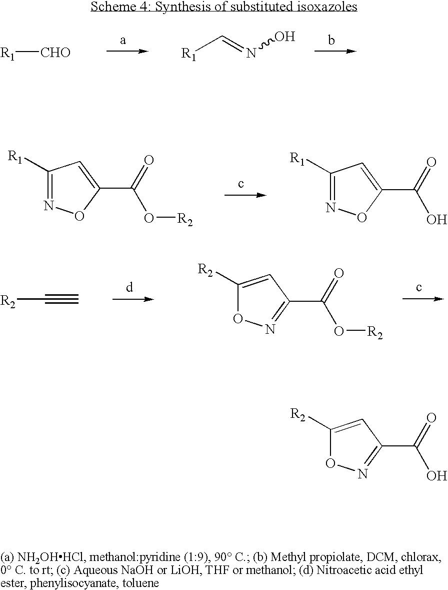

Substituted thioazoles and oxazoles may be synthesized according to the following general procedure:

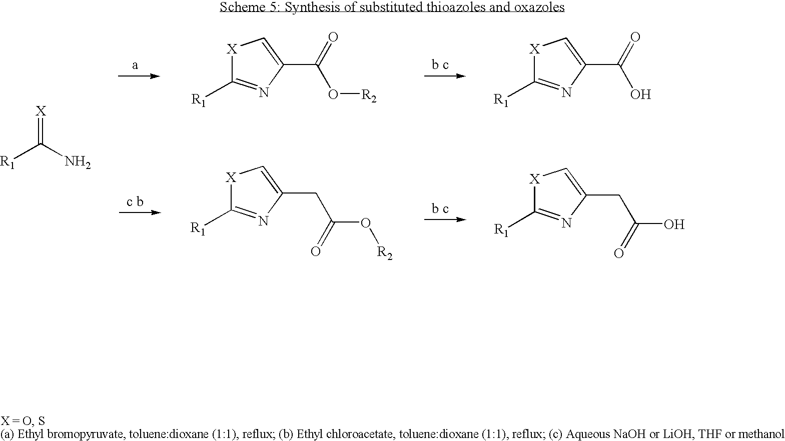

Sulfonamide compounds may be synthesized according to the following general procedures:

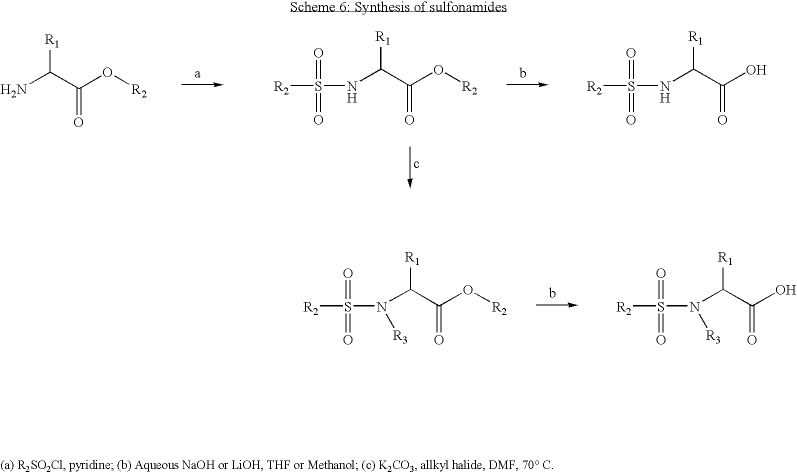

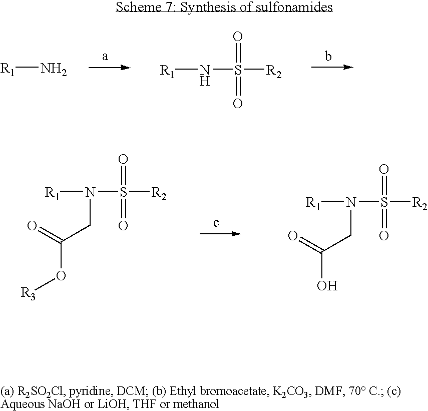

Pyrazoleacetic acids may be synthesized according to the following general procedure:

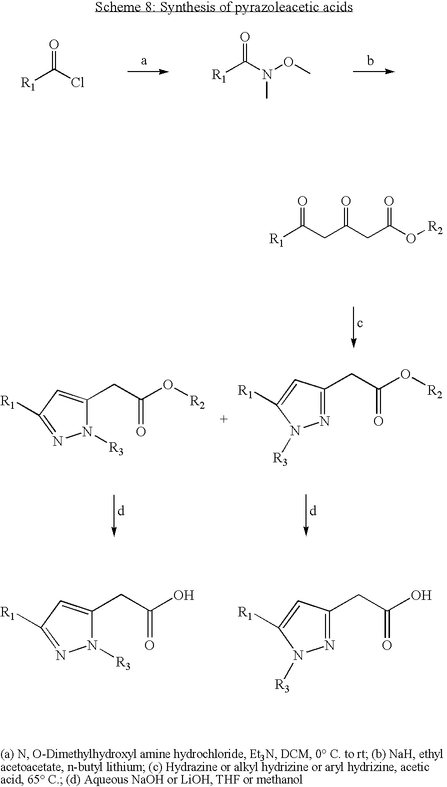

Pyrazoles compounds may be alkylated according to the following general procedure:

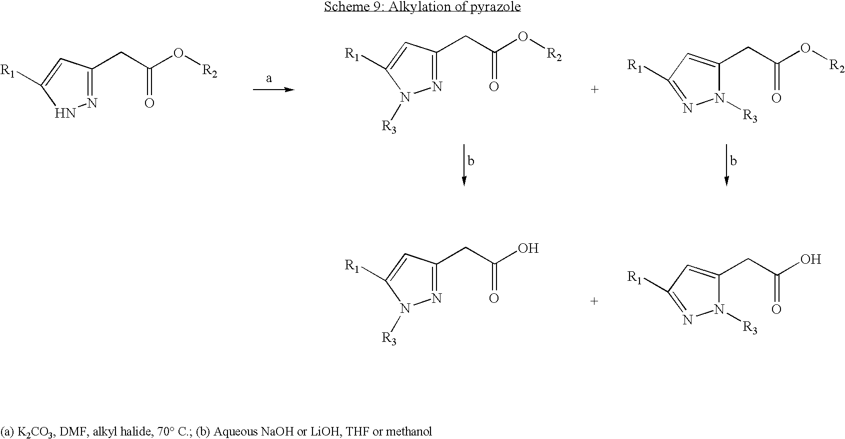

Pyrazolecarboxylic acid compounds may be synthesized according to the following general procedure:

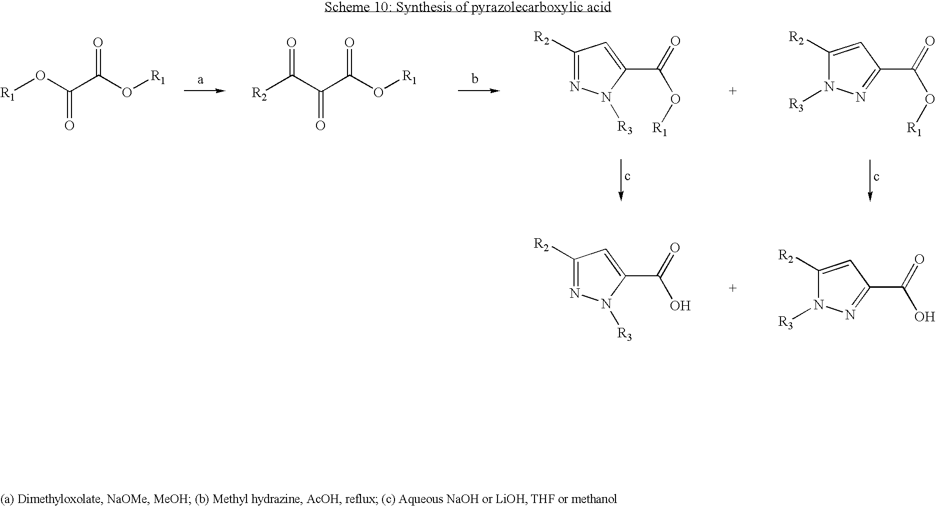

Pyrazoles compounds may be synthesized according to the following general procedures:

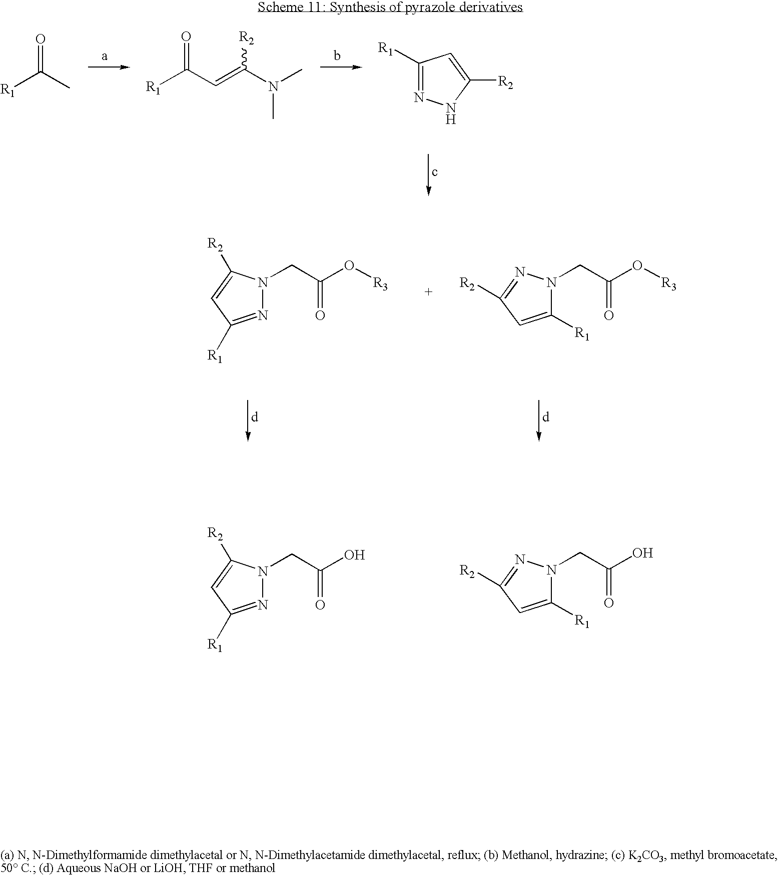

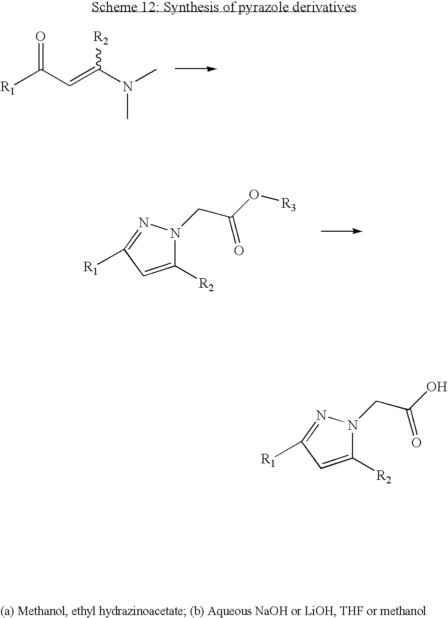

Ramoplanin derivative aglycon compounds may be synthesized in a manner analogous to the following representative procedures:

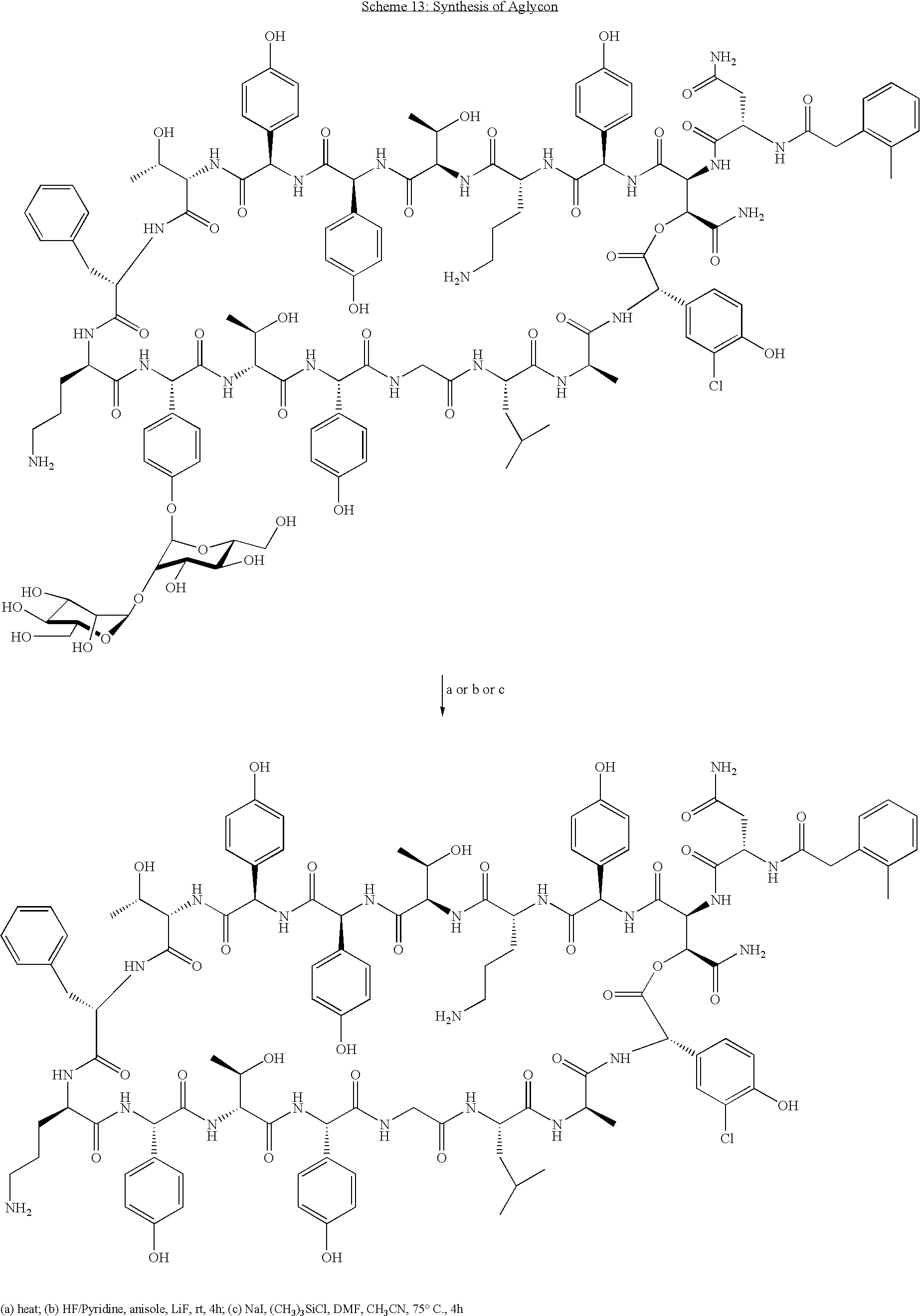

The primary amides may be functionalized according to the following general procedure:

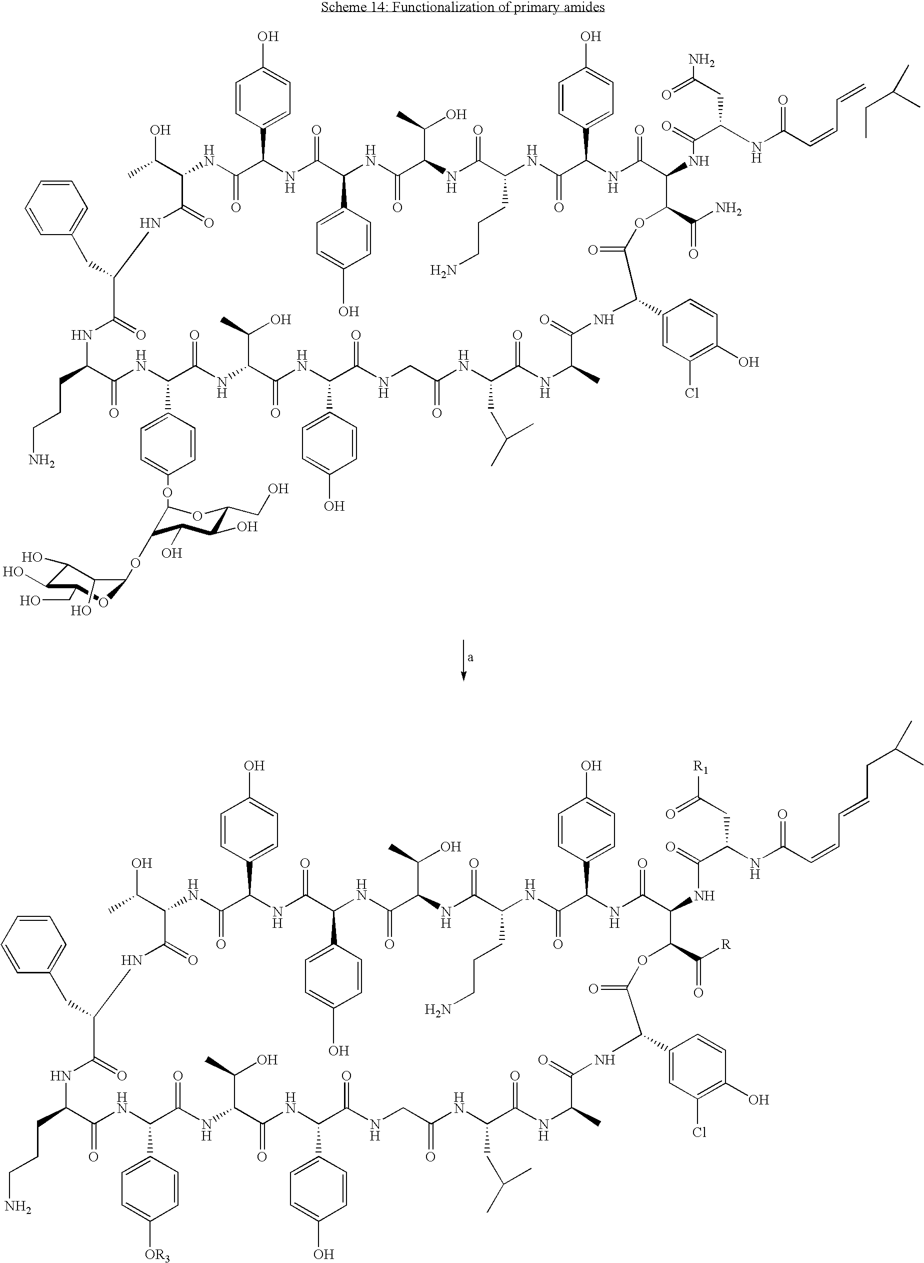

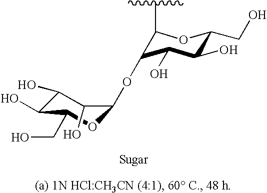

The following reaction products are obtained:
Product 1:R¹=OH, R²=OH, R³=Sugar
Product 2:R¹=OH, R²=OH, R³=H
Product 3:R¹=NH₂, R²=OH, R³=Sugar
Product 4:R¹=NH₂, R²=OH, R³=H
Product 5:R¹=OH, R²=NH₂, R³=Sugar
Product 6:R¹=OH, R²=NH₂, R³=H

Ramoplanin diester derivatives may be obtained in an analogous manner to the following reaction procedure:

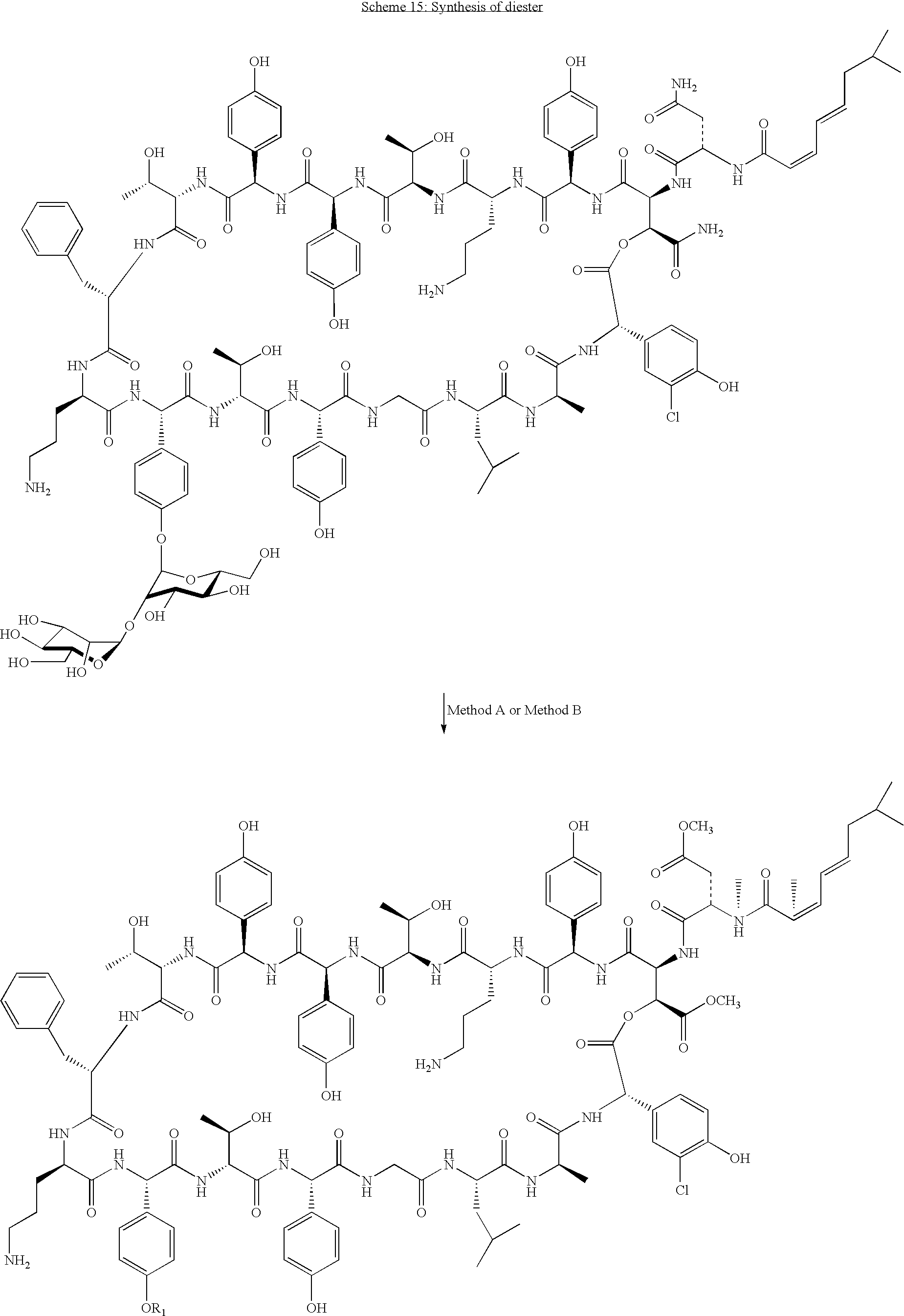

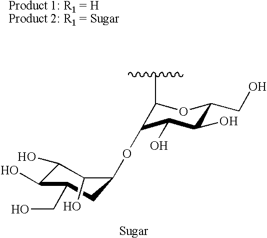

### Method A: Saturated HCl in Methanol, rt, 20 h (Product 1)

### Method B: Methanol: Hydrochloric acid (37%) (3:4), 20 h, rt

Ramoplanin diamide derivatives may be obtained from ramoplanin dicarboxylic acid in an analogous manner to the following reaction procedure:

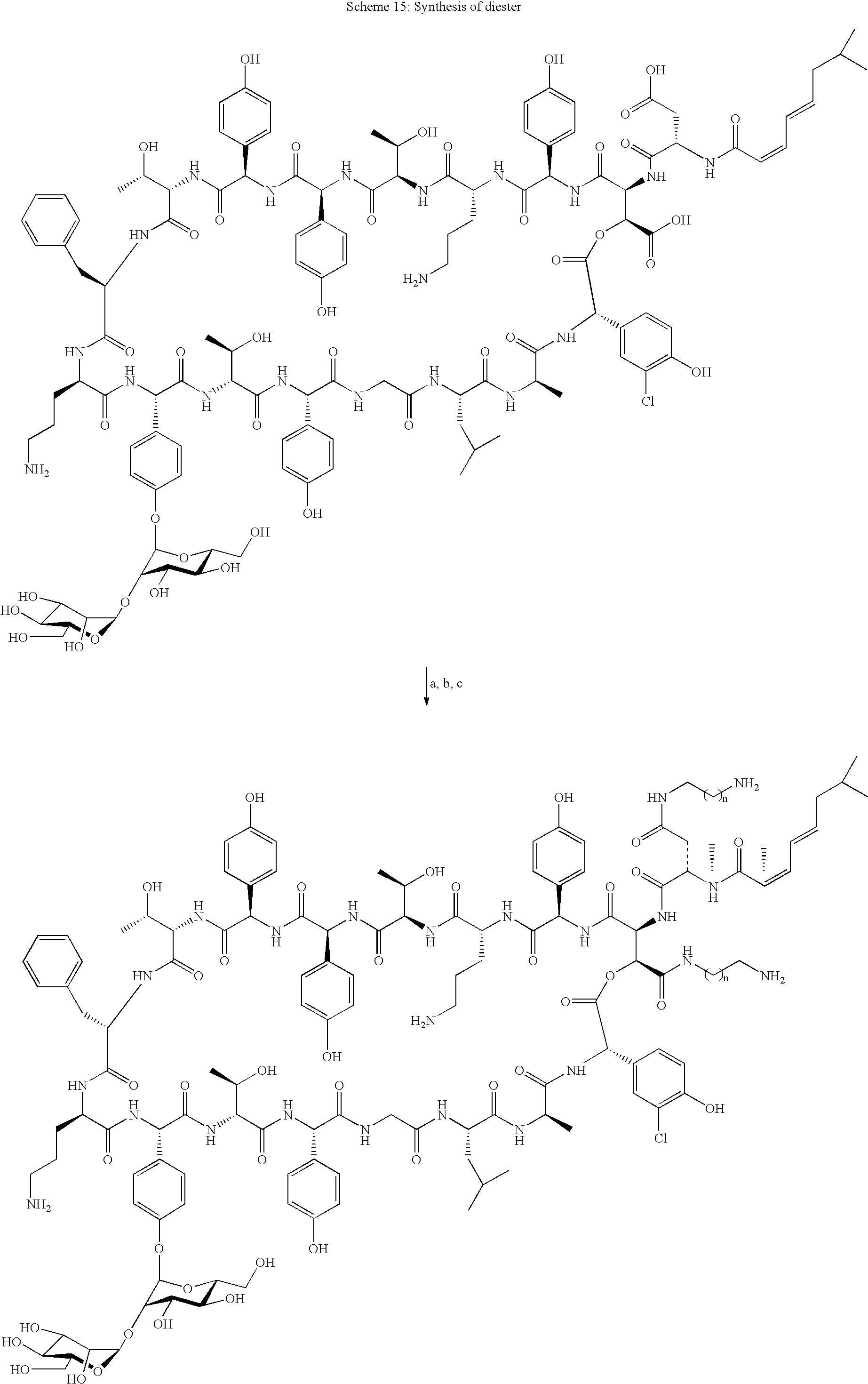

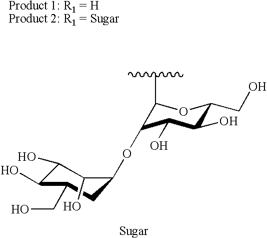

### (a) Suitable monoBoc protected amines are commercially available from Aldrich or Fluka; (b) NH₂CH₂(CH₂)ₙNHBoc, EDC, HOBt, DMAP, DMF, rt, 6 h; (c) TFA:DCM (1:1), rt, 1 h.

Pharmaceutical Formulations

When employed as pharmaceuticals, the compounds of the subject invention are usually administered in the form of pharmaceutical compositions. These compounds can be administered by a variety of routes including oral, parenteral, transdermal, topical, rectal, and intranasal. These compounds are effective as both injectable and oral compositions. Such compositions are prepared in a manner well known in the pharmaceutical art and comprise at least one active compound.

This invention also includes pharmaceutical compositions that contain, as the active ingredient, one or more of the compounds of the subject invention above associated with pharmaceutically acceptable carriers. In making the compositions of this invention, the active ingredient is usually mixed with an excipient, diluted by an excipient or enclosed within such a carrier which can be in the form of a capsule, sachet, paper or other container. The excipient employed is typically an excipient suitable for administration to human subjects or other mammals. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g., about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The quantity of active component, that is the compound according to the subject invention, in the pharmaceutical composition and unit dosage form thereof may be varied or adjusted widely depending upon the particular application, the potency of the particular compound and the desired concentration.

The compositions are preferably formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Preferably, the compound of the subject invention above is employed at no more than about 20 weight percent of the pharmaceutical composition, more preferably no more than about 15 weight percent, with the balance being pharmaceutically inert carrier(s).

The active compound is effective over a wide dosage range and is generally administered in a pharmaceutically or therapeutically effective amount. It will be understood, however, that the amount of the compound actually administered will be determined by a physician, in the light of the relevant circumstances, including the condition to be treated, the severity of the bacterial infection being treated, the chosen route of administration, the actual compound administered, the age, weight, and response of the individual patient, the severity of the patient's symptoms, and the like.

In therapeutic use for treating, or combating, bacterial infections in warm-blooded animals, the compounds or pharmaceutical compositions thereof will be administered orally, topically, transdermally, and/or parenterally at a dosage to obtain and maintain a concentration, that is, an amount, or blood-level of active component in the animal undergoing treatment which will be antibacterially effective. Generally, such antibacterially or therapeutically effective amount of dosage of active component (i.e., an effective dosage) will be in the range of about 0.1 to about 100, more preferably about 1.0 to about 50 mg/kg of body weight/day.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

The following formulation examples illustrate representative pharmaceutical compositions of the present invention.

### FORMULATION EXAMPLE 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### FORMULATION EXAMPLE 2

A tablet formula is prepared using the ingredients below:

| | Quantity |
|---|---|
| Ingredient | (mg/capsule) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### FORMULATION EXAMPLE 3

A dry powder inhaler formulation is prepared containing the following components

| Ingredient | Weight % |
|---|---|
| Active Ingredient | 5 |
| Lactose | 95 |

The active ingredient is mixed with the lactose and the mixture is added to a dry powder inhaling appliance.

### FORMULATION EXAMPLE 4

Tablets, each containing 30 mg of active ingredient, are prepared as follows

| | Quantity | |
|---|---|---|
| Ingredient | (mg/capsule) | |
| Active Ingredient | 30.0 | mg |
| Starch | 45.0 | mg |
| Microcrystalline cellulose | 35.0 | mg |
| Polyvinylpyrrolidone | 4.0 | mg |
| (as 10% solution in sterile water) | | |
| Sodium carboxymethyl starch | 4.5 | mg |
| Magnesium stearate | 0.5 | mg |
| Talc | 1.0 | mg |
| Total | 120 | mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° C. to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### FORMULATION EXAMPLE 5

Capsules, each containing 40 mg of medicament are made as follows:

| | Quantity | |
|---|---|---|
| Ingredient | (mg/capsule) | |
| Active Ingredient | 40.0 | mg |
| Starch | 109.0 | mg |
| Magnesium stearate | 1.0 | mg |
| Total | 150.0 | mg |

The active ingredient, starch and magnesium stearate are blended, passed through a No. 20 mesh U.S. sieve, and filled into hard gelatin capsules in 150 mg quantities.

### FORMULATION EXAMPLE 6

Suppositories, each containing 25 mg of active ingredient are made as follows:

| Ingredient | Amount | |
|---|---|---|
| Active Ingredient | 25 | |
| Saturated fatty acid glycerides to | 2,000 | mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### FORMULATION EXAMPLE 7

Suspensions, each containing 50 mg of medicament per 5.0 mL dose are made as follows:

| Ingredient | Amount | |
|---|---|---|
| Active Ingredient | 50 | mg |
| Xanthan gum | 4.0 | mg |
| Sodium carboxymethyl cellulose (11%) | | |
| Microcrystalline cellulose (89%) | 50.0 | mg |
| Sucrose | 1.75 | g |
| Sodium benzoate | 10.0 | mg |
| Flavor and Color | q.v. | |
| Purified water to | 5.0 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### FORMULATION EXAMPLE 8

| | Quantity | |
|---|---|---|
| Ingredient | (mg/capsule) | |
| Active Ingredient | 15.0 | mg |
| Starch | 407.0 | mg |
| Magnesium stearate | 3.0 | mg |
| Total | 425.0 | mg |

The active ingredient, starch, and magnesium stearate are blended, passed through a No. 20 mesh U.S. sieve, and filled into hard gelatin capsules in 425.0 mg quantities.

### FORMULATION EXAMPLE 9

A subcutaneous formulation may be prepared as follows:

| Ingredient | Quantity | |
|---|---|---|
| Active Ingredient | 5.0 | mg |
| Corn Oil | 1.0 | mL |

### FORMULATION EXAMPLE 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

### FORMULATION EXAMPLE 11

An intravenous formulation may be prepared as follows:

| Ingredient | Quantity |
|---|---|
| Active Ingredient | 250 mg |
| Isotonic saline | 1000 mg |

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 which is herein incorporated by reference.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

Other suitable formulations for use in the present invention can be found in Remington's Pharmaceutical Sciences, Mace Publishing Company, Philadelphia, Pa., 17th ed. (1985).

As noted above, the compounds described herein are suitable for use in a variety of drug delivery systems described above. Additionally, in order to enhance the in vivo serum half-life of the administered compound, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. Nos. 4,235,871, 4,501,728 and 4,837,028 each of which is incorporated herein by reference.

As noted above, the compounds administered to a patient are in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 and 8. It will be understood that use of certain of the foregoing excipients, carriers, or stabilizers will result in the formation of pharmaceutical salts.

In general, the compounds of the subject invention will be administered in a therapeutically effective amount by any of the accepted modes of administration for agents that serve similar utilities. Toxicity and therapeutic efficacy of such compounds can be determined by standard pharmaceutical procedures in cell cultures or experimental animals, e.g. for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range which includes the IC₅₀ (the concentration of the test compound which achieves a half-maximal inhibition of symptoms) as determined in cell culture. Such information can be used to more accurately determine useful doses in humans. Levels in plasma may be measured, for example, by high performance liquid chromatography.

Utility

The compounds, prodrugs and pharmaceutically acceptable salts thereof, as defined herein, have activity against a variety of gram-positive bacteria.

Since the compounds of the subject invention exhibit potent activities against a variety of gram positive bacteria, the compounds of the present invention are useful antimicrobial agents and may be effective against a number of human and veterinary pathogens. The Gram positive organisms against which the compounds of the present invention are effective include Actinomyces spp, Bacillus spp, Bacillus anthracis, Bacillus cereus, Clostridium spp, Clostridium difficile, Clostridium perfringens, Clostridium botulinum, Clostridium tetani, Clostridium ramosum, Clostridium, Corynebacterium spp, Corynebacterium dihpteriae, Enterococcus spp, Enterococcus faecalis, Enterococcus faecium, Enterococcus gallinarum, Enterococcus casseliflavus, Enterococcus avium, Enterococcus durans, Enterococcus raffinosus, Entrerococcus hirae, Enterococcus pseudoavium, Enterococcus malodoratus, Enterococcus mundtii, Erysipelothrix rhusiopathiae, Eubacterium, Gemella haemolysans, Gemella morbillorum, Lactobacillus spp, Lactobacillus rhamnosus, Lactobacillus paracasei, Leuconostoc spp, Leuconostoc mesenteroides, Listeria monocytogenes, Peptostreptococcus magnus, Peptostreptococcus asaccharolyticus, Peptostreptococcus anaerobius, Peptostreptococcus prevotii, Peptostreptococcus micros, Peptostreptococcus hydrogenalis, Propionibacterium acne, Staphylococcus spp, Staphylococcus aureus, Staphylococcus epidermidis, Staphylococcus hominis, Staphylococcus haemolyticus, Staphylococcus saprophyticus, Streptococcus spp, Streptococcus pneumoniae, Streptococcus pyogenes, Streptococcus agalactiae, Streptococcus mutans, Streptococcus sanguis, Streptococcus mitis, Streptococcus bovis, Streptococcus salivarius, Steptococcus anginosus, Streptococcus constellatus, Streptococcus intermedius, and the like.

The compounds of the subject invention may be combined with one or more additional antibacterial agents. One or more of the additional antibacterial agents may be active against gram negative bacteria. Additionally, one or more of the additional antibacterial agents may be active against gram positive bacteria.

The in vitro activity of compounds of the subject invention may be assessed by standard testing procedures such as the determination of minimum inhibitory:concentration (MIC) by agar dilution as described in “Approved Standard. Methods for Dilution Antimicrobial Susceptibility Tests for Bacteria that Grow Aerobically,” 3^{rd} ed., published 1993 by the National Committee for Clinical Laboratory standards, Villanova, Pa., USA.

The amount administered to the mammalian patient will vary depending upon what is being administered, the purpose of the administration, such as prophylaxis or therapy, the state of the patient, the manner of administration, and the like. In therapeutic applications, compositions are administered to a patient already suffering from a disease in an amount sufficient to cure or at least partially arrest the symptoms of the disease and its complications. An amount adequate to accomplish this is defined as “therapeutically effective dose.” Amounts effective for this use will depend on the disease condition being treated as well as by the judgment of the attending clinician depending upon factors such as the severity of the inflammation, the age, weight and general condition of the patient, and the like.

The compositions administered to a patient are in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention will vary according to, for example, the particular use for which the treatment is made, the manner of administration of the compound, the health and condition of the patient, and the judgment of the prescribing physician. For example, for intravenous administration, the dose will typically be in the range of about 20 mg to about 500 mg per kilogram body weight, preferably about 100 mg to about 300 mg per kilogram body weight. Suitable dosage ranges for intranasal administration are generally about 0.1 mg to 100 mg per kilogram body weight. Effective doses can be extrapolated from dose-response curves derived from in vitro or animal model test systems.

The following synthetic and biological examples are offered to illustrate this invention and are not to be construed in any way as limiting the scope of this invention.

### EXAMPLES

In the discussion above and in the examples below, the following abbreviations have the following meanings. If an abbreviation is not defined, it has its generally accepted meaning.
Ac=Acetate
AcOEt=Ethyl acetate
AcOH=Acetic acid
apt=apparent triplet
aq.=aqueous
atm=Atmospheres
Boc=tert-butoxycarbonyl protecting group
br s=broad singlet
n-BuLi=n-butyl lithium
CDCl₃=deuterated chloroform
CD₃OD=deuterated methanol
CFU=colony forming units
d=doublet
dd=doublet of doublets
dt=doublet of triplets
DBU=1,8-diazabicyclo[5.4.0]undec-7-ene
DCM=Dichloromethane
DIEA=Diisopropyethylamine
DMAP=Dimethylaminopyridine
DMF=Dimethylformamide
DMSO=dimethyl sulfoxide
DMSO-d₆=Deuteurated DMSO
ED₅₀=dose therapeutically effective in 50% of the population
EDC=1-(3-dimethylaminopropyl)-3-ethylcarbodiamide HCl
Equiv or eq.=Equivalents
ESMS=electrospray mass spectrometry
Et=Ethyl
EtOAc=ethyl acetate
Et₂O=diethyl ether
FMOC=9-Fluorenyl methoxy carbonyl
g=Grams
h=Hours
HOBt=1-hydroxybenzotriazole hydrate
¹H NMR=Hydrogen Nuclear Magnetic Resonance spectroscopy
HPLC=High pressure liquid chromatography
Hz=Hertz
IC₅₀=concentration of the test compound which achieves a half-maximal inhibition of symptoms
J=coupling constant in hertz
L=Liters
LAH=Lithium aluminum hydride
LCMS=Liquid chromatography mass spectroscopy
LD₅₀=Dose lethal to 50% of the population
LiCl=Lithium chloride
m=Multiplet
M=Molar
Me=Methyl
MeCN=Acetonitrile
MeOH=Methanol
mg=Milligrams
MHz=Megahertz
min=Minutes
mL=Milliliters
mm=Millimeter
mmol=Millimol
m/z=Mass/charge
N=normal
NMR=nuclear magnetic resonance
PBS=phosphate buffered saline
Pd/C=palladium/carbon
PE=polyethylene
Ph=phenyl
Psi=pounds per square inch
Py=pyridine
PyBOP=Benzothiazol-1-yl-oxy-tris-pyrrolidino-phosphonium hexafluorophosphate
q=quartet
q.v.=quantitative
R_{f}=Retention factor
RT or Rₜ=Retention time
rt=room temperature
s=singlet
sat.=saturated
t=triplet
TEA=triethylamine
TFA=trifluoroacetic acid
THF=tetrahydrofuran
TLC=thin layer chromatography
TMS=trimethylsilyl
μg=micrograms
μL=microliters
μm=micromolar
v/v=volume by volume
w/w=weight by weight

Additionally, the term “Aldrich” indicates that the compound or reagent used in the following procedures is commercially available from Aldrich Chemical Company, Inc., 1001 West Saint Paul Avenue, Milwaukee, Wis. 53233 USA; the term “Acros” indicates that the compound or reagent is commercially available from Acros Organics, Morris Plains, N.J.; the term “Fluka” indicates that the compound or reagent is commercially available from Fluka Chemical Corp., 980 South 2nd Street, Ronkonkoma N.Y. 11779 USA; the term “Lancaster” indicates that the compound or reagent is commercially available from Lancaster Synthesis, Inc., P.O. Box 100 Windham, N.H. 03087 USA; the term “Sigma” indicates that the compound or reagent is commercially available from Sigma, P.O. Box 14508, St. Louis Mo. 63178 USA; the term “Chemservice” indicates that the compound or reagent is commercially available from Chemservice Inc., Westchester, Pa., USA; the term “Bachem” indicates that the compound or reagent is commercially available from Bachem Bioscience Inc., 3700 Horizon Drive, Renaissance at Gulph Mills, King of Prussia, Pa. 19406 USA; the term “Maybridge” indicates that the compound or reagent is commercially available from Maybridge Chemical Co.

Trevillett, Tintagel, Cornwall PL34 OHW United Kingdom; the term “RSP” indicates that the compound or reagent is commercially available from RSP Amino Acid Analogs, Inc., 106 South St., Hopkinton, Mass. 01748, USA, and the term “TCI” indicates that the compound or reagent is commercially available from TCI America, 9211 North Harborgate St., Portland, Oreg., 97203, OR, USA; the term “Toronto” indicates that the compound or reagent is commercially available from Toronto Reasearch Chemicals, Inc., 2 Brisbane Rd., New York, ON, Canada M3J2J8; the term “Alfa” indicates that the compound or reagent is commercially available from Johnson Matthey Catalog Company, Inc. 30 Bond Street, Ward Hill, Mass. 018350747; and the term “Nova Biochem” indicates that the compound or reagent is commercially available from NovaBiochem USA, 10933 North Torrey Pines Road, P.O. Box 12087, La Jolla Calif. 92039-2087.

In the examples below, all temperatures are in degrees Celsius (unless otherwise indicated) and the following general procedures are used to prepare the compounds as indicated. It will be appreciated by one of skill in the art that the following general procedures are meant to be illustrative only and that the methods may be broadened to synthesize other compounds of the subject invention.

General Procedures

### Method AA: Method for synthesis of 4,10-diFmoc-deacylramoplanin amine

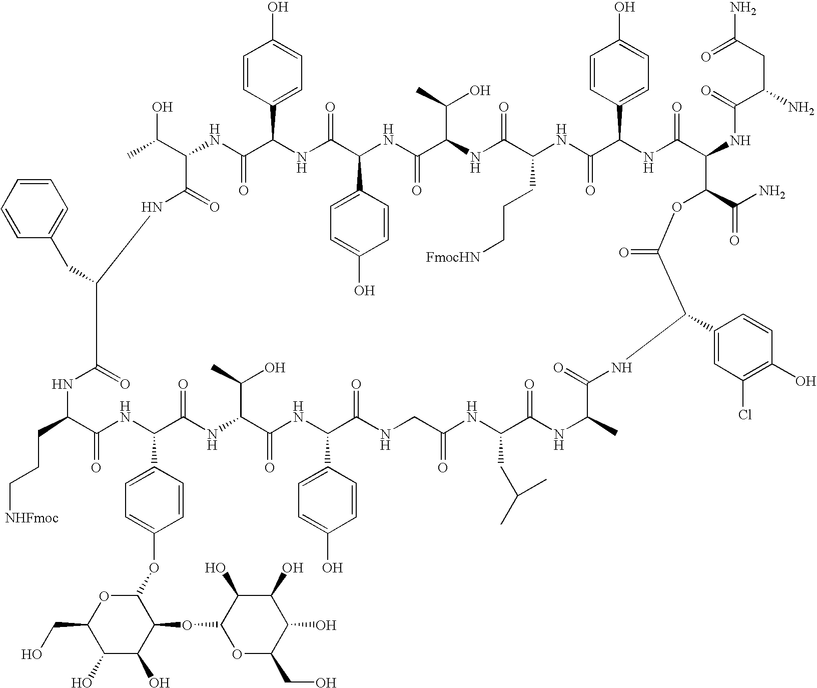

Step I: Protection of the ornithine moieties of ramoplanin. A solution of 95% (w/w) ramoplanin dihydrochloride (110.6 g, 40 mmol) was added to dimethylformamide (500 mL), and was maintained at 0° C. with stirring under nitrogen atmosphere. To this solution N-(9-fluorenylmethoxycarbonyloxy)-succinimide (FMOC-ONSu) (6.8 g, 20 mmol) and TEA (5.8 mL, 41.2 mmol) were added, maintaining the reaction at 0-5° C. After 5 minutes further FMOC-ONSu (6.8 g, 20 mmol) and TEA (5.8 mL, 41.2 mmol) were added. After another 5 minutes, additional FMOC-ONSu (13.6 g, 40 mmol) was added. The reaction temperature was allowed to rise to room temperature. The reaction was monitored by HPLC analysis (retention time 25.6 minutes; Instrument: Shimadzu SCL-6B; Column: Merck Lichrocart 125-4-Lichrosphere 100 RP-18 (5 μm); Flow: 1 ml/min; detector UV λ=270; inj. vol. 10 μl; phase A: HCOONH₄ 0.05M, phase B: MeCN; gradient: time 0 min % B=35; time 15 min % B=40; time 35% B=70). After HPLC control, an addition of a further 10.8 g of FMOC-ONSu was necessary to complete the reaction. After 30 minutes, acetic acid (20 mL) was added, and the reaction mixture was poured into ethyl acetate (6 L). The precipitate was filtered, washed with ethyl acetate (1 L), and dried. 133 grams of a solid product were obtained. The solid was washed while stirring in methanol/water (1:9), and the pH was adjusted to 4.5-5 with acetic acid. The solid was filtered and dried at 35° C. under reduced pressure, obtaining 126.8 grams of a white solid (yield 100%). MS: Lower isotope molecular weight=2996.

Step II: Reductive ozonolysis (synthesis of 4,10-diFmoc-ramoplanin-NHCOCHO). To a solution of 4,10-diFmoc-ramoplanin obtained in the previous step (30 g) in methanol/DMF (9:1, 800 ml), cooled to −78° C., ozone was bubbled (40 mmol, at a flow rate of 100 L/hour of oxygen containing 5% ozone) while stirring. The reaction was maintained at −78° C. for 30 minutes. The reaction was monitored by HPLC analysis (retention time 7.5 minutes; instrument and HPLC conditions as above). The excess ozone was eliminated by bubbling nitrogen into the solution. Triphenylphosphine was added (5.8 g), and the reaction was allowed to reach room temperature. Methanol was evaporated under reduced pressure and the residual DMF solution was poured into ethyl acetate (2 L), with stirring. The precipitate was filtered, washed with ethyl acetate (3×150 mL), and dried at room temperature, obtaining 31.5 grams of a solid (yield 100%). MS: Lower isotope molecular weight=2916.

Step III: Reductive amination (synthesis of 4,10-diFmoc-ramoplanin-NHCOCH₂NHCH₂C₆H₅). To a solution of 4,10-diFmoc-ramoplanin-NHCOCHO (110 g, 38 mmol) and benzylamine hydrobromide (36.5 g, 194 mmol) in anhydrous DMF (925 mL), NaCNBH₃ (3.58 g, 57 mmol) was added while stirring at room temperature. The mixture was stirred for 2 hours. The reaction was monitored by HPLC analysis (retention time 19.6 min; instrument and HPLC conditions as above). The solution was poured into water (9 L). The precipitate was filtered and dried at 35° C. under reduced pressure, obtaining 107 g of crude product. The crude product (107 g) was dissolved at 35° C.-40° C. in 1.5 L of (1:1) acetonitrile:water mixture at pH 2.5 (HCl 1N). To the solution, while stirring, silanized silica gel was added (300 g). After 30 minutes, the acetonitrile was evaporated under reduced pressure, and the water suspension was charged at the top of a silanized silica gel column (diameter 7.5 cm, height 100 cm), previously stabilized with water. The elution was carried out with a water:acetonitrile gradient starting from 85:15 to 1:1. Fractions containing the products were collected and the acetonitrile was evaporated under reduced pressure. The precipitate was filtered, washed with water (100 mL), and dried at 35° C. under reduced pressure, obtaining 20.6 grams of a white solid (total yield for Steps I-III 18%). MS: lower isotope molecular weight=3007.

Step IV: Edman degradation (synthesis of 4,10-diFmoc-deacylramoplanin-amine). To a solution of 4,10-diFmoc-ramoplanin-NHCOCH₂NHCH₂C₆H₅ (17.5 g, 5.65 mmol) in pyridine:water 1:1 (340 mL), phenylisothiocyanate (0.76 mL, 6.35 mmol) was added while stirring at room temperature. The reaction was monitored by HPLC analysis (retention time 24.7 minutes; instrument and HPLC conditions as above). After 1 hour, the solvent was evaporated and the residue was suspended in toluene (50 mL), and evaporated. This operation was repeated twice. The solid was then suspended in dichloromethane (100 mL) and TFA (100 mL) was added. After 15 minutes at 40° C. and HPLC control (retention time 9.5 minutes; instrument and HPLC conditions as above), the mixture was evaporated under reduced pressure, and the oil obtained was triturated with diethyl ether (300 mL). The solid product was filtered, washed with diethyl ether (100 mL), and dried at 35-40° C. under reduced pressure, obtaining 17 grams of solid. The solid was suspended in water, the suspension was stirred at room temperature for 2 hours and filtered; and the solid was dried at 35-40° C. under reduced pressure, obtaining 15 grams of white solid (4,10-diFmoc-deacylramoplanin amine). MS: Lower isotope molecular weight=2860.

### Method A: General Method for Synthesis of Pentafluorophenyl Esters

To a stirred solution of carboxylic acid (1 equivalent) in DCM was added pyridine (1.5 to 10 equivalents) followed by addition of pentafluorophenyl trifluoroacetate (1.2 to 5 equivalents). This mixture was stirred until TLC analysis indicated completion of the reaction (usually 2 to 16 h) at which time the reaction was quenched by addition of 1 N HCl (5 to 10 mL). This mixture was further diluted with DCM. The DCM layer was separated, washed with sat. aqueous NaHCO₃, water, and dried over Na₂SO₄. This dried organic phase was concentrated under reduced pressure to yield relatively pure pentafluorophenyl ester. The crude product was further purified by column chromatography (10 to 30% EtOAc in hexanes) to yield pure pentafluorophenyl ester.

### Method B: General Method for Synthesis of Ramoplanin Analogs

To a 4 mL glass vial charged with 4,10-diFmoc-deacylramoplanin amine (1 equivalent) was added pentafluorophenyl ester (1.1 to 4 equivalents), followed by 300 μL of dry DMF. This mixture was stirred for 2 to 16 h at which time HPLC (0 to 100% of acetonitrile in 0.05 M ammonium formate in water over 10 min, flow rate: 1.5 mL/min, column: Hibar RT 125-4, Merck, injection: 10 μL) indicated completion of the reaction. To this reaction mixture was added piperidine (15 μL) followed by an additional 10 to 15 min of stirring. This reaction was quenched by addition of 200 μL of 1N HCl. This mixture was diluted with water (2.5 mL), followed by further dilution with acetonitrile to a final volume of 3 mL. This mixture was purified via HPLC (5 to 95% of acetonitrile in 0.05 M ammonium formate in water over 45 min, flow rate: 20 mL/min, column: Nova-Pack HR C18, Waters, injection: 1.5 mL or 3 mL).

The final product was characterized using LCMS (0 to 100% of acetonitrile in 0.1 M AcOH in water over 2.7 min, flow rate: 4 mL/min, column: Prevail C-18 ID 17 mm, Alltech, injection: 20 μL, detector: electron spray) and two of the following four HPLC conditions:

HPLC Condition 1: 0 to 100% of acetonitrile in 0.05 M ammonium formate in water over 10 min, flow rate: 1.5 mL/min, column: Hibar RT 125-4, Merck, injection: 10 μL.

HPLC Condition 2: 0 to 100% of 0.1% TFA in acetonitrile in 0.1% TFA in water over 10 min, flow rate: 2 ml/min, column: YMC Propack C-18 AS-300-3, YMC, injection: 10 μL.

HPLC condition 3: 0 to 100% of 0.1% TFA in acetonitrile in 0.1% TFA in water over 20 min, flow rate: 1.5 mL/min, column: YMC Propack C-18 AS-300-3, YMC, injection: 10 μL.

HPLC Condition 4: 0 to 100% of acetonitrile in 0.05 M ammonium formate in water over 20 min, flow rate: 1.5 mL/min, column: Hibar RT 125-4, Merck, injection: 10 μL.

### Method C: General Method for Hydrolysis of Alkyl Esters

To a stirred solution of alkyl ester (1 equivalent) in dioxane was added aqueous NaOH (1N, 1.5 to 10 equivalents). This mixture was stirred until TLC analysis indicated completion of the reaction (usually 30 mins to 16 h) at which time the reaction was extracted with ether. The resulting aqueous layer was separated, acidified by additional of 1N HCl to pH 4. If there was a formation of solid, the solid was filtered, washed with water and air dried to obtain pure acid. Otherwise, the acidified aqueous layer was extracted with EtOAc, the organic layer was dried over Na₂SO₄, concentrated under reduced pressure to yield pure acid.

### Method D: General Method for Reduction of Aldehyde to Alcohol

To a stirred solution of aldehyde (40 mmol) in a mixture of methanol (15 mL) and THF (50 mL) at 0° C. was added solid NaBH₄ (40 mmol) portion wise over a 5 min period. The resultant reaction mixture was stirred at 0° C. for additional 30 min and quenched with addition of saturated NH₄Cl solution. The aqueous layer was extracted with ether, the ether layer was dried over MgSO₄, and concentrated in vacuo to yield the corresponding alcohol. The alcohol was used in the next step without further purification.

### Method E: General Method for Conversion of Alcohol to Mesylate

To a stirred solution of alcohol (40 mmol) and Et₃N (40 mmol) in dichloromethane (30 mL) at 0° C. was slowly added methanesulfonyl chloride (45 mmol). The resultant reaction mixture was continuously stirred at 0° C. for an additional 2 h. The mixture was diluted with dichloromethane, and the organic layer was washed with water. The organic layer was dried over MgSO₄ and concentrated in vacuo to obtain the desired mesylate. The mesylate was used in the subsequent step without purification.

### Method F: General Method for Conversion of Mesylate to Nitrile

To a stirred suspension of potassium cyanide (5.0 g) in DMF (50 mL) was added a solution of mesylate (40 mmol in 5 mL DMF), and the resultant mixture was heated to 80° C. for 2 h. The reaction mixture was cooled to room temperature and diluted with water. The aqueous layer was extracted with ether, dried over MgSO₄ and concentrated in vacuo to yield the desired nitrile derivative.

### Method G: General Method for Conversion of Nitrile to Acid

To a stirred solution of a nitrile (40 mmol) in dioxane (50 mL) was added 20% aqueous potassium hydroxide (50 mL) and the reaction mixture was heated to 100° C. for 16 h. The reaction mixture was concentrated under reduced pressure, the aqueous layer was diluted with water, extracted with ether and the organic layer discarded. The aqueous layer was acidified with 6N hydrochloric acid to pH 3-4. This was extracted with ether, dried over MgSO₄, and concentrated to produce the desired acid.

### Method H: Synthesis of α,β-Unsaturated Acid from Aldehyde

To a stirred solution of aldehyde (40 mmol) in pyridine (50 mL) was added malonic acid (50 mmol) followed by piperidine (2 mL). The reaction mixture was heated to 100° C. for 16 h and concentrated under in vacuo. The resultant residue was poured onto aqueous 1N hydrochloric acid (100 mL). The solid was filtered off and dried under high vacuum.

### Method I: Hydrogenization of α,β-Unsaturated Acids

To a stirred solution of α,β-unsaturated acid (6 mmol) in methanol (20 mL) was added 10% palladium on carbon (Pd—C) and the reaction mixture was subjected to hydrogenation using a balloon pressure of hydrogen for 18 h. The catalyst was filtered through a pad of Celite and washed with methanol. The combined filtrate was concentrated in vacuo to produce the desired acid.

### Method J: Synthesis of Oximes

To a stirred mixture of aldehyde (100 mmol) and hydroxylamine hydrochloride (200 mmol) was added a 1:9 mixture of pyridine:ethanol (150 mL), and the mixture was continuously stirred for 18 h at 90° C. The reaction mixture was concentrated under reduced pressure, the residue dissolved in ether (500 mL), and washed with water. The organic layer was dried over MgSO₄ and concentrated in vacuo to yield the oxime.

### Method K: [3+2] Cycloaddition of Oximes

To a stirred mixture of oxime (30 mmol) and methyl propiolate (10 mL) in dichloromethane (200 mL) was added Chlorax (100 mL) dropwise. The resultant reaction mixture was stirred at room temperature for an additional 1 h (initially the reaction was exothermic). The reaction mixture was diluted with dichloromethane (200 mL), the organic layer was separated, dried over MgSO₄, and concentrated in vacuo.

### Method L: N-Alkylation Using NaH

To a stirred suspension of sodium hydride (11 mmol) in DMF (10 mL) at 0° C. was slowly added a solution of amine (10 mmol in 2 mL of DMF). After completion of addition, the reaction mixture was stirred at room temperature for 30 min, then alkylhalide (11 mmol) was slowly added (exothermic reaction). This was stirred at room temperature for an additional 1 h and the reaction was quenched by adding methanol. The reaction mixture was diluted with ether (300 mL), washed with water, the organic layer was dried over MgSO₄, and the solvent removed in vacuo to produce the desired product.

### Method M: Synthesis of Sulfonamides (aq. NaOH/Dioxane)

To a stirred solution of amine (10 mmol) in a mixture of dioxane (4 mL) and 1N sodium hydroxide (4 mL) at 0° C. was added sulfonyl chloride (1.1 to 3 equiv), and was continuously stirred for 1 h. The reaction mixture was diluted with water (50 mL), extracted with ether, the organic layer dried over MgSO₄, filtered and concentrated in vacuo to yield the desired sulfonamide.

### Method N: Synthesis of Sulfonamides (Pyridine as a Base)

To a stirred solution of amine (10 mmol) in pyridine (8 mL) at 0° C. was added sulfonyl chloride (10 mmol). The resultant reaction mixture was stirred continuously at room temperature for 4 h. The reaction mixture was diluted with water (50 mL), extracted with ether, and the organic layer was washed with 1N hydrochloric acid. The organic layer was dried over MgSO₄, filtered and concentrated in vacuo to yield the desired sulfonamide.

### Method O: N-Alkylation Using Potassium Carbonate

To a stirred solution of amine derivative (10 mmol) in anhydrous DMF (20 mL) was added alkylhalide (11 mmol) followed by anhydrous potassium carbonate (3 g). The resultant reaction mixture was continuously stirred at 70° C. for 16 h. The reaction mixture was diluted with water (100 mL), extracted with ether, and the organic layer was washed with water. The organic layer was dried over MgSO₄, filtered and concentrated in vacuo to yield the desired N-alkylated product.

### Method P: N- or O-Alkylation Using Potassium Carbonate

To a stirred solution of phenol, substituted tetrazole, or sulfonamide compound (10 mmol) in anhydrous DMF (20 mL) was added alkylhalide (11 mmol), followed by anhydrous potassium carbonate (3 g). The resultant reaction mixture was continuously stirred at 70° C. for 16 h. The reaction mixture was diluted with water (100 mL), extracted with ether, and the organic layer was washed with water. The organic layer was dried over MgSO₄, filtered and concentrated in vacuo to yield the desired N- or O-alkylated product.

### Method Q: Hydrogenization of Benzyl Esters

To a stirred solution of benzyl ester (10 mmol) in a 1:1 mixture of ethyl acetate and methanol (100 mL) was added 10% palladium on carbon (400 mg), and the reaction mixture was subjected to hydrogenation using a balloon pressure of hydrogen for 8 h. The catalyst was filtered through a pad of Celite and the Celite pad was washed with methanol. The combined filtrate was concentrated in vacuo to produce the desired acid.

### Method R: General method for preparation of (1-alkyl-5-phenyl-1H-pyrazol-3-yl)acetic acid ethyl ester and (2-alkyl-5-phenyl-2H-pyrazol-3-yl)acetic acid ethyl ester derivatives or (1-aryl-5-phenyl-1H-pyrazol-3-yl)acetic acid ethyl ester and (2-aryl-5-phenyl-2H-pyrazol-3-yl)acetic acid ethyl ester derivatives

Benzoyl chloride (2 g, 14.40 mmol) was added to a suspension of N,O-methylhydroxylamine hydrochloride (1.79 g, 18.46 mmol) in DCM at 0° C. To this mixture was added TEA (4 mL, 28.4 mmol) followed by stirring at rt for 2 h at which time the reaction was quenched by addition of 1N HCl. This mixture was diluted with EtOAc followed by separation of the organic layer. The aqueous phase was further extracted with EtOAc. The combined organic phases were dried over Na₂SO₄, concentrated under reduced pressure to yield relatively pure N-methoxy-N-methyl-benzamide (2.24 g) that was used for next reaction without any further purification. To a stirred suspension of NaH (530 mg, 13.33 mmol, 60% dispersion in oil) in THF (25 mL) at 0° C. was added ethyl acetoacetate (1.5 mL, 12.12 mmol). This mixture was stirred for 30 min at which time the temperature of the reaction was further lowered to −78° C. To this mixture was added BuLi (5 mL, 2.5 M solution in hexanes) and the reaction was stirred for 10 min followed by addition of N-methoxy-N-methyl-benzamide (2.00 g, 12.12 mmol). The reaction was stirred for an additional 30 min at −78° C. followed by warming up to 0° C. over 1 h. The reaction was quenched by addition of sat. aq. NH₄Cl, followed by extraction with EtOAc. The combined organic phases were dried over Na₂SO₄, concentrated under reduced pressure to yield crude 3,5-dioxo-5-phenyl-pentanoic acid ethyl ester (1.19 g) that was used for the next reaction without further purification. To a stirred solution of above 3,5-dioxo-5-phenylpentanoic acid ethyl ester (1 equivalent) in AcOH (24 mL) was added N-alkylhydrazine or N-arylhydrazine (1 equivalent). The resulting reaction was heated to 65° C. for 6 h at which time the reaction was concentrated under reduced pressure and residue was dissolved in EtOAc. This solution was washed with water followed by several portions of sat. aq. NaHCO₃. The organic phase was dried over Na₂SO₄, concentrated under reduced pressure to yield a mixture of two regio isomers of (1-alkyl-5-phenyl-1H-pyrazol-3-yl)acetic acid ethyl ester (minor product) and (2-alkyl-5-phenyl-2H-pyrazol-3-yl)acetic acid ethyl ester (major product), or (1-aryl-5-phenyl-1H-pyrazol-3-yl)acetic acid ethyl ester and (2-aryl-5-phenyl-2H-pyrazol-3-yl)acetic acid ethyl ester. These isomers were purified by silica gel column chromatography (10-20% EtOAc in DCM).

### Method S: General method for preparation of (1-Alkyl-5-phenyl-1H-pyrazol-3-yl)acetic acid ethyl ester derivatives

To a stirred solution of 3,5-dioxo-5-phenyl-pentanoic acid ethyl ester (300 mg, 1.28 mmol, see Method R for preparation) in MeOH (2 mL) was added hydrazine (44 μL, 1.41 mmol). This mixture was stirred at rt for 16 h at which time the reaction was quenched by addition of 1N HCl. The resulting light yellow solid was filtered, washed with several portions of 1N HCl followed by air drying to yield the pure (5-phenyl-1H-pyrazol-3-yl)acetic acid ethyl ester that was used for next reaction without any further purification. To a stirred suspension of (5-phenyl-1H-pyrazol-3-yl)acetic acid ethyl ester (1 equivalent) in DMF (1 mL) was added K₂CO₃ (200 mg) followed by addition of alkylhalide (2 equivalent). This mixture was stirred for 16 h at which time the reaction was diluted with 1N HCl. The resulting solid was filtered, washed with water, dried to obtain the corresponding (1-alkyl-5-phenyl-1H-pyrazol-3-yl)acetic acid ethyl ester.

### Method T: General method for preparation of N-phenyl sulfonamide glycine derivatives

To a stirred solution of glycine-O-Methyl ester hydrochloride (1 equivalent) in 2-5 mL DCM was added pyridine (5 equivalents). The resulting solution was cooled to 0° C. followed by addition of phenylsulfonylchloride (1.2 equivalent). This resulting solution was stirred for 3 h at rt at which time the reaction was quenched by addition of 1N HCl. The resulting mixture was extracted with EtOAc, the organic phase was dried over Na₂SO₄ and concentrated under reduced pressure to yield the crude Benzenesulfonylamino-acetic acid methyl ester in near quantitative yield. To a stirred solution of benzenesulfonylamino-acetic acid methyl ester (1 equivalent) in DMF (2 mL) was added powdered K₂CO₃ (3 equivalents). To this mixture was added alkyl halide (1.5 equivalent) and the resulting mixture was stirred overnight at rt. To this mixture was added 1N NaOH (2 mL), followed by additional stirring for an hour. This mixture was extracted with ether and the resulting aqueous phase was acidified with 1N HCl followed by extraction with EtOAc. The organic phase was dried over Na₂SO₄, and concentrated under reduced pressure to yield relatively pure (Benzenesulfonyl-alkyl-amino)acetic acid.

### Method U: General method for preparation of N-benzyl sulfonamide glycine derivatives

To a stirred solution of Glycine O-methyl ester hydrochloride (1 equivalent) in 2-5 mL DCM was added pyridine (5 equivalent). This mixture was cooled to 0° C., followed by addition of benzylsulfonylchloride (1.2 equivalent). The resulting solution was stirred for 3 h, at which time the reaction was extracted with ether. The resulting aqueous phase was acidified with 1N HCl to pH 3. The resulting mixture was extracted with EtOAc, the organic phase was dried over Na₂SO₄, and concentrated under reduced pressure to yield crude benzylsulfonylamino-acetic acid methyl ester in near quantitative yield. To a stirred solution of benzylsulfonylaminoacetic acid methyl ester (1 equivalent) in DMF (2 mL) was added powdered K₂CO₃ (3 equivalent). To this mixture was added alkyl halide (1.5 equivalent) and the resulting mixture was stirred for 5-16 h at rt. To this mixture was added 1N NaOH (2 mL) followed by additional stirring for an hour. This mixture was extracted with ether and the resulting aqueous phase was acidified with 1N HCl followed by extraction with EtOAc. The organic phase was dried over Na₂SO₄, and concentrated under reduced pressure to yield relatively pure (alkyl-benzylsulfonyl-amino)acetic acid.

### Method V: General method for preparation of N-acyl analogs of deacyldeacyl-ramoplanin analogs

4,10-diFmoc-deacylramoplanin amine (150 mg, 52.4 μmol) was suspended in water (2 mL). To this suspension was added pyridine (2 mL). The resulting mixture was shaken until it became a clear solution. To this solution was added phenylisothiocyanate (10 μL, 78.6 μmol) and the resulting solution was shaken for an additional hour when HPLC (condition 2) indicated complete consumption of the starting material. This mixture was concentrated under reduced pressure to dryness followed suspension of residue in benzene (2 mL). This suspension was concentrated under reduced pressure to yield a white solid. This process was repeated once more followed by suspending the residue in DCM (5 mL). To this suspension was added TFA (5 mL) at which time the solution became clear. This mixture was shaken for 1 h at rt when HPLC (condition 2) indicated complete consumption of the starting thiourea The reaction was concentrated under reduced pressure to yield an oil that was triturated with ether to give an off-white solid. This off-white solid was filtered, washed with ether, and re-suspended in water. This suspension was shaken for 2 h at which time the solid in the reaction mixture was filtered, washed with several portions of water and air-dried overnight to yield relatively pure 4,10-diFmoc-deacyldeacyl ramoplanin amine (100 mg):

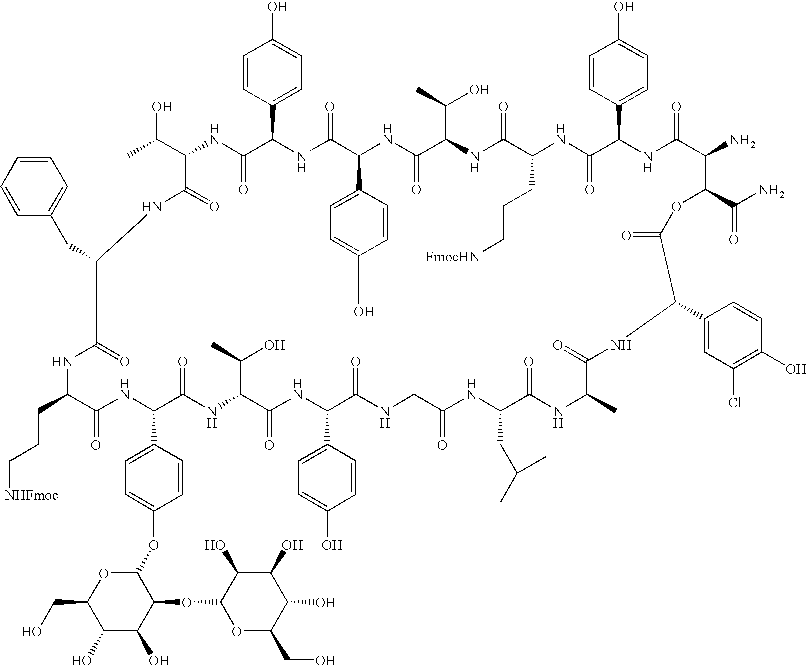

To a solution of 4,10-diFmoc-deacyldeacylramoplanin amine (15-20 mg) and alkyl, aryl or heteroaryl pentafluorophenyl ester (3-5 mg) in DMF (300 μL) was added pyridine (15 μL). The resulting mixture was monitored by HPLC (condition 1 and/or condition 2) until the starting material was completely consumed (usually 1-2 hr). To this mixture was added piperidine (15 μL) and after 10 min. the reaction was quenched by addition of 1N HCl (200 μL). This mixture was diluted with water (2.5 mL) followed by further dilution with acetonitrile to a final volume of 3 mL. This mixture was purified via HPLC (5 to 95% of acetonitrile in 0.05 M ammonium formate in water over 45 min, flow rate: 20 mL/min, column: Nova-Pack HR C18, Waters, injection: 1.5 mL or 3 mL).

### Method W: General Method for the Procedure of Ramoplanin Aglycon Analogs

The 2-O-α-D-mannopyranosyl-α-D-mannopyranosyl moiety of ramoplanin analogs described herein is replaced by a hydrogen in an analogous manner to the one or more of the following three syntheses of VIC-200603-aglycon from VIC-200603, described hereinbelow.

Synthesis of VIC-200603-aglycon from VIC-200603. VIC-200603: 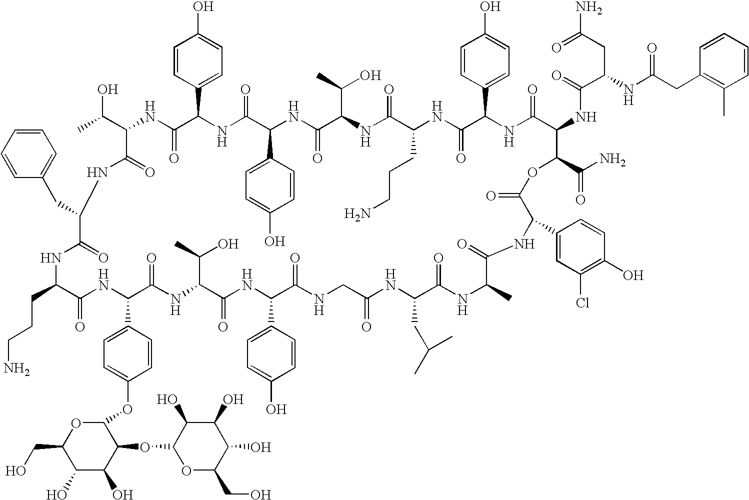
was converted to VIC-200603-aglycon: 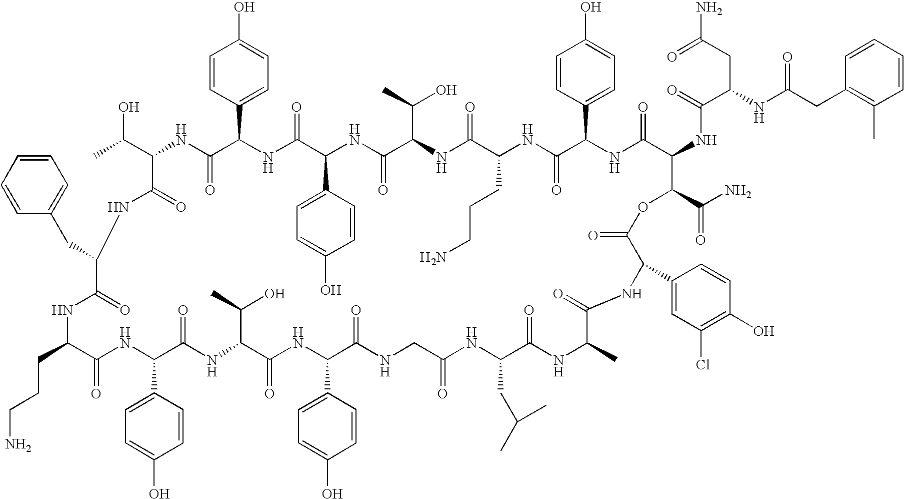
according to the following three methods.
Method 1

150 mg of VIC-200603 were heated with the following temperature steps: (1) 67 h at 60° C.; (2) 47 h at 80° C.; (3) 24 h at 120° C. The reaction was monitored by HPLC analysis (retention time=2.3 min (Instrument: Shimadzu LC 2010A (CLASS-VP6); column: Merck Lichrocart 125-4 Lichrospher 100 RP 8 (5 μm); flow: 1 ml/min; detector UV λ=254 nm; inj. vol. 10 μl; phase A: HCOONH₄ 0.05M; phase B: CH₃CN; gradient: time 0 min % B=5; time 20 min % B=30; time 30 min % B=50; time 40 min % B=70; time 41 min % B=5; time 50 min % B=5). The purified desired product was obtained via purification by preparative HPLC and lyophilization. (Exact Mass=2228).

Method 2

In a small PE bottle, to a solution of anisole (100 μl) in HF/Py 65-70% (3 ml), VIC-200603 (200 mg) and LiF (31 mg, for a final solution concentration of 0.4M) were added with stirring at room temperature. The reaction was monitored by HPLC analysis according to the same HPLC conditions as in Method 1. The mixture was allowed to react at room temperature for 4 hours and then was kept under N₂ stream for 6 hours. CaCO₃ was added, and the suspension was filtered. The filtered solution was acidified with HCl 37% to pH=2.4 and desalted on the poly-acrylic resin XAD 7 HP. The desalted solution was dried under reduced pressure, and the crude product was obtained as a white solid. The purified product was obtained via purification by preparative HPLC and precipitation from Et₂O.

Method 3

To a solution of NaI (0.29 mmoles) in DMF/CH₃CN 1/1 (21 ml), VIC-200603 (0.35 mmoles) and Me₃SiCl (14.2 mmoles) were added with stirring at 75° C. The reaction was monitored by HPLC analysis, using the same HPLC conditions as in Method 1. The mixture was allowed to react at 75° C. for 3 h 45 min. H₂O (21 ml) was added and the solution was brought to pH=4 by adding NaHCO₃. The purified desired product was obtained via purification by preparative HPLC.

### Method X: Synthesis of α-D-mannopyranosyl ramoplanin derivatives

Native ramoplanin is produced as a mixture of α-D-mannopyranosyl and 2-O-α-D-mannopyranosyl-α-D-mannopyranosyl analogues (A′1, A′2, A′3 and A1, A2, A3, respectively), which may be isolated by preparative HPLC according to the methods described herein, as well as methods described in the art (see, for example, European Patent No. 0318680 and U.S. Pat. No. 4,427,656, herein incorporated by reference in their entirety). European Patent No. 0318680 describes the isolation of the α-D-mannopyranosyl analogues of ramoplanin, and further describes a method for enriching the production of the α-D-mannopyranosyl analogues versus production of the 2-O-α-D-mannopyranosyl-α-D-mannopyranosyl analogues. U.S. Pat. No. 4,427,656 describes examples of separation and purification operations, for example, using C-18 alkyl silanized silica gel column and an eluent mixture of aqueous ammonium formate and acetonitrile.

The α-D-mannopyranosyl ramoplanin derivatives of the invention may be synthesized in a similar manner to the 2-O-α-D-mannopyranosyl-α-D-mannopyranosyl derivatives, starting with the α-D-mannopyranosyl ramoplanin analogues. For example, the α-D-mannopyranosyl ramoplanin analogues may be used to make the intermediate compound 4,10-diFmoc-deacylramoplanin amine (α-D-mannopyranosyl analogue) in a similar manner to that shown for 4,10-diFmoc-deacylramoplanin amine (2-O-α-D-mannopyranosyl-α-D-mannopyranosyl analogue) in Method AA.

Alternatively, a mixture of native ramoplanin including both α-D-mannopyranosyl and 2-O-α-D-mannopyranosyl-α-D-mannopyranosyl analogues may be used to synthesize a mixed-saccharide compound of the invention, and the two analogues may be separated by preparative HPLC according to the methods described herein.

In the following Examples, 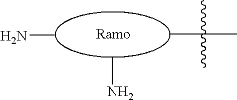
indicates the following base structure:

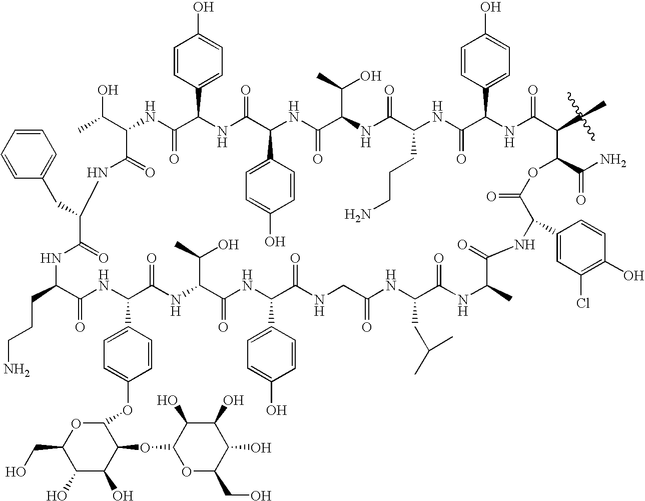

### FORMULA 1A EXAMPLES

### Example 1

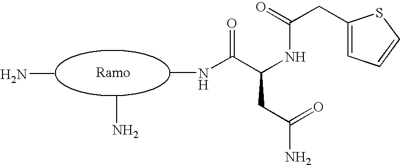

Thiophen-2-ylacetic acid pentafluorophenyl ester was prepared from thiophen-2-ylacetic acid according to Method A in 89% yield. ¹H NMR (300 MHz, CDCl₃): δ 7.29 (dd, J=1.2, 4.8 Hz, 1H), 7.80-7.50 (m, 1H), 7.02 (dd, J=3.6, 5.1 Hz, 1H), 4.20 (s, 2H).

Example 1 was prepared by reacting thiophen-2-ylacetic acid pentafluorophenyl ester with 4,10-diFmoc-deacylramoplanin amine according to Method B. HPLC: Rₜ=4.71 min (Condition 1); Rₜ=3.779 min (Condition 2). ESMS: m/z 1271 [(M+2H)/2].

### Example 2

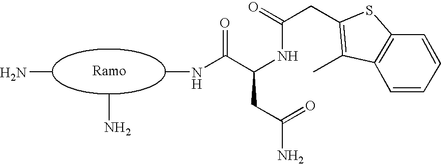

(3-Methylbenzo[b]thiophen-2-yl)acetic acid pentafluorophenyl ester was prepared from (3-methylbenzo[b]thiophen-2-yl)acetic acid according to Method A in 91% yield. ¹H NMR (300 MHz, CDCl₃): δ 7.83-7.79 (m, 1H), 7.72-7.68 (m, 1H), 7.44-7.32 (m, 2H), 4.21 (s, 2H), 2.42 (s, 3H).

Example 2 was prepared by reacting (3-methylbenzo[b]thiophen-2-yl)acetic acid pentafluorophenyl ester with 4,10-diFmoc-deacylramoplanin amine according to Method B. HPLC: Rₜ=5.149 min (Condition 1); Rₜ=4.196 min (Condition 2). ESMS: m/z 1303.2 [(M+2H)/2].

### Example 3

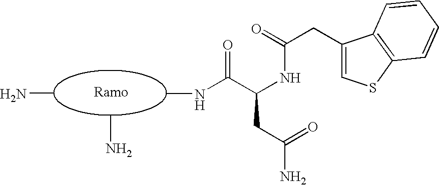

Benzo[b]thiophen-3-ylacetic acid pentafluorophenyl ester was prepared from benzo[b]thiophen-3-ylacetic acid according to Method A in 96% yield. ¹H NMR (300 MHz, CDCl₃): δ 7.92-7.88 (m, 1H), 7.81-7.76 (m, 1H), 7.49 (s, 1H), 7.49-7.37 (m, 2H), 4.22 (s, 2H).

Example 3 was prepared by reacting benzo[b]thiophen-3-ylacetic acid pentafluorophenyl ester with 4,10-diFmoc-deacylramoplanin amine according to Method B. HPLC: Rₜ=5.032 min (Condition 1); Rₜ=4.106 min (Condition 2). ESMS: m/z 1295.8 [(M+2H)/2].

### Example 4

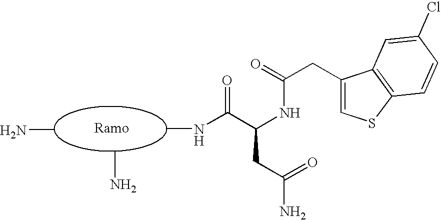

(5-Chlorobenzo[b]thiophen-3-yl)acetic acid pentafluorophenyl ester was prepared from (5-chlorobenzo[b]thiophen-3-yl)acetic acid according to Method A in 86% yield. ¹H NMR (300 MHz, CDCl₃): δ 7.80 (d, J=8.7 Hz, 1H), 7.76 (d, J=1.8 Hz, 1H), 7.55 (s, 1H), 7.37 (dd, J=1.8, 8.7 Hz, 1H), 4.18 (s, 2H).

Example 4 was prepared by reacting (5-chlorobenzo[b]thiophen-3-yl)acetic acid pentafluorophenyl ester with 4,10-diFmoc-deacylramoplanin amine according to Method B. HPLC: Rₜ=5.253 min (Condition 1); Rₜ=4.227 min (Condition 2). ESMS: m/z 1314.7 [(M+2H)/2].

### Example 5

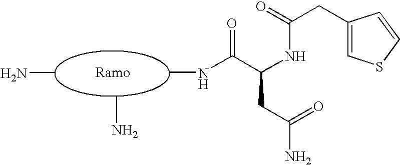

Thiophen-3-ylacetic acid pentafluorophenyl ester was prepared from thiophen-3-ylacetic acid according to Method A in 92% yield. ¹H NMR (300 MHz, CDCl₃): δ 7.36 (dd, J=2.7, 5.1 Hz, 1H), 7.29-7.26 (m, 1H), 7.01 (dd, J=1.2, 5.1 Hz, 1H), 4.18 (s, 2H).

Example 5 was prepared by reacting thiophen-3-ylacetic acid pentafluorophenyl ester with 4,10-diFmoc-deacylramoplanin amine according to Method B. HPLC: Rₜ=4.739 min (Condition 1); Rₜ=3.799 min (Condition 2). ESMS: m/z 1271.3 [(M+2H)/2].

### Example 6

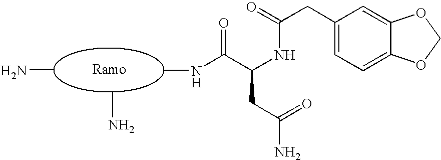

Benzo[1,3]dioxol-5-ylacetic acid pentafluorophenyl ester was prepared from benzo[1,3]dioxol-5-ylacetic acid according to Method A in 93% yield. ¹H NMR (300 MHz, CDCl₃): δ 6.84 (s, 1H), 6.80 (s, 2H), 5.97 (s, 2H), 3.87 (s, 2H).

Example 6 was prepared by reacting benzo[1,3]dioxol-5-ylacetic acid pentafluorophenyl ester with 4,10-diFmoc-deacylramoplanin amine according to Method B. HPLC: Rₜ=4.824 min (Condition 1); Rₜ=3.861 min (Condition 2). ESMS: m/z 1290.3 [(M+2H)/2].

### Example 7

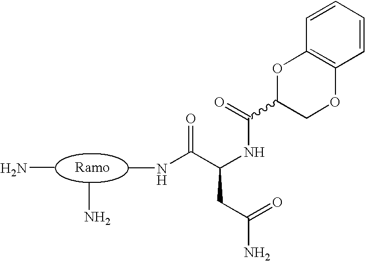

(±)-2,3-Dihydrobenzo[1,4]dioxine-2-carboxylic acid pentafluorophenyl ester was prepared from 2,3-dihydrobenzo[1,4]dioxine-2-carboxylic acid according to Method A in 85% yield. ¹H NMR (300 MHz, CDCl₃): δ 7.06-7.00 (m, 1H), 6.96-6.88 (m, 3H), 5.26-5.22 (m, 1H), 4.62 (dd, J=3.9, 11.4 Hz, 1H), 4.49 (dd, J=2.7, 11.7 Hz, 1H).

Example 7 was prepared by reacting (±)-2,3-dihydrobenzo[1,4]dioxine-2-carboxylic acid pentafluorophenyl ester with 4,10-diFmoc-deacylramoplanin amine according to Method B. HPLC: Rₜ=5.343 min (Condition 1); Rₜ=3.931 min (Condition 2). ESMS: m/z 1290.9 [(M+2H)/2].

### Example 8

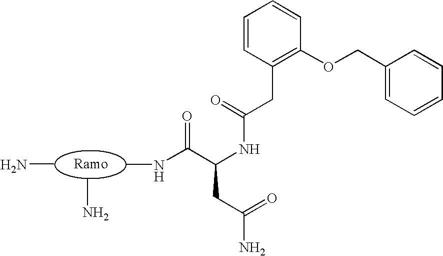

(2-Benzyloxyphenyl)acetic acid pentafluorophenyl ester was prepared from (2-benzyloxyphenyl)acetic acid according to Method A in 90% yield. ¹H NMR (300 MHz, CDCl₃): δ 7.44-7.26 (m, 7H), 6.98 (t, J=7.8 Hz, 2H), 5.14 (s, 2H), 4.02 (s, 2H).

Example 8 was prepared by reacting (2-benzyloxyphenyl)acetic acid pentafluorophenyl ester with 4,10-diFmoc-deacylramoplanin amine according to Method B. HPLC: Rₜ=4.986 min (Condition 1); Rₜ=4.309 min (Condition 2). ESMS: m/z 1321.1 [(M+2H)/2].

### Example 9

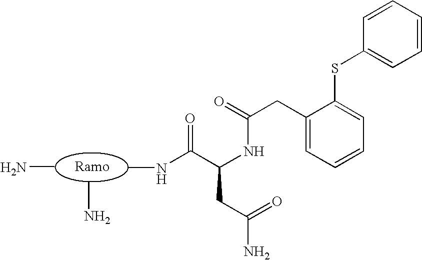

(2-Phenylsulfanylphenyl)acetic acid pentafluorophenyl ester was prepared from (2-phenylsulfanylphenyl)acetic acid according to Method A in 81% yield. ¹H NMR (300 MHz, CDCl₃): δ 7.49-7.17 (m, 9H), 4.17 (s, 2H).

Example 9 was prepared by reacting (2-phenylsulfanylphenyl)acetic acid pentafluorophenyl ester with 4,10-diFmoc-deacylramoplanin amine according to Method B. HPLC: Rₜ=5.446 min (Condition 1); Rₜ=9.098 min (Condition 3). ESMS: m/z 1322.8 [(M+2H)/2].

### Example 10

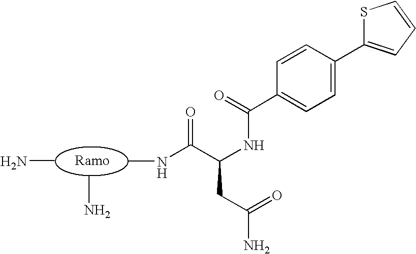

4-Thiophen-2-ylbenzoic acid pentafluorophenyl ester was prepared from (4-thiophen-2-ylbenzoic acid according to Method A in 95% yield. ¹H NMR (300 MHz, CDCl₃): δ 8.23 (d, J=8.1 Hz, 2H), 7.77 (d, J=8.1 Hz, 2H), 7.65 (t, J=2.4 Hz, 1H), 7.47 (d, J=1.8 Hz, 2H).

Example 10 was prepared by reacting 4-thiophen-2-ylbenzoic acid pentafluorophenyl ester with 4,10-diFmoc-deacylramoplanin amine according to Method B. HPLC: Rₜ=5.299 min (Condition 1); Rₜ=8.589 min (Condition 3). ESMS: m/z 1302.8 [(M+2H)/2].

### Example 11

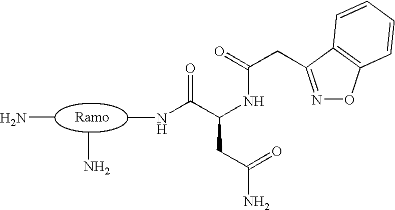

Benzo[d]isoxazol-3-ylacetic acid pentafluorophenyl ester was prepared from benzo[d]isoxazol-3-ylacetic acid according to Method A in 88% yield. ¹H NMR (300 MHz, CDCl₃): δ 7.71 (d, J=7.8 Hz, 1H), 7.63 (m, 2H), 7.38 (t, J=7.8 Hz, 1H).

Example 11 was prepared by reacting benzo[d]isoxazol-3-ylacetic acid pentafluorophenyl ester with 4,10-diFmoc-deacylramoplanin amine according to Method B. HPLC: Rₜ=4.494 min (Condition 1); Rₜ=7.762 min (Condition 3). ESMS: m/z 1288.9 [(M+2H)/2].

### Example 12

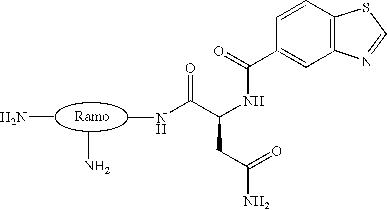

Benzothiazole-5-carboxylic acid pentafluorophenyl ester was prepared from benzothiazole-5-carboxylic acid according to Method A in 81% yield. ¹H NMR (300 MHz, CDCl₃): δ 9.26 (s, 1H), 8.90 (s, 1H), 8.33 (m, 2H).

Example 12 was prepared by reacting benzothiazole-5-carboxylic acid pentafluorophenyl ester with 4,10-diFmoc-deacylramoplanin amine according to Method B. HPLC: Rₜ=5.254 min (Condition 1); Rₜ=6.882 min (Condition 3). ESMS: m/z 1290.3 [(M+2H)/2].

### Example 13

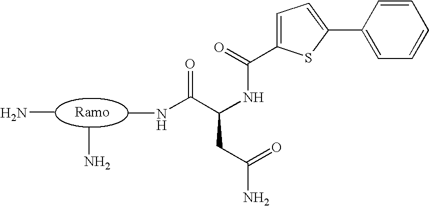

5-Phenylthiophene-2-carboxylic acid pentafluorophenyl ester was prepared from 5-phenylthiophene-2-carboxylic acid according to Method A in 81% yield. ¹H NMR (300 MHz, CDCl₃): δ 8.02 (d, J=4.2 Hz, 1H), 7.67 (dd, J=8.1 Hz, 1.5 Hz, 2H), 7.46 (m, 4H).

Example 13 was prepared by reacting 5-phenylthiophene-2-carboxylic acid pentafluorophenyl ester with 4,10-diFmoc-deacylramoplanin amine according to Method B. HPLC: Rₜ=8.878 min (Condition 3). ESMS: m/z 1302.9 [(M+2H)/2].

### Example 14

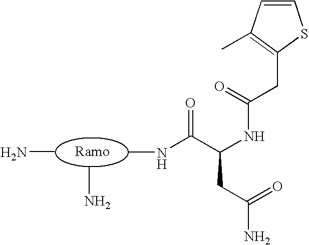

(3-Methylthiophen-2-yl)methanol was obtained from 3-methylthiophene-2-carboxaldehyde in 89% yield according to Method D. Methanesulfonic acid 3-methylthiophen-2-ylmethyl ester was obtained from (3-methylthiophen-2-yl)methanol in 64% yield according to Method E.

(3-Methylthiophen-2-yl)acetonitrile was obtained from methanesulfonic acid 3-methylthiophen-2-ylmethyl ester according to Method F. (3-Methylthiophene-2-yl)acetic acid was obtained from (3-methylthiophen-2-yl)acetonitrile according to Method G. The total % yield for these two steps combined was 4%.

(3-Methylthiophen-2-yl)acetic acid pentafluorophenyl ester was prepared from (3-methylthiophen-2-yl)acetic acid according to Method A in 63% yield. ¹H NMR (300 MHz, CDCl₃): δ 7.15 (d, J=5.22 Hz, 1H), 6.83 (d, J=5.22 Hz, 1H), 4.05 (s, 2H), 2.21 (s, 3H).

Example 14 was prepared by reacting (3-methylthiophen-2-yl)acetic acid pentafluorophenyl ester with 4,10-diFmoc-deacylramoplanin amine according to Method B. HPLC: Rₜ=4.876 min (Condition 1); Rₜ=7.83 min (Condition 3). ESMS: m/z 1278.8 [(M+2H)/2].

### Example 15

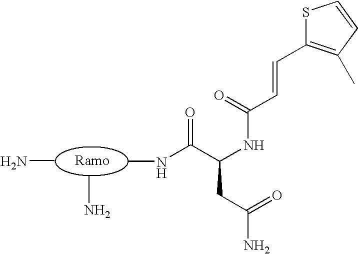

3-(3-Methylthiophen-2-yl)acrylic acid (E-isomer) was prepared from 3-methylthiophene-2-carboxaldehyde following Method H in 91% yield. ¹H NMR (300 MHz, CDCl₃): 7.28 (d, J-=5.21 Hz, 1H), 6.86 (d, J=5.21 Hz, 1H), 6.14 (d, J=15.78 Hz, 1H), 2.34 (s, 3H).

3-(3-Methylthiophen-2-yl)acrylic acid pentafluorophenyl ester (E-isomer) was prepared from 3-methylthiophen-2-yl)acrylic acid (E-isomer) according to Method A in 61% yield. ¹H NMR (300 MHz, CDCl₃): δ 8.06 (d, J=16.2 Hz, 1H), 7.35 (d, J=4.39 Hz, 1H), 6.9 (d, J=5.76 Hz, 1H), 6.32 (d, J=15.65 Hz, 1H), 2.37 (s, 3H).

Example 15 was prepared by reacting 3-(3-methylthiophen-2-yl)acrylic acid pentafluorophenyl ester (E-isomer) with 4,10-diFmoc-deacylramoplanin amine according to Method B. HPLC: Rₜ=5.003 min (Condition 1); Rₜ=8.110 min (Condition 3). ESMS: m/z 1284.7 [(M+2H)/2].

### Example 16

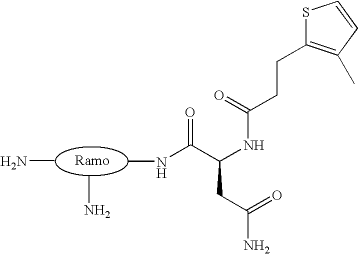

3-(3-Methylthiophen-2-yl)propionic acid was prepared from 3-(3-methylthiophen-2-yl)acrylic acid (from Example 15, first step) following Method I in 91% yield.

3-(3-Methylthiophen-2-yl)propionic acid pentafluorophenyl ester was prepared from 3-(3-methylthiophen-2-yl)propionic acid according to Method A in 67% yield. ¹H NMR (300 MHz, CDCl₃): δ 7.23 (d, J=4.94 Hz, 1H), 6.78 (d, J=4.94 Hz, 1H), 3.17 (t, J=7.41 Hz, 2H), 2.96 (t, J=8.24 Hz, 2H), 2.18 (s, 3H).

Example 16 was prepared by reacting (3-methylthiophen-2-yl)propionic acid pentafluorophenyl ester with 4,10-diFmoc-deacylramoplanin amine according to Method B. HPLC: Rₜ=5.034 min (Condition 1); Rₜ=8.202 min (Condition 3). ESMS: m/z 1285.7 [(M+2H)/2].

### Example 17

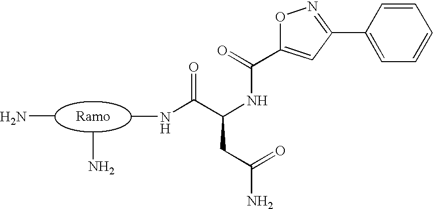

Benzaldehyde oxime was prepared from benzaldehyde according to Method J in 90% yield.

3-Phenylisoxazole-5-carboxylic acid methyl ester was prepared from benzaldehyde oxime and methyl propiolate according to Method K in 48% yield after purification by silica gel column chromatography using hexane/ethyl acetate mixture (8:2) as an eluent.

3-Phenylisoxazole-5-carboxylic acid was prepared from 3-phenylisoxazole-5-carboxylic acid methyl ester according to Method C in 80% yield using LiOH as a base and 1:1 mixture of MEOH:THF as a solvent.

3-Phenylisoxazole-5-carboxylic acid pentafluorophenyl ester was prepared from 3-phenylisoxazole-5-carboxylic acid according to Method A in 60% yield. NMR (300 MHz, CDCl₃): δ 7.81 (m, 2H), 7.44 (m, 3H), 7.16 (s, 1H).

Example 17 was prepared by reacting 3-phenylisoxazole-5-carboxylic acid pentafluorophenyl ester with 4,10-diFmoc-deacylramoplanin amine according to Method B. HPLC: Rₜ=5.027 min (Condition 1); Rₜ=8.072 min (Condition 3). ESMS: m/z 1294.8 [(M+2H)/2].

### Example 18

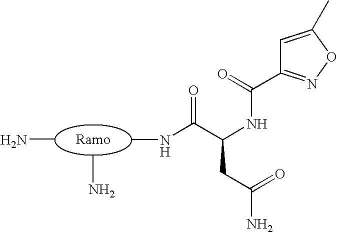

5-Methylisoxazole-3-carboxylic acid pentafluorophenyl ester was prepared from 5-methylisoxazole-3-carboxylic acid according to Method A in 40% yield.

Example 18 was prepared by reacting 5-methylisoxazole-3-carboxylic acid pentafluorophenyl ester with 4,10-diFmoc-deacylramoplanin amine according to Method B. HPLC: Rₜ=4.527 min (Condition 1); Rₜ=6.853 min (Condition 3). ESMS: m/z 1264.4 [(M+2H)/2].

### Example 19

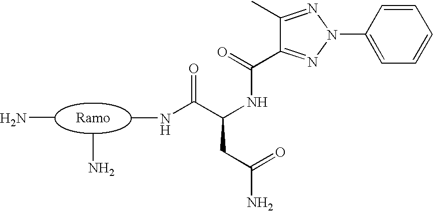

5-Methyl-2-phenyl-2H-[1,2,3]triazole-4-carboxylic acid pentafluorophenyl ester was prepared from 5-methyl-2-phenyl-2H-[1,2,3]triazole-4-carboxylic acid according to Method A in 53% yield.

Example 19 was prepared by reacting 5-methyl-2-phenyl-2H-[1,2,3]triazole-4-carboxylic acid pentafluorophenyl ester with 4,10-diFmoc-deacylramoplanin amine according to Method B. HPLC: Rₜ=5.168 min (Condition 1); Rₜ=8.40 min (Condition 3). ESMS: m/z 1302.2 [(M+2H)/2].

### Example 20

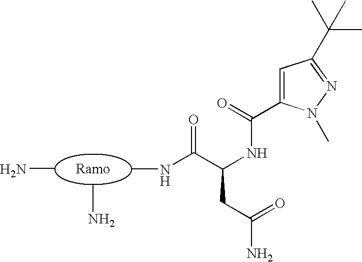

5-tert-Butyl-2-methyl-2H-pyrazole-3-carboxylic acid pentafluorophenyl ester was prepared from 5-tert-butyl-2-methyl-2H-pyrazole-3-carboxylic acid according to Method A in 53% yield.

Example 20 was prepared by reacting 5-tert-Butyl-2-methyl-2H-pyrazole-3-carboxylic acid pentafluorophenyl ester with 4,10-diFmoc-deacylramoplanin amine according to Method B. HPLC: Rₜ=7.749 min (Condition 3). ESMS: m/z 1292.1 [(M+2H)/2].

### Example 21

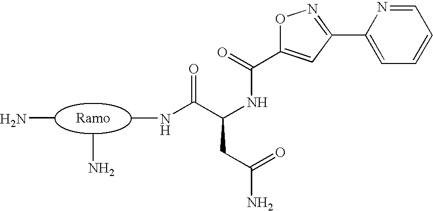

Pyridine-2-carboxaldehyde oxime was prepared from pyridine-2-carboxaldehyde following Method J. The reaction mixture was used in the subsequent step without further work up. 3-Pyridin-2-ylisoxazole-5-carboxylic acid methyl ester was prepared from pyridine-2-carboxaldehyde oxime and methyl propiolate following Method K in 46% yield (for previous two steps combined) after purification of the desired product by silica gel column chromatography using 1:1 hexane/ethyl acetate as an eluent.

3-Pyridin-2-ylisoxazole-5-carboxylic acid was prepared from 3-pyridin-2-ylisoxazole-5-carboxylic acid methyl ester according to Method C in 92% yield using LiOH as base and methanol as a solvent.

3-Pyridin-2-ylisoxazole-5-carboxylic acid pentafluorophenyl ester was prepared from 3-pyridin-2-ylisoxazole-5-carboxylic acid according to Method A in quantative yield. NMR (300 MHz, CDCl₃): δ 8.70 (m, 1H), 8.16 (m, 1H), 7.86 (s, 1H), 7.84 (m, 1H), 7.41 (m, 1H).

Example 21 was prepared by reacting 3-pyridin-2-ylisoxazole-5-carboxylic acid pentafluorophenyl ester with 4,10-diFmoc-deacylramoplanin amine according to Method B. HPLC: Rₜ=4.787 min (Condition 1); Rₜ=7.081 min (Condition 3). ESMS: m/z 1295.9 [(M+2H)/2].

### Example 22

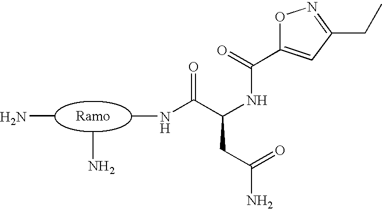

Propionaldehyde oxime was prepared from propionaldehyde following Method J using pyridine as a base but without the use of a co-solvent.

3-Ethylisoxazole-5-carboxylic acid methyl ester was prepared from propionaldehyde oxime and methyl propiolate following Method K in 77% yield after purification of the desired product by silica gel column chromatography using 9:1 hexane/ethyl acetate as an eluent.

3-Ethylisoxazole-5-carboxylic acid was prepared 3-ethylisoxazole-5-carboxylic acid methyl ester according to Method C in 93% yield using LiOH as base and methanol as a solvent.

3-Ethylisoxazole-5-carboxylic acid pentafluorophenyl ester was prepared from 3-ethylisoxazole-5-carboxylic acid according to Method A in 84% yield. NMR (300 MHz, CDCl₃): δ 7.08 (s, 1H), 2.80 (q, J=7.69 Hz, 2H), 1.32 (t, J=7.69 Hz, 3H),

Example 22 was prepared by reacting 3-ethylisoxazole-5-carboxylic acid pentafluorophenyl ester with 4,10-diFmoc-deacylramoplanin amine according to Method B. HPLC: Rₜ=4.771 min (Condition 1); Rₜ=7.047 min (Condition 3). ESMS: m/z 1271.4 [(M+2H)/2].

### Example 23

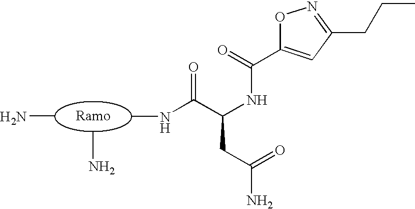

Butyraldehyde oxime was prepared from butyraldehyde following Method J using pyridine as a base but without the use of a co-solvent.

3-Propylisoxazole-5-carboxylic acid methyl ester was prepared from butyraldehyde oxime and methyl propiolate following Method K in 75% yield after purification of the desired product by silica gel column chromatography using 9:1 hexane/ethyl acetate as an eluent.

3-Propylisoxazole-5-carboxylic acid was prepared from 3-propylisoxazole-5-carboxylic acid methyl ester according to Method C in quantitative yield using LiOH as base and methanol as a solvent.

3-Propylisoxazole-5-carboxylic acid pentafluorophenyl ester was prepared from 3-propylisoxazole-5-carboxylic acid according to Method A in 74% yield. NMR (300 MHz, CDCl₃): δ 7.06 (s, 1H), 2.75 (t, J=7.69 Hz, 2H), 1.74 (m, 2H), 0.98 (t, J=7.41 Hz, 3H).

Example 23 was prepared by reacting 3-propylisoxazole-5-carboxylic acid pentafluorophenyl ester with 4,10-diFmoc-deacylramoplanin amine according to Method B. HPLC: Rₜ=4.95 min (Condition 1); Rₜ=7.459 min (Condition 3). ESMS: m/z 1278.4 [(M+2H)/2].

### Example 24

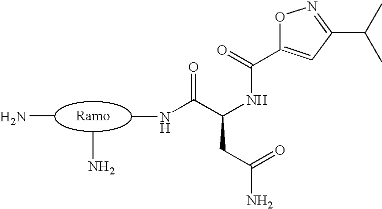

2-Methylpropionaldehyde oxime was prepared from 2-methylpropionaldehyde following Method J using pyridine as a base but without the use of a co-solvent.

3-Isopropylisoxazole-5-carboxylic acid methyl ester was prepared from 2-methylpropionaldehyde oxime and methyl propiolate following Method K in 78% yield after purification of the desired product by silica gel column chromatography using 9:1 hexane/ethyl acetate as an eluent.

3-Isopropylisoxazole-5-carboxylic acid was prepared from 3-isopropylisoxazole-5-carboxylic acid methyl ester according to Method C in 80% yield using LiOH as base and methanol as a solvent.

3-Isopropylisoxazole-5-carboxylic acid pentafluorophenyl ester was prepared from 3-isopropylisoxazole-5-carboxylic acid according to Method A in 64% yield. NMR (300 MHz, CDCl₃): δ 7.09 (s, 1H), 3.16 (m, 1H), 1.33 (d, J=6.8 Hz, 6H).

Example 24 was prepared by reacting 3-isopropylisoxazole-5-carboxylic acid pentafluorophenyl ester with 4,10-diFmoc-deacylramoplanin amine according to Method B. HPLC: Rₜ=5.04 min (Condition 1); Rₜ=7.578 min (Condition 3). ESMS: m/z 1278.4 [(M+2H)/2].

### Example 25

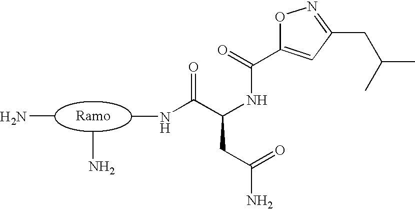

3-Methylbutyraldehyde oxime was prepared from 3-methylbutyraldehyde following Method J using pyridine as a base but without the use of a co-solvent.

3-Isobutylisoxazole-5-carboxylic acid methyl ester was prepared from 3-methylbutyraldehyde oxime and methyl propiolate following Method K in 76% yield after purification of the desired product by silica gel column chromatography using 9:1 hexane/ethyl acetate as an eluent.

3-Isobutylisoxazole-5-carboxylic acid was prepared from 3-isobutylisoxazole-5-carboxylic acid methyl ester according to Method C in quantitative yield using LiOH as base and methanol as a solvent.

3-Isobutylisoxazole-5-carboxylic acid pentafluorophenyl ester was prepared from 3-isobutylisoxazole-5-carboxylic acid according to Method A in 65% yield. NMR (300 MHz, CDCl₃): δ 7.04 (s, 1H), 2.64 (d, J=7.2 Hz, 2H), 2.01 (m, 1H), 0.98 (d, J=6.6 Hz, 6H).

Example 25 was prepared by reacting 3-isobutylisoxazole-5-carboxylic acid pentafluorophenyl ester with 4,10-diFmoc-deacylramoplanin amine according to Method B. HPLC: Rₜ=5.214 min (Condition 1), 7.953 min (Condition 3). ESMS: m/z 1285.4 [(M+2H)/2].

### Example 26

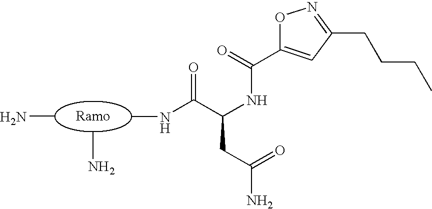

Pentanal oxime was prepared from Pentanal following Method J using pyridine as a base but without the use of a co-solvent.

3-Butylisoxazole-5-carboxylic acid methyl ester was prepared from pentanal oxime and methyl propiolate following Method K in 63% yield after purification of the desired product by silica gel column chromatography using 9:1 hexane/ethyl acetate as an eluent.

3-Butylisoxazole-5-carboxylic acid was prepared from 3-butylisoxazole-5-carboxylic acid methyl ester according to Method C in 94% yield using LiOH as base and methanol as a solvent.

3-Butylisoxazole-5-carboxylic acid pentafluorophenyl ester was prepared from 3-butylisoxazole-5-carboxylic acid according to Method A in 69% yield. NMR (300 MHz, CDCl₃): δ 7.06 (s, 1H), 2.77 (t, J=7.42 Hz, 2H), 1.68 (m, 2H), 1.39 (m, 2H), 0.93 (t, J=6.3 Hz, 3H)

Example 26 was prepared by reacting 3-butylisoxazole-5-carboxylic acid pentafluorophenyl ester with 4,10-diFmoc-deacylramoplanin amine according to Method B. HPLC: Rₜ=5.261 min (Condition 1), 8.077 min (Condition 3). ESMS: m/z 1285.4 [(M+2H)/2].

### Example 27

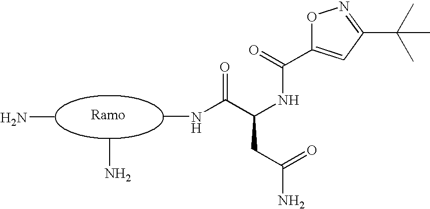

2,2-Dimethylpropionaldehyde oxime was prepared from 2,2-dimethylpropionaldehyde following Method J using pyridine as a base but without the use of a co-solvent.

3-t-Butylisoxazole-5-carboxylic acid methyl ester was prepared from 2,2-dimethylpropionaldehyde oxime and methyl propiolate following Method K in 71% yield after purification of the desired product by silica gel column chromatography using 9:1 hexane/ethyl acetate as an eluent.

3-t-Butylisoxazole-5-arboxylic acid was prepared from 3-t-butylisoxazole-5-carboxylic acid methyl ester according to Method C in 87% yield using LiOH as base and methanol as a solvent.

3-t-Butylisoxazole-5-carboxylic acid pentafluorophenyl ester was prepared from 3-t-butylisoxazole-5-carboxylic acid according to Method A in 68% yield. NMR (300 MHz, CDCl₃): δ 7.12 (s, 1H), 1.37 (s, 9H).

Example 27 was prepared by reacting 3-t-butylisoxazole-5-carboxylic acid pentafluorophenyl ester with 4,10-diFmoc-deacylramoplanin amine according to Method B. HPLC: Rₜ=5.199 min (Condition 1), 7.984 min (Condition 3). ESMS: m/z 1285.4 [(M+2H)/2].

### Example 28

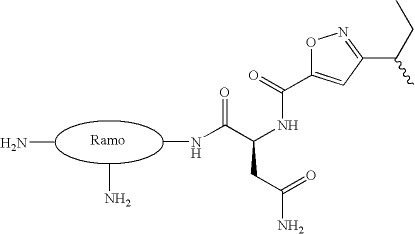

(±)-2-methylbutyraldehyde oxime was prepared from (±)-2-methylbutyraldehyde following Method J using pyridine as a base but without the use of a co-solvent.

(±)-3-sec-Butylisoxazole-5-arboxylic acid methyl ester was prepared from (±)-2-methylbutyraldehyde oxime and methyl propiolate following Method K in 70% yield after purification of the desired product by silica gel column chromatography using 9:1 hexane/ethyl acetate as an eluent.

(±)-3-sec-Butylisoxazole-5-carboxylic acid was prepared from (±)-3-sec-butylisoxazole-5-carboxylic acid methyl ester according to Method C in 97% yield using LiOH as base and methanol as a solvent.

(±)-3-sec-Butylisoxazole-5-carboxylic acid pentafluorophenyl ester was prepared from (±)-3-sec-butylisoxazole-5-carboxylic acid according to Method A in 79% yield. NMR (300 MHz, CDCl₃): δ 7.06 (s, 1H), 2.97 (m, 1H), 1.69 (m, 2H), 1.30 (d, J=7.14 Hz, 3H), 0.91 (t, J=7.41 Hz, 3H).

Example 28 was prepared by reacting 3-sec-butylisoxazole-5-carboxylic acid pentafluorophenyl ester with 4,10-diFmoc-deacylramoplanin amine according to Method B. HPLC: Rₜ=5.202 min (Condition 1), 7.959 min (Condition 3). ESMS: m/z 1285.4 [(M+2H)/2].

### Example 29

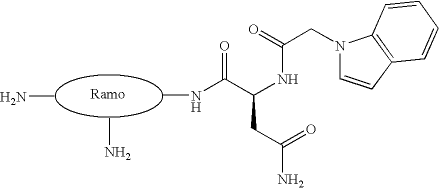

Indol-1-ylacetic acid tert-butyl ester was prepared from indole and tert-butyl bromoacetate following Method L in 62% yield after purifying the product by silica gel chromatography using hexane/ethyl acetate (9:1 mixture) as an eluent.

To a stirred solution of indol-1-ylacetic acid tert-butyl ester (0.46 g) in methanol (3 mL) was added solid potassium hydroxide (1 g) followed by water (0.1 mL). The reaction mixture was stirred at room temperature for 16 h, diluted with water (50 mL), extracted with ether, and the ether layer discarded. The aqueous layer was acidified to pH 3-4 using 6N hydrochloric acid, then extracted with ether. The combined organic layer was dried over MgSO₄, filtered and concentrated to produce Indol-1-ylacetic acid (0.3 g, 85% yield).

Indol-1-ylacetic acid pentafluorophenyl ester was prepared from indol-1-ylacetic acid according to Method A in 82% yield. NMR (300 MHz, CDCl₃): δ 7.63 (d, J=7.69 Hz, 1H), 7.11-7.27 (m, 4H), 6.59 (d, J=3.29 Hz, 1H), 5.19 (s, 2H).

Example 29 was prepared by reacting indol-1-ylacetic acid pentafluorophenyl ester with 4,10-diFmoc-deacylramoplanin amine according to Method B. HPLC: Rₜ=5.250 min (Condition 1), 8.131 min (Condition 3). ESMS: m/z 1287.8 [(M+2H)/2].

### Example 30

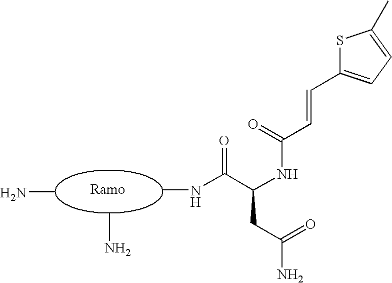

3-(5-Methylthiophen-2-yl)acrylic acid (E-isomer) was prepared from 5-methylthiophene-2-carboxaldehyde following Method H in 93% yield.

3-(5-Methylthiophen-2-yl)acrylic acid pentafluorophenyl ester (E-isomer) was prepared from 3-(5-Methylthiophen-2-yl)acrylic acid (E-isomer) according to Method A in 83% yield. ¹H NMR (300 MHz, CDCl₃): δ 7.91 (d, J=15.65 Hz, 1H), 7.5 (d, J=3.57 Hz, 1H), 76.75 (d, J=3.57 Hz, 1H), 6.23 (d, J=15.65 Hz, 1H), 1.52 (s, 3H).

Example 30 was prepared by reacting 3-(5-methylthiophen-2-yl)acrylic acid pentafluorophenyl ester (E-isomer) with 4,10-diFmoc-deacylramoplanin amine according to Method B. HPLC: Rₜ=5.333 min (Condition 1), 8.173 min (Condition 3). ESMS: m/z 1284.7 [(M+2H)/2].

### Example 31

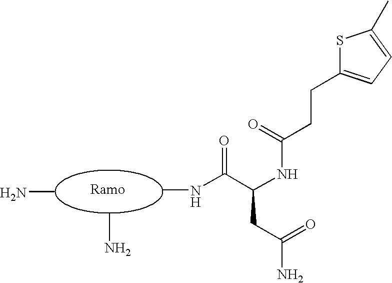

3-(5-Methylthiophen-2-yl)propionic acid was prepared from 3-(5-methylthiophen-2-yl)acrylic acid (as prepared in Example 30) following Method I in 93% yield.

3-(5-Methylthiophen-2-yl)propionic acid pentafluorophenyl ester was prepared from 3-(5-methylthiophen-2-yl)propionic acid according to Method A in 94% yield. ¹H NMR (300 MHz, CDCl₃): δ 6.61 (d, J=3.29 Hz, 1H), 6.54 (d, J=3.29 Hz, 1H), 3.16 (t, J=7.41 Hz, 2H), 2.97 (t, J=7.41 Hz, 2H), 2.41 (s, 3H).

Example 31 was prepared by reacting 3-(5-methylthiophen-2-yl)propionic acid pentafluorophenyl ester with 4,10-diFmoc-deacylramoplanin amine according to Method B. HPLC: Rₜ=5.328 min (Condition 1), 8.196 min (Condition 3). ESMS: m/z 1285.7 [(M+2H)/2].

### Example 32

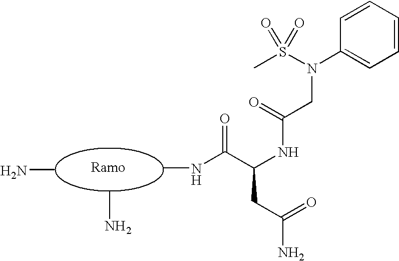

N-Phenylmethanesulfonamide was prepared from aniline and methanesulfonyl chloride following Method M in 95% yield (crude).

(Methanesulfonylphenylamino)acetic acid methyl ester was prepared by reacting N-phenylmethanesulfonamide with methyl bromoacetate following Method O in 53% yield after purification by column chromatography on silica gel using hexane/ethyl acetate as an eluent.

(Methanesulfonylphenylamino)acetic acid was prepared from (methanesulfonylphenylamino)acetic acid methyl ester following Method C using LiOH as base in 90% yield.

(Methanesulfonylphenylamino)acetic acid pentafluorophenyl ester was prepared from (methanesulfonylphenylamino)acetic acid according to Method A in 91% yield. NMR (300 MHz, CDCl₃): δ 7.23-7.48 (m, 5H), 4.81 (s, 2H), 3.07 (s, 3H).

Example 32 was prepared by reacting (methanesulfonylphenylamino)acetic acid pentafluorophenyl ester with 4,10-diFmoc-deacylramoplanin amine according to Method B. HPLC: Rₜ=5.005 min (Condition 1), 7.189 min (Condition 3). ESMS: m/z 1315.1 [(M+2H)/2].

### Example 33

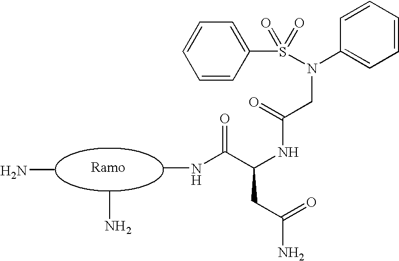

N-Phenylbenzenesulfonamide was prepared from aniline and benzenesulfonyl chloride following Method N in 90% yield (crude).

(Benzenesulfonylphenylamino)acetic acid methyl ester was prepared by reacting N-phenylbenzenesulfonamide with methyl bromoacetate following Method O in 65% yield after purification by column chromatography on silica gel using hexane/ethyl acetate (8:2) as an eluent.

(Benzenesulfonylphenylamino)acetic acid was prepared from (benzenesulfonylphenylamino)acetic acid methyl ester following Method C using LiOH as base in 97% yield.

(Benzenesulfonylphenylamino)acetic acid pentafluorophenyl ester was prepared from (benzenesulfonylphenylamino)acetic acid according to Method A in 67% yield. NMR (300 MHz, CDCl₃): δ 7.16-7.66 (m, 10H), 4.76 (s, 2H).

Example 33 was prepared by reacting (benzenesulfonylphenylamino)acetic acid pentafluorophenyl ester with 4,10-diFmoc-deacylramoplanin amine according to Method B. HPLC: Rₜ=5.498 min (Condition 1); Rₜ=8.425 min (Condition 3). ESMS: m/z 1346.3 [(M+2H)/2].

### Example 34

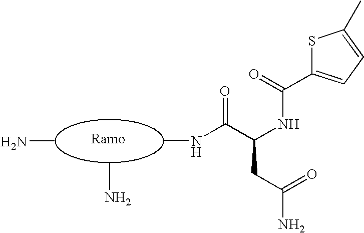

5-Methylthiophene-2-carboxylic acid pentafluorophenyl ester was prepared from 5-methylthiophene-2-carboxylic acid according to Method A in 81% yield. ¹H NMR (300 MHz, CDCl₃): δ 7.83 (d, J=3.57 Hz, 1H), 6.86 (d, J=3.02 Hz, 1H), 2.57 (s, 3H).

Example 34 was prepared by reacting 5-methylthiophene-2-carboxylic acid pentafluorophenyl ester with 4,10-diFmoc-deacylramoplanin amine according to Method B. HPLC: Rₜ=5.162 min (Condition 1); Rₜ=7.335 min (Condition 3). ESMS: m/z 1271 [(M+2H)/2].

### Example 35

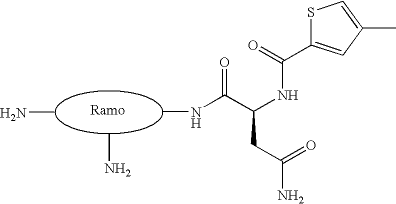

To a stirred solution of 3-methylthiophene (4.9 g, 50 mmol) in anhydrous THF (100 mL) at −78° C. was added n-BuLi (22 mL, 2.5 M solution in hexanes, 55 mmol) drop wise. After completion of addition the reaction mixture was stirred at −78° C. for an additional 1 h, then quenched with di-tert-butyldicarbonate (15 g). The reaction mixture was allowed to attain room temperature, concentrated in vacuo, and the residue was suspended in water. The aqueous layer was extracted with ether, the organic layer dried over MgSO₄, filtered and concentrated. The resultant residue was purified on silica gel column chromatography using 1% ethyl acetate in hexanes as an eluent to afford 4-methylthiophene-2-carboxylic acid tert-butyl ester (1.6 g, 16% yield, Higher R_{f} material) and 3-methylthiophene-2-carboxylic acid tert-butyl ester (1.5 g, 15% yield, Lower R_{f} material).

A solution of 4-methylthiophene-2-carboxylic acid tert-butyl ester (1.5 g) in 2M potassium hydroxide in methanol (15 mL) was stirred at 70° C. for 2 h. The reaction mixture was concentrated in vacuo and the residue was suspended in water. The aqueous layer was extracted with ether and the organic layer discarded. The aqueous layer was acidified to pH 3-4 with 6N hydrochloric acid, then extracted with 1:1 mixture of ethyl acetate and ether (200 mL). The organic layer was dried over MgSO₄ and concentrated to yield 4-methylthiophene-2-carboxylic acid (1.01 g, 93% yield).

4-Methylthiophene-2-carboxylic acid pentafluorophenyl ester was prepared from 4-methylthiophene-2-carboxylic acid according to Method A in 68% yield. ¹H NMR (300 MHz CDCl₃): δ 7.81 (d, J=1.09 Hz, 1H), 7.33 (d, J=1.09 Hz, 1H), 2.31 (s, 3H).

Example 35 was prepared by reacting 4-methylthiophene-2-carboxylic acid pentafluorophenyl ester with 4,10-diFmoc-deacylramoplanin amine according to Method B. HPLC: Rₜ=5.164 min (Condition 1); Rₜ=7.369 min (Condition 3). ESMS: m/z 1271.3 [(M+2H)/2].

### Example 36

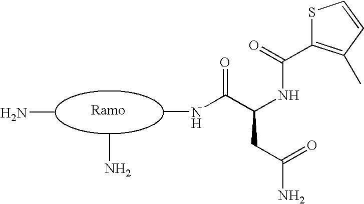

3-Methylthiophene-2-carboxylic acid pentafluorophenyl ester was prepared from 3-methylthiophene-2-carboxylic acid according to Method A in 87% yield. ¹H NMR (300 MHz, CDCl₃): δ 7.57 (d, J=4.94 Hz, 1H), 6.95 (d, J=4.95 Hz, 1H), 2.58 (s, 3H).

Example 36 was prepared by reacting 3-methylthiophene-2-carboxylic acid pentafluorophenyl ester with 4,10-diFmoc-deacylramoplanin amine according to Method B. HPLC: Rₜ=5.068 min (Condition 1); Rₜ=7.149 min (Condition 3). ESMS: m/z 1271 [(M+2H)/2].

### Example 37

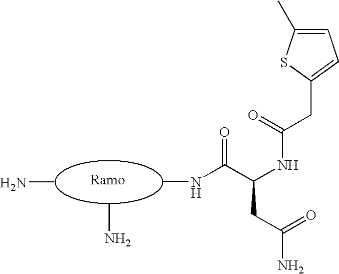

To a stirred solution of oxalyl chloride (2M solution in dichloromethane, 15 mL) was added 5-methylthiophene-2-carboxylic acid (2.8 g) in one lot followed by a drop of DMF. The reaction was continuously stirred at room temperature for 4 h and concentrated in vacuo. The residue was dissolved in toluene (20 mL) and the solvent removed to afford 5-methylthiophene-2-carbonyl chloride, which was used in the subsequent step without purification. To a stirred mixture of TMSCH₂N₂ (15 mL of 2M solution in hexanes) and triethylamine (3.5 mL) in THF (30 mL) and acetonitrile (30 mL) at 0° C. was added 5-methylthiophene-2-carbonyl chloride, which was stirred continuously at 0° C. for 30 h. The reaction mixture was concentrated in vacuo. Benzyl alcohol (10 mL) and 2,4,6-trimethylpyridine (10 mL) were added to the evaporated residue and the mixture was stirred at 180-185° C. for 10 minutes. The reaction mixture was cooled to room temperature, diluted with ether and washed successively with 10% aqueous citric acid, water and saturated aqueous sodium chloride. The organic layer was dried over MgSO₄ and concentrated in vacuo. The residue was dissolved in 15 mL of 2M solution potassium hydroxide in methanol, stirred at room temperature for 18 h and concentrated in vacuo. The residue was suspended in water and the aqueous layer extracted with ether, and the organic layer discarded. The aqueous layer was acidified to pH 34 with 6N hydrochloric acid, then extracted with ether to obtain (5-methylthiophen-2-yl)acetic acid (1.8 g, 62% yield).

(5-Methylthiophen-2-yl)acetic acid pentafluorophenyl ester was prepared from (5-methylthiophen-2-yl)acetic acid according to Method A in 72% yield. ¹H NMR (300 MHz, CDCl₃): δ 6.79 (d, J=3.29 Hz, 1H), 6.61 (d, J=3.29 Hz, 1H), 4.06 (s, 2H), 2.44 (s, 3H).

Example 37 was prepared by reacting (5-methylthiophen-2-yl)acetic acid pentafluorophenyl ester with 4,10-diFmoc-deacylramoplanin amine according to Method B. HPLC: Rₜ=5.258 min (Condition 1); Rₜ 7.56 min (Condition 3). ESMS: m/z 1278 [(M+2H)/2].

### Example 38

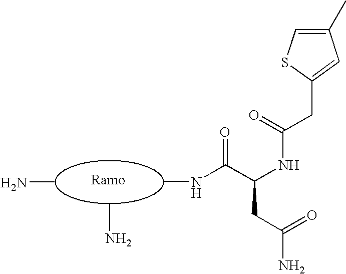

To a stirred solution of oxalyl chloride (2M solution in dichloromethane, 10 mL) was added 4-methylthiophene-2-carboxylic acid (1.4 g) in one lot followed by a drop of DMF. The reaction was continuously stirred at room temperature for 8 h and concentrated in vacuo. The residue was dissolved in toluene (20 mL) and the solvent removed to afford 4-methylthiophene-2-carbonyl chloride, which was used in the subsequent step without purification. To a stirred mixture of TMSCH₂N₂ (10 mL of 2M solution in hexanes) and triethylamine (2 mL) in THF (15 mL) and acetonitrile (15 mL) at 0° C. was added 4-methylthiophene-2-carbonyl chloride, which was continuously stirred at 0° C. for 30 h. The reaction mixture was concentrated in vacuo. Benzyl alcohol (5 mL) and 2,4,6-trimethylpyridine (5 mL) were added to the evaporated residue and the mixture was stirred at 180-185° C. for 10 minutes. The reaction mixture was cooled to room temperature, diluted with ether and washed successively with 10% aqueous citric acid, water and saturated aqueous sodium chloride. The organic layer was dried over MgSO₄ and concentrated in vacuo. The residue was dissolved in 15 mL of 2M solution potassium hydroxide in methanol, stirred at room temperature for 18 h and concentrated in vacuo. The residue was suspended in water and the aqueous layer extracted with ether, and the organic layer discarded. The aqueous layer was acidified to pH 3-4 with 6N hydrochloric acid, then extracted with ether to obtain (4-methylthiophen-2-yl)acetic acid (0.8 g).

(4-Methylthiophen-2-yl)acetic acid pentafluorophenyl ester was prepared from (4-methylthiophen-2-yl)acetic acid according to Method A in 17% yield. ¹H NMR (300 MHz, CDCl₃): δ 6.82 (m, 2H), 4.09 (s, 2H), 2.21 (s, 3H).

Example 38 was prepared by reacting (4-methylthiophen-2-yl)acetic acid pentafluorophenyl ester with 4,1-diFmoc-deacylramoplanin amine according to Method B. HPLC: Rₜ=5.24 min (Condition 1); Rₜ=7.549 min (Condition 3). ESMS: m/z 1278.7 [(M+2H)/2].

### Example 39

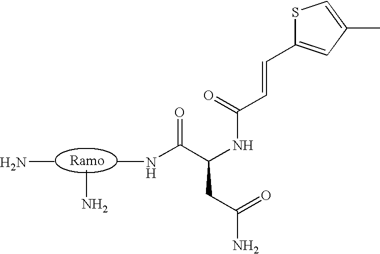

To a stirred solution of 3-methylthiophene (9.8 g, 100 mmol) in anhydrous THF (100 mL) at −78° C. was added n-BuLi (44 mL, 2.5 M solution in hexanes, 110 mmol) drop wise. After completion of addition, the reaction mixture was stirred at −78° C. for an additional 1 h, then quenched with DMF (20 mL). The reaction mixture was allowed to attain room temperature, concentrated in vacuo, and the residue was suspended in water. The aqueous layer was extracted with ether, the organic layer dried over MgSO₄, filtered and concentrated. The resultant residue was purified on silica gel column chromatography using 10% ethyl acetate in hexanes as an eluent to afford 4-methylthiophene-2-carboxaldehyde (major product) and 3-methylthiophene-2-carboxaldehyde (minor product) (6.5 g, 3:1 ratio). This product was used without further purification in the subsequent step.

3-(4-Methylthiophen-2-yl)acrylic acid (E-isomer) was prepared from 4-methylthiophene-2-carboxaldehyde following Method H in 62% yield. The product was purified by recrystallization using hexane/ethyl acetate mixture to afford 3-(4-methylthiophen-2-yl)acrylic acid (E-isomer).

3-(4-Methylthiophen-2-yl)acrylic acid pentafluorophenyl ester (E-isomer) was prepared from 3-(4-methylthiophen-2-yl)acrylic acid (E-isomer) according to Method A in 78% yield. ¹H NMR (300 MHz, CDCl₃): δ 7.94 (d, J=15.65 Hz, 1H), 7.15 (s, 1H), 7.05 (s, 1H), 6.34 (d, J=15.65 Hz, 1H), 2.25 (s, 3H).

Example 39 was prepared by reacting 3-(4-methylthiophen-2-yl)acrylic acid pentafluorophenyl ester (E-isomer) with 4,10-diFmoc-deacylramoplanin amine according to Method B. HPLC: Rₜ 5.424 min (Condition 1); Rₜ=7.885 min (Condition 3). ESMS: m/z 1284.7 [(M+2H)/2].

### Example 40

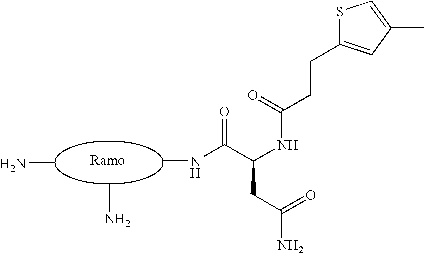

3-(4-Methylthiophen-2-yl)propionic acid was prepared from 3-(4-methylthiophen-2-yl)acrylic acid (Example 39) following Method I in quantitative yield.

3-(4-Methylthiophen-2-yl)propionic acid pentafluorophenyl ester was prepared from 3-(4-methylthiophen-2-yl)propionic acid according to Method A in 70% yield. ¹H NMR (300 MHz, CDCl₃): δ 6.7 (s, 1H), 6.66 (s, 1H), 3.19 (t, J=7.94 Hz, 2H), 2.97 (t, J=6.86 Hz, 2H), 2.18 (s, 3H).

Example 40 was prepared by reacting 3-(4-methylthiophen-2-yl)propionic acid pentafluorophenyl ester with 4,10-diFmoc-deacylramoplanin amine according to Method B. HPLC: Rₜ=5.423 min (Condition 1); Rₜ=7.948 min (Condition 3). ESMS: m/z 1286.1 [(M+2H)/2].

### Example 41

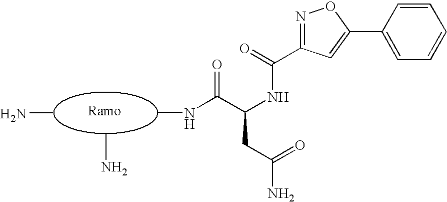

A mixture of nitroacetic acid ethyl ester (1.47 g), ethynylbenzene (1.02 g), and phenylisocyanate (2.4 g) in toluene were taken in a sealed tube. This was stirred at room temperature for 1 h followed by at 110° C. for 4 h. The reaction mixture was cooled to room temperature, diluted with ethyl acetate (50 mL) and the solid was filtered off. The filtrate was diluted with ether (250 mL) and washed with 1N sodium hydroxide (50 mL×3). The organic layer was dried over MgSO₄, filtered, concentrated in vacuo, and the residue was chromatographed on a silica gel column using hexane/ethyl acetate mixture (8:2) as an eluent to afford 5-phenylisoxazole-3-carboxylic acid ethyl ester (1.1 g, 51% yield).

5-Phenylisoxazole-3-carboxylic acid was prepared from 5-phenylisoxazole-3-carboxylic acid ethyl ester following Method C in 94% yield using potassium hydroxide as a base and methanol as a solvent.

5-Phenylisoxazole-3-carboxylic acid pentafluorophenyl ester was prepared from 5-phenylisoxazole-3-carboxylic acid according to Method A in 56% yield. ¹H NMR (300 MHz, CDCl₃): δ 7.82 (m, 2H), 7.50 (m, 3H), 7.06 (s, 1H).

Example 41 was prepared by reacting 5-phenylisoxazole-3-carboxylic acid pentafluorophenyl ester with 4,10-diFmoc-deacylramoplanin amine according to Method B. HPLC: Rₜ=5.478 min (Condition 1); Rₜ 7.948 min (Condition 3). ESMS: m/z 1294.8 [(M+2H)/2].

### Example 42

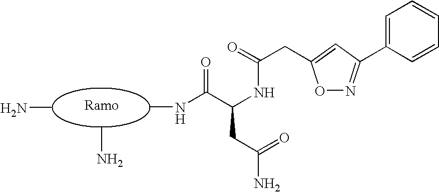

(3-Phenylisoxazol-5-yl)methanol was prepared from benzaldehyde oxime (Example 17, step 1) and propargyl alcohol following Method K. The reaction was initially conducted at 0° C. for 30 min. and then at room temperature for 4 h. The product was purified by silica gel column chromatography using ethyl acetate/hexanes (1:1) as an eluent to afford (3-phenylisoxazol-5-yl)methanol in 62% yield.

(3-Phenylisoxazol-5-yl)acetonitrile was prepared from (3-phenylisoxazol-5-yl)methanol following Method E then Method F in 28% yield after purification of product by silica gel column chromatography using hexane/ethyl acetate (8:2) as an eluent.

(3-Phenylisoxazol-5-yl)acetic acid pentafluorophenyl ester was prepared from (3-phenylisoxazol-5-yl)acetonitrile following Method G then Method A in 5% yield after purification of product by silica gel column chromatography using hexane/ethyl acetate (9:1) as an eluent. ¹H NMR (300 MHz, CDCl₃): δ 7.82 (m, 2H), 7.47 (m, 3H), 6.71 (s, 1H), 4.25 (s, 2H).

Example 42 was prepared by reacting (3-phenylisoxazol-5-yl)acetic acid pentafluorophenyl ester with 4,10-diFmoc-deacylramoplanin amine according to Method B. HPLC: Rₜ=5.436 min (Condition 1); Rₜ=7.747 min (Condition 3). ESMS: m/z 1301.8 [(M+2H)/2].

### Example 43

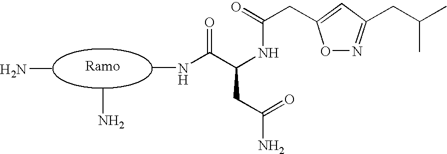

(3-Isobutylisoxazol-5-yl)methanol was prepared from 3-methylbutyraldehyde oxime (Example 25, Step 1) and propargyl alcohol following Method K. The reaction was initially conducted at 0° C. for 30 min. and then at room temperature for 18 h. The product was purified by silica gel column chromatography using ethyl acetate/hexanes (1:1) as an eluent to afford (3-isobutylisoxazol-5-yl)methanol in 34% yield.

(3-Isobutylisoxazol-5-yl)acetonitrile was prepared from (3-isobutylisoxazol-5-yl)methanol following Method E then Method F in 42% yield after purification of product by silica gel column chromatography using hexane/ethyl acetate (8:2) as an eluent.

(3-Isobutylisoxazol-5-yl)acetic acid was prepared from (3-isobutylisoxazol-5-yl)acetonitrile following Method G in 51% yield (crude product).

(3-Isobutylisoxazol-5-yl)acetic acid pentafluorophenyl ester was prepared from (3-isobutylisoxazol-5-yl)acetic acid following Method A in 15% yield. ¹H NMR (300 MHz, CDCl₃): δ 6.18 (s, 1H), 4.13 (s, 2H), 2.52 (d, J=7.14 Hz, 2H), 1.92 (m, 1H), 0.93 (d, J=6.59 Hz, 6H).

Example 43 was prepared by reacting (3-isobutylisoxazol-5-yl)acetic acid pentafluorophenyl ester with 4,10-diFmoc-deacylramoplanin amine according to Method B. HPLC: Rₜ=4.916 min (Condition 1); Rₜ=7.583 min (Condition 3). ESMS: m/z 1292 [(M+2H)/2].

### Example 44

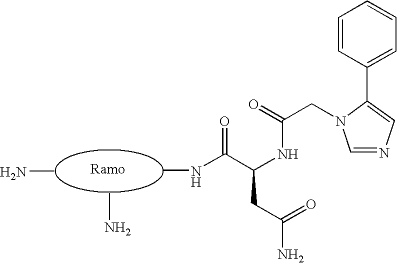

To a stirred solution of 5-phenyl-1H-imidazole (2.88 g, 20 mmol) in DMF (25 mL) was added benzyl bromoacetate (4.60 g) followed by solid potassium carbonate (5 g). The reaction was kept stirring at 75° C. for 4 h. This was cooled to room temperature, diluted with water and extracted, with ether. The combined organic layer was washed with water, dried over MgSO₄, filtered and concentrated in vacuo. The residue was chromatographed on silica gel using ethyl acetate/hexanes (8:2) to afford (5-phenylimidazol-1-yl)acetic acid benzyl ester (3.7 g, 63% yield).

To a stirred solution of (5-phenylimidazol-1-yl)acetic acid benzyl ester (1.5 g, 5.63 mmol) in methanol (25 mL) was added 10% palladium on carbon (300 mg). The resultant reaction mixture was subjected to hydrogenation using a balloon full of hydrogen at room temperature for 8 h. This was filtered through a pad of Celite, the Celite pad was washed with methanol, and the combined filtrate was concentrated in vacuo to obtain (5-phenylimidazol-1-yl)acetic acid (0.7 g, 61% yield). NMR (300 MHz, CD₃OD): δ 8.43 (s, 1H), 7.71-7.68 (m, 3H), 7.32-7.46 (m, 3H), 4.88 (s, 2H).

To a stirred solution of (5-phenylimidazol-1-yl)acetic acid (40.4 mg) in DMF (0.5 mL) was added EDC (39 mg), and the resultant mixture was stirred at room temperature for 30 min. An aliquot (100 μL) of this reaction mixture was added to a solution of 4,10-diFmoc-deacylramoplanin amine (30 mg) in DMF (300 μL). This was stirred at room temperature for 8 h, the reaction mixture was diluted with water (3 mL), and then the precipitated solid was filtered off and dried under high vacuum (25 mg). This was dissolved in DMF (300 μL), piperidine was added (15 μL), and the mixture was stirred for 10 min. The reaction mixture was diluted with acetonitrile (300 μL) followed by addition of 0.5 N hydrochloric acid (600 μL). The product was purified by preparative HPLC to obtain Example 44. HPLC: Rₜ=4.709 min (Condition 1); Rₜ=6.664 min (Condition 3). ESMS: m/z 1301.8 [(M+2H)/2].

### Example 45

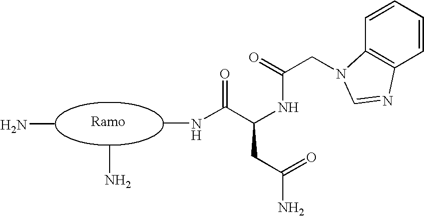

Benzimidazol-1-ylacetic acid benzyl ester was prepared from 1H-benzimidazole and benzyl bromoacetate according to Method L in 39% yield after purifying the product by silica gel column chromatography using hexane/ethyl acetate (1:1) as an eluent.

To a stirred solution of benzimidazol-1-ylacetic acid benzyl ester (1.25 g, 4.69 mmol) in methanol (30 mL) was added 10% palladium on carbon (130 mg). The resultant reaction mixture was subjected to hydrogenation using a balloon full of hydrogen at room temperature for 14 h. This was filtered through a pad of Celite, the Celite pad was washed with excess methanol, and the combined filtrate was concentrated to get benzimidazol-1-ylacetic acid (0.4 g, 48% yield). NMR (300 MHz, DMSO-d₆): δ 8.18 (s, 1H), 7.65 (d, J=8.51 Hz, 1H), 7.52 (d, J=8.24 Hz, 1H), 7.17-7.27 (m, 2H), 5.13 (s, 2H).

To a stirred solution of benzimidazol-1-ylacetic acid (35 mg) in DMF (0.5 mL) was added EDC (39 mg), and the resultant mixture was stirred at room temperature for 30 min. An aliquot (50 μL) of this reaction mixture was added to a solution of 4,10-diFmoc-deacylramoplanin amine (20 mg) in DMF (300 μL). This was stirred at room temperature for 8 h, the reaction mixture was diluted with water (3 mL), and then the precipitated solid was filtered off and dried under high vacuum (15 mg). This was dissolved in DMF (300 μL) and piperidine was added (15 μL), and the mixture was stirred for 12 min. The reaction mixture was diluted with acetonitrile (500 μL) followed by addition of 0.5 N hydrochloric acid (600 μL). The product was purified by preparative HPLC to obtain Example 45. HPLC: Rₜ=4.523 min (Condition 1); Rₜ=5.888 min (Condition 3). ESMS: m/z 1288.5 [(M+2H)/2].

### Example 46

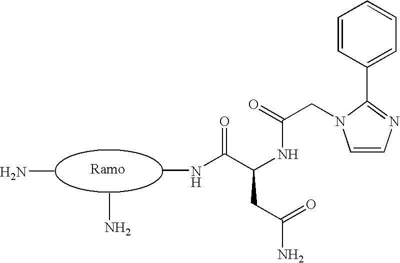

To a stirred solution of 2-phenyl-1H-imidazole (2.88 g, 20 mmol) in DMF (25 mL) was added benzyl bromoacetate (4.60 g) followed by solid potassium carbonate (5 g). The reaction was kept stirring at 75° C. for 4 h. This was cooled to room temperature, diluted with water and extracted with ether. The combined organic layer was washed with water, dried over MgSO₄, filtered and concentrated. The residue was chromatographed on silica gel using ethyl acetate/hexanes (8:2) to afford (2-phenylimidazol-1-yl)acetic acid benzyl ester 2.8 g, 48% yield).

To a stirred solution of (2-phenylimidazol-1-yl)acetic acid benzyl ester (1.2 g, 4.1 mmol) in methanol (30 mL) was added 10% palladium on carbon (130 mg). The resultant reaction mixture was subjected to hydrogenation using a balloon full of hydrogen at room temperature for 14 h. This was filtered through a pad of Celite, the Celite pad was washed with methanol, and the combined filtrate was concentrated to get (2-phenylimidazol-1-yl)acetic acid (0.56 g, 67% yield). NMR (300 MHz, DMSO-d₆): δ 7.42-7.55 (m, 5H), 7.29 (, J=1.37 Hz, 1H), 7.01 (d, J=1.37 Hz, 1H), 4.85 (s, 2H).

To a stirred solution of (2-phenylimidazol-1-yl)acetic acid (40 mg) in DMF (0.5 mL) was added EDC (40 mg), and the resultant mixture was stirred at room temperature for 30 min. An aliquot (50 μL) of this reaction mixture was added to a solution of 4,10-diFmoc-deacylramoplanin amine (20 mg) in DMF (200 μL). This was stirred at room temperature for 14 h, the reaction mixture was diluted with water (3 mL), and then the precipitated solid was filtered off and dried under high vacuum (25 mg). This was dissolved in DMF (300 μL), piperidine was added (15 μL), and the mixture was stirred for 10 min. The reaction mixture was diluted with acetonitrile (300 μL) followed by addition of 0.5 N hydrochloric acid (600 μL). The product was purified by preparative HPLC to obtain Example 46. HPLC: Rₜ=4.494 min (Condition 1); Rₜ=5.616 min (Condition 3). ESMS: m/z 1301.4 [(M+2H)/2].

### Example 47

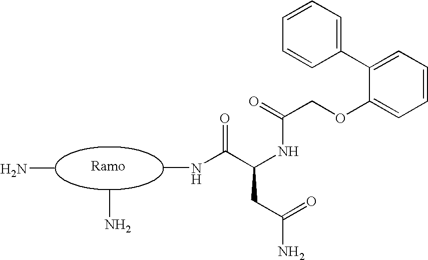

(Biphenyl-2-yloxy)acetic acid benzyl ester was prepared from biphenyl-2-ol and bromo benzylacetate according to Method P in 91% yield.

(Biphenyl-2-yloxy)acetic acid was prepared from (biphenyl-2-yloxy)acetic acid benzyl ester according to Method Q in 77% yield.

(Biphenyl-2-yloxy)acetic acid pentafluorophenyl ester was prepared from (biphenyl-2-yloxy)acetic acid according to Method A in 87% yield. ¹H NMR (300 MHz, CDCl₃): δ 7.23-7.57 (m, 7H), 7.11 (m, 1H), 6.92 (t, J=7.96 Hz, 1H), 4.91 (s, 2H).

Example 47 was prepared by reacting (biphenyl-2-yloxy)acetic acid pentafluorophenyl ester with 4,10-diFmoc-deacylramoplanin amine according to Method B. HPLC: Rₜ=5.634 min (Condition 1); Rₜ=8.655 min (Condition 3). ESMS: m/z 1314.4 [(M+2H)/2].

### Example 48

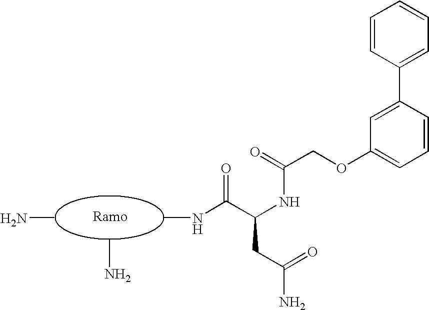

(Biphenyl-3-yloxy)acetic acid benzyl ester was prepared from biphenyl-3-ol and bromo benzylacetate according to Method P in 85% yield.

(Biphenyl-3-yloxy)acetic acid was prepared from (biphenyl-3-yloxy)acetic acid benzyl ester according to Method Q in 87% yield.

(Biphenyl-3-yloxy)acetic acid pentafluorophenyl ester was prepared from (biphenyl-3-yloxy)acetic acid according to Method A in 91% yield. ¹H NMR (300 MHz, CDCl₃): δ 7.02-7.56 (m, 8H), 6.92 (m, 1H), 5.03 (s, 2H).

Example 48 was prepared by reacting (biphenyl-3-yloxy)acetic acid pentafluorophenyl ester with 4,10-diFmoc-deacylramoplanin amine according to Method B. HPLC: Rₜ 5.541 min (Condition 1); Rₜ 8.871 min (Condition 3). ESMS: m/z 1314.4 [(M+2H)/2].

### Example 49

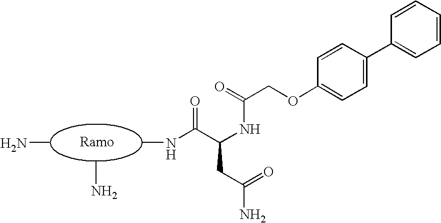

(Biphenyl-4-yloxy)acetic acid benzyl ester was prepared from biphenyl-4-ol and bromo benzylacetate according to Method P in 81% yield.

(Biphenyl-4-yloxy)acetic acid was prepared from (biphenyl-4-yloxy)acetic acid benzyl ester according to Method Q in 80% yield.

(Biphenylyloxy)acetic acid pentafluorophenyl ester was prepared from (biphenyl-4-yloxy)acetic acid according to Method A in 90% yield. ¹H NMR (300 MHz, CDCl₃): δ 7.26-7.56 (m, 7H), 7.02 (m, 2H), 5.01 (s, 2H).

Example 49 was prepared by reacting (biphenyl-4-yloxy)acetic acid pentafluorophenyl ester with 4,10-diFmoc-deacylramoplanin amine according to Method B. HPLC: Rₜ=5.554 min (Condition 1); Rₜ=8.905 min (Condition 3). ESMS: m/z 1314.7 [(M+2H)/2].

### Example 50

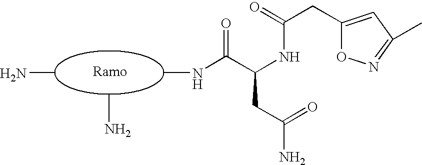

(3-Methyl-isoxazol-5-yl)acetic acid pentafluorophenyl ester was prepared from (3-methyl-isoxazol-5-yl)acetic acid according to Method A in 68% yield. ¹H NMR (300 MHz, CDCl₃) δ 6.43 (s, 1H), 4.38 (s, 2H), 2.46 (s, 3H).

4,10-diFmoc-deacylramoplanin amine was reacted with (3-methyl-isoxazol-5-yl)acetic acid pentafluorophenyl ester according to Method B to obtain Example 50. HPLC: Rₜ=4.37 min (Condition 1). ESMS: m/z 1271.0 [(M+2H)/2].

### Example 51

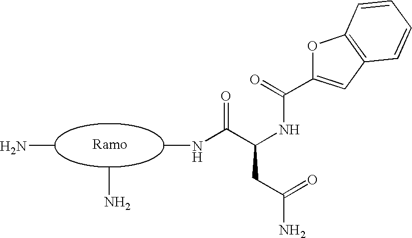

Benzofuran-2-carboxylic acid pentafluorophenyl ester was prepared from benzofuran-2-carboxylic acid according to Method A in 98% yield. ¹H NMR (300 MHz, CDCl₃) δ 7.86 (s, 1H), 7.78 (d, J=7.8 Hz, 1H), 7.65 (d, J=7.2 Hz, 1H), 7.56 (t, J=7.2 Hz, 1H), 7.39 (t, J=7.2 Hz, 1H).

4,10-diFmoc-deacylramoplanin amine was reacted with benzofuran-2-carboxylic acid pentafluorophenyl ester according to Method B to obtain Example 51. HPLC: Rₜ=4.96 min (Condition 1). ESMS: m/z 1282.2 [(M+2H)/2].

### Example 52

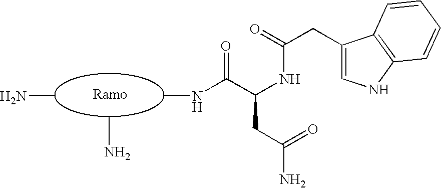

(1H-Indol-3-yl)acetic acid pentafluorophenyl ester was prepared from (1H-indol-3-yl)acetic acid according to Method A in 98% yield. ¹H NMR (300 MHz, CDCl₃) δ 8.17 (bs, 1H), 7.64 (d, J=7.8 Hz, 1H), 7.39 (d, J=7.2 Hz, 1H), 7.15-7.26 (m, 3H), 4.14 (s, 2H).

4,10-diFmoc-deacylramoplanin amine was reacted with (1H-indol-3-yl)acetic acid pentafluorophenyl ester according to Method B to obtain Example 52. HPLC: Rₜ=4.88 min (Condition 1). ESMS: m/z 1288.5 [(M+2H)/2].

### Example 53

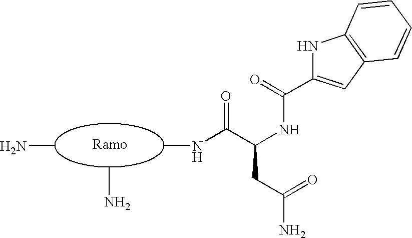

1H-Indole-2-carboxylic acid pentafluorophenyl ester was prepared from 1H-indole-2-carboxylic acid according to Method A in 80% yield. ¹H NMR (300 MHz, CDCl₃) δ 8.98 (bs, 1H), 7.77 (d, J=8.4 Hz, 1H), 7.49 (s, 1H), 7.27-7.49 (m, 3H).

4,10-diFmoc-deacylramoplanin amine was reacted with 1H-indole-2-carboxylic acid pentafluorophenyl ester according to Method B to obtain Example 53. HPLC: Rₜ=5.08 min (condition 1). ESMS: m/z 1281.8 [(M+2H)/2].

### Example 54

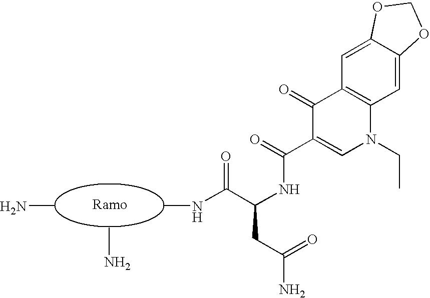

Oxolinic acid pentafluorophenyl ester was prepared from oxolinic acid according to Method A in 49% yield. ¹H NMR (300 MHz, CDCl₃) δ 8.57 (s, 1H), 7.93 (s, 1H), 6.92 (s, 1H), 6.15 (s, 2H), 4.25 (q, J=7.5 Hz, 2H), 1.57 (t, J=7.5 Hz, 3H).

4,10-diFmoc-deacylramoplanin amine was reacted with oxolinic acid pentafluorophenyl ester according to Method B to obtain Example 54. HPLC: Rₜ=5.17 min (Condition 1). ESMS: m/z 1331.2 [(M+2H)/2].

### Example 55

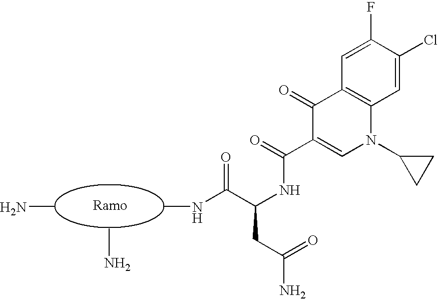

7-chloro-1-cyclopropyl-6-fluoro-4-oxohydroquinoline-3-carboxylic acid pentafluorophenyl ester was prepared from 7-chloro-1-cyclopropyl-6-fluoro-4-oxohydroquinoline-3-carboxylic acid according to Method A in 98% yield. ¹H NMR (300 MHz, CDCl₃) δ 8.75 (s, 1H), 8.24 (d, J=9.0 Hz, 1H), 8.06 (d, J=6.0 Hz, 1H), 3.57-3.52 (m, 1H), 1.48-1.41 (m, 2H), 1.28-1.19 (m, 2H).

4,10-diFmoc-deacylramoplanin amine was reacted with 7-chloro-1-cyclopropyl-6-fluoro-4-oxohydroquinoline-3-carboxylic acid pentafluorophenyl ester according to Method B to obtain Example 55. HPLC: Rₜ=5.67 min (condition 1). ESMS: m/z 1341.0 [(M+2H)/2].

### Example 56

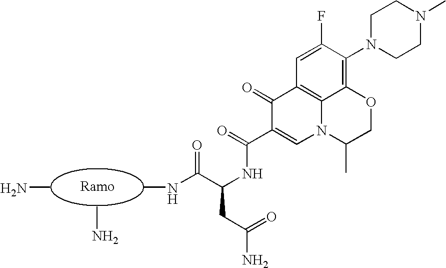

8-Fluoro-3-methyl-9-(4-methyl-piperazin-1-yl)-2,3-dihydro-1-oxa-3a-aza-phenalen-6-one-5-carboxylic acid pentafluorophenyl ester was prepared from 8-Fluoro-3-methyl-9-(4-methyl-piperazin-1-yl)-2,3-dihydro-1-oxa-3a-aza-phenalen-6-one-5-carboxylic acid according to Method A.

4,10-diFmoc-deacylramoplanin amine was reacted with 8-Fluoro-3-methyl-9-(4 methyl-piperazin-1-yl)-2,3-dihydro-1-oxa-3a-aza-phenalen-6-one-5-carboxylic acid pentafluorophenyl ester according to Method B to obtain Example 56.

### Example 57

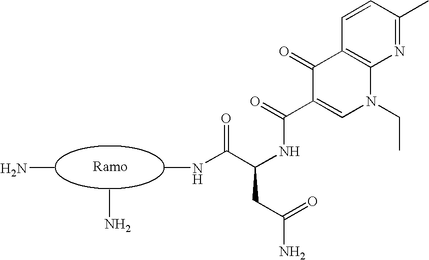

Nalidixic acid pentafluorophenyl ester was prepared from nalidixic acid according to Method A in 99% yield. ¹H NMR (300 MHz, CDCl₃) δ 8.80 (s, 1H), 8.68 (d, J=8.1 Hz, 1H), 7.32 (d, J=8.4 Hz, 1H), 4.55 (q, J=7.2 Hz, 2H), 2.70 (s, 3H), 1.56 (t, J=7.2 Hz, 3H).

4,10-diFmoc-deacylramoplanin amine was reacted with nalidixic acid pentafluorophenyl ester according to Method B to obtain Example 57. HPLC: Rₜ=5.45 min (condition 1). ESMS: m/z 1316.5 [(M+2H)/2].

### Example 58

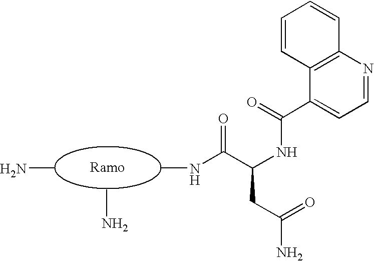

4-Quinolinecarboxylic acid pentafluorophenyl ester was prepared from 4-quiolinecarboxylic acid according to Method A in 99% yield. ¹H NMR (300 MHz, CDCl₃) δ 9.15 (d, J=4.5 Hz, 1H), 8.79 (d, J=8.4 Hz, 1H), 8.24 (d, J=8.4 Hz, 1H), 8.21 (d, J=4.5 Hz, 1H), 7.86 (t, J=6.9 Hz, 1H), 7.75 (t, J=6.9 Hz, 1H).

4,10-diFmoc-deacylramoplanin amine was reacted with 4-quinolinecarboxylic acid pentafluorophenyl ester according to Method B to obtain Example 58. HPLC: P=4.90 min (Condition 1). ESMS: m/z 1287.1 [(M+2H)/2].

### Example 59

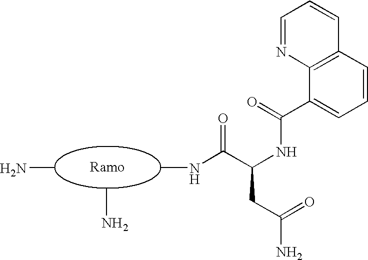

8-Quinolinecarboxylic acid pentafluorophenyl ester was prepared from 8-quinolinecarboxylic acid according to Method A in 99% yield. ¹H NMR (300 MHz, CDCl₃) δ 9.16-9.13 (m, 1H), 8.46 (d, J=7.5 Hz, 1H), 8.28 (d, J=8.4 Hz, 1H), 8.12 (d, J=8.1 Hz, 1H), 7.69 (t, J=8.1 Hz, 1H), 7.53-7.57 (m 1H).

4,10-diFmoc-deacylramoplanin amine was reacted with 8-quinolinecarboxylic acid pentafluorophenyl ester according to Method B to obtain Example 59. HPLC: Rₜ=5.16 min (Condition 1). ESMS: m/z 1287.4 [(M+2H)/2].

### Example 60

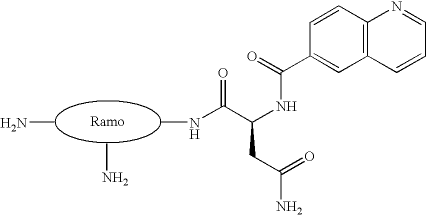

6-Quinolinecarboxylic acid pentafluorophenyl ester was prepared from 6-quinolinecarboxylic acid according to Method A in 99% yield. ¹H NMR (300 MHz, CDCl₃) δ 9.08 (m, 1H), 8.81 (s, 1H), 8.25-8.44 (m, 3H), 7.54-7.59 (m, 1H).

4,10-diFmoc-deacylramoplanin amine was reacted with 6-quinolinecarboxylic acid pentafluorophenyl ester according to Method B to obtain Example 60. HPLC: Rₜ=4.89 min (Condition 1). ESMS: m/z 1287.4 [(M+2H)/2].

### Example 61

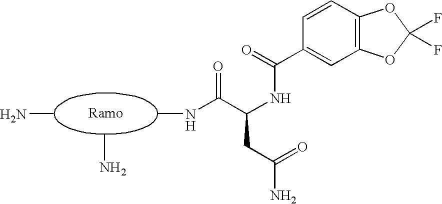

2,2-Difluoro-1,3-benzodioxole-5-carboxylic acid pentafluorophenyl ester was prepared from 2,2-difluoro-1,3-benzodioxole-5-carboxylic acid according to Method A in 99% yield. ¹H NMR (300 MHz, CDCl₃) δ 8.06 (d, J=8.4 Hz, 1H), 7.89 (s, 1H), 7.25 (d, J=8.4 Hz, 1H).

4,10-diFmoc-deacylramoplanin amine was reacted with 2,2-difluoro-1,3-benzodioxole-5-carboxylic acid pentafluorophenyl ester according to Method B to obtain Example 61. HPLC: Rₜ=5.53 min (Condition 1). ESMS: m/z 1310.9 [(M+2H)/2].

### Example 62

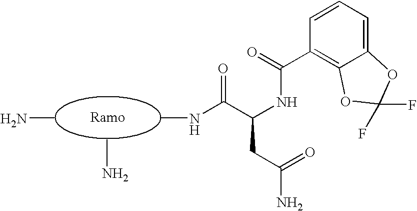

2,2-Difluoro-1,3-benzodioxole-4-carboxylic acid pentafluorophenyl ester was prepared from 2,2-difluoro-1,3-benzodioxole-4-carboxylic acid according to Method A in 99% yield. ¹H NMR (300 MHz, CDCl₃) δ 7.83 (d, J=8.1 Hz, 1H), 7.39 (d, J=8.4 Hz, 1H), 7.26 (t, J=8.4 Hz, 1H).

4,10-diFmoc-deacylramoplanin amine was reacted with 2,2-difluoro-1,3-benzodioxole-4-carboxylic acid pentafluorophenyl ester according to Method B to obtain Example 62. HPLC: Rₜ=5.38 min (Condition 1). ESMS: m/z 1310.6 [(M+2H)/2].

### Example 63

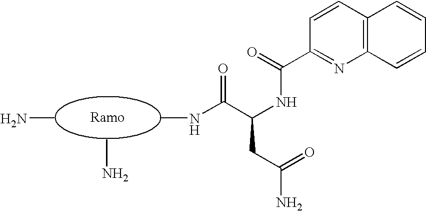

Quinoline-2-carboxylic acid pentafluorophenyl ester was prepared from quinoline-2-carboxylic acid according to Method A in 89% yield. ¹H NMR (300 MHz, CDCl₃) δ 8.5-8.64 (m, 3H), 8.05-8.18 (m, 2H), 7.94 (t, J=6.9 Hz, 1H).

4,10-diFmoc-deacylramoplanin amine was reacted with quinoline-2-arboxylic acid pentafluorophenyl ester according to Method B to obtain Example 63. HPLC: Rₜ=5.38 min (Condition 1). ESMS: m/z 1296.9 [(M+Na)/2].

### Example 64

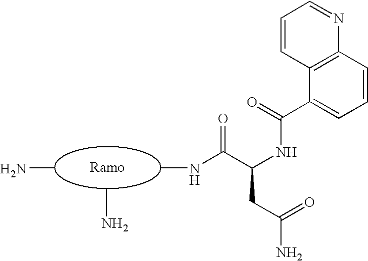

5-Quinolinecarboxylic acid pentafluorophenyl ester was prepared from 5-quinolinecarboxylic acid according to Method A in 84% yield. ¹H NMR (300 MHz, CDCl₃): δ 9.36 (d, J=8.1 Hz, 1H), 9.02-9.03 (m, 1H), 8.64 (d, J=7.5 Hz, 1H), 8.47 (d, J=8.4 Hz, 1H), 7.88 (t, J=7.5 Hz, 1H), 7.61-7.65 (m, 1H).

4,10-diFmoc-deacylramoplanin amine was reacted with 5-quinolinecarboxylic acid pentafluorophenyl ester according to Method B to obtain Example 64. HPLC: Rₜ=4.85 min (Condition 1). ESMS: m/z 1287.4 [(M+2H)/2].

### Example 65

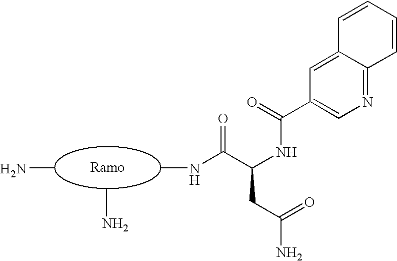

Quinoline-3-carboxylic acid pentafluorophenyl ester was prepared from quinoline-3-carboxylic acid according to Method A in 87% yield. ¹H NMR (300 MHz, CDCl₃): δ 9.55 (s, 1H), 9.12 (s, 1H), 8.28 (d, J=8.4 Hz, 1H), 8.05 (d, J=8.4 Hz, 1H), 7.95 (t, J=8.4 Hz, 1H), 7.75 (t, J=7.8 Hz, 1H).

4,10-diFmoc-deacylramoplanin amine was reacted with quinoline-3-carboxylic acid pentafluorophenyl ester according to Method B to obtain Example 65. HPLC: Rₜ=5.14 min (Condition 1). ESMS: m/z 1287.1 [(M+2H)/2].

### Example 66

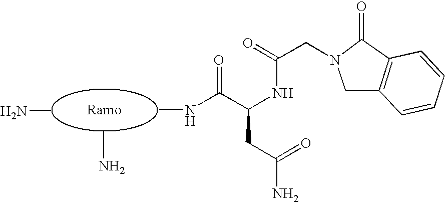

(1-Oxo-1,3-dihydroisoindol-2-yl)acetic acid pentafluorophenyl ester was prepared from (1-oxo-1,3-dihydroisoindol-2-yl)acetic acid according to Method A in 89% yield. ¹H NMR (300 MHz, CDCl₃): δ 7.91 (d, J=7.8 Hz, 1H), 7.48-7.63 (m, 3H), 4.80 (s, 2H), 4.60 (s, 2H).

4,10-diFmoc-deacylramoplanin amine was reacted with (1-oxo-1,3-dihydroisoindol-2-yl)acetic acid pentafluorophenyl ester according to Method B to obtain Example 66. HPLC: Rₜ=4.97 min (Condition 1). ESMS: m/z 1296.2 [(M+2H)/2].

### Example 67

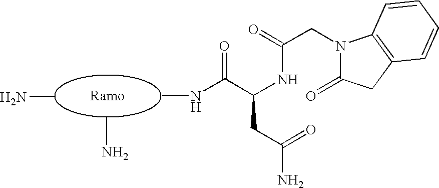

To a stirred solution of oxyindole (200 mg, 1.50 mmol) in DMF (5 mL) at 0° C. was added NaH (60% in mineral oil, 72 mg, 1.80 mmol) and the resulting solution was stirred for 5 min. The reaction was warmed to room temperature and tert-butyl bromoacetate (413 μL, 2.80 mmol) was added to the reaction mixture, while stirring. After 17 h, the reaction mixture was diluted with ethyl acetate (100 mL) and washed with 1.0 N HCl (100 mL), saturated aqueous NaHCO3 (100 mL), then brine (100 mL). The organic layer was dried (MgSO₄), concentrated in vacuo and the residue was purified by silica gel column chromatography (5-50% EtOAc in hexanes) to yield (2-oxo-2,3-dihydroindol-1-yl)acetic acid tert-butyl ester (259 mg, 70%).

To a portion of (2-oxo-2,3-dihydroindol-1-yl)acetic acid tert-butyl ester (50 mg, 0.20 mmol) was added 30% TFA in methylene chloride (7 mL) and the reaction mixture was stirred for 5 h. This was concentrated in vacuo to yield (2-oxo-2,3-dihydroindol-1-yl)acetic acid (79 mg TFA salt, 100%).

(2-Oxo-2,3-dihydroindol-1-yl)acetic acid pentafluorophenyl ester was prepared from (2-oxo-2,3-dihydroindol-1-yl)acetic acid according to Method A in 95% yield. ¹H NMR (300 MHz, CDCl₃) δ 7.29 (m, 2H), 7.11 (t, J=6.9 Hz, 1H), 6.77 (d, J=8.4 Hz, 1H), 4.86 (s, 2H), 3.66 (s, 2H).

4,10-diFmoc-deacylramoplanin amine was reacted with (2-oxo-2,3-dihydroindol-1-yl)acetic acid pentafluorophenyl ester according to Method B to obtain Example 67. HPLC: Rₜ=5.03 min (Condition 1). ESMS: m/z 1296.2 [(M+2H)/2].

### Example 68

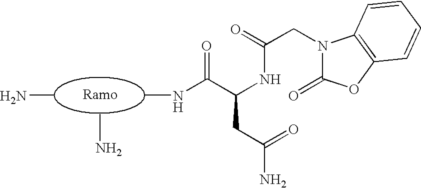

To a stirred solution of benzoxazolinone (405 mg, 3.0 mmol) in DMF (5 mL) at 0° C. was added NaH (60% in mineral oil, 144 mg, 3.6 mmol) and the resulting solution was stirred for 5 min. The reaction was warmed to room temperature and tert-butyl bromoacetate (856 μL, 5.80 mmol) was added to the reaction mixture, while stirring. After 17 h, the reaction mixture was diluted with ethyl acetate (100 mL) and washed with 1.0 N HCl (100 mL), saturated aqueous NaHCO₃ (100 mL), then brine (100 mL). The organic layer was dried (MgSO₄), concentrated in vacuo and the residue was purified by silica gel column chromatography (5-50% EtOAc in hexanes) to yield (2-oxobenzooxazol-3-yl)acetic acid tert-butyl ester (657 mg, 88%).

To a portion of (2-oxobenzooxazol-3-yl)acetic acid tert-butyl ester (250 mg, 1.0 mmol) was added 30% TFA in methylene chloride (12 mL) and the reaction mixture was stirred for 5 h. The reaction mixture was concentrated in vacuo to yield (2-oxobenzooxazol-3-yl)acetic acid (193 mg, 100%).

(2-Oxobenzooxazole-3-yl)acetic acid pentafluorophenyl ester was prepared from (2-oxobenzooxazole-3-yl)acetic acid according to Method A in 95% yield. ¹H NMR (300 MHz, CDCl₃) δ 7.16-7.29 (m, 3H), 6.96 (d, J=8.4 Hz, 1H), 4.97 (s, 2H).

4,10-diFmoc-deacylramoplanin amine was reacted with (2-oxobenzooxazole-3-yl)acetic acid pentafluorophenyl ester according to Method B to obtain Example 68. HPLC: Rₜ=5.19 min (Condition 1). ESMS: m/z 1297.3 [(M+2H)/2].

### Example 69

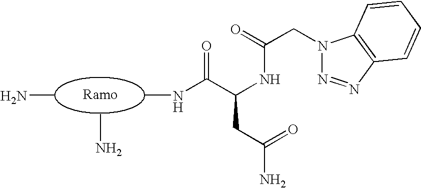

Benzotriazol-1-ylacetic acid tert-butyl ester was prepared from benzotriazole and tert-butyl bromoacetate following Method L in 17% yield after purifying the product by silica gel chromatography using hexane/ethyl acetate (6:4) as an eluent.

To a portion of benzotriazol-1-ylacetic acid tert-butyl ester (300 mg) was added 50% TFA in methylene chloride (20 mL) and the reaction mixture was stirred for 8 h. The reaction mixture was concentrated in vacuo to yield benzotriazol-1-ylacetic acid (220 mg, 96%).

Benzotriazol-1-ylacetic acid pentafluorophenyl ester was prepared from Benzotriazol-1-ylacetic acid according to Method A in 88% yield. ¹H NMR (300 MHz, CDCl₃) δ 8.09 (d, J=8.7 Hz, 1H), 7.53-7.55 (m, 2H), 7.37-7.41 (m, 1H), 5.57 (s, 2H).

4,10-diFmoc-deacylramoplanin amine was reacted with benzotriazol-1-ylacetic acid pentafluorophenyl ester according to Method B to obtain Example 69. HPLC: Rₜ=4.91 min (Condition 1). ESMS: m/z 1288.8 [(M+2H)/2].

### Example 70

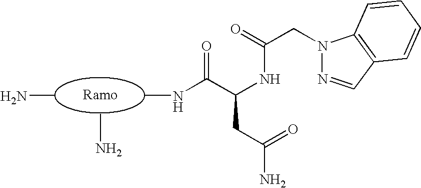

Indazol-1-ylacetic acid tert-butyl ester was prepared from indazole and tert-butyl bromoacetate following Method L in 69% yield after purifying the product by silica gel chromatography using hexane/ethyl acetate (6:4) as an eluent.

To a portion of indazol-1-ylacetic acid tert-butyl ester (300 mg) was added 50% TFA in methylene chloride (20 mL) and the reaction mixture was stirred for 8 h. The reaction mixture was concentrated in vacuo to yield indazol-1-ylacetic acid (225 mg, 98%).

Indazol-1-ylacetic acid pentafluorophenyl ester was prepared from indazol-1-ylacetic acid according to Method A in 72% yield. ¹H NMR (300 MHz, CDCl₃): δ 8.11 (s, 1H), 7.80-7.77 (m, 1H), 7.70-7.26 (m, 2H), 7.29-7.21 (m, 1H), 5.53 (s, 2H).

4,10-diFmoc-deacylramoplanin amine was reacted with indazol-1-ylacetic acid pentafluorophenyl ester according to Method B to obtain Example 70. HPLC: Rₜ=5.34 min (Condition 1). ESMS: m/z 1288.5 [(M+2H)/2].

### Example 71

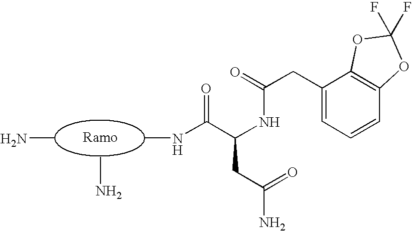

2,2-Difluoro-1,3-benzodioxole (0.56 mL, 5.0 mmol) was added to a solution of sec-butyllithium (in THF, 10 mL, 5.0 mmol) in cyclohexane at −78° C. The reaction mixture was treated with ethylene oxide (2.2 g, 50 mmol) and allowed to warm to 23° C.

After 2 h, the reaction mixture was diluted with ether (100 mL), washed with water (5×25 mL) then brine (2×25 mL). The organic layer was dried (Na₂SO₄), concentrated in vacuo and the residue was purified by silica gel column chromatography (5-50% EtOAc in hexanes) to yield 2-(2,2-difluorobenzo[1,3]dioxol-4-yl)ethanol (286 mg, 38%).

To a suspension of NaIO₄ (1.16 g, 5.4 mmol) in H₂O (7 mL) at 23° C. was added RuCl₃.H₂O (47 mg, 0.4 mmol), followed by a solution of 2-(2,2-difluorobenzo[1,3]dioxol-4-yl)ethanol (110 mg, 0.54 mmol) in acetone (7 mL). The reaction mixture was stirred overnight at 23° C. and poured into ethyl ether (100 mL). The organic layer was separated and discarded. The aqueous layer was acidified with 1.0 N HCl to pH 2 and ethyl acetate (120 mL) was added. The organic layer was separated and washed with H₂O (2×100 mL), then brine (150 mL), dried (Na₂SO₄), and concentrated to yield (2,2-difluorobenzo[1,3]dioxol-4-yl)acetic acid (56 mg, 48%).

(2,2-Difluorobenzo[1,3]dioxol-4-yl)acetic acid pentafluorophenyl ester was prepared from (2,2-difluorobenzo[1,3]dioxol-4-yl)acetic acid according to Method A in 45% yield. ¹H NMR (300 MHz, CDCl₃): δ 7.15-7.00 (m, 3H), 4.04 (s, 2H).

4,10-diFmoc-deacylramoplanin amine was reacted with (2,2-difluorobenzo[1,3]dioxolyl)acetic acid pentafluorophenyl ester according to Method B to obtain Example 71. HPLC: Rₜ=5.78 min (Condition 1). ESMS: m/z 1308.4 [(M+2H)/2].

### Example 72

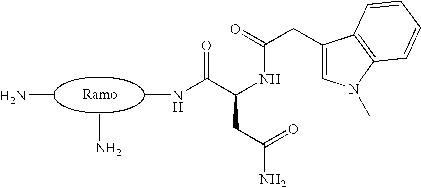

(1-Methyl-1H-indol-3-yl)acetic acid pentafluorophenyl ester was prepared from (1-methyl-1H-indol-3-yl)acetic acid according to Method A in 89% yield. ¹H NMR (300 MHz, CDCl₃): δ 7.63-7.60 (m, 1H), 7.32-7.25 (m, 2H), 7.19-7.12 (m, 2H), 4.12 (s, 2H), 3.79 (s, 3H).

4,10-diFmoc-deacylramoplanin amine was reacted with (1-methyl-1H-indol-3-yl)acetic acid pentafluorophenyl ester according to Method B to obtain Example 72. HPLC: Rₜ=5.70 min (Condition 1). ESMS: m/z 1295.5 [(M+2H)/2].

### Example 73

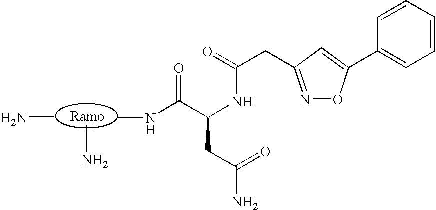

To a stirred suspension of LAH in THF (10 mL, 1M solution in THF) at 0° C. was added a solution of 5-phenylisoxazole-3-carboxylic acid (0.9 g in 5 mL of THF) and after completion of addition the reaction was slowly warmed to room temperature (30 min). The reaction mixture was cooled to 0° C. and ethyl acetate was added (30 mL), followed by slow addition of saturated sodium sulfate solution. The solid was rinsed with ether several times and the solvent decanted. The combined organic layer was dried over MgSO₄, filtered and concentrated in vacuo to get (5-phenylisoxazol-3-yl)methanol (0.8 g, 96% yield).

(5-Phenylisoxazol-3-yl)acetonitrile was prepared from (5-phenylisoxazol-3-yl)methanol following Method E then Method F in 29% yield after purification of product by silica gel column chromatography using hexane/ethyl acetate (8:2) as an eluent

(5-Phenylisoxazol-3-yl)acetic acid was prepared from (5-phenylisoxazol-3-yl)acetonitrile following Method G in 55% yield which was used in the next step without purification.

(5-Phenylisoxazol-3-yl)acetic acid pentafluorophenyl ester was prepared from (5-phenylisoxazol-3-yl)acetic acid following Method A in 65% yield after purification of product by silica gel column chromatography using hexane/ethyl acetate (9:1) as an eluent. ¹H NMR (300 MHz, CDCl₃): 7.78 (m, 2H), 7.47 (m, 3H), 6.65 (s, 1H), 4.16 (s, 2H).

4,10-diFmoc-deacylramoplanin amine was reacted with (5-phenylisoxazol-3-yl)-acetic acid pentafluorophenyl ester according to Method B to obtain Example 73. HPLC: Rₜ=5.64 min (Condition 1). ESMS: m/z 1301.8 [(M+2H)/2].

### Example 74

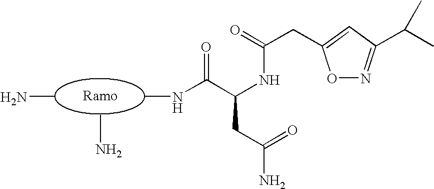

(3-Isopropylisoxazol-5-yl)methanol was prepared from 2-methylpropionaldehyde oxime (Example 24) and propargyl alcohol following Method K. The reaction was initially conducted at 0° C. for 30 min and then at room temperature for 4 h. The product was purified by silica gel column chromatography using ethyl acetate/hexanes (1:1) as an eluent to afford (3-isopropylisoxazol-5-yl)methanol in 36% yield.

(3-Isopropylisoxazol-5-yl)acetonitrile was prepared from (3-isopropylisoxazol-5-yl)methanol following Method E then Method F in 32% yield after purification of product by silica gel column chromatography using hexane/ethyl acetate (10:1) as an eluent.

(3-Isopropylisoxazol-5-yl)acetic acid pentafluorophenyl ester was prepared from (3-isopropylisoxazol-5-yl)acetonitrile following Method G then Method A in 30% yield after purification of product by silica gel column chromatography using hexane/ethyl acetate (10:1) as an eluent. ¹H NMR (300 MHz, CDCl₃): 6.22 (s, 1H), 4.12 (s, 2H), 3.04 (m, 1H), 1.27 (d, J=6.87, 6H).

Example 74 was prepared by reacting (3-isopropylisoxazol-5-yl)acetic acid pentafluorophenyl ester with 4,10-diFmoc-deacylramoplanin amine according to Method B. HPLC: Rₜ=7.216 min (Condition 3). ESMS: m/z 1284.7 [(M+2H)/2].

### Example 75

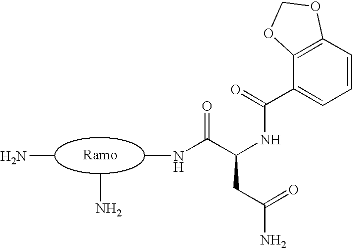

1,3-Benzodioxole-4-carboxylic acid pentafluorophenyl ester was prepared from 1,3-benzodioxole-4-carboxylic acid according to Method A in 84% yield. ¹H NMR (300 MHz, CDCl₃): δ 7.56-7.53 (m, 1H), 7.18-7.08 (m, 1H), 6.95 (t, J=9.00 Hz, 1H), 6.16 (s, 2H).

4,10-diFmoc-deacylramoplanin amine was reacted with 1,3-benzodioxole-4-carboxylic acid pentafluorophenyl ester according to Method B to obtain Example 75. HPLC: Rₜ=7.34 min (Condition 3). ESMS: m/z 1283.9 [(M+2H)/2].

### Example 76

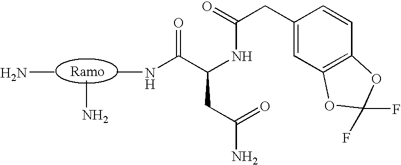

To a stirred solution of oxalyl chloride (2M solution in dichloromethane, 10 mL) was added 2,2-difluorobenzo[1,3]dioxole-5-carboxylic acid (1.01 g) in one lot followed by a drop of DMF. The reaction was stirred continuously at room temperature for 8 h and concentrated in vacuo. The residue was dissolved in toluene (20 mL), and the solvent removed to afford 2,2-difluorobenzo[1,3]dioxole-5-carbonyl chloride, which was used in the subsequent step without purification. To a stirred mixture of TMSCH₂N₂ (10 mL of 2M solution in hexanes) and triethylamine (2.5 mL) in THF (20 mL) and acetonitrile (20 mL) at 0° C. was added 2,2-difluorobenzo[1,3]dioxole-5-carbonyl chloride, and continued stirring at 0° C. for 30 h. The reaction mixture was concentrated in vacuo. Benzyl alcohol (4 mL) and 2,4,6-trimethylpyridine (4 mL) were added to the evaporated residue and the mixture was stirred at 180-185° C. for 10 minutes. The reaction mixture was cooled to room temperature, diluted with ether and washed successively with 10% aqueous citric acid, water and saturated aqueous sodium chloride. The organic layer was dried over MgSO₄, and concentrated in vacuo. The residue was disolved in 15 mL of 2M solution sodium hydroxide in methanol, stirred at room temperature for 18 h and concentrated in vacuo. The residue was suspended in water, and the aqueous layer was extracted with ether, and the organic layer was discarded. The aqueous layer was acidified to pH 3-4 with 6N hydrochloric acid then extracted with ether to yield (2,2-difluorobenzo[1,3]dioxole-5-yl)acetic acid. (2,2-difluorobenzo[1,3]dioxole-5-yl)acetic acid pentafluorophenyl ester was prepared from (2,2-difluorobenzo[1,3]dioxole-5-yl)acetic acid following Method A (0.1 g, 5% yield for 5 steps).

4,10-diFmoc-deacylramoplanin amine was reacted with (2,2-difluorobenzo[1,3]dioxole-5-yl)acetic acid pentafluorophenyl ester according to Method B to obtain Example 76. HPLC: Rₜ=8.24 min (Condition 3). ESMS: m/z 1308.8 [(M+2H)/2].

### Example 77

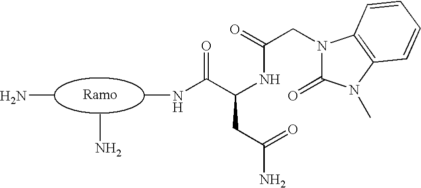

To a solution of 2-hydroxybenzimidazole (2.68 g, 20.0 mmol) in DMF (25 mL) at 0° C. was added NaH (60% in mineral oil, 880 mg, 22.0 mmol) and the reaction mixture was stirred for 5 min. Benzyl-2-bromoacetate (3.48 mL, 22 mmol) was slowly added to the reaction mixture while stirring. After 3 h, the reaction mixture was diluted with ethyl acetate (200 mL) and washed with 0.5 N HCl (200 mL), then brine (200 mL), and then dried (Na₂SO₄) and concentrated to yield the crude product which was purified by silica gel column chromatography (20-50% EtOAc in hexanes) to yield (2-oxo-2,3-dihydrobenzoimidazol-1-yl)acetic acid benzyl ester (1.86 g, 33%).

To a solution of (2-oxo-2,3-dihydrobenzoimidazol-1-yl)acetic acid benzyl ester (94 mg, 0.35 mmol) in DMF (5 mL) at 0° C. was added NaH (60% in mineral oil, 17 mg, 0.42 mmol), and the reaction mixture was stirred for 5 min. Methyl iodide (44 μL, 0.7 mmol) was slowly added to the reaction mixture while stirring. After 17 h, the reaction mixture was diluted with ethyl acetate (100 mL) and washed with 0.5 N HCl (100 mL), saturated aqueous NaHCO₃ (100 mL), then brine (100 mL), then subsequently dried (Na₂SO₄) and concentrated to yield the crude product which was purified by silica gel column chromatography (20-50% EtOAc in hexanes) to yield the desired (3-methyl-2-oxo-2,3-dihydrobenzoimidazol-1-yl)acetic acid benzyl ester (80 mg, 77%).

To solution of (3-methyl-2-oxo-2,3-dihydrobenzoimidazol-1-yl)acetic acid benzyl ester (80 mg, 0.27 mmol) in ethyl acetate (6 mL) at 23° C. was added Pd/C (10 wt %, 26 mg). The reaction mixture was deoxygenated via vacuum to pump, then purged with hydrogen in a balloon. After 3 h, the reaction mixture was filtered through a pad of Celite and washed with ethyl acetate (100 mL). The filtrate was concentrated in vacuo to yield (3-methyl-2-oxo-2,3-dihydrobenzoimidazol-1-yl)acetic acid (55 mg, 100%).

(3-Methyl-2-oxo-2,3-dihydrobenzoimidazol-1-yl)acetic acid pentafluorophenyl ester was prepared from (3-methyl-2-oxo-2,3-dihydrobenzoimidazol-1-yl)acetic acid according to Method A in 77% yield. ¹H NMR (300 MHz, CDCl₃): δ 7.17-7.14 (m, 2H), 7.05-7.02 (m, 1H), 6.94 (d, J=8.40 Hz, 1H), 5.02 (s, 2H), 3.46 (s, 3H).

4,10-diFmoc-deacylramoplanin amine was reacted with (3-methyl-2-oxo-2,3-dihydrobenzoimidazol-1-yl)acetic acid pentafluorophenyl ester according to Method B to obtain Example 77. HPLC: Rₜ=7.22 min (Condition 3). ESMS: m/z 1303.6 [(M+2H)/2].

### Example 78

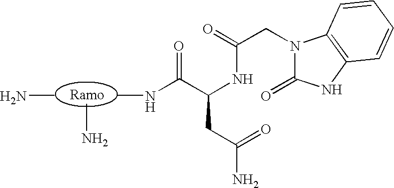

To a portion of (2-oxo-2,3-dihydrobenzoimidazol-1-yl)acetic acid benzylester (Example 77; 194 mg, 0.35 mmol) in ethyl acetate (5 mL) at 23° C. was added Pd/C (10 wt %, 19 mg) and the reaction mixture was deoxygenated via vacuum pump, then purged with hydrogen in a balloon. After 3 h, the reaction mixture was filtered through a pad of Celite and rinsed with ethyl acetate (100 mL). The filtrate was concentrated in vacuo to produce (2-oxo-2,3-dihydrobenzoimidazol-1-yl)acetic acid (68 mg, 100%).

(2-oxo-2,3-dihydrobenzoimidazol-1-yl)acetic acid pentafluorophenyl ester was prepared from (2-oxo-2,3-dihydrobenzoimidazol-1-yl)acetic acid according to Method A in 75% yield. ¹H NMR (300 MHz, CDCl₃): δ 8.69 (br s, 1H), 7.6-7.13 (m, 3H), 7.00-6.95 (m, 1H), 5.01 (s, 2H).

4,10-diFmoc-deacylramoplanin amine was reacted with (2-oxo-2,3-dihydrobenzoimidazol-1-yl)acetic acid pentafluorophenyl ester according to Method B to obtain Example 78. HPLC: Rₜ=6.61 min (Condition 3). ESMS: m/z 1296.6 [(M+2H)/2].

### Example 79

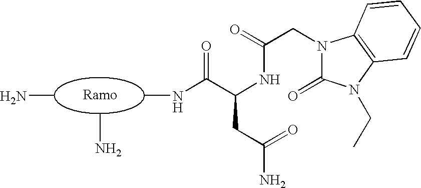

To a solution of (2-oxo-2,3-dihydrobenzoimidazol-1-yl)acetic acid benzylester (Example 77; 94 mg, 0.35 mmol) in DMF (5 mL) at 0° C. was added NaH (60% in mineral oil, 17 mg, 0.42 mmol) and the reaction mixture was stirred for 5 min. Ethyl iodide (56 μL, 0.7 mmol) was slowly added to the reaction mixture, while stirring. After 17 h, the reaction mixture was diluted with ethyl acetate (100 mL) and washed with 0.5 N HCl (100 mL), saturated aqueous NaHCO₃ (100 mL), then brine (100 mL), and then dried (Na₂SO₄) and concentrated to yield the crude product that was purified by silica gel column chromatography (20-50% EtOAc in hexanes) to yield (3-ethyl-2-oxo-2,3-dihydrobenzoimidazol-1-yl)acetic acid benzyl ester (88 mg, 81%).

To a stirred solution of (3-ethyl-2-oxo-2,3-dihydrobenzoimidazol-1-yl)acetic acid benzyl ester (75 mg, 0.24 mmol) in ethyl acetate (5 mL) at 23° C. was added Pd/C (10 wt %, 23 mg) and the reaction mixture was deoxygenated via vacuum pump, then purged with hydrogen in a balloon. After 3 h, the reaction mixture was filtered through a pad of Celite and rinsed with ethyl acetate (100 mL). The filtrate was concentrated in vacuo to yield (3-ethyl-2-oxo-2,3-dihydrobenzoimidazol-1-yl)acetic acid (53 mg, 100%).

(3-ethyl-2-oxo-2,3-dihydrobenzoimidazol-1-yl)acetic acid pentafluorophenyl ester was prepared from (3-ethyl-2-oxo-2,3-dihydrobenzoimidazol-1-yl)acetic acid according to Method A in 80% yield. ¹H NMR (300 MHz, CDCl₃): δ 7.18-7.06 (m, 3H), 6.98-6.95 (m, 1H), 5.03 (s, 2H), 3.99 (q, J=7.2 Hz, 2H), 1.37 (t, 7.5 Hz, 3H).

4,10-diFmoc-deacylramoplanin amine was reacted with (3-ethyl-2-oxo-2,3-dihydrobenzoimidazol-1-yl)acetic acid pentafluorophenyl ester according to Method B to obtain Example 79. HPLC: Rₜ=7.68 min (Condition 3). ESMS: m/z 1310.2 [(M+2H)/2].

### Example 80

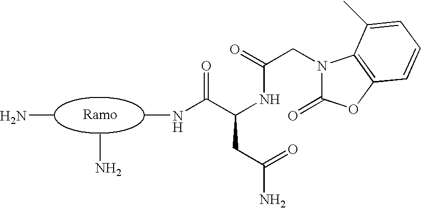

To a solution of 3-methyl-2-nitrophenol (1.0 g, 6.53 mmol) in methanol (10 mL) at 23° C. was added Pd/C (10 wt %, 400 mg) and the reaction mixture was deoxygenated under vacuum, then purged with hydrogen in a balloon. After stirring for 5 h, the reaction mixture was filtered through a pad of Celite, and the Celite pad was rinsed with methanol (100 mL). The filtrate was concentrated in vacuo to yield 2-amino-3-methylphenol (803 mg, 100%).

To a solution of 2-amino-3-methylphenol (777 mg, 6.32 mmol) in acetonitrile (27 mL) at 23° C. was added 1,1′-carbonyldiimidazole (3.07 g, 18.9 mmol). The reaction mixture was heated to 70° C., and stirred overnight. The reaction mixture was cooled to room temperature and partitioned between ethyl acetate (150 mL) and H₂O (100 mL). The organic layer was separated and washed with brine (100 mL), dried (MgSO₄), and concentrated to yield the crude product, which was purified by silica gel column chromatography (20-50% EtOAc in hexanes) to yield 4-methyl-3H-benzooxazol-2-one (931 mg, 99%).

To a portion of 4-methyl-3H-benzooxazol-2-one (450 mg, 3.02 mmol) in DMF (10 mL) at 23° C. was added K₂CO₃ (900 mg, 6.51 mmol), and the reaction mixture was stirred for 30 min. Benzyl-2-bromoacetate (909 μL, 5.74 mmol) was slowly added to the reaction mixture, while stirring. After 17 h, the reaction mixture was diluted with ethyl acetate (100 mL) and washed with 1.0 N HCl (100 mL), saturated aqueous NaHCO₃ (100 mL), then brine (100 mL), then dried (Na₂SO₄) and concentrated to yield the crude product which was purified by silica gel column chromatography (20-50% EtOAc in hexanes) to yield (4-methyl-2-oxobenzooxazol-3-yl)acetic acid benzyl ester (601 mg, 67%).

To a solution of (4-methyl-2-oxobenzooxazol-3-yl)acetic acid benzyl ester (500 mg, 1.68 mmol) in methanol (20 mL) at 23° C. was added Pd/C (10 wt %, 150 mg), and the reaction mixture was deoxygenated via vacuum pump, then purged with hydrogen in a balloon. After stirring for 5 h, the reaction mixture was filtered through a pad of Celite and rinsed with methanol (100 mL). The filtrate was concentrated in vacuo to yield (4-methyl-2-oxobenzooxazol-3-yl)acetic acid (347 mg, 100%).

(4-Methyl-2-oxobenzooxazol-3-yl)acetic acid pentafluorophenyl ester was prepared from (4-methyl-2-oxobenzooxazol-3-yl)acetic acid according to Method A in 82% yield. ¹H NMR (300 MHz, CDCl₃) δ 7.10-6.97 (m, 3H), 5.14 (s, 2H), 2.49 (s, 3H).

4,10-diFmoc-deacylramoplanin amine was reacted with (4-methyl-2-oxobenzooxazol-3-yl)acetic acid pentafluorophenyl ester according to Method B to obtain Example 80. HPLC: Rₜ=4.85 min (Condition 1). ESMS: m/z 1303.9 [(M+2H)/2].

### Example 81

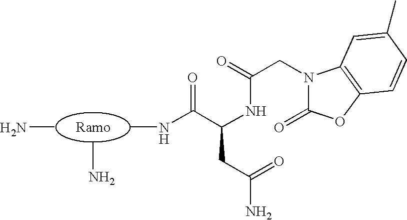

To a solution of 4-methyl-2-nitrophenol (1.0 g, 6.53 mmol) in methanol (10 mL) at 23° C. was added Pd/C (10 wt %, 400 mg), and the reaction mixture was deoxygenated under vacuum, then purged with hydrogen in a balloon. After stirring for 5 h, the reaction mixture was filtered through a pad of Celite and the Celite pad was rinsed with methanol (100 mL). The filtrate was concentrated in vacuo to yield 2-amino-4-methylphenol (803 mg, 100%).

To a solution of 2-amino-4-methylphenol (500 mg, 4.06 mmol) in acetonitrile (25 mL) at 23° C. was added 1,1′-carbonyldiimidazole (1.98 g, 12.2 mmol). The reaction mixture was heated to 70° C., and stirred overnight. The reaction mixture was cooled to room temperature and partitioned between ethyl acetate (150 mL) and H₂O (100 mL). The organic layer was separated and washed with brine (100 mL), then dried (MgSO₄) and concentrated to yield the crude product which was purified by silica gel column chromatography (20-50% EtOAc in hexanes) to yield 5-methyl-3H-benzooxazol-2-one (601 mg, 99%).

To a portion of 5-methyl-3H-benzooxazol-2-one (450 mg, 3.02 mmol) in DMF (10 mL) at 23° C. was added K₂CO₃ (900 mg, 6.51 mmol), and the reaction mixture was stirred for 30 min. Benzyl-2-bromoacetate (909 mL, 5.74 mmol) was slowly added to the reaction mixture, while stirring. After 17 h, the reaction mixture was diluted with ethyl acetate (100 mL) and washed with 1.0 N HCl (100 mL), saturated aqueous NaHCO₃ (100 mL), then brine (100 mL), dried (Na₂SO₄) and concentrated to yield the crude product that was purified by silica gel column chromatography (20-50% EtOAc in hexanes) to yield the desired (5-methyl-2-oxobenzooxazol-3-yl)acetic acid benzyl ester (585 mg, 65%).

To a solution of (5-methyl-2-oxobenzooxazol-3-yl)acetic acid benzyl ester (120 mg, 0.40 mmol) in ethyl acetate (7 mL) at 23° C. was added Pd/C (10 wt %, 90 mg) and the reaction mixture was deoxygenated via vacuum pump, then purged with hydrogen in a balloon. After stirring for 5 h, the reaction mixture was filtered through a pad of Celite and the Celite pad was rinsed with ethyl acetate (100 mL). The filtrate was concentrated in vacuo to yield (5-methyl-2-oxobenzooxazol-3-yl)acetic acid (82 mg, 100%).

(5-Methyl-2-oxobenzooxazol-3-yl)acetic acid pentafluorophenyl ester was prepared from (5-methyl-2-oxobenzooxazol-3-yl)acetic acid according to Method A in 77% yield. ¹H NMR (300 MHz, CDCl₃): δ 7.14 (d, J=8.40 Hz, 1H), 6.97 (d, J=7.20 Hz, 1H), 6.75 (s, 1H), 4.88 (s, 2H), 2.14 (s, 3H).

4,10-diFmoc-deacylramoplanin amine was reacted with (5-methyl-2-oxobenzooxazol-3-yl)acetic acid pentafluorophenyl ester according to Method B to obtain Example 81. HPLC: Rₜ=4.95 min (Condition 1). ESMS: m/z 1304.3 [(M+2H)/2].

### Example 82

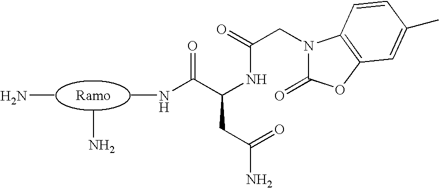

To a solution of 5-methyl-2-nitrophenol (1.0 g, 6.53 mmol) in methanol (10 mL) at 23° C. was added Pd/C (10 wt %, 400 mg), and the reaction mixture was deoxygenated under vacuum, then purged with hydrogen in a balloon. After stirring for 5 h, the reaction mixture was filtered through a pad of Celite and the Celite pad was rinsed with methanol (100 mL). The filtrate was concentrated in vacuo to yield 2-amino-5-methylphenol (801 mg, 100%).

To a solution of 2-amino-5-methylphenol (777 mg, 6.32 mmol) in acetonitrile (27 mL) at 23° C. was added 1,1′-carbonyldiimidazole (3.07 g, 18.9 mmol) and the reaction mixture was heated to 70° C., and stirred overnight. The reaction mixture was cooled to room temperature and partitioned between ethyl acetate (150 mL) and H₂O (100 mL). The organic layer was separated and washed with brine (100 mL), dried (MgSO₄) and concentrated to yield the crude product which was purified by silica gel column chromatography (20-50% EtOAc in hexanes) to yield 6-methyl-3H-benzooxazol-2-one (853 mg, 90%).

To a portion of 6-methyl-3H-benzooxazol-2-one (450 mg, 3.02 mmol) in DMF (10 mL) at 23° C. was added K₂CO₃ (900 mg, 6.51 mmol), and the reaction mixture was stirred for 30 min. Benzyl-2-bromoacetate (909 μL, 5.74 mmol) was slowly added to the reaction mixture, while stirring. After 17 h, the reaction mixture was diluted with ethyl acetate (100 mL) and washed with 1.0 N HCl (100 mL), saturate aqueous NaHCO₃ (100 mL), then brine (100 mL), and then was dried (Na₂SO₄) and concentrated to yield the crude product that was purified by silica gel column chromatography (20-50% EtOAc in hexanes) to afford (6-methyl-2-oxobenzooxazol-3-yl)acetic acid benzyl ester (512 mg, 57%).

To a solution of (6-methyl-2-oxobenzooxazol-3-yl)acetic acid benzyl ester (150 mg, 0.50 mmol) in ethyl acetate (7 mL) at 23° C. was added Pd/C (10 wt %, 90 mg), and the reaction mixture was deoxygenated via vacuum pump, then purged with hydrogen in a balloon. After stirring for 5 h, the reaction mixture was filtered through a pad of Celite and the Celite pad was rinsed with ethyl acetate (100 mL). The filtrate was concentrated in vacuo to yield (6-methyl-2-oxobenzooxazol-3-yl)acetic acid (102 mg, 100%).

(6-Methyl-2-oxobenzooxazol-3-yl)acetic acid pentafluorophenyl ester was prepared from (6-methyl-2-oxobenzooxazol-3-yl)acetic acid according to Method A in 75% yield. ¹H NMR (300 MHz, CDCl₃): δ 7.08 (s, 1H), 7.03 (d, J=8.10 Hz, 1H), 6.82 (d, J=8.70 Hz, 111), 4.93 (s, 2H), 2.40 (s, 3H).

4,10-diFmoc-deacylramoplanin amine was reacted with (6-methyl-2-oxobenzooxazol-3-yl)acetic acid pentafluorophenyl ester according to Method B to obtain Example 82. HPLC: Rₜ=4.96 min (Condition 1). ESMS: m/z 1303.9 [(M+2H)/2].

### Example 83

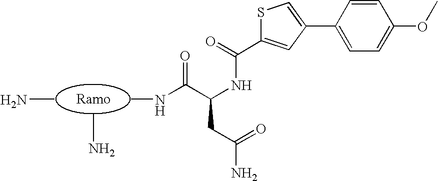

4-(4-Methoxyphenyl)thiophene-2-carboxylic acid pentafluorophenyl ester was prepared from 4-(4-methoxyphenyl)thiophene-2-carboxylic acid according to Method A in 91% yield. ¹H NMR (300 MHz, CDCl₃): δ 8.25 (s, 1H), 7.74 (s, 1H), 7.55-7.52 (m, 2H), 6.98-6.95 (m, 2H), 3.85 (s, 3H).

4,10-diFmoc-deacylramoplanin amine was reacted with 4-(4-methoxyphenyl)-thiophene-2-carboxylic acid pentafluorophenyl ester according to Method B to obtain Example 83. HPLC: Rₜ=5.31 min (Condition 1). ESMS: m/z 1317.6 [(M+2H)/2].

### Example 84

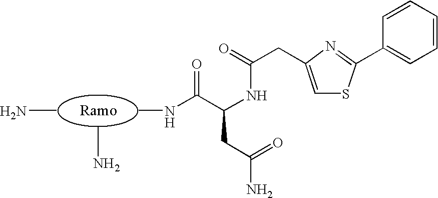

To a stirred suspension of thiobenzamide (1.37 g, 10 mmol) in methanol (20 mL) was added ethyl chloroacetoacetate (1.7 g, 10.36 mmol), and the reaction mixture was heated to reflux for 24 h. It was cooled to room temperature and a solution of LiOH (1 g) in water (4 mL) was added. This was stirred at room temperature for 4 h, concentrated in vacuo, and the residue was suspended in water. The aqueous layer was extracted with ether and the organic layer discarded. The aqueous layer was acidified to pH 3-4, extracted with ether, the organic layer was dried over MgSO₄, filtered and concentrated to produce (2-phenylthiazol-4-yl)acetic acid (1.1 g, 50% yield).

(2-Phenylthiazol-4-yl)acetic acid pentafluorophenyl ester was prepared from (2-phenylthiazol-4-yl)acetic acid following Method A in 53% yield after purification of product by silica gel column chromatography using hexane/ethyl acetate (10:1) as an eluent. ¹H NMR (300 MHz, CDCl₃): 7.94 (m, 2H), 7.44 (m, 3H), 7.28 (s, 1H), 4.25 (s, 2H).

Example 84 was prepared by reacting (2-phenylthiazol-4-yl)acetic acid pentafluorophenyl ester with 4,10-diFmoc-deacylramoplanin amine according to Method B. HPLC: Rₜ=5.147 min (Condition 1); Rₜ=8.27 min (Condition 3). ESMS: m/z 1310.2 [(M+2H)/2].

### Example 85

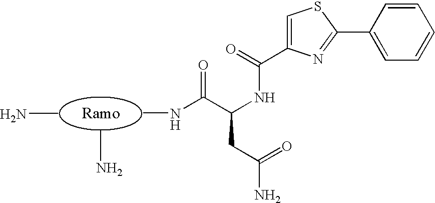

(2-phenylthiazolyl)carboxylic acid pentafluorophenyl ester was prepared from (2-phenylthiazol-yl)carboxylic acid according to Method A in 86% yield. ¹H NMR (300 MHz, CDCl₃): δ 8.47 (s, 1H), 8.05-8.02 (m, 2H), 7.51-7.48 (m, 3H).

4,10-diFmoc-deacylramoplanin amine was reacted with (2-phenylthiazol-4-yl)carboxylic acid pentafluorophenyl ester according to Method B to obtain Example 85. HPLC: Rₜ=5.23 min (Condition 1). ESMS: m/z 1303.2 [(M+2H)/2].

### Example 86

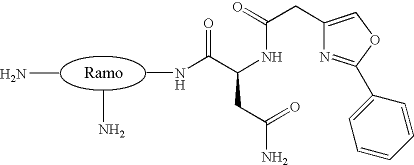

To a stirred solution of benzamide (1.21 g, 10 mmol) in a mixture of dioxane (10 mL) and toluene (10 mL) was added ethyl chloroacetoacetate (3.28 g, 20 mmol) and the reaction mixture was heated to 120° C. for 24 h. The solvent was removed in vacuo and the residue was chromatographed on silica gel using hexane/ethyl acetate mixture (7:3) as an eluent to afford (2-phenyloxazol-4-yl)acetic acid ethyl ester (0.6 g, 26% yield).

(2-Phenyloxazol-4-yl)acetic acid was obtained from (2-phenyloxazol-4-yl)acetic acid ethyl ester following Method C in quantitative yield uing LiOH as a base and aqueous methanol as a solvent.

(2-Phenyloxazol-4-yl)acetic acid pentafluorophenyl ester was prepared from (2-phenyloxazol-4-yl)acetic acid following Method A in 46% yield. ¹H NMR (300 MHz, CDCl₃): δ 7.74 (d, J=8.10 Hz, 1H), 7.64 (s, 1H), 7.44-7.43 (m, 2H), 7.26-7.09 (m, 2H), 4.09 (s, 2H).

4,10-diFmoc-deacylramoplanin amine was reacted with (2-phenyloxazol-4-yl) acetic acid pentafluorophenyl ester according to Method B to obtain Example 86. HPLC: Rₜ=5.08 min (Condition 1). ESMS: m/z 1301.8 [(M+2H)/2].

### Example 87

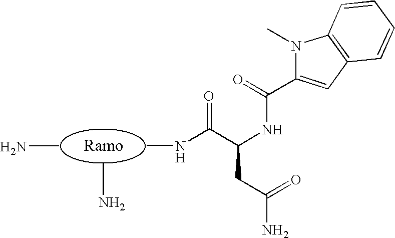

To a solution of indole-2-carboxylic acid (450 mg, 2.79 mmol) in methanol (15 mL) at 23° C. was added concentrated H₂SO₄ (2 drops) and the reaction mixture was heated to reflux, while stirring. After 48 h, the reaction mixture was cooled to room temperature and concentrated in vacuo to yield indole-2-carboxylic acid methyl ester (444 mg, 91%).

To a solution of indole-2-carboxylic acid methyl ester (300 mg, 1.71 mmol) in DMF (5 mL) at 23° C. was added K₂CO₃ (600 mg, 4.34 mmol), and the reaction mixture was stirred for 30 min. Methyl iodide (213 μL, 3.42 mmol) was slowly added to the reaction mixture. After stirring for 17 h, the reaction mixture was diluted with ethyl acetate (100 mL) and washed with 1.0 N HCl (100 mL), saturated aqueous NaHCO₃ (100 mL), then brine (100 mL), and was then dried (Na₂SO₄) and concentrated to yield the crude product that was purified by silica gel column chromatography (20-50% EtOAc in hexanes) to yield 1-methyl-1H-indole-2-carboxylic acid methyl ester (283 mg, 87%).

To a solution of 1-methyl-1H-indole-2-carboxylic acid methyl ester (250 mg, 1.32 mmol) in THF/H₂O (2:1, 15 mL) at 23° C. was added LiOH.H₂O (291 mg, 6.94 mmol) and the reaction mixture was stirred overnight. The reaction mixture was concentrated in vacuo and partitioned between H₂O (100 mL) and ethyl ether (100 mL). The aqueous layer was acidified to pH 2-3 with 1.0 N HCl, then extracted with ethyl acetate (2×100 mL). The organic layer was dried (MgSO₄) and concentrated to yield 1-methyl-1H-indole-2-carboxylic acid (210 mg, 91%).

1-Methyl-1H-indole-2-carboxylic acid pentafluorophenyl ester was prepared from 1-methyl-1H-indole-2-carboxylic acid according to Method A in 65% yield. ¹H NMR (300 MHz, CDCl₃): δ 7.60-7.73 (m, 1H), 7.64 (s, 1H), 7.45-7.43 (m, 1H), 7.26-7.20 (m, 2H), 4.10 (s, 3H).

4,10-diFmoc-deacylramoplanin amine was reacted with 1-methyl-1H-indole-2-carboxylic acid pentafluorophenyl ester according to Method B to obtain Example 87. HPLC: Rₜ=5.16 min (Condition 1). ESMS: m/z 1288.8 [(M+2H)/2].

### Example 88

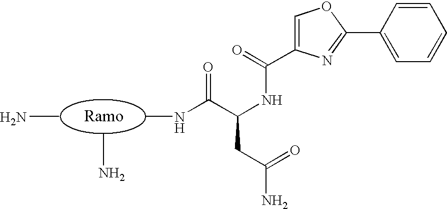

To a solution of benzamide (1.0 g, 8.25 mmol) in toluene/dioxane (1:1, 40 mL) at 23° C. was added ethyl bromopyruvate (3.12 mL, 24.76 mmol), and the reaction mixture was heated to reflux while stirring. After 17 h, the reaction mixture was cooled to room temperature and concentrated in vacuo to yield the crude product that was purified by silica gel column chromatography (30% EtOAc in hexanes) to produce (2-phenyloxazol-4-yl)carboxylic acid ethyl ester (872 mg, 49%).

To a solution of (2-phenyloxazol-4-yl)carboxylic acid ethyl ester (300 mg, 1.38 mmol) in THF/H₂O (2:1, 15 mL) at 23° C. was added LiOH.H₂O (305 mg, 7.26 mmol) and the reaction mixture was stirred overnight. The reaction mixture was concentrated in vacuo and partitioned between H₂O (100 mL) and ethyl ether (100 mL). The aqueous layer was acidified to pH 2-3 with. 1.0 N HCl, then extracted with ethyl acetate (2×100 mL). The organic layer was dried (MgSO₄) and concentrated to afford (2-phenyloxazol-4-yl)carboxylic acid (249 mg, 96%).

(2-Phenyloxazol-4-yl)-carboxylic acid pentafluorophenyl ester was prepared from (2-phenyl oxazol-4-yl)carboxylic acid according to Method A in 84% yield. ¹H NMR (300 MHz, CDCl₃): δ 8.54 (s, 1H), 8.20-8.10 (m, 2H), 7.60-7.45 (m, 3H).

4,10-diFmoc-deacylramoplanin amine was reacted with (2-phenyloxazol-4-yl)carboxylic acid pentafluorophenyl ester according to Method B to obtain Example 88. HPLC: Rₜ=5.15 min (Condition 1). ESMS: m/z 1294.8 [(M+2H)/2].

### Example 89

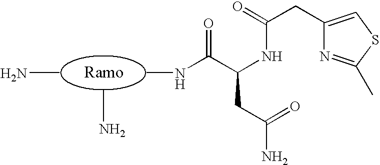

To a solution of thioacetamide (1.0 g, 13.3 mmol) in toluene/dioxane (1:1, 40 mL) at 23° C. was added ethyl chloroacetoacetate (5.39 mL, 39.3 mmol) and the reaction mixture was heated to reflux, while stirring. After 17 h, the reaction mixture was cooled to room temperature and concentrated in vacuo to yield the crude product that was purified by silica gel column chromatography (30% EtOAc in hexanes) to yield (2-methylthiazol-4-yl)acetic acid ethyl ester (785 mg, 32%).

To a solution of (2-methylthiazol-4-yl)acetic acid ethyl ester (300 mg, 1.62 mmol) in THF/H₂O (2:1, 15 mL) at 23° C. was added LiOH.H₂O (357 mg, 8.51 mmol) and the reaction mixture was stirred overnight. The reaction mixture was concentrated in vacuo and partitioned between H₂O (100 mL) and ethyl ether (100 mL). The aqueous layer was acidified to pH 2-3 with 1.0 N HCl, then extracted with ethyl acetate (2×100 mL). The organic layer was dried (MgSO₄) and concentrated to yield the desired (2-methylthiazol-4-yl)acetic acid (228 mg, 90%).

(2-Methylthiazol-4-yl)acetic acid pentafluorophenyl ester was prepared from (2-methylthiazol-4-yl)acetic acid according to Method A in 72% yield. ¹H NMR (300 MHz, CDCl₃) δ 7.18 (s, 1H), 4.16 (s, 2H), 2.74 (s, 3H).

4,10-diFmoc-deacylramoplanin amine was reacted with (2-methylthiazol-4-yl)acetic acid pentafluorophenyl ester according to Method B to obtain Example 89. HPLC: Rₜ=4.66 min (Condition 1). ESMS: m/z 1279.4 [(M+2H)/2].

### Example 90

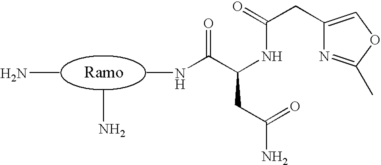

To a solution of acetamide (785 mg, 13.3 mmol) in toluene/dioxane (1:1, 40 mL) at 23° C. was added ethyl chloroacetoacetate (5.39 mL, 39.3 mmol) and the reaction mixture was heated to reflux, while stirring. After 17 h, the reaction mixture was cooled to room temperature and concentrated in vacuo to yield the crude product which was purified by silica gel column chromatography (30% EtOAc in hexanes) to yield (2-methyloxazol-4-yl)acetic acid ethyl ester (607 mg, 27%).

To a solution of (2-methyloxazol-4-yl)acetic acid ethyl ester (250 mg, 1.48 mmol) in THF/H₂O (2:1, 15 mL) at 23° C. was added LiOH.H₂O (326 mg, 7.76 mmol) and the reaction mixture was stirred overnight. The reaction mixture was concentrated in vacuo and partitioned between H₂O (100 mL) and ethyl ether (100 mL). The aqueous layer was acidified to pH 2-3 with 1.0 N HCl, then extracted with ethyl acetate (2×100 mL). The organic layer was dried (MgSO₄) and concentrated to yield the desired (2-methyl oxazol-4-yl)acetic acid (207 mg, 99%).

(2-Methyloxazol-4-yl)acetic acid pentafluorophenyl ester was prepared from (2-methyloxazol-4-yl)acetic acid according to Method A in 81% yield. ¹H NMR (300 MHz, CDCl₃) δ 7.62 (s, 1H), 3.95 (s, 2H), 2.52 (s, 3H).

4,10-diFmoc-deacylramoplanin amine was reacted with (2-methyloxazol-4-yl)acetic acid pentafluorophenyl ester according to Method B to obtain Example 90. HPLC: Rₜ=4.51 min (Condition 1). ESMS: m/z 1271.0 [(M+2H)/2].

### Example 91

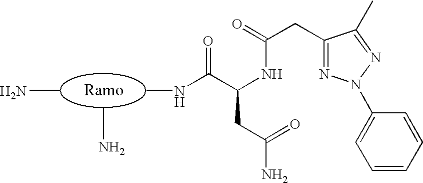

To a solution of 4-(bromomethyl)-5-methyl-2-phenyl-2H-1,2,3-triazole (1.0 g, 3.97 mmol) in DMF (8 mL) at 23° C. was added NaCN (1.02 g, 20.82 mmol). The reaction mixture was heated to 60° C., while stirring. After 17 h, the reaction mixture was cooled to room temperature and partitioned between H₂O (100 mL) and ethyl acetate (100 mL). The organic layer was washed with brine (100 mL), dried (MgSO₄) and concentrated to yield the crude product which was purified by silica gel column chromatography (30% EtOAc in hexanes) to afford (5-methyl-2-phenyl-2H-[1,2,3]triazol-4-yl)acetonitrile (752 mg, 95%).

A solution of (5-methyl-2-phenyl-2H-[1,2,3]triazol-4-yl)acetonitrile (400 mg, 2.02 mmol) in 5% NaOH in MeOH (10 mL) at 23° C. was slowly heated to reflux. After stirring for 17 h, the reaction mixture was cooled to room temperature and concentrated in vacuo. The crude oil was partitioned between H₂O (100 mL) and ethyl ether (100 mL).

The aqueous layer was acidified to pH 2-3 with 1.0 N HCl, then extracted with ethyl acetate (2×100 mL). The organic layer was dried (Na₂SO₄) and concentrated to yield the desired (5-methyl-2-phenyl-2H-[1,2,3]triazol-4-yl)-acetic acid (288 mg, 66%).

(5-Methyl-2-phenyl-2H[1,2,3]triazol-4-yl)acetic acid pentafluorophenyl ester was prepared from (5-methyl-2-phenyl-2H[1,2,3]triazol-4-yl)acetic acid according to Method A in 74% yield. ¹H NMR (300 MHz, CDCl₃) δ 8.03-7.98 (m, 2H), 7.49-7.43 (m, 2H), 7.35-7.31 (m, 1H), 4.14 (s, 2H), 2.40 (s, 3H).

4,10-diFmoc-deacylramoplanin amine was reacted with (5-methyl-2-phenyl-2H[1,2,3]triazol-4-yl)acetic acid pentafluorophenyl ester according to Method B to obtain Example 91. HPLC: Rₜ 5.08 min (Condition 1). ESMS: m/z 1309.2 [(M+2H)/2].

### Example 92

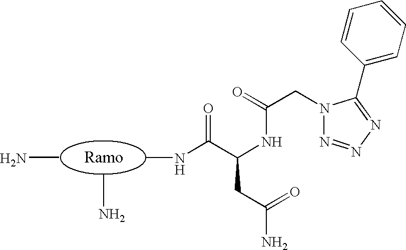

(5-Phenyltetrazol-1-yl)acetic acid benzyl ester was prepared from 5-phenyl-1H-tetrazole and benzyl bromoacetate according to Method P. The reaction was conducted at room temperature for 4 h, and the crude product was purified by silica gel column chromatography using hexane/ethyl acetate (8:2) as an eluent to afford (5-phenyltetrazol-1-yl)acetic acid benzyl ester in 81% yield.

(5-Phenyltetrazol-1-yl)acetic acid was prepared from (5-Phenyltetrazol-1-yl)acetic acid benzyl ester following Method Q in quantitative yield.

(5-Phenyltetrazol-1-yl)acetic acid pentafluorophenyl ester was prepared from (5-phenyltetrazol-1-yl)acetic acid according to Method A in 89% yield. ¹H NMR (300 MHz, CDCl₃) δ 8.18 (m, 2H), 7.52 (m, 3H), 5.84 (s, 2H).

4,10-diFmoc-deacylramoplanin amine was reacted with (5-phenyltetrazol-1-yl)acetic acid pentafluorophenyl ester according to Method B to obtain Example 92. HPLC: Rₜ=4.999 min (Condition 1). ESMS: m/z 1302.5 [(M+2H)/2].

### Example 93

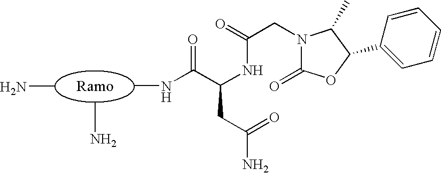

To a solution of (4R,5S)-(+)-4-methyl-5-phenyl-2-oxazolidinone (400 mg, 2.26 mmol) in DM (5 mL) at 0° C. was added NaH (60% in mineral oil, 108 mg, 2.71 mmol) and the reaction mixture was stirred for 5 min. Benzyl-2-bromoacetate (680 μL, 4.29 mmol) was slowly added to the reaction mixture, while stirring. After 17 h, the reaction mixture was diluted with ethyl acetate (100 mL) and washed with 0.5 N HCl (100 mL), saturated aqueous NaHCO₃ (100 mL), then brine (100 mL). The reaction mixture was dried (Na₂SO₄) and concentrated to yield the crude product, which was purified by silica gel column chromatography (30% EtOAc in hexanes) to yield (4R,5S)-(+)-(4-methyl-2-oxo-5-phenyloxazolidin-3-yl)acetic acid benzyl ester (640 mg, 87%).

To a solution of (4R,5S)-(+)-(4-methyl-2-oxo-5-phenyloxazolidin-3-yl)acetic acid benzyl ester (320 mg, 0.98 mmol) in ethyl acetate (5 mL) at 23° C. was added Pd/C (10 wt %, 160 mg) and the reaction mixture was deoxygenated via vacuum pump, then purged with hydrogen in a balloon. After stirring for 1 h, the reaction mixture was filtered through a pad of Celite, and the Celite pad was rinsed with ethyl acetate (100 mL). The filtrate was concentrated in vacuo to yield (4R,5S)-(+)-(4-methyl-2-oxo-5-phenyloxazolidin-3-yl)acetic acid (153 mg, 66%).

(4R,5S)-(+)-(4-methyl-2-oxo-5-phenyloxazolidin-3-yl)acetic acid pentafluorophenyl ester was prepared from (4R,5S)-(+)-(4-methyl-2-oxo-5-phenyloxazolidin-3-yl)acetic acid according to Method A in 82% yield. ¹H NMR (300 MHz, CDCl₃) δ 7.44-7.41 (m, 2H), 7.38-7.26 (m, 3H), 5.71 (d, J=8.1 Hz, 1H), 4.70 (d, J=18.3 Hz, 1H), 4.35 (m, 1H), 4.18 (d, J=18.3 Hz, 1H), 0.83 (d, J=6.6 Hz, 3H).

4,10-diFmoc-deacylramoplanin amine was reacted with (4R,5S)-(+)-(4-methyl-2-oxo-5-phenyloxazolidin-3-yl)acetic acid pentafluorophenyl ester according to Method B to obtain Example 93. HPLC: Rₜ=5.09 min (Condition 1). ESMS: m/z 1317.5 [(M+2H)/2].

### Example 94

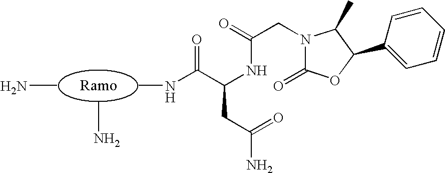

To a solution of (4S,5R)-(−)-4-methyl-5-phenyl-2-oxazolidinone (400 mg, 2.26 mmol) in DMF (5 mL) at 0° C. was added NaH (60% in mineral oil, 108 mg, 2.71 mmol), and the reaction mixture was stirred for 5 min. Benzyl-2-bromoacetate (680 μL, 4.29 mmol) was slowly added to the reaction mixture, while stirring. After 17 h, the reaction mixture was diluted with ethyl acetate (100 mL), and washed with 0.5 N HCl (100 mL), saturated aqueous NaHCO₃ (100 mL), then brine (100 mL). Dried (Na₂SO₄) and concentrated to yield the crude product, which was purified by silica gel column chromatography (30% EtOAc in hexanes) to yield (4S,5R)-(−)-(4-methyl-2-oxo-5-phenyloxazolidin-3-yl)acetic acid benzylester (731 mg, 99%).

To a solution of (4S,5R)-(−)-(4-methyl-2-oxo-5-phenyloxazolidin-3-yl)acetic acid benzyl ester (380 mg, 1.17 mmol) in ethyl acetate (5 mL) at 23° C. was added Pd/C (10 wt %, 190 mg) and the reaction mixture was deoxygenated via vacuum pump, then purged with hydrogen in a balloon. After stirring for 1 h, the reaction mixture was filtered through a pad of Celite, and the Celite pad was rinsed with ethyl acetate (100 mL). The filtrate was concentrated in vacuo to yield (4S,5R)-(−)-(4-methyl-2-oxo-5-phenyloxazolidin-3-yl)acetic acid (203 mg, 74%).

(4S,5R)-(−)-(4-methyl-2-oxo-5-phenyloxazolidin-3-yl)acetic acid pentafluorophenyl ester was prepared from (4S,5R)-(−)-(4-methyl-2-oxo-5-phenyloxazolidin-3-yl)acetic acid according to Method A in 76% yield. ¹H NMR (300 MHz, CDCl₃) δ 7.44-7.36 (m, 2H), 7.30-7.26 (m, 3H), 5.71 (d, J=8.1 Hz, 1H), 4.70 (d, J=18.3 Hz, 1H), 4.35 (m, 1H); 4.18 (d, J=18.3 Hz, 1H), 0.83 (d, J=6.6 Hz, 3H).

4,10-diFmoc-deacylramoplanin amine was reacted with (4S,5R)-(−)-(4-methyl-2-oxo-5-phenyloxazolidin-3-yl)acetic acid pentafluorophenyl ester according to Method B to obtain Example 94. HPLC: Rₜ=5.00 min (Condition 1). ESMS: m/z 1317.6 [(M+2H)/2].

### Example 95

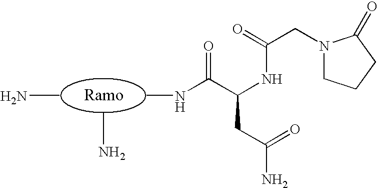

To a solution of pyrollidone (2.5 mL, 26.3 mmol) in DMF (60 mL) at 0° C. was added NaH (60% in mineral oil, 1.15 g, 28.8 mmol) and the reaction mixture was stirred for 5 min. The reaction mixture was heated to reflux for 30 min with tetrabutyl ammonium iodide (cat. amount, 218 mg) then cooled to room temperatre. Benzyl-2-bromoacetate (4.16 mL, 26.3 mmol) was added and the reaction mixture was refluxed for 2 h. The reaction mixture was cooled, poured into brine (100 mL), extracted with ethyl acetate (2×100 mL), dried over Na₂SO₄, and the solvent removed in vacuo to yield a brown oil that was purified by silica gel column chromatography (0-80% EtOAc in hexanes) to yield (2-oxopyrollidin-1-yl)acetic acid benzyl ester (1.47 g, 24%).

To a portion of (2-oxopyrollidin-1-yl)acetic acid benzyl ester (233 mg, 1.0 mmol) in methanol (5 mL) at 23° C. was added Pd/C (10 wt %, 100 mg), and the reaction mixture was deoxygenated via vacuum pump, then purged with hydrogen in a balloon. After 3 h, the reaction mixture was filtered through a pad of Celite, and the Celite pad was rinsed with methanol (100 mL). The filtrate was concentrated in vacuo to yield (2-oxopyrollidin-1-yl)acetic acid (143 mg, 100%).

(2-Oxopyrollidin-1-yl)acetic acid pentafluorophenyl ester was prepared from (2-oxopyrollidin-1-yl)acetic according to Method A in 72% yield. ¹H NMR (300 MHz, CDCl₃) δ 4.47 (s, 2H), 3.58 (t, J=7.2 Hz, 2H), 2.51 (t, J=8.1 Hz, 2H), 2.16 (m, 2H).

4,10-diFmoc-deacylramoplanin amine was reacted with (2-oxopyrollidin-1-yl)acetic acid pentafluorophenyl ester according to Method B to obtain Example 95. HPLC: Rₜ=4.36 min (Condition 1). ESMS: m/z 1271.7 [(M+2H)/2].

### Example 96

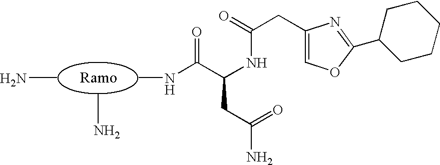

To a solution of cyclohexane carboxamide (2.0 g, 15.7 mmol) in toluene/dioxane (1:1, 40 mL) at 23° C. was added ethyl chloroacetoacetate (6.37 mL, 47.2 mmol) and the reaction mixture was heated to reflux, while stirring. After 17 h, the reaction mixture was cooled to room temperature and concentrated in vacuo to yield the product, which was purified by silica gel column chromatography (30% EtOAc in hexanes) to yield (2-cyclohexyloxazol-4-yl)acetic acid ethyl ester (1.95 g, 52%).

To a portion of (2-cyclohexyloxazol-4-yl)acetic acid ethyl ester (300 mg, 1.26 mmol) in MeOH/H₂O (2:1, 15 mL) at 23° C. was added LiOH.H₂O (265 mg, 6.32 mmol) and the reaction mixture was stirred overnight. The reaction mixture was concentrated in vacuo and partitioned between H₂O (100 mL) and ethyl ether (100 mL). The aqueous layer was acidified to pH 2-3 with 1.0 N HCl, then extracted with ethyl acetate (2×100 mL). The organic layer was dried (MgSO₄) and concentrated to yield the desired (2-cyclohexyloxazol-4-yl)acetic acid (263 mg, 100%).

(2-Cyclohexyloxazol-4-yl)acetic acid pentafluorophenyl ester was prepared from (2-cyclohexyloxazol-4-yl)acetic acid according to Method A in 69% yield. ¹H NMR (300 MHz, CDCl₃) δ 7.61 (s, 1H), 3.96 (s, 2H), 2.80 (m, 1H), 2.07-1.25 (m, 10H).

4,10-diFmoc-deacylramoplanin amine was reacted with (2-cyclohexyloxazol-4-yl)acetic acid pentafluorophenyl ester according to Method B to obtain Example 96. HPLC: Rₜ=5.39 min (Condition 1). ESMS: m/z 1305.3 [(M+2H)/2].

### Example 97

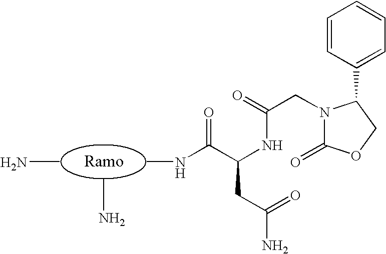

To a solution of (4R)-phenyl-2-oxazolidinone (400 mg, 2.45 mmol) in DMF (5 mL) at 0° C. was added NaH (60% in mineral oil, 117 mg, 2.94 mmol) and the reaction mixture was stirred for 5 min. Benzyl-2-bromoacetate (737 μL, 4.65 mmol) was added and the reaction mixture was stirred at 23° C. for 17 h. The reaction mixture was partitioned between 1.0 N HCl (100 mL) and ethyl acetate (200 mL). The organic layer was washed with saturated aqueous NaHCO₃ (100 mL), then brine (100 mL), dried over Na₂SO₄, and concentrated in vacuo to yield a brown oil that was purified by silica gel column chromatography (0-50% EtOAc in hexanes) to yield (2-oxo-[4R]-phenyloxazolidin-3-yl)acetic acid benzyl ester (622 mg, 82%).

To a solution of (2-oxo-[4R]-phenyloxazolidin-3-yl)acetic acid benzyl ester (311 mg, 1.0 mmol) in ethyl acetate (5 mL) at 23° C. was added Pd/C (10 wt %, 31 mg) and the reaction mixture was deoxygenated via vacuum pump, then purged with hydrogen in a balloon. After 3 h, the reaction mixture was filtered through a pad of Celite, and the Celite pad was rinsed with ethyl acetate (100 mL). The filtrate was concentrated in vacuo to yield (2-oxo-[4R]-phenyloxazolidin-3-yl)acetic acid (221 mg, 100%).

(2-oxo-[4R]-phenyloxazolidin-3-yl)acetic acid pentafluorophenyl ester was prepared from (2-oxo-[4R]-phenyloxazolidin-3-yl)acetic acid according to Method A in 99% yield. ¹H NMR (300 MHz, CDCl₃) δ 7.51-7.44 (m, 3H), 7.36-7.32 (m, 2H), 5.05 (t, J=9.6 Hz, 1H), 4.75 (m, 2H), 4.26 (t, J=7.8 Hz, 1H), 3.73 (d, J=24.3 Hz, 1H), 2.94 (d, J=24 Hz, 1H).

4,10-diFmoc-deacylramoplanin amine was reacted with (2-oxo-[4R]-phenyloxazolidin-3-yl) acetic acid pentafluorophenyl ester according to Method B to obtain Example 97. HPLC: Rₜ=5.00 min (Condition 1). ESMS: m/z 1310.6 [(M+2H)/2].

### Example 98

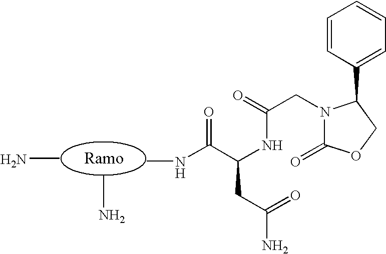

To a solution of (4S)-phenyl-2-oxazolidinone (400 mg, 2.45 mmol) in DMF (5 mL) at 0° C. was added NaH (60% in mineral oil, 117 mg, 2.94 mmol), and the reaction mixture was stirred for 5 min. Benzyl-2-bromoacetate (737 μL, 4.65 mmol) was added and the reaction mixture was stirred at 23° C. for 17 h. The reaction mixture was partitioned between 1.0 N HCl (100 mL) and ethyl acetate (200 mL). The organic layer was washed with saturated aqueous NaHCO₃ (100 mL), brine (100 mL), dried over Na₂SO₄, and concentrated in vacuo to yield a brown oil that was purified by silica gel column chromatography (0-50% EtOAc in hexanes) to yield (2-oxo-[4S]-phenyloxazolidin-3-yl) acetic acid benzyl ester (635 mg, 83%).

To a portion of (2-oxo-[4S]-phenyloxazolidin-3-yl) acetic acid benzyl ester (311 mg, 1.0 mmol) in ethyl acetate (5 mL) at 23° C. was added Pd/C (10 wt %, 31 mg) and the reaction mixture was deoxygenated via vacuum pump, then purged with hydrogen in a balloon. After 3 h, the reaction mixture was filtered through a pad of Celite, and the Celite pad was rinsed with ethyl acetate (100 mL). The filtrate was concentrated in vacuo to yield (2-oxo-[4S]-phenyloxazolidin-3-yl)acetic acid (220 mg, 100%).

(2-oxo-[4S]-phenyloxazolidin-3-yl)acetic acid pentafluorophenyl ester was prepared from (2-oxo-[4S]-phenyloxazolidin-3-yl)acetic acid according to Method A in 99% yield. ¹H NMR (300 MHz, CDCl₃) δ 7.49-7.44 (m, 3H), 7.36-7.32 (m, 2H), 5.05 (t, J=9.6 Hz, 1H), 4.75 (m, 2H), 4.26 (t, J=7.8 Hz, 1H), 3.73 (d, J=24.3 Hz, 1H), 2.94 (d, J=24 Hz, 1H).

4,10-diFmoc-deacylramoplanin amine was reacted with (2-oxo-[4S]-phenyloxazolidin-3-yl) acetic acid pentafluorophenyl ester according to Method B to obtain Example 98. HPLC: Rₜ=4.91 min (Condition 1). ESMS: m/z 1310.9 [(M+2H)/2].

### Example 99

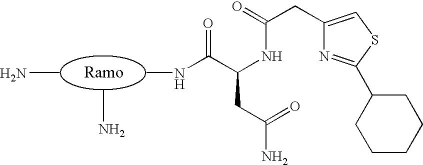

A mixture of cyclohexane carboxamide (1.1 g, 8.56 mmol) and Lawesson's reagent (2.08 g, 5.14 mmol) in THF (35 mL) was stirred at 50° C. for 5 h. The reaction mixture was cooled, concentrated in vacuo, and purified by silica gel column chromatography (0-50% EtOAc in hexanes) to yield cyclohexane thioamide (938 mg, 77%).

To a solution of cyclohexane thioamide (500 mg, 3.49 mmol) in toluene/dioxane (1:1, 20 mL) at 23° C. was added ethyl chloroacetoacetate (1.42 mL, 10.48 mmol) and the reaction mixture was heated to reflux, while stirring. After 17 h, the reaction mixture was cooled to room temperature and concentrated in vacuo to yield the product, which was purified by silica gel column chromatography (0-50% EtOAc in hexanes) to afford (2-cyclohexylthiazol-4-yl)acetic acid ethyl ester (645 mg, 73%).

To a portion of (2-cyclohexylthiazol-4-yl)acetic acid ethyl ester (300 mg, 1.18 mmol) in MeOH/H₂O (2:1, 15 mL) at 23° C. was added LiOH.H₂O (249 mg, 5.93 mmol) and the reaction mixture was stirred overnight. The reaction mixture was concentrated in vacuo and partitioned between H₂O (100 mL) and ethyl ether (100 mL). The aqueous layer was acidified to pH 2-3 with 1.0 N HCl, then extracted with ethyl acetate (2×100 mL). The organic layer was dried (MgSO₄) and concentrated to produce (2-cyclohexylthiazol-4-yl)acetic acid (266 mg, 100%).

(2-Cyclohexylthiazol-4-yl)acetic acid pentafluorophenyl ester was prepared from (2-cyclohexylthiazol-4-yl)acetic acid according to Method A in 86% yield. ¹H NMR (300 MHz, CDCl₃) δ 7.16 (s, 1H), 4.14 (s, 2H), 2.99 (m, 1H), 2.15-1.24 (m, 10H).

4,10-diFmoc-deacylramoplanin amine was reacted with (2-cyclohexylthiazol-4-yl)acetic acid pentafluorophenyl ester according to Method B to obtain Example 99. HPLC: Rₜ=5.63 min (Condition 1). ESMS: m/z 1313.0 [(M+2H)/2].

### Example 100

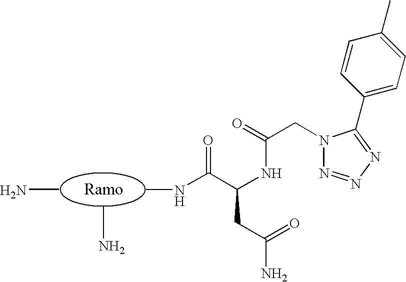

To a solution of the 5-(4-methylphenyl)-1H-tetrazole (400 mg, 2.50 mmol) in DMF (5 mL) at 23° C. was added K₂CO₃ (656 mg, 4.74 mmol) and the reaction mixture was stirred for 30 min. Benzyl-2-bromoacetate (751 μL, 4.74 mmol) was slowly added to the reaction mixture. After stirring for 17 h, the reaction mixture was diluted with ethyl acetate (100 mL) and washed with 1.0 N HCl (100 mL), saturated aqueous NaHCO₃ (100 mL), then brine (100 mL). The reaction mixture was then dried (Na₂SO₄) and concentrated in vacuo and the residue was purified by silica gel column chromatography (0-50% EtOAc in hexanes) to afford (5-p-tolyltetrazol-1-yl)acetic acid benzyl ester (677 mg, 88%).

To a solution of (5-p-tolyltetrazol-1-yl)acetic acid benzyl ester (437 mg, 1.42 mmol) in methanol (5 mL) at 23° C. was added Pd/C (10 wt %, 77 mg) and the reaction mixture was deoxygenated via vacuum pump, then purged with hydrogen in a balloon. After stirring for 3 h, the reaction mixture was filtered through a pad of Celite, and the Celite pad was rinsed with methanol (100 mL). The filtrate was concentrated in vacuo to yield (5-p-tolyltetrazol-1-yl)acetic acid (286 mg, 92%).

(5-p-Tolyltetrazol-1-yl)acetic acid pentafluorophenyl ester was prepared from (5-p-tolyltetrazol-1-yl)acetic acid according to Method A in 85% yield. ¹H NMR (300 MHz, CDCl₃) δ 8.08-8.05 (d, J=8.1 Hz, 2H), 7.33-7.30 (d, J=7.8 Hz, 2H), 5.83 (s, 2H), 2.43 (s, 3H).

4,10-diFmoc-deacylramoplanin amine was reacted with (5-p-tolyltetrazol-1-yl)acetic acid pentafluorophenyl ester according to Method B to obtain Example 100. HPLC: Rₜ=8.16 min (Condition 3). ESMS: m/z 1309.5 [(M+2H)/2].

### Example 101

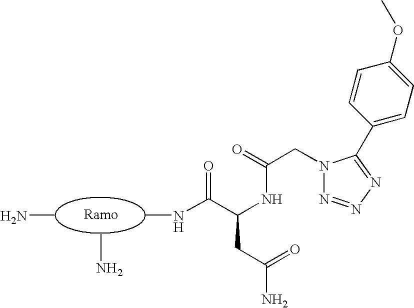

To a solution of the 5-(4-methoxyphenyl)-1H-tetrazole (400 mg, 2.27 mmol) in DMF 5 mL) at 23° C. was added K₂CO₃ (683 mg, 4.31 mmol), and the reaction mixture was stirred for 30 min. Benzyl-2-bromoacetate (683 μL, 4.31 mmol) was slowly added to the reaction mixture. After stirring for 17 h, the reaction mixture was diluted with ethyl acetate (100 mL) and washed with 1.0 N HCl (100 mL), saturated aqueous NaHCO₃ (100 mL), then brine (100 mL). The reaction mixture was then dried (Na₂SO₄) and concentrated to yield the product, which was purified by silica gel column chromatography (0-50% EtOAc in hexanes) to yield [5-(4-methoxyphenyl)tetrazol-1-yl]-acetic acid benzyl ester (693 mg, 94%).

To a solution of [5-(4-methoxyphenyl)tetrazol-1-yl]acetic acid benzyl ester (348 mg, 1.07 mmol) in methanol (5 mL) at 23° C. was added Pd/C (10 wt %, 77 mg) and the reaction mixture was deoxygenated via vacuum pump, then purged with hydrogen in a balloon. After stirring for 3 h, the reaction mixture was filtered through a pad of Celite, and the Celite pad was rinsed with methanol (100 mL). The filtrate was concentrated in vacuo to yield [5-(4-methoxyphenyl)tetrazol-1-yl]-acetic acid (222 mg, 89%).

[5-(4-methoxyphenyl)tetrazol-1-yl]-acetic acid pentafluorophenyl ester was prepared from [5-(4-methoxyphenyl)tetrazol-1-yl]acetic acid according to Method A in 87% yield. ¹H NMR (300 MHz, CDCl₃) δ 8.12-8.09 (m, 2H), 7.04-7.01 (m, 2H), 5.82 (s, 2H), 3.88 (s, 3H).

4,10-diFmoc-deacylramoplanin amine was reacted with [5-(4-methoxyphenyl) tetrazol-1-yl]acetic acid pentafluorophenyl ester according to Method B to obtain Example 101. HPLC: Rₜ=7.71 min (Condition 1). ESMS: m/z 1317.6 [(M+2H)/2].

### Example 102

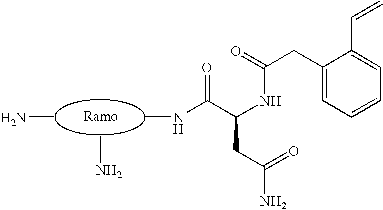

To a stirred solution of 2-iodophenyl acetic acid (1 g, 3.81 mmol) in DMF (20 mL) was added K₂CO₃ (1.5 g) followed by MeI (475 μl). The resulting suspension was stirred for 2 h at which time the reaction was poured into water. This mixture was extracted with ether (20 mL×3) followed by washing of combined ether layers with sat. aq. NaHCO₃, then sat. aq. NaCl. The organic phase was dried over Na₂SO₄, concentrated under reduced pressure to yield pure (2-iodo-phenyl)acetic acid methyl ester which was used for next reaction without any further purification.

To a stirred solution of (2-iodophenyl)acetic acid methyl ester (1 g, 3.62 mmol), LiCl (460 mg, 10.86 mmol), tributylvinyl tin (1.76 mL, 6.02 mmol) and 2,6 ditert-butylphenol (20 mg) in dioxane (50 mL) was added Pd(PPh₃)₄ (250 mg, 5 mol %) under nitrogen atmosphere. The reaction flask was purged with N₂ several times and the resulting mixture was heated to reflux for 4 h at which time the reaction was cooled to rt and quenched by addition of water, MeOH and solid KF. This mixture was further stirred for 20 min followed by extraction with ether. The organic layer was separated, washed with 1N HCl, sat. aq. NaCl and dried over Na₂SO₄. This dried organic phase was concentrated under reduced pressure to yield crude product that was purified by silica gel column chromatography (2.5 to 5% EtOAc in hexanes) to yield (2-vinyl-phenyl)acetic acid methyl ester in 25% yield.

(2-Vinyl-phenyl)acetic acid methyl ester was converted to the corresponding pentafluorophenyl ester according to Method C followed by Method A in 66% yield. ¹H NMR (300 MHz, CDCl₃): δ 7.53 (d, J=6.9 Hz, 1H), 7.33-7.23 (m, 3H), 6.93 (dd, J=10.8 Hz. and 16 Hz, 1H), 5.67 (d, J=18 Hz, 1H), 5.39 (d, J=18 Hz, 1H), 4.03 (s, 2H).

4,10-diFmoc-deacylramoplanin amine was reacted with the above pentafluorophenyl ester according to Method B to obtain Example 102. HPLC: Rₜ=8.00 min (condition 3); Rₜ=7.99 min. (Condition 4). ESMS: m/z 1281.9 [(M+2H)/2].

### Example 103

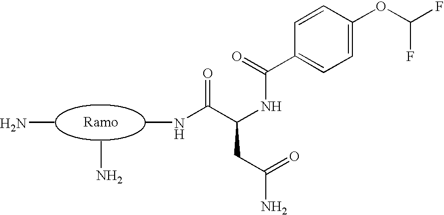

4-Difluoromethoxybenzoic acid pentafluorophenyl ester was prepared from 4-difluoromethoxybenzoic acid according to Method A in 68% yield. ¹H NMR (300 MHz, CDCl₃): δ 8.23-8.18 (m, 2H), 7.26-7.23 (m, 2H), 6.62 (t, J=72.3 Hz, 1H).

4,10-diFmoc-deacylramoplanin amine was reacted with the above pentafluorophenyl ester according to Method B to obtain Example 103. HPLC: Rₜ=8.50 min (Condition 3); Rₜ=3.98 min (Condition 2). ESMS: m/z 1293.8 [(M+2H)/2].

### Example 104

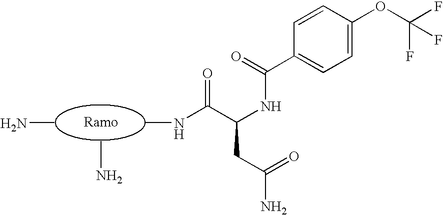

4-Trifluoromethoxybenzoic acid pentafluorophenyl ester was prepared from 4-trifluoromethoxybenzoic acid according to Method A in 52% yield. ¹H NMR (300 MHz, CDCl₃): δ 8.26-8.23 (m, 2H), 7.37-7.34 (m, 2H).

4,10-diFmoc-deacylramoplanin amine was reacted with the above pentafluorophenyl ester according to Method B to obtain Example 104. HPLC: Rₜ=4.10 min (Condition 2). ESMS: m/z 1303.2 [(M+2H)/2].

### Example 105

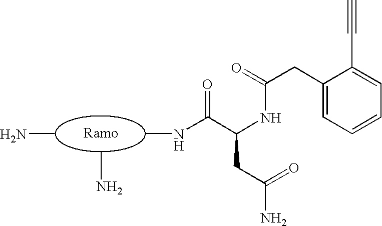

To a stirred solution of 2-iodophenylacetic acid methyl ester (231 mg, 0.84 mmol) in DMF (5 mL) was added CuI (14 mg, 0.07 mmol), trimethylsilylacetylene (107 mL, 0.76 mmol) and Pd(PPh₃)₄ (17 mg, 15 mol %). This mixture was stirred at room temperature for 16 h at which time the reaction was quenched by addition of water, followed by extraction with EtOAc. The organic phase was washed with sat. aq. NaCl, dried over Na₂SO₄ and concentrated under reduced pressure to yield crude product that was purified via silica gel chromatography (2.5% to 5% EtOAc in hexanes) to yield (2-ethynylphenyl)acetic acid methyl ester in 52% yield.

The above (2-ethynylphenyl)acetic acid methyl ester was converted to corresponding pentafluorophenyl ester according to Method C followed by Method A in 68% yield. ¹H NMR (300 MHz CDCl₃): δ 7.55-7.28 (m, 4H), 4.16 (s, 2H), 3.34 (s, 1H).

4,10-diFmoc-deacylramoplanin amine was reacted with above pentafluorophenyl ester according to Method B to obtain Example 105. HPLC: Rₜ=5.17 min (Condition 1); Rₜ=8.31 min (Condition 3). ESMS: m/z 1280.8 [(M+2H)/2].

### Example 106

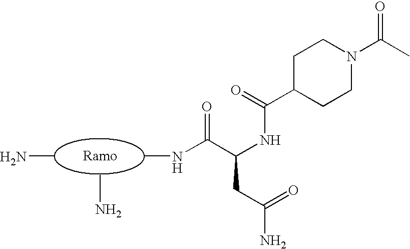

1-Acetylpiperidine-4-carboxylic acid was converted into the corresponding pentafluorophenyl ester using Method A in 46% yield. ¹H NMR (300 MHz, CDCl₃) δ 4.41-4.36 (br m, 2H), 3.85-3.75 (br m, 1H), 3.30-3.12 (br m, 1H), 3.03-2.82 (m, 2H), 2.12-1.40 (m, 7H).

4,10-diFmoc-deacylramoplanin amine was reacted with the above pentafluorophenyl ester according to Method B to obtain Example 106. HPLC: Rₜ=11.60 min (Condition 3); Rₜ=5.71 min (Condition 1). ESMS: m/z 1309.2 [(M+2H)/2].

### Example 107

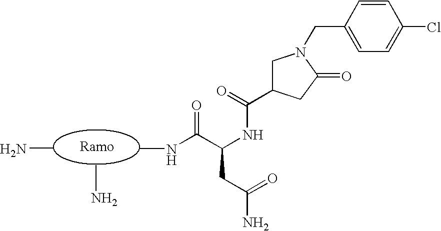

1-(4-Chloro-benzyl)-5-oxopyrrolidine-3-carboxylic acid was converted into the corresponding pentafluorophenyl ester using Method A in 20% yield. ¹H NMR (300 MHz, CDCl₃): δ 7.30-7.10 (m, 4H), 4.50 (s, 2H), 3.70-3.50 (m, 3H), 2.95 (d, J=9.0 Hz, 2H).

4,10-diFmoc-deacylramoplanin amine was reacted with the above pentafluorophenyl ester according to Method B to obtain Example 107. HPLC: Rₜ=5.19 min (Condition 1); Rₜ=7.95 min (Condition 3). ESMS: m/z 1327.3 [(M+2H)/2].

### Example 108

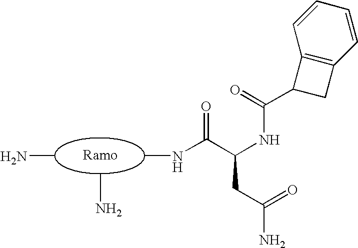

(±)Bicyclo[4.2.0]octa-1(6),2,4-triene-7-carboxylic acid was converted into the corresponding pentafluorophenyl ester using Method A in 64% yield. ¹H NMR (300 MHz, CDCl₃): δ 7.26-7.09 (m, 4H), 4.58 (t, J=4.2 Hz, 1H), 3.57 (d, J=4.2 Hz, 2H).

4,10-diFmoc-deacylramoplanin amine was reacted with the above pentafluorophenyl ester according to Method B to obtain Example 108. HPLC: Rₜ=5.47 min (Condition 1); Rₜ=3.89 and 3.93 min (Condition 2). ESMS: m/z 1274.5 [(M+2H)/2].

### Example 109

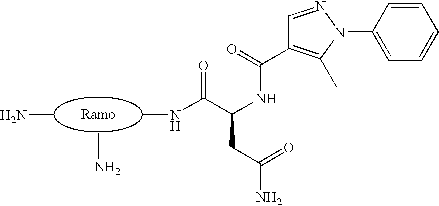

Ethyl acetoacetate (5 g, 38.42 mmol) and N,N-Dimethylformamide dimethylacetal (5.10 mL, 38.42 mmol) were heated to reflux for 3 h, at which time the reaction mixture was concentrated under reduced pressure to yield 2-acetyl-3-dimethylaminoacrylic acid ethyl ester (6.39 g). This 2-acetyl-3-dimethylaminoacrylic acid ethyl ester was dissolved in EtOH (25 mL) followed by addition of phenylhydrazine (3.4 mL). The resulting mixture was heated to reflux for 4 h at which time the reaction was concentrated under reduced pressure to yield (5-methyl-1-phenyl-1H-pyrazol-4-yl)acetic acid ethyl ester in 42% yield.

The above (5-methyl-1-phenyl-1H-pyrazol-4-yl)acetic acid ethyl ester was converted to the corresponding pentafluorophenyl ester according to Method C followed by Method A in 26% yield. ¹H NMR (300 MHz, CDCl₃): δ 8.20 (s, 1H), 7.53-7.41 (m, 5H), 2.59 (s, 3H).

4,10-diFmoc-deacylramoplanin amine was reacted with the above pentafluorophenyl ester according to Method B to obtain Example 109. HPLC: Rₜ=8.26 min (Condition 3); Rₜ=5.25 min (Condition 1). ESMS: m/z 1301.4 [(M+2H)/2].

### Example 110

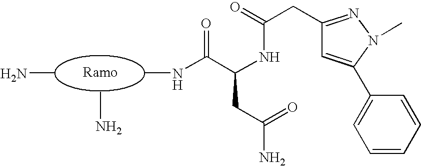

(1-Methyl-5-phenyl-1H-pyrazol-3-yl)acetic acid ethyl ester was synthesized as described in Method R using methylhydrazine. This ester was converted into the corresponding pentafluorophenyl ester using Method C followed by Method A in 28% yield. ¹H NMR (300 z, CDCl₃): δ 7.75 (d, J=7.2 Hz, 2H), 7.39-7.28 (m, 3H), 6.59 (s, 1H), 4.07 (s, 2H), 3.99 (s, 3H).

4,10-diFmoc-deacylramoplanin amine was reacted with above pentafluorophenyl ester according to Method B to obtain Example 110. HPLC: Rₜ=7.85 min (Condition 3); Rₜ=5.26 min (condition 1). ESMS: m/z 1308.4 [(M+2H)/2].

### Example 111

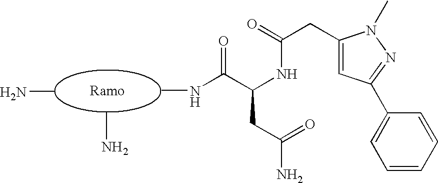

(2-Methyl-5-phenyl-2H-pyrazol-3-yl)acetic acid ethyl ester was synthesized as described in Method R using methylhydrazine. This ester was converted into the corresponding pentafluorophenyl ester using Method C followed by Method A in 36% yield. ¹H NMR (300 MHz, CDCl₃): δ 7.51-7.41 (m, 5H), 6.46 (s, 1H), 4.19 (s, 2H), 3.93 (s, 3H).

4,10-diFmoc-deacylramoplanin amine was reacted with the above pentafluorophenyl ester according to Method B to obtain Example 111. HPLC: Rₜ=8.08 min (Condition 3); Rₜ=5.34 min (Condition 1). ESMS: m/z 1308.3 [(M+2H)/2].

### Example 112

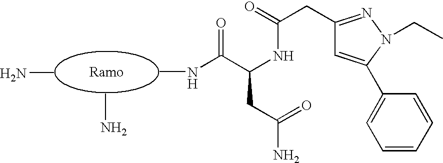

(1-Ethyl-5-phenyl-1H-pyrazol-3-yl)acetic acid ethyl ester was synthesized as described in Method R using ethylhydrazine. This ester was converted into the corresponding pentafluorophenyl ester using Method C followed by Method A in 28% yield. ¹H NMR (300 MHz, CDCl₃): δ 8.58-8.56 (m, 1H), 7.48-7.23 (m, 4H), 6.32 (s, 1H), 4.13 (q, J=7.2 Hz, 2H), 4.05 (s, 2H), 1.37 (t, J=7.0 Hz, 3H).

4,10-diFmoc-deacylramoplanin amine was reacted with the above pentafluorophenyl ester according to Method B to obtain Example 112. HPLC: Rₜ=4.96 min (Condition 2); Rₜ=5.75 min (Condition 1). ESMS: m/z 1315.4 [(M+2H)/2].

### Example 113

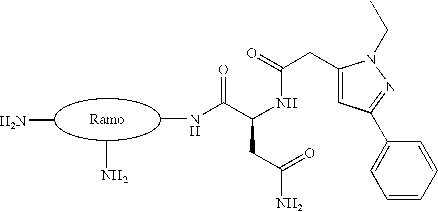

(2-Ethyl-5-phenyl-2H-pyrazol-3-yl)acetic acid ethyl ester was synthesized as described in Method R using ethylhydrazine. This ester was converted into the corresponding pentafluorophenyl ester using Method C followed by Method A in 36% yield. ¹H NMR (300 MHz, CDCl₃): δ 7.78-7.75 (m, 2H), 7.40-7.24 (m, 3H), 6.57 (s, 1H), 4.17 (q, J=7.2 Hz, 2H), 4.06 (s, 2H), 1.50 (t, J=6.0 Hz, 3H)

4,10-diFmoc-deacylramoplanin amine was reacted with the above pentafluorophenyl ester according to Method B to obtain Example 113. HPLC: Rₜ=8.26 min (Condition 3); Rₜ=5.57 min (Condition 1). ESMS: m/z 1315.4 [(M+2H)/2].

### Example 114

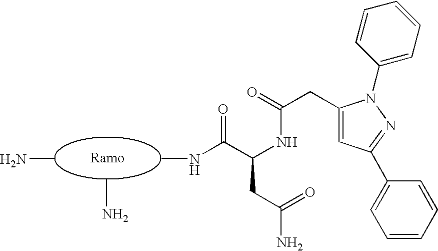

(2,5-Diphenyl-2H-pyrazol-3-yl)acetic acid ethyl ester was synthesized as described in Method R using phenylhydrazine. This ester was converted into the corresponding pentafluorophenyl ester using Method C followed by Method A in 28% yield. ¹H NMR (300 MHz, CDCl₃): 7.32-7.22 (m, 10H), 6.58 (s, 1H), 4.15 (s, 2H).

4,10-diFmoc-deacylramoplanin amine was reacted with the above pentafluorophenyl ester according to Method B to obtain Example 114. HPLC: R=4.11 min (Condition 2); Rₜ=5.20 min (Condition 1). ESMS: m/z 1339.6 [(M+2H)/2].

### Example 115

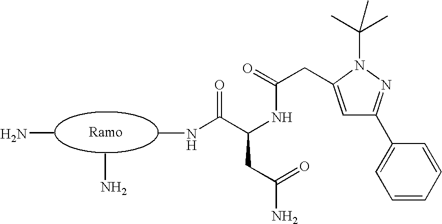

(2-t-Butyl-5-phenyl-2H-pyrazol-3-yl)acetic acid ethyl ester was synthesized as described in Method R using t-butyl hydrazine. This ester was converted into corresponding pentafluorophenyl ester using Method C followed by Method A in 28% yield. ¹H NMR (300 MHz, CDCl₃): δ 7.39-7.34 (m, 5H), 6.19 (s, 1H), 4.04 (s, 2H), 1.45 (s, 9H).

4,10-diFmoc-deacylramoplanin amine was reacted with above pentafluorophenyl ester according to Method B to obtain Example 115. HPLC: Rₜ=6.19 min (Condition 1); Rₜ=4.30 min (Condition 2). ESMS: m/z 1329.4 [(M+2H)/2].

### Example 116

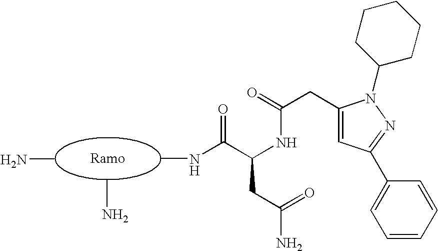

(2-Cyclohexyl-5-phenyl-2H-pyrazol-3-yl)acetic acid ethyl ester was synthesized as described in Method R using cyclohexyl hydrazine. This ester was converted into the corresponding pentafluorophenyl ester using Method C followed by Method A in 28% yield. ¹H NMR (300 MHz, CDCl₃): δ 7.47-7.32 (m, 5H), 6.26 (s, 1H), 4.09-4.02 (m, 3H), 2.02-1.62 (m, 6H), 1.26-1.20 (m, 4H).

4,10-diFmoc-deacylramoplanin amine was reacted with the above pentafluorophenyl ester according to Method B to obtain Example 116. HPLC: Rₜ=6.44 min (Condition 1); 4.45 min (Condition 2). ESMS: m/z 1342.1 [(+2H)/2].

### Example 117

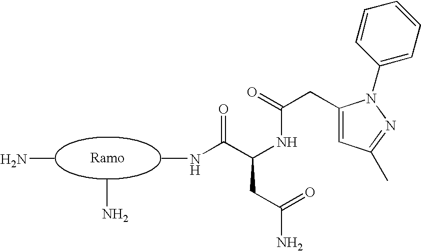

To a solution of Meldrum's acid (7.83 g, 54.36 mmol) in DCM under N₂ was added TEA (7.57 mL) followed by cooling of the reaction to 0° C. To this mixture was added diketene (5 mL, 65.24 mmol) and the resulting mixture was stirred for 1 h at 0° C. and then for 4 h at rt. The reaction was then concentrated under reduced pressure to yield pure 5-(1-Hydroxy-3-oxobutylidene)-2,2-dimethyl-[1,3]dioxane-4,6-dione as yellow solid in quantitative yield that was used for next reaction without any further purification.

To a stirred solution of 5-(1-hydroxy-3-oxobutylidene)-2,2-dimethyl-[1,3]dioxane-4,6-dione (456 mg, 2 mmol) in EtOH was added phenylhydrazine (196 μL, 2 mmol) and the reaction mixture was heated to 60° C. for 16 h. At this time the reaction was diluted with EtOAc followed by washing with 1N HCl. The organic phase was separated, dried over Na₂SO₄ and concentrated under reduced pressure to yield (5-methyl-2-phenyl-2H-pyrazol-3-yl)acetic acid ethyl ester.

(5-Methyl-2-phenyl-2H-pyrazol-3-yl)acetic acid ethyl ester was converted into the corresponding pentafluorophenyl ester using Method C followed by Method A in 28% yield. ¹H NMR (300 MHz, CDCl₃): δ 7.47-7.37 (m, 5H), 6.30 (s, 1H), 4.00 (s, 2H), 2.34 (s, 3H).

4,10-diFmoc-deacylramoplanin amine was reacted with the above pentafluorophenyl ester according to Method B to obtain Example 117. HPLC: Rₜ=5.14 min (Condition 1); 3.68 min (Condition 2). ESMS: m/z 1308.4 [(M+2H)/2].

### Example 118

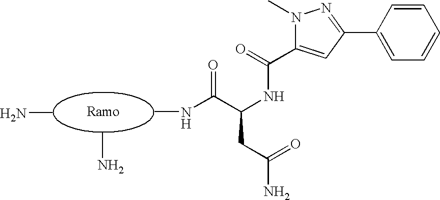

To a solution of dimethyloxalate (590 mg, 5 mmol) and NaOMe (40 mL, 0.5 M solution in THF, 20 mmol) in MeOH was added acetophenone (292 μL, 2.5 mmol). The resulting mixture was stirred for 2 h at which time the reaction was quenched with 1N HCl. This mixture was extracted with EtOAc, the organic phase was dried over Na₂SO₄ followed by concentration under reduced pressure to yield crude 2,4-dioxo-4-phenyl-butyric acid methyl ester in 64% yield.

To a stirred solution of 2,4-dioxo-4-phenyl-butyric acid methyl ester (1 g, 4.58 mmol) in AcOH was added methylhydrazine (243 mL, 4.58 mmol) and the resulting reaction was heated to reflux for 1 h at which time the reaction was stopped by concentrating under reduced pressure. The residue was dissolved in EtOAc, washed with sat. aq. NaHCO₃, then water, followed by dryng over Na₂SO₄. The dried organic phase was concentrated under reduced pressure to yield crude product that was purified by silica gel column chromatography (0-20% EtOAc in DCM) to afford pure 1-Methyl-5-phenyl-1H-pyrazole-3-carboxylic acid methyl ester and 2-methyl-5-phenyl-2H-pyrazole-3-carboxylic acid methyl ester.

2-Methyl-5-phenyl-2H-pyrazole-3-carboxylic acid methyl ester was converted into the corresponding pentafluorophenyl ester using Method C followed by Method A in 51% yield. ¹H NMR (300 MHz, CDCl₃): δ 7.50-7.41 (m, 5H), 7.00 (s, 1H), 4.00 (s, 3H).

4,10-diFmoc-deacylramoplanin amine was reacted with the above pentafluorophenyl ester according to Method B to obtain Example 118. HPLC: Rₜ=4.49 min (Condition 1); Rₜ=4.03 min (Condition 2). ESMS: m/z 1301.4 [(M+2H)/2].

### Example 119

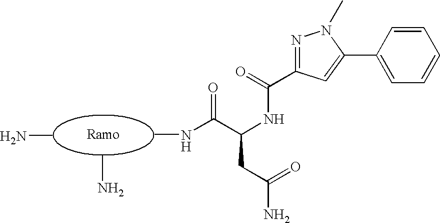

1-Methyl-5-phenyl-1H-pyrazole-3-carboxylic acid methyl ester (see Example 118) was converted into the corresponding pentafluorophenyl ester using Method C followed by Method A in 51% yield. ¹H NMR (300 MHz, CDCl₃) δ 7.65 (d, J=8.1 Hz, 2H), 7.29-7.19 (m, 3H), 7.08 (s, 1H) 4.10 (s, 3H).

4,10-diFmoc-deacylramoplanin amine was reacted with the above pentafluorophenyl ester according to Method B to obtain Example 119. HPLC: Rₜ=4.03 min (Condition 2). ESMS: m/z 1301.4 [(M+2H)/2].

### Example 120

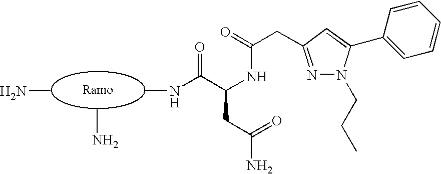

(5-Phenyl-1-propyl-1H-pyrazol-3-yl)acetic acid ethyl ester was synthesized as described in Method S using iodopropane. This ester was converted into the corresponding pentafluorophenyl ester using Method C followed by Method A in 46% yield. ¹H NMR (300 MHz, CDCl₃): δ 7.79-7.76 (m, 2H), 7.40-7.28 (m, 3H), 6.59 (s, 1H), 4.12-4.07 (m, 4H), 1.98-1.90 (m, 2H), 0.94 (t, J=7.5 Hz, 3H).

4,10-diFmoc-deacylramoplanin amine was reacted with the above pentafluorophenyl ester according to Method B to obtain Example 120. HPLC: Rₜ=5.24 min (Condition 1); Rₜ=4.25 min (Condition 2). ESMS: m/z 1322.4 [(M+2H)/2].

### Example 121

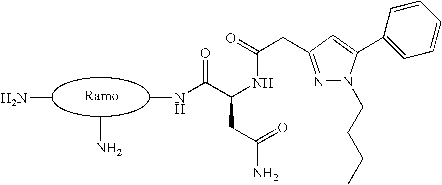

(1-Butyl-5-phenyl-1H-pyrazol-3-yl)acetic acid ethyl ester was synthesized as described in Method S using iodobutane. This ester was converted into the corresponding pentafluorophenyl ester using Method C followed by Method A in 47% yield. ¹H NMR (300 MHz, CDCl₃): δ 7.76-7.73 (m, 2H), 7.39-7.28 (m, 3H), 6.59 (s, 1H), 4.16-4.07 (m, 4H), 1.93-1.83 (m, 2H), 1.42-1.35 (m, 2H), 0.95 (t, J=7.5 Hz, 3H)

4,10-diFmoc-deacylramoplanin amine was reacted with the above pentafluorophenyl ester according to Method B to obtain Example 121. HPLC: Rₜ=5.12 min (Condition 1); Rₜ=4.11 min (Condition 2). ESMS: m/z 1329.4 [(M+2H)/2].

### Example 122

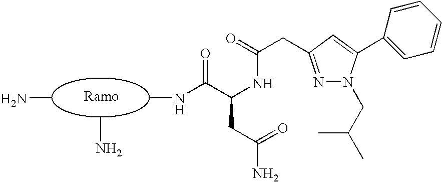

(1-Isobutyl-5-phenyl-1H-pyrazol-3-yl)acetic acid ethyl ester was synthesized as described in Method S using isobutyliodide. This ester was converted into the corresponding pentafluorophenyl ester using Method C followed by Method A in 47% yield. ¹H NMR (300 MHz, CDCl₃): δ 7.95-7.93 (m, 2H), 7.47-7.41 (m, 3H), 6.75 (s, 1H), 4.24-4.21 (d, J=8.1 Hz, 2H), 4.11-4.09 (m, 2H), 2.46-2.43 (m, 1H), 1.00 (d, J=6.6 Hz, 6H).

4,10-diFmoc-deacylramoplanin amine was reacted with the above pentafluorophenyl ester according to Method B to obtain Example 122. HPLC: Rₜ=5.27 min (Condition 1); Rₜ=4.23 min (Condition 2). ESMS: m/z 1329.4 [(M+2H)/2].

### Example 123

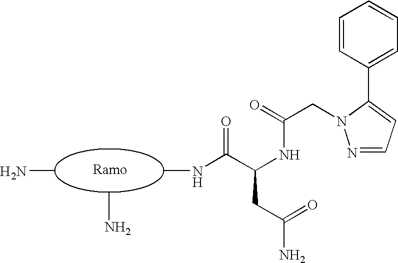

Acetophenone (4.5 mL, 38.42 mmol) and N,N-dimethylformamide dimethylacetal (5.10 mL, 38.42 mmol) were heated to reflux for 3 h at which time the reaction mixture was concentrated under reduced pressure to afford 3-dimethylamino-1-phenylpropenone (5.98 g). To a solution of 3-Dimethylamino-1-phenyl-propenone in MeOH (25 mL) was added hydrazine (4 mL). The resulting mixture was stirred for 16 h at which time the reaction was concentrated under reduced pressure to yield crude 5-phenyl-1H-pyrazole in 51% yield.

To a stirred solution of 5-phenyl-1H-pyrazole (1 g, 6.94 mmol) in DMF (10 mL) was added K₂CO₃ (1 g) followed by addition of methylbromoacetate (1.30 mL, 13.88 mmol). This mixture was heated to 50° C. for 4 h at which time the reaction was diluted with 1N HCl. The resulting mixture was extracted with EtOAc, the organic phase was dried over Na₂SO₄ followed by concentration under reduced pressure to yield crude (5-Phenyl-pyrazol-1-yl)acetic acid methyl ester that was used for next reaction without any further purification.

The above (5-phenylpyrazol-1-yl)acetic acid methyl ester was converted to the corresponding pentafluorophenyl ester according to Method C followed by Method A in 56% yield. ¹H NMR (300 MHz, DMSO) δ 7.92 (d, J=3.0 Hz, 1H), 7.81-7.79 (m, 2H), 7.42-7.30 (m, 3H), 6.80 (d, J=3.0 Hz, 1H), 5.71 (s, 2H).

4,10-diFmoc-deacylramoplanin amine was reacted with the above pentafluorophenyl ester according to Method B to obtain Example 123. HPLC: Rₜ=5.03 min (Condition 1); Rₜ=4.10 min (Condition 2). ESMS: m/z 1301.4 [(M+2H)/2].

### Example 124

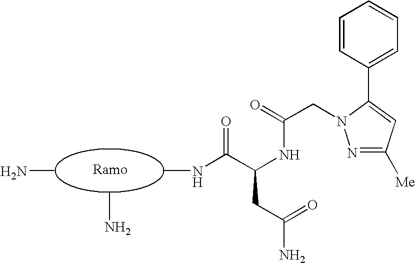

(3-Methyl-5-phenyl-pyrazol-1-yl)acetic acid methyl ester as prepared by similar method described for Example 123, substituting N,N-dimethylacetamide dimethylacetal for N,N-dimethylformamide dimethylacetal.

The above (3-methyl-5-phenylpyrazol-1-yl)acetic acid methyl ester was converted to the corresponding pentafluorophenyl ester according to Method C followed by Method A in 64% yield. ¹H NMR (300 MHz, DMSO): δ 7.76-7.73 (m, 2H), 7.40-7.28 (m, 3H), 6.59 (s, 1H), 5.67 (s, 2H), 2.30 (s, 3H).

4,10-diFmoc-deacylramoplanin amine was reacted with the above pentafluorophenyl ester according to Method B to obtain Example 124. HPLC: Rₜ=5.09 min (Condition 1); Rₜ=4.03 min (Condition 2). ESMS: m/z 1308.8 [(M+2H)/2].

### Example 125

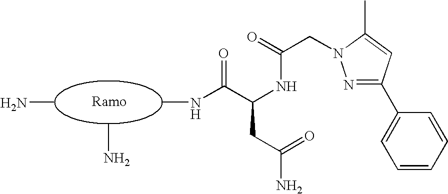

(5-Methyl-3-phenyl-1H-pyrazol-1-yl)acetic acid ethyl ester was prepared by a similar method described for Example 123 substituting N,N-Dimethylacetamide dimethylacetal for N,N-dimethylformamide dimethylacetal and ethylhydrazinoacetate for hydrazine.

The above (5-Methyl-3-phenyl-1H-pyrazol-1-yl)acetic acid ethyl ester was converted to the corresponding pentafluorophenyl ester according to Method C followed by Method A in 53% yield. ¹H NMR (300 MHz, DMSO) δ 7.47-7.44 (m, 5H), 6.28 (s, 1H), 5.50 (s, 2H), 2.20 (s, 3H).

4,10-diFmoc-deacylramoplanin amine was reacted with the above pentafluorophenyl ester according to Method B to obtain Example 125. HPLC: Rₜ=5.09 min (Condition 1); 4.10 min (Condition 2). ESMS: m/z 1308.8 [(M+2H)/2].

### Example 126

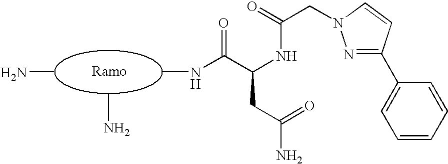

(3-Phenylpyrazol-1-yl)acetic acid ethyl ester was prepared by a similar method described for Example 123 using N,N-dimethylformamide dimethylacetal and substituting ethylhydrazinoacetate for hydrazine.

The above (3-phenylpyrazol-1-yl)acetic acid ethyl ester was converted to the corresponding pentafluorophenyl ester according to Method C followed by Method A in 42% yield. ¹H NMR (300 MHz, DMSO): δ 7.63 (s, 1H), 7.50-7.48 (m, 5H), 6.49 (s, 1H), 5.62 (s, 2H).

4,10-diFmoc-deacylramoplanin amine was reacted with the above pentafluorophenyl ester according to Method B to obtain Example 126. HPLC: Rₜ=5.11 min (Condition 1); 4.03 min (Condition 2). ESMS: m/z 1308.4 [(M+2H)/2].

### Example 127

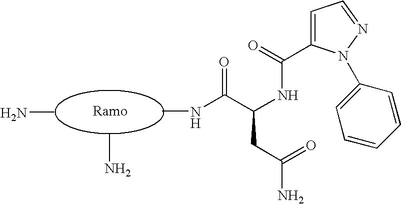

2-Phenyl-2H-pyrazole-3-carboxylic acid was converted to the corresponding pentafluorophenyl ester according to Method A in 35% yield. ¹H NMR (300 MHz, DMSO): δ 8.00 (s, 1H), 7.55-7.48 (m, 6H).

4,10-diFmoc-deacylramoplanin amine was reacted with the above pentafluorophenyl ester according to Method B to obtain Example 127. HPLC: Rₜ=4.74 min (Condition 1); 3.73 min (Condition 2). ESMS: m/z 1294.4 [(M+2H)/2].

### Example 128

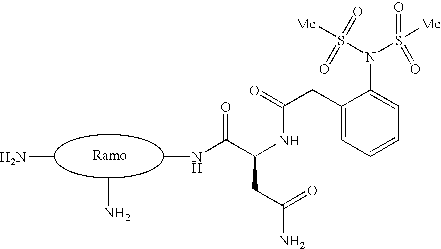

To a stirred solution of 2-aminophenylacetic acid methyl ester (300 mg, 1.81 mmol) in pyridine at 0° C. was slowly added methanesulfonyl chloride (281 μL, 3.63 mmol). The solution was stirred for 4 h at which time the reaction was quenched by addition of 1N HCl. The resulting solution was extracted with EtOAc, and the organic phase was dried over Na₂SO₄, and concentrated under reduced pressure to yield [2-(bis-methanesulfonylamino)phenyl]acetic acid methyl ester.

The above [2-(bis-methanesulfonylamino)phenyl]acetic acid methyl ester was converted to the corresponding pentafluorophenyl ester according to Method C followed by Method A in 64% yield. ¹H NMR (300 MHz, CDCl₃): δ 7.60-7.40 (m, 4H), 4.15 (s, 2H), 3.45 (s, 6H).

4,10-diFmoc-deacylramoplanin amine was reacted with the above pentafluorophenyl ester according to Method B to obtain Example 128. HPLC: Rₜ=5.09 min (Condition 1); Rₜ=7.57 min (Condition 2). ESMS: m/z 1353.9 [(M+2H)/2].

### Example 129

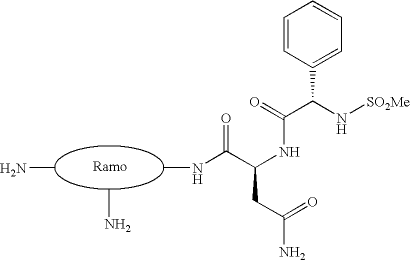

To a stirred solution of L-Phenyl glycine hydrochloride (1 equivalent) in 1N NaOH at 0° C. was added methanesulfonylchloride (1.2 equivalent). The resulting solution was stirred for 3 h at which time the reaction was extracted with ether. The resulting aqueous phase was acidified with 1N HCl to pH 3. This was extracted with EtOAc, and the organic phase was dried over Na₂SO₄, and concentrated under reduced pressure to yield methanesulfonylamino-(L)-phenyl-acetic acid in near quantitative yield.

Methanesulfonylamino-(L)-phenylacetic acid was converted to the corresponding pentafluorophenyl ester according to Method A in 23% yield. ¹H NMR (300 MHz, CDCl₃): δ 7.49-7.44 (m, 5H), 5.59 (d, J=6.6 Hz, 1H), 5.39 (br d, J=6.6 Hz, 1H), 2.84 (s, 3H).

4,10-diFmoc-deacylramoplanin amine was reacted with the above pentafluorophenyl ester according to Method B to obtain Example 129. HPLC: Rₜ=7.24 min (Condition 4); Rₜ=4.93 min (Condition 1). ESMS: m/z 1315.1 [(M+2H)/2].

### Example 130

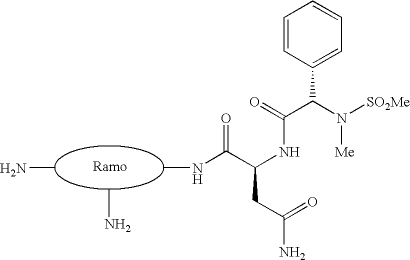

To a stirred solution of methanesulfonylamino-(L)-phenylacetic acid (1 equivalent, see Example 129) in MeOH at 0° C. was slowly added TMSCHN₂ in hexane (10 equivalent). The resulting solution was allowed to warm up to rt over 20 to 30 min followed by concentration of the reaction under reduced pressure to yield (methanesulfonylmethylamino)-(L)-phenyl-acetic acid methyl ester.

(Methanesulfonylmethylamino)-(L)-phenylacetic acid methyl ester was converted to the corresponding pentafluorophenyl ester according to Method C followed by Method A in 12% yield. ¹H NMR (300 MHz, CDCl₃) δ 7.46-7.38 (m, 5H), 6.21 (s, 1H), 2.94 (s, 3H), 2.08 (s, 3H).

4,10-diFmoc-deacylramoplanin amine was reacted with the above pentafluorophenyl ester according to Method B to obtain Example 130. HPLC: Rₜ=5.15 min (Condition 1); 7.74 min (Condition 4). ESMS: m/z 1321.8 [(M+2H)/2].

### Example 131

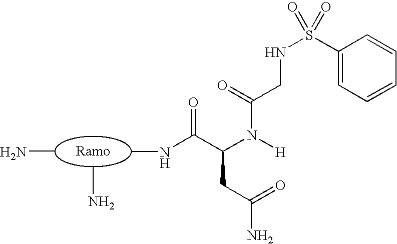

Benzenesulfonylaminoacetic acid was prepared according to Method T followed by conversion to the corresponding pentafluorophenyl ester according to Method A in 46% yield. ¹H NMR (300 MHz, DMSO) δ 8.58 (t, J=6.3 Hz, 1H), 7.98 (d, J=8.7 Hz, 1H), 7.84-7.55 (m, 4H), 4.26 (d, J=6.0 Hz, 2H).

4,10-diFmoc-deacylramoplanin amine was reacted with the above pentafluorophenyl ester according to Method B to obtain Example 131. HPLC: Rₜ=7.21 min (Condition 3); 7.26 min (Condition 4). ESMS: m/z 1307.8 [(M+2H)/2].

### Example 132

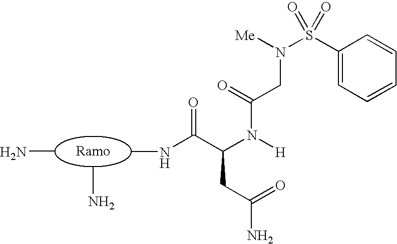

(Benzenesulfonylmethylamino)acetic acid was prepared according to Method T using MeI followed by conversion to the corresponding pentafluorophenyl ester according to Method A in 43% yield. ¹H NMR (300 MHz, CDCl₃) δ 7.84-7.80 (m, 2H), 7.58-7.48 (m; 3H), 4.39 (s, 2H), 2.96 (s, 3H).

4,10-diFmoc-deacylramoplanin amine was reacted with the above pentafluorophenyl ester according to Method B to obtain Example 132. HPLC: Rₜ=5.13 min (Condition 1); 8.09 min (Condition 4). ESMS: m/z 1315.5 [(M+2H)/2].

### Example 133

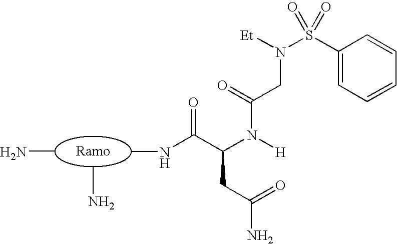

(Benzenesulfonyl-ethyl-amino)acetic acid was prepared according to Method T using ethyl iodide, followed by conversion to the corresponding pentafluorophenyl ester according to Method A in 47% yield. ¹H NMR (300 MHz, DMSO): δ 7.89-7.85 (m, 2H), 7.71-7.57 (m, 3H), 4.62 (s, 2H), 3.27 (q, J=6.9 Hz, 2H), 1.04 (t, J=7.2 Hz, 3H).

4,10-diFmoc-deacylramoplanin amine was reacted with the above pentafluorophenyl ester according to Method B to obtain Example 133. HPLC: Rₜ=5.24 min (Condition 1); 7.87 min (Condition 4). ESMS: m/z 1321.8 [(M+2H)/2].

### Example 134

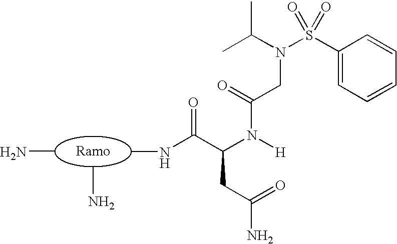

(Benzenesulfonylisopropylamino)acetic acid was prepared according to Method T using isopropyl iodide, followed by conversion to the corresponding pentafluorophenyl ester according to Method A in 54% yield. ¹H NMR (300 MHz, CDCl₃): δ 7.95-7.92 (m, 2H), 7.59-7.49 (m, 3H), 4.35 (s, 2H), 4.04 (m, 1H), 1.08 (d, J=6 Hz, 6H).

4,10-diFmoc-deacylramoplanin amine was reacted with the above pentafluorophenyl ester according to Method B to obtain Example 134. HPLC: Rₜ=5.36 min (Condition 1); Rₜ=8.15 min (Condition 4). ESMS: m/z 1329.1 [(M+2H)/2].

### Example 135

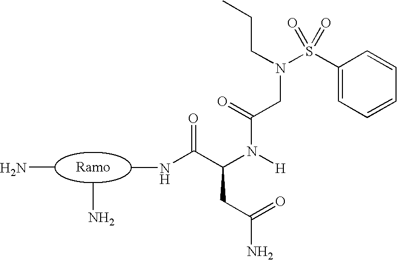

(Benzenesulfonylpropylamino)acetic acid was prepared according to Method T using 1-iodopropane, followed by conversion to the corresponding pentafluorophenyl ester according to Method A in 42% yield. ¹H NMR (300 MHz, CDCl₃): δ 7.85-7.82 (m, 2H), 7.58-7.48 (m, 3H), 4.43 (s, 2H), 3.24 (t, J=7.2 Hz, 2H), 1.62-1.54 (m, 2H), 0.89 (t, J=7.5 Hz, 3H).

4,10-diFmoc-deacylramoplanin amine was reacted with the above pentafluorophenyl ester according to Method B to obtain Example 135. HPLC: Rₜ=5.40 min (Condition 1); 8.25 min (Condition 4). ESMS: m/z 1329.1 [(M+2H)/2].

### Example 136

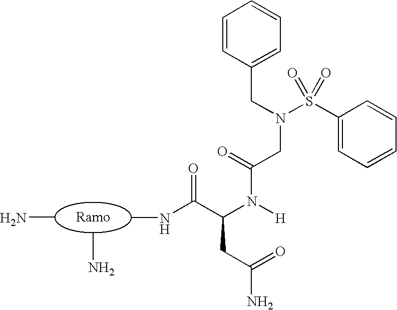

(Benzenesulfonylbenzylamino)acetic acid was prepared according to Method T using benzylbromide, followed by conversion to the corresponding pentafluorophenyl ester according to Method A in 73% yield. ¹H NMR (300 MHz, CDCl₃): δ 7.90-7.87 (m, 2H), 7.61-7.32 (m, 3H), 7.36-7.22 (m, 5H), 4.50 (s, 2H), 4.28 (s, 2H).

4,10-diFmoc-deacylramoplanin amine was reacted with the above pentafluorophenyl ester according to Method B to obtain Example 136. HPLC: Rₜ=5.62 min (Condition 1); Rₜ=8.76 min (Condition 4). ESMS: m/z 1353.3 [(M+2H)/2].

### Example 137

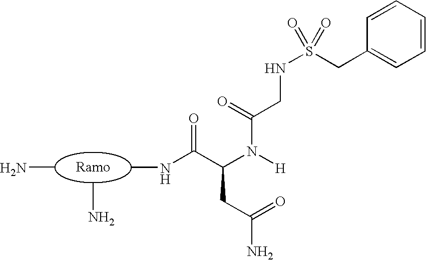

Benzylsulfonylamino-acetic acid was prepared according to Method U followed by conversion to corresponding pentafluorophenyl ester according to Method A in 24% yield. ¹H NMR (300 MHz, CDCl₃): δ 7.70-7.45 (m, 5H), 5.05-4.95 (m, 1H), 4.60 (s, 2H), 4.30 (d, J=12 Hz, 2H).

4,10-diFmoc-deacylramoplanin amine was reacted with above pentafluorophenyl ester according to Method B to obtain Example 137. HPLC: Rₜ=5.12 min (Condition 1); Rₜ=7.81 min (Condition 4). ESMS: m/z 1315.1 [(M+2H)/2].

### Example 138

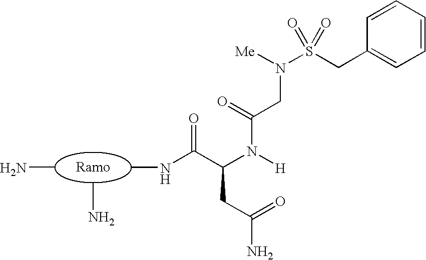

(Methylphenylmethanesulfonylamino)acetic acid was prepared according to Method U using methyl iodide followed by conversion to corresponding pentafluorophenyl ester according to Method A in 32% yield. ¹H NMR (300 MHz, CDCl₃): δ 7.44-7.36 (m, 5H), 4.32 (s, 2H), 4.20 (s, 2H), 2.87 (s, 3H).

4,10-diFmoc-deacylramoplanin amine was reacted with above pentafluorophenyl ester according to Method B to obtain Example 138. HPLC: Rₜ=5.28 min (Condition 1); Rₜ=8.23 min (Condition 4). ESMS: m/z 1322.1 [(M+2H)/2].

### Example 139

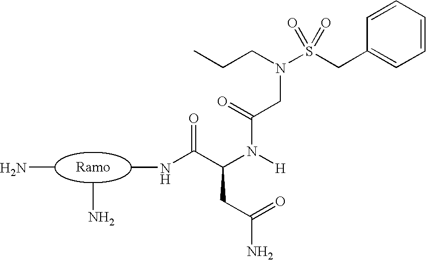

(Propyl-benzylsulfonyl-amino)acetic acid was prepared according to Method U using I-iodopropane, followed by conversion to the corresponding pentafluorophenyl ester according to Method A in 43% yield. ¹H NMR (300 MHz, CDCl₃): δ 7.70-7.50 (m, 5H), 4.60 (s, 2H), 4.20 (s, 2H), 3.40-3.20 (m, 2H), 1.80-1.60 (m, 2H), 1.10 (t, J=8.5 Hz, 3H).

4,10-diFmoc-deacylramoplanin amine was reacted with the above pentafluorophenyl ester according to Method B to obtain Example 139. HPLC: Rₜ=5.65 min (Condition 1); Rₜ=8.28 min (Condition 4). ESMS: m/z 1336.4 [(M+2H)/2].

### Example 140

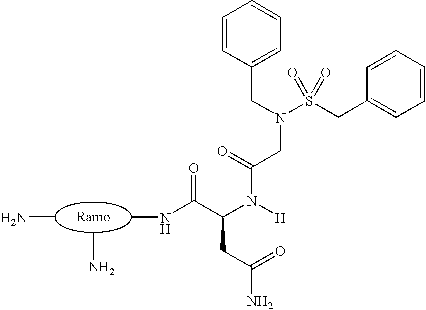

(Benzyl-benzylsulfonyl-amino)acetic acid was prepared according to Method U using benzylbromide, followed by conversion to the corresponding pentafluorophenyl ester according to Method A in 78% yield. ¹H NMR (300 MHz, CDCl₃): δ 7.49-7.23 (m, 10H), 4.40 (s, 2H), 4.22 (s, 2H), 4.20 (s, 2H).

4,10-diFmoc-deacylramoplanin amine was reacted with the above pentafluorophenyl ester according to Method B to obtain Example 140. HPLC: Rₜ=5.79 min (Condition 1); 8.86 min (Condition 4). ESMS: m/z 1359.9 [(M+2H)/2].

### Example 141

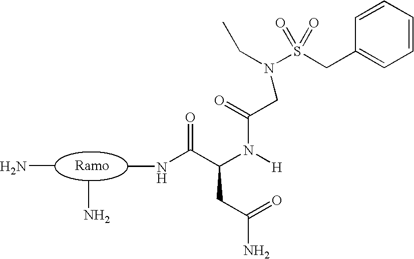

(Ethyl-benzylsulfonyl-amino)acetic acid was prepared according to Method U using ethyl iodide, followed by conversion to the corresponding pentafluorophenyl ester according to Method A in 78% yield. ¹H NMR (300 MHz, CDCl₃): δ 7.52-7.35 (m, 5H), 4.32 (s, 2H), 4.28 (s, 2H), 3.19 (q, J=6.9 Hz, 2H), 1.15 (t, J=6.0 Hz, 3H).

4,10-diFmoc-deacylramoplanin amine was reacted with the above pentafluorophenyl ester according to Method B to obtain Example 141. HPLC: Rₜ=5.37 min (Condition 1); Rₜ=8.50 min (Condition 4). ESMS: m/z 1328.7 [(M+2H)/2].

### Example 142

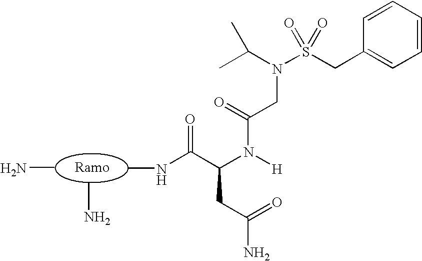

(Isopropyl-benzylsulfonyl-amino)acetic acid was prepared according to Method U using 2-iodopropane, followed by conversion to the corresponding pentafluorophenyl ester according to Method A in 43% yield. ¹H NMR (300 MHz, CDCl₃): δ 7.73-7.56 (m, 5H), 4.61 (s, 2H), 4.55 (s, 2H), 3.86-3.82 (m, 1H), 1.28 (d, J=6.9 Hz, 6H).

4,10-diFmoc-deacylramoplanin amine was reacted with the above pentafluorophenyl ester according to Method B to obtain Example 142. HPLC: Rₜ=5.47 min (Condition 1); Rₜ=8.53 min (Condition 4). ESMS: m/z 1336.1 [(M+2H)/2].

### Example 143

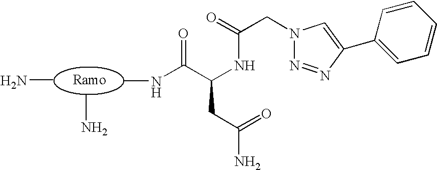

To a stirred solution of benzyl-2-bromoacetate (5 g, 21.8 mmol) in dry DMF was added NaN₃ (14 g, 218 mmol). The resulting mixture was heated to 50° C. behind a safety shield. After 4 h, the reaction was cooled to rt, diluted with water and extracted with EtOAc. The combined organic layer was dried over Na₂SO₄, followed by filtering through a short silica gel column. The column was washed with additional EtOAc followed by concentration under reduced pressure to afford benzyl-2-azidoacetate in 96% yield.

Benzyl-2-azidoacetate (1.5 g, 7.85 mmol) and phenylacetylene (860 μL, 7.85 mmol) were dissolved in toluene (10 mL) followed by heating the reaction mixture to reflux for 5 h. The reaction was concentrated under reduced pressure, and the residue was purified by column chromatography (5% EtOAc in DCM) to yield two regio isomers (4-phenyl[1,2,3]triazol-1-yl)acetic acid benzyl ester and (5-phenyl-[1,2,3]triazol-1-yl)acetic acid benzyl ester as pure products.

(4-Phenyl-[1,2,3]triazol-1-yl)acetic acid benzyl ester was converted to the corresponding pentafluorophenyl ester according to Method C followed by Method A in 42% yield. ¹H NMR (300 MHz, DMSO): δ 8.01 (s, 1H), 7.56-7.51 (m, 5H), 6.12 (s, 2H).

4,10-diFmoc-deacylramoplanin amine was reacted with the above pentafluorophenyl ester according to Method B to obtain Example 143. HPLC:: Rₜ=4.76 min (Condition 1); 3.73 min (Condition 2). ESMS: m/z 1302.2 [(M+2H)/2].

### Example 144

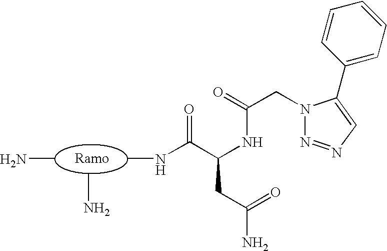

(5-Phenyl-[1,2,3]triazol-1-yl)acetic acid benzyl ester (see Example 143) was converted to the corresponding pentafluorophenyl ester according to Method C followed by Method A in 55% yield. ¹H NMR (300 MHz, DMSO): δ 8.69 (s, 1H), 7.88-7.85 (m, 2H), 7.48-7.32 (m, 3H), 6.09 (s, 2H).

4,10-diFmoc-deacylramoplanin amine was reacted with the above pentafluorophenyl ester according to Method B to obtain Example 144. HPLC: Rₜ=4.86 min (Condition 1); 3.80 min (Condition 2). ESMS: m/z 1301.8 [(M+2H)/2].

### Example 145

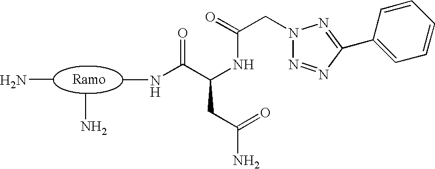

5-Phenyl-1H-tetrazole (1 equivalent) was dissolved in a refluxing 0.5 M solution of NaOMe in MeOH (1 equivalent). To this refluxing solution was added methyl bromoacetate (1 equivalent) in 4 equal portions over 30 min. The resulting reaction was further refluxed for 16 h at which time the reaction was cooled to rt and the resulting solid was filtered. This solid was washed with several portions of MeOH, and the combined filtrate was concentrated under reduced pressure to yield oil that was purified by column chromatography (5-20% EtOAc in DCM) to yield pure isomers: (5-phenyl-tetrazol-1-yl)acetic acid methyl ester and (5-phenyl-tetrazol-2-yl)acetic acid methyl ester in a 4:1 ratio.

(5-Phenyl-tetrazol-2-yl)acetic acid methyl ester was converted to the corresponding pentafluorophenyl ester according to Method C followed by Method A in 43% yield. ¹H NMR (300 MHz, DMSO): δ 7.81-7.79 (m, 2H), 7.68-7.60 (m, 3H), 6.35 (s, 2H).

4,10-diFmoc-deacylramoplanin amine was reacted with the above pentafluorophenyl ester according to Method B to obtain Example 145. HPLC: Rₜ=4.67 min (Condition 1);: Rₜ=3.61 min (Condition 2). ESMS: m/z 1302.5 [(M+2H)/2].

### Example 146

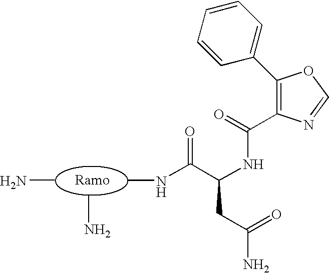

5-Phenyl-oxazole-4-carboxylic acid was converted to the corresponding pentafluorophenyl ester according to Method A in 64% yield. ¹H NMR (300 MHz, DMSO): δ 8.77 (s, 1H), 7.99-7.96 (m, 2H), 7.57-7.55 (m, 3H).

4,10-diFmoc-deacylramoplanin amine was reacted with the above pentafluorophenyl ester according to Method B to obtain Example 146. HPLC: Rₜ=5.08 min (Condition 1); Rₜ=4.01 min (Condition 2). ESMS: m/z 1295.5 [(M+2H)/2].

### Example 147

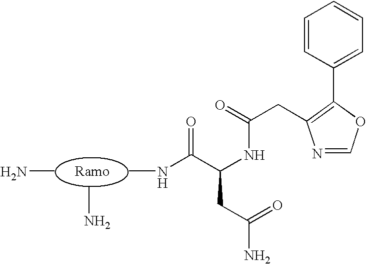

4-Bromomethyl-5-phenyl-oxazole (1 equivalent) was treated with NaCN (5 equivalents) in DMF at 60° C. for 4 h at which time the reaction was diluted with water and extracted with EtOAc. The organic phase was dried over Na₂SO₄, and passed through a short silica column. This column was eluted with EtOAc. The combined eluants were concentrated under reduced pressure to yield (5-phenyloxazol-4-yl)acetonitrile that was used for the next reaction without any further purification.

A solution of (5-phenyloxazol-4-yl)acetonitrile in 5% NaOH in aqueous MeOH was heated to 50° C. for 3 h followed by stirring for 16 h at rt. The reaction was concentrated under reduced pressure to yield an off-white solid that was converted to the corresponding pentafluorophenyl ester according to Method A in 21% yield. ¹H NMR (300 MHz, DMSO): δ 8.49 (s, 1H), 7.68-7.44 (m, 5H), 4.40 (s, 2H).

4,10-diFmoc-deacylramoplanin amine was reacted with the above pentafluorophenyl ester according to Method B to obtain Example 147. HPLC: Rₜ=5.10 min (Condition 1); Rₜ=3.98 min (Condition 2). ESMS: m/z 1302.2 [(M+2H)/2].

### Examples 148-157

### Example 148

### N-valeryl glycine

To a solution of glycine tert-butyl ester hydrochloride (1.19 mmol) in DMF (5 ml) the pentafluorophenyl ester of valeric acid (1.19 mmol) was added, followed by TEA (1.19 mmol) and a catalytic amount of 1-hydroxybenzotriazole. The mixture was allowed to react at room temperature for 2 h, then poured in 150 ml of ethyl acetate. The solution was washed with 1 N aqueous hydrochloric acid (3×150 ml), 5% aqueous sodium hydrogen carbonate (3×150 ml), and water (150 ml). The organic extract was dried over sodium sulfate, filtered and the solvent was removed under reduced pressure. N-valeryl glycine tert-butyl ester was obtained as an oily residue (1.13 mmol).

N-valeryl glycine tert-butyl ester (1.13 mmol) was dissolved in 3 ml of a 1:1 mixture of trifluoroacetic acid and dichloromethane, and allowed to stir at room temperature for 2 h. The solvent was removed under reduced pressure, and N-valeryl glycine (1.13 mmol) was obtained as a white solid.

¹H-NMR (AA refers to the aminoacid, A to the valeryl moiety): (CDCl₃, 500 MHz) chemical shift p.p.m. 0.93 (t, 3H, A CH₂ CH₃); 1.38 (m, 2H, A CH₂CH₃); 1.64 (m, 2H, A CH₂CH₂CO); 2.36 (t, 2H, A CH₂CO); 4.12 (dd, 2H, AA CH₂COOH); 6.62 (dd, 1H, AA NH); 10.52 (s, 1H, AA COOH).

### Example 149

### N-valeryl-L-Phenylalanine

N-valeryl-L-phenylalanine was produced in an analogous manner to N-valeryl glycine, substituting L-phenylalanine tert-butyl ester hydrochloride for glycine tert-butyl ester hydrochloride.

¹H-NMR (AA refers to the aminoacid, A to the valeryl moiety): (CDCl₃, 500 MHz) chemical shift p.p.m. 0.88 (t, 3H, A CH₂ CH₃); 1.29 (m, 2H, A CH₂CH₃); 1.53 (m, 2H, A CH₂CH₂CO); 2.26 (t, 2H, A CH₂CO); 3.14 (dd, 1H, AA CHH₂Ph); 3.26 (dd, 1H, AA CH₂Ph); 4.94 (dd, 1H, AA CHCOOH); 6.34 (dd, 1H, AA NH); 10.57 (s, 1H, AA COOH).

### Example 150

### N-(2-ethyl-hexanoyl)-Glycine

N-(2-ethyl-hexanoyl)-glycine was produced in an analogous manner to N-valeryl glycine, substituting 2-ethyl hexanoic acid pentafluorophenylester for valeric acid pentafluorophenylester.

¹H-NMR (AA refers to the aminoacid, A to the 2-ethyl-hexanoyl moiety): (CDCl₃, 500 MHz) chemical shift p.p.m. 0.87 (t, 3H, A CH₂ CH₃); 0.91 (t, 3H, A CH₂CH₃); 1.2 (m, 4H, A CH₂CH₂CH₃); 1.5 (m, 4H, A CH₂CHCO); 2.11 (m, 1H, A CHCO); 4.11 (m, 2H, AA CH₂COOH); 6.44 (dd, 1H, AA NH); 8.37 (s, 1H, AA COOH).

### Example 151

### N-(o-methyl)-phenylacetyl-L-Phenylalanine

N-(o-methyl)-phenylacetyl-L-phenylalanine was produced in an analogous manner to N-valeryl glycine, substituting L-phenylalanine tert-butyl ester hydrochloride for glycine tert-butyl ester hydrochloride, and substituting 2-methylbenzyl carboxylic acid pentafluorophenylester for valeric acid pentafluorophenylester.

¹H-NMR (AA refers to the aminoacid, A to the o-methyl-phenylacetyl moiety): (CDCl₃, 500 MHz) chemical shift p.p.m. 2.10 (s, 3H, A ArCH₃); 2.96 (m, 1H, AA CH₂Ph); 3.12 (m, 1H, AA CHH₂Ph); 3.62 (m, 2H, A CH₂Ph); 4.80 (m, 1H, AA CHCOOH); 5.83 (m, 1H, AA NH); 6.86 (m, 2H, A ArH); 7.06 (m, 1H, A ArH); 7.2 (m, 6H, A+AA ArH).

### Example 152

### N-valeryl-D-Alanine

N-valeryl-D-alanine was produced in an analogous manner to N-valeryl glycine, substituting D-alanine tert-butyl ester hydrochloride for glycine tert-butyl ester hydrochloride.

¹H-NMR (AA refers to the aminoacid, A to the valeryl moiety): (CDCl₃, 500 MHz) chemical shift p.p.m. 0.92 (t, 3H, A CH₂ CH₃); 1.35 (m, 2H, A CH₂CH₃); 1.49 (d, 3H, AA CHCH₃); 1.62 (m, 2H, A CH₂CH₂CO); 2.33 (t, 2H, A CH₂CO); 4.62 (m, 1H, AA CHCOOH); 6.72 (dd, 1H, AA NH); 11.19 (s, 1H, AA COOH).

### Example 153

### N-(o-methyl)-phenylacetyl-D-Alanine

N-(o-methyl)-phenylacetyl-D-Alanine was produced in an analogous manner to N-valeryl glycine, substituting D-alanine tert-butyl ester hydrochloride for glycine tert-butyl ester hydrochloride, and substituting 2-methylbenzyl carboxylic acid pentafluorophenylester for valeric acid pentafluorophenylester.

¹H-NMR (AA refers to the aminoacid, A to the o-methyl-phenylacetyl moiety): (CDCl₃, 500 MHz) chemical shift p.p.m. 1.40 (d, 3H, AA CHCH₃); 2.29 (s, 3H, A ArCH₃); 3.72 (s, 2H, A CH₃Ph); 4.46 (m, 1H, AA CHCOOH); 5.98 (m, 1H, AA NH); 7.2 (m, 4H, A ArH).

### Example 154

### N-(o-methyl)-phenylacetyl-Glycine

N-(o-methyl)-phenylacetyl-glycine was produced in an analogous manner to N-valeryl glycine, substituting 2-methylbenzyl carboxylic acid pentafluorophenylester for valeric acid pentafluorophenylester.

¹H-NMR (AA refers to the aminoacid, A to the o-methyl-phenylacetyl moiety): (CDCl₃, 500 MHz) chemical shift p.p.m. 2.29 (s, 3H, A ArCH₃); 3.73 (s, 2H, A CH₂Ph); 4.00 (d, 2H, AA CH₂COOH); 6.34 (t, 1H, AA NH); 7.23 (m, 4H, A ArH); 10.29 (s, 1H, AA COOH).

### Example 155

### N-(2-ethyl-hexanoyl)-D-Alanine

N-(2-ethyl-hexanoyl)-D-alanine was produced in an analogous manner to N-valeryl glycine, substituting D-alanine tert-butyl ester hydrochloride for glycine tert-butyl ester hydrochloride, and substituting 2-ethylhexanoic acid pentafluorophenylester for valeric acid pentafluorophenylester.

¹H-NMR (AA refers to the aminoacid, A to the 2-ethyl-hexanoyl moiety): (CDCl₃, 500 MHz) chemical shift p.p.m. 0.92 (t, 3H, A CH₂ CH₃); 0.92 (t, 3H, A CH₂ CH₃); 1.3 (m, 4H, A CH₂CH₂CH₃); 1.5 (m, 4H, A CH₂CHCO); 1.5 (d, 3H, AA CH₃CH); 2.12 (m, 1H, A CHCO); 4.68 (m, 1H, AA CHCOOH); 6.50 (d, 1H, AA NH); 10.36 (s, 1H, AA COOH).

### Example 156

### N-(2-ethyl-hexanoyl)-L-Phenylalanine

N-(2-ethyl-hexanoyl)-D-alanine was produced in an analogous manner to N-valeryl glycine, substituting L-phenylalanine tert-butyl ester hydrochloride for glycine tert-butyl ester hydrochloride, and substituting 2-ethylhexanoic acid pentafluorophenylester for valeric acid pentafluorophenylester.

¹H-NMR (AA refers to the aminoacid, A to the 2-ethyl-hexanoyl moiety): (CDCl₃, 500 MHz) chemical shift p.p.m. 0.85 (t, 3H, A CH₂ CHH₃); 0.9 (t, 3H, A CH₂ CH₃); 1.2 (m, 4H, A CH₂CH₂CH₃); 1.5 (m, 4H, A CH₂CHCO); 2.02 (m, 1H, A CHCO); 3.14 (m, 1H, AA CH₂Ph); 3.27 (m, 1H, AA CH₂Ph); 5.01 (m, 1H, AA CHCOOH); 6.22 (m, 1H, AA NH); 7.18 (m, 2H, AA ArH); 7.3 (m, 3H, AA ArH); 10.71 (s, 1H, AA COOH).

### Example 157

### N-(o-methyl)-phenylacetyl-L-Isoleucine

N-(o-methyl)-phenylacetyl-L-isoleucine was produced in an analogous manner to N-valeryl glycine, substituting L-isoleucine tert-butyl ester hydrochloride for glycine tert-butyl ester hydrochloride, and substituting 2-methylbenzyl carboxylic acid pentafluorophenylester for valeric acid pentafluorophenylester.

The above amino acid derivatives (e.g., N-valeryl glycine) were converted to the corresponding pentafluorophenyl esters according to Method A. 4,10-diFmoc-deacylramoplanin amine was treated with the above pentafluorophenyl esters according to Method B to obtain 148-157.

The HPLC conditions were as follows: ¹Varian 9010; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: (reaction) time 0 min % B=35; time 15 min % B=50; time 35 min % B=70; (deprotection) time 0 min % B=20; time 30 min % B=40.

Masses were obtained using the following conditions: ThermoFinnigan LCQ^{Advantage} ion trap mass spectrometry equipped with an ESI source with LC Surveyor and auto sampler. Sample Inlet Conditions: Capillary Temperature(° C.): 200; Sheat Gas (N₂, arbitrary units): 20. Sample Inlet Voltage Settings: Polarity: positive; Spray Voltage (kV): 4.7; Capillary Voltage (V): 39; Tube Lens Offset (V): +55. Full Scan conditions: Scan range (amu): 200-2000 (double charge ion was detected if MW>2000); Number of microscans: 3; Maximum ion time (ms): 50. Compounds were analysed both by direct infusion (the compound was dissolved in TFA 0.1%-MeCN 1:1) and with HPLC-MS equipment (using the HPLC methods described above).

Retention times and masses are reported in Table 1 below. Compounds were obtained as a mixture of diastereomers (racemisation was observed in the amino amid residues during amidation), and in most cases the diastereomers show a different retention time.

TABLE 1

| | | | RT | RT¹ | |
|---|---|---|---|---|---|
| Ex | | | (diFMOC | (final | Exact |
| No. | Derivative | R^{x} | derivative | product) | Mass |
| 148 | N-valeryl glycine | 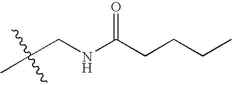 | 7.3 | 10.6 | 2557 |
| 149 | N-valeryl- phenylalanine | 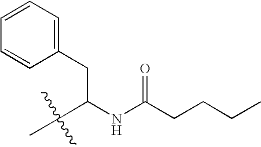 | 11.6 + 11.9 | 20.3 + 20.9 | 2647 |
| 150 | N-(2-ethyl- hexanoyl)-glycine | 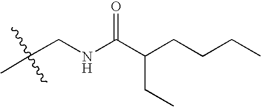 | 10.8 | 17 + 17.2 | 2599 |
| 151 | N-(o-methyl)- phenylacetyl- phenylalanine | 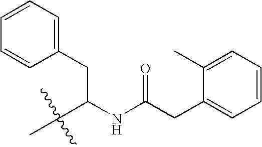 | 14.5 + 14.9 | 25.0 + 25.8 | 2695 |
| 152 | N-valeryl-alanine | 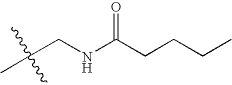 | 8 + 8.6 | 10.8 + 12.6 | 2571 |
| 153 | N-(o-methyl)- phenylacetyl- alanine | 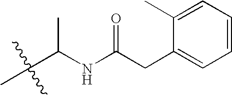 | 9.5 + 10.2 | 15.2 + 16.9 | 2619 |
| 154 | N-(o-methyl)- phenylacetyl- glycine | 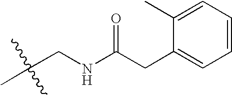 | 9.2 | 14.5 | 2605 |
| 155 | N-(2- ethylhexanoyl)- alanine | 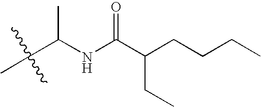 | 11.2 + 11.9 +12.3 + 12.7 | 18.8 + 20.5 +21.5 + 22.0 | 2613 |
| 156 | N-(2- ethylhexanoyl)- phenylalanine | 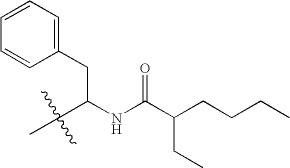 | 15.5 + 16.0 | 29.3 + 29.7 | 2689 |
| 157 | N-(o-methyl)- phenylacetyl- isoleucine | 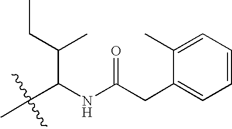 | NA | NA | NA |

The mixtures of diastereomers obtained were evaluated in the in vitro antimicrobial assay in Example B.

### Examples 158-176

The following aliphatic carbocyclic acids (RCOOH in Table 2) were reacted with 4,10-diFmoc-deacylramoplanin amine according to the following method:

To a solution of 4,10-diFmoc-deacylramoplanin amine (0.35 mmol), TEA (1.05 mmol) and the appropriate carboxylic acid (RCOOH in Table 2) (0.525 mmol) in DMF (12.5 mL), PyBOP was added with stirring at room temperature. The reaction was monitored by HPLC analysis. The mixture was allowed to react at room temperature and, after 5 hours, piperidine (625 μL) or, alternatively, 2,2,6,6-tetremethylpiperidine (1.875 mL) was added to remove the protecting group from the ornithine moieties. The reaction was maintained under stirring at room temperature and monitored by HPLC, and after 30 minutes, diluted HCl was added (6.5 mL of a 1M solution). The crude products were used to test the compounds according to Example B.

The desired purified product is obtained by purification using preparative HPLC and lyophilization.

The HPLC conditions were as follows: Varian 9010; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: (reaction) time 0 min % B=35; time 15 min % B=50; time 35 min % B=70; (deprotection) time 0 min % B=20; time 30 min % B=40.

Masses were obtained using the following conditions: ThermoFinnigan LCQ^{Advantage} ion trap mass spectrometry equipped with an ESI source with LC Surveyor and auto sampler. Sample Inlet Conditions: Capillary Temperature(° C.): 200; Sheat Gas (N₂, arbitrary units): 20. Sample Inlet Voltage Settings: Polarity: positive; Spray Voltage (kV): 4.7; Capillary Voltage (V): 39; Tube Lens Offset (V): +55. Full Scan conditions: Scan range (amu): 200-2000 (double charge ion was detected if MW>2000); Number of microscans: 3; Maximum ion time (ms): 50. Compounds were analysed both by direct infusion (the compound was dissolved in TFA 0.1%-MeCN 1:1) and with HPLC-MS equipment (using the HPLC methods described above).

Retention times and masses are reported in Table 2. In some cases compounds were obtained as a mixture of diastereoisomers showing different retention times.

TABLE 2

| | | | RT | RT | |
|---|---|---|---|---|---|
| Ex. | | | (diFMOC | (final | Exact |
| No. | RCOOH | R^{x} | derivative) | product) | Mass |
| 158 | Cyclopentyl-COOH | 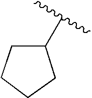 | 10 | 15.4 | 2512 |
| 159 | Cyclopentyl-methyl- COOH | 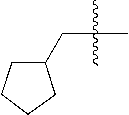 | 11 | 17.9 | 2526 |
| 160 | 2-cyclopentyl-ethyl- COOH | 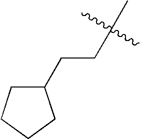 | 12.6 | 21.9 | 2541 |
| 161 | 1-phenylcyclopentyl- COOH | 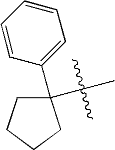 | 13.6 | 23.6 | 2588 |
| 162 | Bicyclo[2.2.1]heptyl- methyl-COOH | 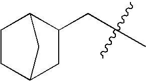 | 12.5 | 21.6 | 2552 |
| 163 | Cyclohexylmethyl- COOH | 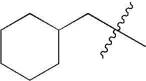 | 12 | 21 | 2540 |
| 164 | 4-methyl-cyclohexyl- methyl-COOH | 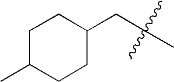 | 11.1 | 18.3 | 2554 |
| 165 | 2-methyl-cyclohexyl- methyl-COOH (mixture of cis +trans) | 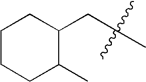 | 11.9 + 12 | 19.2 | 2554 |
| 166 | 4-pentyl-cyclohexyl- methyl-COOH (trans) | 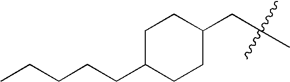 | 18.8 | 25.3 | 2610 |
| 167 | cycloheptyl-COOH | 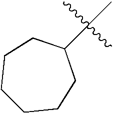 | 12.3 | 20.8 | 2540 |
| 168 | Cyclopropyl-COOH | 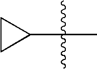 | 7.5 | 8.9 | 2484 |
| 169 | 2-methylcyclopropyl- COOH (mixture of cis + trans) | 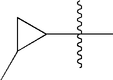 | 8.7 | 13.1 | 2498 |
| 170 | 1-methylcyclopropyl- COOH | 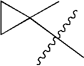 | 8.8 | 11.9 | 2498 |
| 171 | 2,2,3,3-tetramethyl- cyclopropyl-COOH | 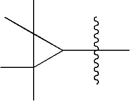 | 12.2 | 20.8 | 2539 |
| 172 | 2-(2-methyl-prop-1- enyl)-3,3-dimethyl- cyclopropyl-COOH (mixture of cis + trans) | 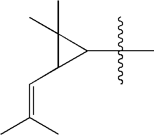 | 13.5 + 14.8 | Not available | 2566 |
| 173 | 2-phenyl-cyclopropyl- COOH (trans) | 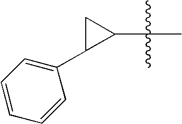 | 11 | 19.7 + 21.4 | 2560 |
| 174 | 1-phenyl-cyclopropyl- COOH | 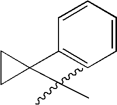 | 11.4 | 18.4 | 2560 |
| 175 | Cyclobutyl-COOH | 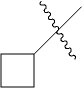 | 8.6 | 12.2 | 2498 |
| 176 | Cyclohexen-3-yl- COOH | 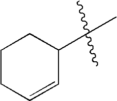 | 10 | 15.7 | 2524 |

### FORMULA IB EXAMPLES

### Examples 177-189

To a solution of 4,10-diFmoc-deacylramoplanin amine (3.15 μmol) (suitably protected at the (4,10) ornithine residues) in DMF (500 μl), TEA (3.15 μmol) and a suitable isothiocyanate (RNCS) (3.15 μmol, were added while stirring at room temperature. The mixture was allowed to react at room temperature for 2 hours. The reaction was monitored by HPLC analysis, and the retention times of the diFmoc protected reaction products are shown below in Table 3 as “RT¹ (diFMOC derivative)”.

Piperidine (12.5 μt) was added to remove the protecting group from the ornithine moieties. The reaction was maintained under stirring at room temperature for 30 minutes, and monitored by HPLC. Diluted HCl was added (310 μl of 0.5M solution) to quench the reaction. The resulting crude product solution was used as is for the microbiological tests in Example B. The retention time of the final crude product (after quenching with HCl) is shown below in Table 3 as “RT² (final crude product)”.

The desired product is also obtained as powder through purification by preparative HPLC and lyophilization.

The HPLC conditions for both the diFMOC derivative and the deprotected product were as follows: Varian 9010; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: reaction (RT¹) time 0 min % B=35; time 15 min % B=50; time 35 min % B=70; deprotected (RT²) 0 min % B=20; time 30 min % B=40.

Masses were obtained using the following conditions: ThermoFinnigan LCQ^{Advantage} ion trap mass spectrometry equipped with an ESI source with LC Surveyor and auto sampler. Sample Inlet Conditions: Capillary Temperature(° C.): 200; Sheat Gas (N₂, arbitrary units): 20. Sample Inlet Voltage Settings: Polarity: positive; Spray Voltage (kV): 4.7; Capillary Voltage (V): 39; Tube Lens Offset (V): +55. Full Scan conditions: Scan range (amu): 200-2000 (double charge ion was detected if MW>2000); Number of microscans: 3; Maximum ion time (ms): 50. Compounds were analysed both by direct infusion (the compound was dissolved in TFA 0.1%-MeCN 1:1) and with HPLC-MS equipment (using the HPLC methods described above).

Table 3 below shows the isocyanate used for the reaction (RNCS), the retention times for the diFMOC derivative and the final crude product, and the mass of the final crude product.

TABLE 3

| Ex. No. | RNCS | 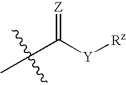 | RT¹ (min) (diFMOC derivative) | RT² (min) (final crude product) | Exact Mass |
|---|---|---|---|---|---|
| 177 | 2-methylphenyl-NCS | 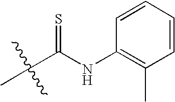 | 9.2 | 16.2 | 2565 |
| 178 | 3-methylphenyl- NCS | 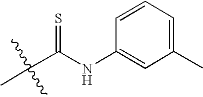 | 10.1 | 16.4 | 2565 |
| 179 | 4-methylphenyl- NCS | 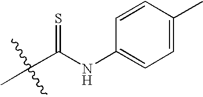 | 10.2 | 16.7 | 2565 |
| 180 | 2-fluorophenyl-NCS | 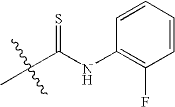 | 9.6 | 14.5 | 2569 |
| 181 | 3-fluorophenyl-NCS | 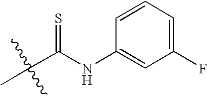 | 9.9 | 16.4 | 2569 |
| 182 | 4-fluorophenyl-NCS | 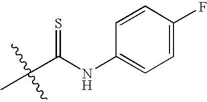 | 9.8 | 15.1 | 2569 |
| 183 | 2,6-difluorophenyl- NCS | 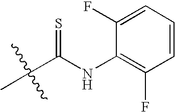 | 9.8 | 14.6 | 2587 |
| 184 | Benzyl-NCS | 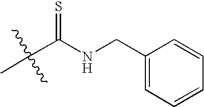 | 10.6 | 20.9 | 2565 |
| 185 | 2-phenylethyl-NCS | 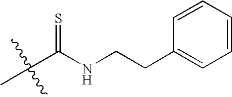 | 9.9 | 29.8 | 2579 |
| 186 | Napth-1-yl-NCS | 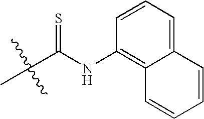 | 10.9 | 23.4 | 2601 |
| 187 | Cyclohexyl-NCS | 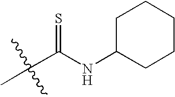 | 6.1 | 22.7 | 2557 |
| 188 | 4′-propyl-4- cyclohexyl-phenyl- NCS | 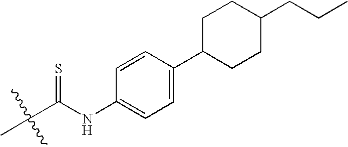 | 19.6 | 37 | 2675 |
| 189 | Phenyl-NCS | 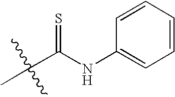 | 9.1 | 13.3 | 2551 |

### Examples 190-202

To a solution of 4,10-diFmoc-deacylramoplanin amine (suitably protected at the (4,10) ornithine residues) (3.15 μmol) in DMF (500 μl), TEA (3.15 μmol) and a suitable isocyanate (RNCO) (3.15 μmol) were added while stirring at room temperature. The mixture was allowed to react at room temperature for 2 hours. The reaction was monitored by HPLC analysis, and the retention times of the diFmoc protected reaction products are shown below in Table 4 as “RT¹ (diFMOC derivative)”.

Piperidine (12.5 μl) was added to remove the protecting group from the ornithine moieties. The reaction was maintained under stirring at room temperature for 30 minutes, and monitored by HPLC. Diluted HCl was added (310 μl of 0.5M solution) to quench the reaction. The resulting crude product solution was used as is for the microbiological tests in Example B. The retention time of the final crude product (after quenching with HCl) is shown below in Table 4 as “RT² (final crude product)”.

The desired product is also obtained as powder through purification by preparative HPLC and lyophilization.

The HPLC conditions for both the diFMOC derivative and the deprotected product were as follows: Varian 9010; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CH; Gradient: reaction (RT¹) time 0 min % B=35; time 15 min % B=50; time 35 min % B=70; deprotected (RT²) 0 min % B=20; time 30 min % B=40.

Masses were obtained using the following conditions: ThermoFinnigan LCQ^{Advantage} ion trap mass spectrometry equipped with an ESI source with LC Surveyor and auto sampler. Sample Inlet Conditions: Capillary Temperature(° C.): 200; Sheat Gas (N₂, arbitrary units): 20. Sample Inlet Voltage Settings: Polarity: positive; Spray Voltage (kV): 4.7; Capillary Voltage (V); 39; Tube Lens Offset (V): +55. Full Scan conditions: Scan range (amu): 200-2000 (double charge ion was detected if MW>2000); Number of microscans: 3; Maximum ion time (ms): 50. Compounds were analysed both by direct infusion (the compound was dissolved in TFA 0.1%-MeCN 1:1) and with HPLC-MS equipment (using the HPLC methods described above).

Table 4 below shows the cyanate used for the reaction (RNCO), the retention times for the diFMOC derivative and the final crude product, and the mass of the final crude product.

TABLE 4

| Ex. No. | RNC0 | 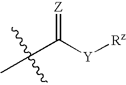 | RT¹ (min) (diFMOC derivative) | RT² (min) (final crude product) | Exact Mass |
|---|---|---|---|---|---|
| 190 | n-Butyl-NCO | 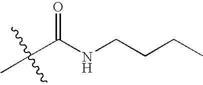 | 8.9 | 16.1 | 2515 |
| 191 | n-octyl-NCO | 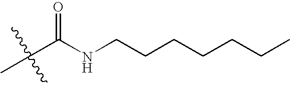 | 14.5 | 19.2 | 2571 |
| 192 | Cyclohexyl-NCO | 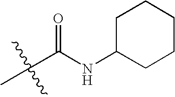 | 9.8 | 19.1 | 2541 |
| 193 | Benzyl-NCO | 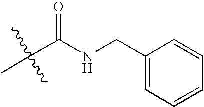 | 9.2 | 16.8 | 2549 |
| 194 | Phenyl-NCO | 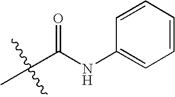 | 8.7 | 15.6 | 2535 |
| 195 | 2-trifluoro-methyl- phenyl-NCO | 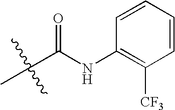 | 10.4 | 16.3 | 2603 |
| 196 | 3-trifluoro-methyl- phenyl-NCO | 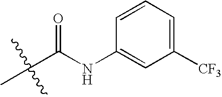 | 11.6 | 16.4 | 2603 |
| 197 | 4-trifluoro-methyl- phenyl-NCO | 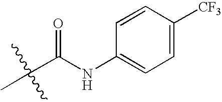 | 11.6 | 17.5 | 2603 |
| 198 | 2-methoxy-phenyl- NCO | 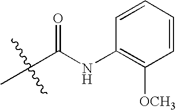 | 9.3 | 17.5 | 2565 |
| 199 | 2,6-dimethyl-phenyl- NCO | 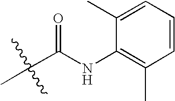 | 9.5 | 17.3 | 2563 |
| 200 | Napth-1-yl-NCO | 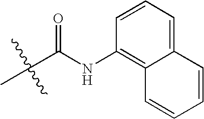 | 10.3 | 20.6 | 2585 |
| 201 | 1-Napth-1-yl-ethyl- NCO | 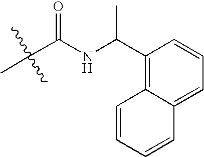 | 11.6 | 23.8 | 2613 |
| 202 | 2-methylphenyl- NCO | 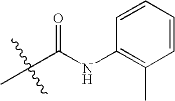 | 9.3 | 16.5 | 2563 |

### Examples 203-235

To a solution of 4,10-diFmoc-deacylramoplanin amine (2.1 μmol) in DMF (250 μl), TEA (8.4 μmol) and a suitable chloroformate (ROCOCl) (6.3 μmol) were added while stirring at room temperature. The mixture was allowed to react at room temperature for 2 hours. The reaction was monitored by HPLC analysis, and the retention times of the diFmoc protected reaction products are shown below in Table 5 as “RT¹ (diFMOC derivative)”.

Piperidine (12.5 μl) was added to remove the protecting group from the ornithine moieties. The reaction was maintained under stirring at room temperature for 30 minutes, and diluted HCl was added (310 μl of 0.5M solution). The reaction was monitored by HPLC. The resulting crude product solution was used as is for the microbiological tests in Example B. The retention time of the final crude product (after quenching with HCl) is shown below in Table 5 as “RT² (final crude product)”.

The desired product is also obtained as powder through purification by preparative HPLC and lyophilization. Compound 23CB3 was resynthesized as powder and retested in the microbiological tests in Example B.

The HPLC conditions for both the diFMOC derivative and the deprotected product were as follows: Varian 9010; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: reaction (RT¹) time 0 min % B=35; time 15 min % B=50; time 35 min % B=70; deprotected (RT²) 0 min % B=20; time 30 min % B=40.

Masses were obtained using the following conditions: ThermoFinnigan LCQ^{Advantage} ion trap mass spectrometry equipped with an ESI source with LC Surveyor and auto sampler. Sample Inlet Conditions: Capillary Temperature(° C.): 200; Sheat Gas (N₂, arbitrary units): 20. Sample Inlet Voltage Settings: Polarity: positive; Spray Voltage (kV): 4.7; Capillary Voltage (V): 39; Tube Lens Offset (V): +55. Full Scan conditions: Scan range (amu): 200-2000 (double charge ion was detected if MW>2000); Number of microscans: 3; Maximum ion time (ms): 50. Compounds were analysed both by direct infusion (the compound was dissolved in TFA 0.1%-MeCN 1:1) and with HPLC-MS equipment (using the HPLC methods described above).

Table 5 below shows the chloroformate used for the reaction (ROCOCl), the retention times for the diFMOC derivative and the final crude product, and the mass of the final crude product.

TABLE 5

| Example No. | ROCOCl | 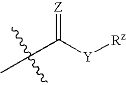 | RT¹ (min) (diFMOC derivative) | RT² (min) (final crude product) | Exact Mass |
|---|---|---|---|---|---|
| 203 | Propyl-OCOCl | 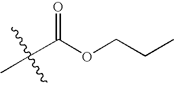 | 12.4 | 11.1 | 2502 |
| 204 | butyl-OCOCl | 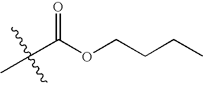 | 13.7 | 13.7 | 2516 |
| 205 | hexyl-OCOCl | 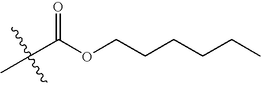 | 19.2 | 20.8 | 2544 |
| 206 | octyl-OCOCl | 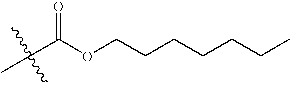 | 24.4 | 26.5 | 2572 |
| 207 | decyl-OCOCl | 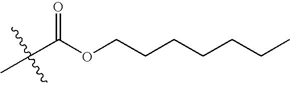 | 30.2 | 32.1 | 2600 |
| 208 | isopropyl-OCOCl | 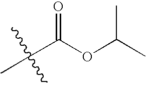 | 11.2 | 10.2 | 2502 |
| 209 | isobutyl-OCOCl | 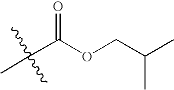 | 13.9 | 14.4 | 2516 |
| 210 | 2,2-dimethyl- propyl-OCOCl | 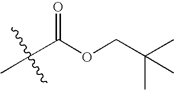 | 16.7 | 14.1 | 2530 |
| 211 | 2-ethyl-hexyl- OCOCl | 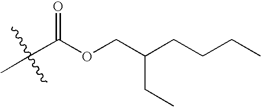 | 23.3 | 25.7 | 2572 |
| 212 | (1S,2R,5S)-2- isopropyl-5- methyl-cyclohex- 1-yl-OCOCl | 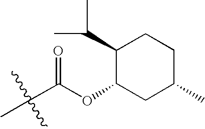 | 25.7 | 25.7 | 2598 |
| 213 | (1R,2S,5R)-2- isopropyl-5- methyl-cyclohex- 1-yl-OCOCl | 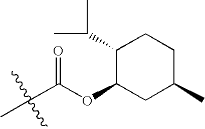 | 25.4 | 27.4 | 2598 |
| 214 | Ethenyl-OCOCl | 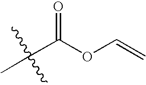 | 6 | 8 | 2486 |
| 215 | prop-2-enyl- OCOCl | 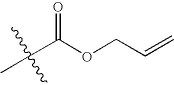 | 10.6 | 9.3 | 2500 |
| 216 | But-3-enyl- OCOCl | 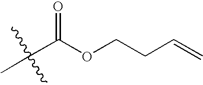 | 12.7 | 12.8 | 2514 |
| 217 | 1-methyl-ethenyl- OCOCl | 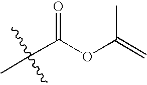 | 5.8 | 9.1 | 2500 |
| 218 | But-3-ynyl- OCOCl | 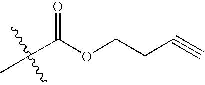 | 10.9 | 10.4 | 2512 |
| 219 | But-2-ynyl- OCOCl | 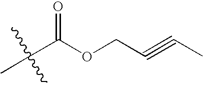 | 11.2 | 11 | 2512 |
| 220 | 4-fluorophenyl- OCOCl | 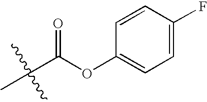 | 6 | 8.7 | 2554 |
| 221 | 4-bromophenyl- OCOCl | 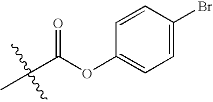 | 6 | 17.7 | 2615 |
| 222 | 4-nitrophenyl- OCOCl | 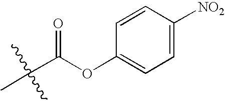 | 5.9 | 8.6 | 2581 |
| 223 | 4- methoxycarbonyl- phenyl-OCOCl | 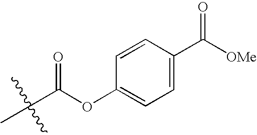 | 5.9 | 8.8 | 2594 |
| 224 | 2-chlorophenyl- OCOCl | 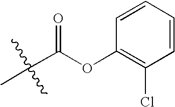 | Not available | 8.3 | 2570 |
| 225 | 4-chlorophenyl- OCOCl | 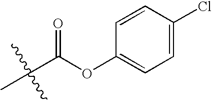 | Not available | 10.9 | 2570 |
| 226 | 2-methoxy- phenyl-OCOCl | 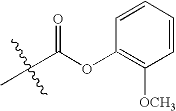 | Not available | 4.7 | 2566 |
| 227 | 4-methoxy- phenyl-OCOCl | 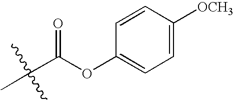 | Not available | 4.0 | 2566 |
| 228 | 4-methyl-phenyl- OCOCl | 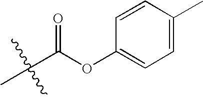 | Not available | 7.0 | 2550 |
| 229 | 2-nitro-phenyl- OCOCl | 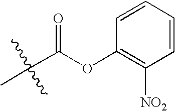 | Not available | 7.7 | 2581 |
| 230 | 3-trifluoromethyl- phenyl-OCOCl | 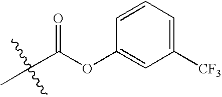 | Not available | 16.9 | 2604 |
| 231 | 2-nitro-3,4- dimethoxy- phenyl-OCOCl | 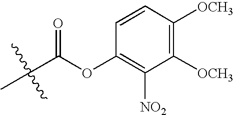 | Not available | Not available | 2641 |
| 232 | benzyl-OCOCl | 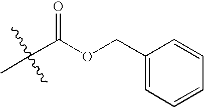 | Not available | 15.2 | 2550 |
| 233 | 2-chlorophenyl- methyl-OCOCl | 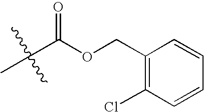 | Not available | 18.2 | 2584 |
| 234 | (2-trifluoromethyl- phenyl)-chloro- methyl-OCOCl | 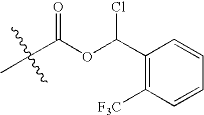 | Not available | 20.1 | 2652 |
| 235 | (4-nitro-phenyl)- methyl-OCOCl | 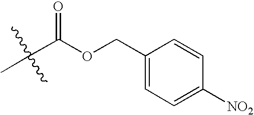 | Not available | 15.5 | 2595 |

### FORMULA 1C EXAMPLES

### Examples 236-247

To a solution of 4,10-diFmoc-deacylramoplanin amine (2.1 μmol) in a mixture of 1:1 THF:water (250 μl), a suitable aldehyde (RCHO) (6.27 μmol) and NaCNBH₃ (10.45 μmol) are added with stirring at rt. The mixture is allowed to react at rt for 5 hours. The reaction was monitored by HPLC analysis, and the retention times of the diFmoc protected reaction products are shown below in Table 6 as “RT¹ (diFMOC derivative)”.

The reaction mixture is evaporated to dryness and dissolved in 245 μL of DMF. Piperidine (5 μl) was added to remove the protecting group from the ornithine moieties. The reaction was maintained under stirring at room temperature for 30 minutes then diluted HCl was added (310 μl of 0.5M solution). The resulting crude product solution was used as is for the microbiological tests in Example B.

The desired product is also obtained as powder through purification by preparative HPLC and lyophilization.

The HPLC conditions for the diFMOC derivatives were as follows: Shimadzu LC 2010A (CLASS-VP6); column: Merck Lichrocart 125-4 Lichrospher 100 RP 18 (5 μm); flow: 1 ml/min; detector UV λ=270 nm; inj. vol. 10 μl; phase A: HCOONH₄ 0.05M; phase B: CH₃CN; Gradient: reaction (RT¹) time 0 min % B=35; time 15 min % B=40; time 35 min % B=70).

Masses were obtained using the following conditions: ThermoFinnigan LCQ^{Advantage} ion trap mass spectrometry equipped with an ESI source with LC Surveyor and auto sampler. Sample Inlet Conditions: Capillary Temperature(° C.): 200; Sheat Gas (N₂, arbitrary units): 20. Sample Inlet Voltage Settings: Polarity: positive; Spray Voltage (kV): 4.7; Capillary Voltage (V): 39; Tube Lens Offset (V): +55. Full Scan conditions: Scan range (amu): 200-2000 (double charge ion was detected if MW>2000); Number of microscans: 3; Maximum ion time (ms): 50. Compounds were analysed both by direct infusion (the compound was dissolved in TFA 0.1%-MeCN 1:1) and with HPLC-MS equipment (using the HPLC methods described above).

Table 6 below shows the aldehyde used for the reaction (RCHO), the retention times for the diFMOC derivative and the final crude product, and the mass of the final crude product.

TABLE 6

| | | | | RT² (min) | |
|---|---|---|---|---|---|
| | | | RT¹ (min) | (final | |
| Example | | | (diFMOC | crude | Exact |
| No. | RCHO | R′ | derivative) | product) | Mass |
| 236 | 4-n-Butoxyphenyl- CHO | 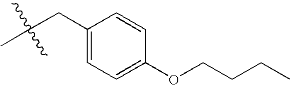 | 24.34 | Not Available | 2578 |
| 237 | 3,6-difluorophenyl- CHO | 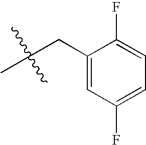 | 29.21 | Not Available | 2542 |
| 238 | 3,6-dimethylphenyl- CHO | 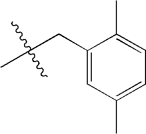 | 22.8 | Not Available | 2534 |
| 239 | 2,3-dihydro- benzo[1,4]dioxin-6- yl-CHO | 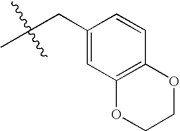 | 18.17 | Not Available | 2564 |
| 240 | 2-phenylethyl-CHO | 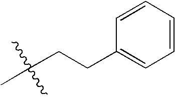 | 22.8 | Not Available | 2520 |
| 241 | Cyclohexyl-CHO | 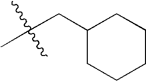 | 22.3 | Not Available | 2512 |
| 242 | n-octyl-CHO | 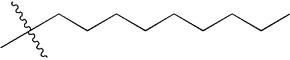 | 24.6 | Not Available | 2528 |
| 243 | n-hexyl-CHO | 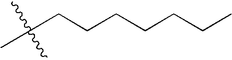 | 22.6 | Not Available | 2500 |
| 244 | 1-phenyl-ethyl-CHO | 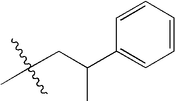 | 22.7 | Not Available | 2534 |
| 245 | 4-Bromo-phenyl- CHO | 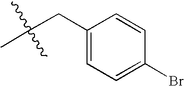 | 22.3 | Not Available | 2584 |
| 246 | Napth-2-yl-CHO | 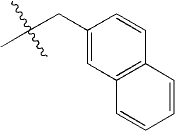 | 22.7 | Not Available | 2556 |
| 247 | 4-Phenoxy-phenyl- CHO | 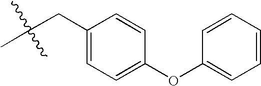 | 24.3 | Not Available | 2498 |

### FORMULA 1D EXAMPLES

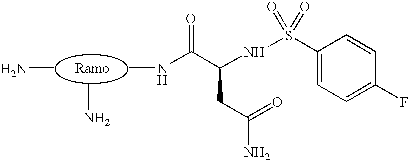

### Example 248

To a solution of 4,10-diFmoc-deacylramoplanin amine (8.7 μmol) in DMF (1.5 ml), TEA (43.5 μmol) and 4-fluorobenzene sulfonyl chloride (17.5 μmol) were added with stirring at room temperature. The reaction was monitored by HPLC analysis (the retention time of desired product was 9.5 minutes). Instrument: Varian 9010; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient time: reaction 0 min % B=35; time 15 min % B=50; time 35 min % B=70).

The mixture was allowed to react at room temperature overnight, then additional TEA (43.5 μmol) and 4-fluorobenzene sulfonyl chloride (17.5 μmol) were added, and the mixture was allowed to react at room temperature for an additional 2 hours. Piperidine (75 μL) was added to remove the protecting group from the ornithine moieties. The reaction was maintained under stirring at room temperature for 30 minutes, and then diluted HCl was added (940 μL of 1M solution). The reaction was monitored by HPLC (the retention time of desired product was 12.4 minutes). Instrument: Varian 9010; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: time 0 min % B=20; time 30 min % B=40). The desired product was obtained as powder via purification by preparative HPLC and lyophilization. Yield 14%. The purified product was tested according to the methods in Example B.

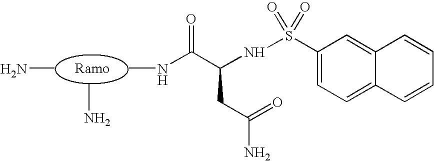

### Example 249

To a solution of 4,10-diFmoc-deacylramoplanin amine (1.75 μmol) in DMF/LiCl 0.4M (100 μL), TEA (8.75 μmol) and naphthalene sulfonyl chloride (3.5 μmol) were added with stirring at room temperature. The reaction was monitored by HPLC analysis (the retention time of desired product was 11.3 minutes). Instrument Varian 9010; column: Merck Lichrocart 125-4 Lichrospher 100 RP 8 (5 μm); flow: 1 ml/min; detector UV λ=270 ml; inj. vol. 10 μl; phase A: HCOONH₄ 0.05M; phase B: CH₃CN; Gradient time: reaction 0 min % B=35; time 15 min % B=50; time 35 min % B=70.

The mixture was allowed to react at room temperature for 10 minutes. Piperidine (5.7 μL) was added to remove the protecting group from the ornithine moieties. The reaction was maintained under stirring at room temperature for 30 minutes and monitored by HPLC (the retention time of desired product was 18.6 minutes). Instrument Varian 9010; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: time 0 min % B=20; time 30 min % B=40). Diluted HCl was added (142.5 μL of 0.5M solution). The desired product was obtained as powder by purification by preparative HPLC and lyophilization. Yield 48%. The purified product was tested according to the methods in Example B.

### FORMULA 1E EXAMPLES

### Examples 250-258

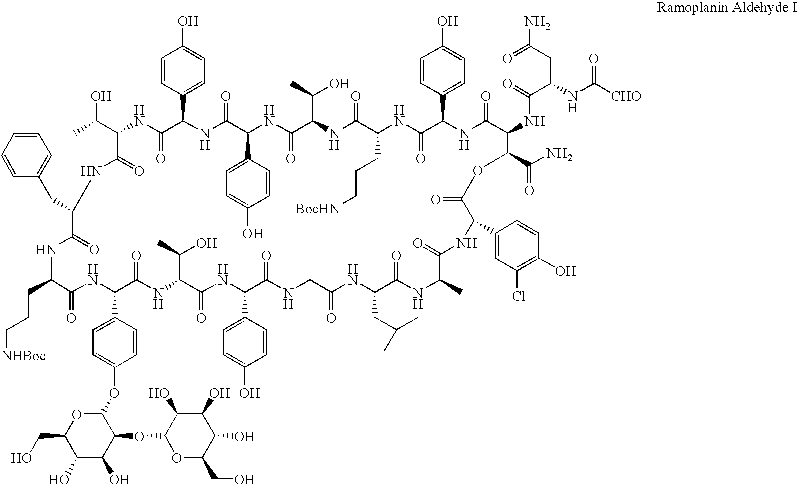

Ramoplanin aldehyde I was synthesized from ramoplanin according to the following protocol:

Step 1: Protection of the ornithine moieties of ramoplanin (synthesis of 4,10-diBoc protected ramoplanin). To a solution of ramoplanin dihydrochoride (5 g, 1.96 mmol) in dry DMF (30 mL), TEA (160 μL, 2.15 mmol) and (Boc)₂O (1.065 g, 4.9 mmol) were added while stirring at 0° C. The mixture was allowed to reach room temperature and react for 1 h. Additional TEA (160 μL, 2.15 mmol) and (Boc)₂O (213 mg, 0.98 mmol) were added, and the reaction was reacted at room temperature overnight.

The reaction was monitored by HPLC analysis (Instrument LC Shimadzu 2010; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: time 0 minutes % B=30; time 35% B=50; time 40% B=70). The retention time of the starting material was 11.20 minutes, and the retention time of the desired product (4,10-diBoc protected ramoplanin) was 15.49 minutes.

The reaction mixture was then poured into AcOEt (400 mL), and the white solid precipitate was filtered off and washed with acetone, obtaining 5.3 g of a white solid.

Step 2: Reductive ozonolysis (synthesis of 4,10-diFmoc-ramoplanin-NHCOCHO). To a solution of 4,10-diBoc-ramoplanin obtained in the previous step (30 g) in methanol/DMF (9:1, 800 ml), cooled to −78° C., ozone was bubbled (40 mmol, at a flow rate of 100 L/hour of oxygen containing 5% ozone) while stirring. The reaction was maintained at −78° C. for 30 minutes. The reaction was monitored by HPLC analysis (retention time 7.5 minutes; instrument and HPLC conditions as above). The excess ozone was eliminated by bubbling nitrogen into the solution. Triphenylphosphine was added (5.8 g), and the reaction was allowed to reach room temperature. Methanol was evaporated under reduced pressure and the residual DMF solution was poured into ethyl acetate (2 L), with stirring. The precipitate was filtered, washed with ethyl acetate (3×150 mL), and dried at room temperature, obtaining 31.5 grams of a solid (yield 100%). MS: Lower isotope molecular weight=2916.

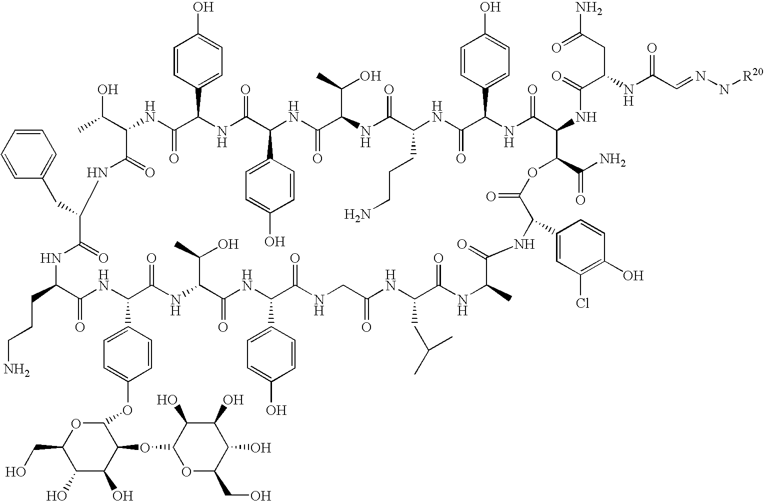

### Ramoplanin Semcarbazone or Hydrazone III

A solution of ramoplanin aldehyde I (6 mg, 2.23 μmol) and a suitable semicarbazone or hydrazone II (R²⁰—NH—NH₂) (20 eq, 22.3 μmol) in DMF (600 μmol) was warmed up to 40° C. overnight. DMF was removed by stripping under nitrogen stream and the crude product of the reaction was treated with TFA for 30 min. After TFA evaporation, the residue was dried well under high-vacuum.

TABLE 7

| Example No. | Semicarbazone or Hydrazone II |
|---|---|
| 250 | 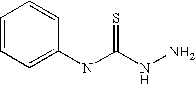 |
| 251 | 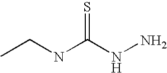 |
| 252 | 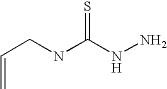 |
| 253 | 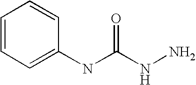 |
| 254 | 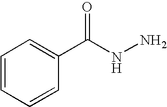 |
| 255 | 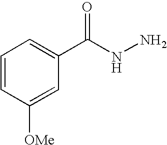 |
| 256 | 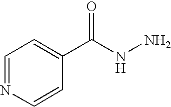 |
| 257 | 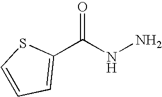 |
| 258 | 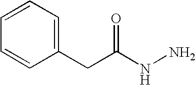 |

The resulting product III was dissolved in 600 μmol of DMF and used as such for the microbiological tests in Example B.

Alternatively, the resulting product is purified by preparative HPLC.

HPLC conditions: (After removal of Boc group under acidic conditions) Thermo-Finningan LC-MS (Surveyor LC-LCQ Advantage; software:Xcalibur); column: Merck Lichrocart 125-4 Lichrospher 100 RP 18 (5 μm); flow: 1 ml/min; inj. vol. 20 μl; phase A: HCOONH₄ 0.05M; phase B: CH₃CN; gradient: time 0 min % B=23; time 10 min % B=28; time 28 min % B=58; time 38 min % B=72.

Masses were obtained using the following conditions: ThermoFinnigan LCQ^{Advantage} ion trap mass spectrometry equipped with an ESI source with LC Surveyor and auto sampler. Sample Inlet Conditions: Capillary Temperature(° C.): 200; Sheat Gas (N₂, arbitrary units): 20. Sample Inlet Voltage Settings: Polarity: positive; Spray Voltage (kV): 4.7; Capillary Voltage (V): 39; Tube Lens Offset (V): +55. Full Scan conditions: Scan range (amu): 200-2000 (double charge ion was detected if MW>2000); Number of microscans: 3; Maximum ion time (ms): 501. Compounds were analysed both by direct infusion (the compound was dissolved in TFA 0.1%-MeCN 1:1) and with HPLC-MS equipment (using the HPLC methods described above).

TABLE 8

| Example | | RT (final | Molecular | Exact |
|---|---|---|---|---|
| No. | R²⁰ | product) | Weight | Mass |
| 250 | Phenylaminothiocarbonyl | 14.5 | 2623.17 | 2620 |
| 251 | N-ethylaminothiocarbonyl | 8.7 | 2575.12 | 2572 |
| 252 | N-prop-2-enylamino- | 9.7 | 2587.13 | 2584 |
| | thiocarbonyl | | | |
| 253 | Phenylaminocarbonyl | 11.0 | 2607.10 | 2604 |
| 254 | Phenylcarbonyl | 11.0 | 2592.09 | 2589 |
| 255 | 3-methoxy-phenylcarbonyl | 13.1 | 2622.12 | 2619 |
| 256 | Pyridine-4-yl-carbonyl | 4.7 | 2593.08 | 2590 |
| 257 | Thiophen-2-ylcarbonyl | 9.5 | 2598.11 | 2595 |
| 258 | Benzylcarbonyl | 11.7 | | 2604 |

### FORMULA IF EXAMPLES

### Examples 259-271

TABLE 9

| | | | | | Exact |
|---|---|---|---|---|---|
| Ex. No. | R^{y} | R² | R⁵ | Rₜ | Mass |
| 259 | —CH₂COOH (L-Asp) | —OH | 2-O-α-D- | 10.3 | 2554 |
| | | | mannopyranosyl-α-D- | | |
| | | | mannopyranosyl | | |
| 260 | —CH₂COOH (L-Asp) | —OH | H | 11.7 | 2230 |
| 261 | —CH₂CONH₂ (L-Asn) | —OH | 2-O-α-D- | 14.9 | 2553 |
| | | | mannopyranosyl-α-D- | or | |
| | | | mannopyranosyl | 20.2 | |
| 262 | —CH₂COOH (L-Asp) | —NH₂ | 2-O-α-D- | 14.9 | 2553 |
| | | | mannopyranosyl-α-D- | or | |
| | | | mannopyranosyl | 20.2 | |
| 263 | —CH₂CONH₂ (L-Asn) | —OH | H | 17.0 | 2229 |
| | | | | or | |
| | | | | 21.6 | |
| 264 | —CH₂COOH (L-Asp) | —NH₂ | H | 17.0 | 2229 |
| | | | | or | |
| | | | | 21.6 | |
| 265 | —CH₂COOCH₃ | —OCH₃ | 2-O-α-D- | 20.7 | 2582 |
| | | | mannopyranosyl-α-D | | |
| | | | mannopyranosyl | | |
| 266 | —CH₂COOCH₃ | —OCH₃ | H | 23.7 | 2258 |
| 267 and | —CH₂CONH₂ (L-Asn) | —OCH₃ | 2-O-α-D- | 18.5 | 2567 |
| 268 | | | mannopyranosyl-α-D- | | |
| (mixture) | | | mannopyranosyl | | |
| | —CH₂COOCH₃ | —NH₂ | 2-O-α-D- | 18.5 | 2567 |
| | | | mannopyranosyl-α-D- | | |
| | | | mannopyranosyl | | |
| 269 | —CH₂CONHCH₂CH— | —CH₂CONH— | 2-O-α-D- | 26.6 | 2664 |
| | (CH₃)₂ | CH₂CH(CH₃)₂ | mannopyranosyl-α-D- | | |
| | | | mannopyranosyl | | |
| 270 | —CH₂CONHCH₂CH₂NH | —CH₂CONH— | 2-O-α-D- | 28.0 | 2838 |
| | —Boc | CH₂CH₂NH— | mannopyranosyl-α-D- | | |
| | | Boc | mannopyranosyl | | |
| 271 | —CH₂CONHCH₂CH₂NH₂ | —CH₂CONH— | 2-O-α-D- | 18.3 | 2638 |
| | | CH₂CH₂NH₂ | mannopyranosyl-α-D | | |
| | | | mannopyranosyl | | |

HPLC conditions (Compounds V1-V6): Shimadzu LC 2010A (CLASS-VP6); column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: time 0 (minutes) % B=20; time 30% B=40; time 35% B=70.

HPLC conditions (Compounds V7-V13): Shimadzu LC 2010A (CLASS-VP6); column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: time 0 (minutes) % B=25; time 35% B=55; time 40% B=70.

### Examples 259-264

Ramoplanin dicarboxylic acid, ramoplanin mono-carboxylic acids, and corresponding aglycons. 2 g of native ramoplanin (0.78 mmol) were dissolved in 100 ml of a 4:1 mixture of hydrochloric acid (1N) and acetonitrile. The solution was allowed to stir at 60° C. for 48 h. The reaction was monitored by HPLC until the native ramoplanin peak had disappeared. The raw mixture was directly injected into preparative HPLC for purification; 6 major peaks were noted in the HPLC procedure at 10.3, 11.7, 14.9, 17.0, 20.2, and 21.6 minutes.

A 75:25 mixture of ramoplanin dicarboxylic acid (Example 259) and the corresponding aglycon (Example 260), respectively, was separated from Examples 261-264 using HPLC. Lyophilisation resulted in a white solid.

In the same HPLC procedure, each mono-acid Ramoplanin derivative (Examples 261 and 262) was also obtained in a mixture with its corresponding aglycon (Examples 263 and 264, respectively).

The derivative mixtures (259 and 260; 261 and 263; 262 and 264) were characterized by ¹H-NMR and MS spectrometry.

### Example 265

Di-methyl-ester ramoplanin. 1 g of native ramoplanin (0.78 mmol) was dissolved in 70 ml of a 3:4 mixture of methanol and hydrochloric acid 37%. The solution was allowed to stir at room temperature for 20 h, and was monitored by HPLC analysis. NaOH (3N) was added until the pH reached 4-5. Purified di-methyl-ester ramoplanin was obtained by preparative HPLC followed by lyophilisation.

The derivative was characterized by ¹H-NMR and MS spectrometry.

### Example 266

Di-methyl-ester ramoplanin aglycon. 1 g of native ramoplanin (0.78 mmol) was dissolved in 70 ml of methanol saturated with hydrochloric acid. The solution was allowed to stir at room temperature for 20 h, and the reaction was monitored by HPLC analysis. NaOH (3N) was added until the pH reached 4-5. Di-methyl-ester ramoplanin aglycon was obtained by preparative HPLC followed by lyophilisation.

The derivative was characterized by ¹H-NMR and MS spectrometry.

### Examples 267-268

Mixture of mono-methyl-esters of ramoplanin. 1 g of native ramoplanin (0.78 mmol) was dissolved in 70 ml of a 5:2 mixture of methanol and hydrochloric acid 37%. The solution was allowed to stir at room temperature for 20 h, then NaOH 3N was added until the pH reached 4-5. A mixture of Examples 267 and 268 was obtained by purification through preparative HPLC and lyophilisation.

The derivative was characterized by ¹H-NMR and MS spectrometry.

### Example 269

Compound 269 (VIC 200088) was obtained following the same procedure as in following Example 270, substituting isobutyl amine for mono-Boc-1,2-ethylenediamine.

### Examples 270-271

To a solution of 259 (di-carboxylic acid ramoplanin) (0.35 mmol) in DMF (40 ml), HOBt (2.8 mmol), mono-Boc-1,2-ethylenediamine (2.8 mmol) and DMAP (0.035 mmol) were added. The resulting basic pH was acidified to pH 5 by adding HOBt (about 2.8 mmol), and then EDC (2.8 mmol) was added. The solution was allowed to stir at room temperature for 6 h and the reaction was monitored by HPLC analysis. 270 (Boc-protected 271) was obtained by purification by preparative HPLC followed by lyophilisation.

The derivative was characterized by ¹H-NMR and MS spectrometry.

500 mg of 270 (Boc-protected 271) (0.39 mmol) were dissolved in 15 ml of a 1:1 mixture of TFA and dichloromethane. The solution was allowed to stir at room temperature for 1 h, then the solvent was removed with a continuous flow of nitrogen gas. The residue was redissolved in water, and 271 was obtained as a white solid by lyophilisation.

The derivative was characterized by MS spectrometry.

### FORMULA IG EXAMPLES

TABLE 10
Examples OR0-OR3

| Ex | | | | |
|---|---|---|---|---|
| No. | R² | R³ | R⁴ | R⁵ |
| OR0 | —NH₂ | (Fluoren-9-yl- | (Fluoren-9-yl- | 2-O-α-D- |
| | | methyl-oxy- | methyl-oxy- | mannopyranosyl-α- |
| | | carbonyl)amino | carbonyl)amino | D-mannopyranosyl |
| OR1 | —NH₂ | —NH₂ | t-butoxy- | 2-O-α-D- |
| | | | carbonylamino | mannopyranosyl-α- |
| | | | | D-mannopyranosyl |
| OR2 | —NH₂ | t-butoxy- | —NH₂ | 2-O-α-D- |
| | | carbonylamino | | mannopyranosyl-α |
| | | | | D-mannopyranosyl |
| OR3 | —NH₂ | t-butoxy- | t-butoxy- | 2-O-α-D- |
| | | carbonylamino | carbonylamino | mannopyranosyl-α |
| | | | | D-mannopyranosyl |

### Examples OR1-OR2

Preparation of 10-Boc protected Ramoplanin (OR1) and 4-Boc protected Ramoplanin (OR2). To a solution of ramoplanin dihydrochoride (5 g, 1.96 mmol) in dry DMF (30 mL), TEA (160 μL, 2.15 mmol) and (Boc)₂O (213 mg, 0.98 mmol) were added while stirring at 0° C. The mixture was allowed to reach room temperature and react for 1 h. Additional TEA (160 μL, 2.15 mmol) and (Boc)₂O (213 mg, 0.98 mmol) were added, and the reaction was reacted at room temperature overnight.

The reaction was monitored by HPLC analysis (Instrument LC Shimadzu 2010; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: time 0 minutes % B=30; time 35% B=50; time 40% B=70). The retention time of the starting material was 11.20 minutes, and the retention time of the desired product (10-Boc protected ramoplanin) was 14.88 minutes. During the reaction, smaller quantities of 4-Boc protected ramoplanin (RT 13.41 minutes) and 4,10-diBoc protected Ramoplanin (RT 15.49 minutes) were also formed.

The reaction mixture was then poured into AcOEt (400 mL), and the white solid precipitate was filtered off and purified by low-pressure C₁₈-reverse-phase preparative column (Instrument: ISCO Combiflash; column: RediSep C₁₈ by ISCO flow: 25 mL/min; detector UV λ=270 nm; phase A: HCOONH₄ 0.05 M; phase B: CH₃CN; Gradient: time 0 min % B=30; time 50 min % B=30; Time 33 min % B=80), obtaining the main product, 10-Boc protected ramoplanin (OR1), as a white solid, and OR2 (4-Boc protected ramoplanin) as a minor compound.

### Example OR3

Preparation of 4,10-diBoc protected ramoplanin. Following the same procedure for the preparation of OR1 and OR2, but using 2.5 equivalents of Boc₂O (4.9 mmol, 1.065 g for 5 g of ramoplanin dihydrochloride) the 4,10-diBoc protected ramoplanin (OR3) was obtained (retention time for the same HPLC conditions as in Examples OR1-OR2 was 15.49 minutes). The reaction mixture was then poured in AcOEt (400 mL) and the white solid precipitate was filtered off and washed with acetone, obtaining 5.3 of a white solid.

### Example 272

To a solution of ramoplanin dihydrochoride (500 mg, 0.2 mmol) in dry DMF (5 mL) N-Guanyl-3,5-dimethyl-pyrazole nitrate (800 mg) was added, and 620 μL (4.4 mmol) of TEA were added to reach pH 8.5. The mixture was reacted overnight at room temperature. The reaction was monitored by HPLC analysis (Instrument LC Shimadzu 2010; column: Merck Lichrocart 125-4 Lichrospher 100 RP 18 (51 μm); flow: 1 l/min; detector UV A=270 nm; inj. vol. 10 μl; phase A: HCOONH₄ 0.05M; phase B: CH₃CN; Gradient: time 0 minutes % B=30; time 40% B=35; time 45% B=90; Retention time of the desired product 17.6 minutes). The reaction mixture was poured into ethyl acetate, and the desired product was filtered off and purified by preparative HPLC followed by lyophilization, obtaining a white solid (138 mg).

### Example 273

Following the same procedure as in Example 272, but using only 40 mg of N-Guanyl-3,5-dimethyl-pyrazole nitrate, the desired mono-guanylated compound (retention time 34.4 minutes) was the major product. HPLC conditions: (Instrument LC Shimadzu 2010; column: Merck Lichrocart 125-4 Lichrospher 100 RP 18 (5 μm); flow: 1 ml/min; detector UV λ=270 nm; inj. vol. 10 μl; phase A: HCOONH₄ 0.05M; phase B: CH₃CN; Gradient: time 0 minutes % B=25; time 40% B=33; time 45% B=90).

### Example 274

Following the same procedure as in Example 272, but substituting OR1 (10-Boc-protected ramoplanin) for ramoplanin dihydrochloride, the desired Boc-protected mono-guanylated compound was obtained.

Treatment of the Boc-protected mono-guanylated compound with TFA:DCM 1:1 for 30 min at room temperature, followed by evaporation under reduced pressure yielded the desired mono-guanylated product 274. Retention time (HPLC conditions as in Example 273)=35 minutes.

### Example 275

To a solution of ramoplanin dihydrochoride (100 mg, 0.039 mmol) in dry DMF (2 mL), TEA was added to reach pH 8.5. To this solution, diBoc-Lysine-succinimidyl ester (diBoc-LysCOOSu) (Fluka catalogue number 15131) (17 mg, 0.039 mmol) was added while stirring at room temperature. An additional amount of (□,N)-diBocLysCOOOSu (17 mg, 0.039 mmol) was added after 1.5 h, and the mixture was reacted overnight. The reaction was monitored by HPLC analysis (Instrument LC Shimadzu 2010; column: Merck Lichrocart 125-4 Lichrospher 100 RP 18 (5 μm); flow: 1 ml/min; detector WV A=270 nm; inj. vol. 10 μl; phase A: HCOONH₄ 0.05M; phase B: CH3CN; Gradient: time 0 minutes % B=20; time 20% B=60; Retention time of the desired product 19.12 minutes). TFA (2 mL) was added and reacted for 30 minutes at room temperature to remove the Boc protecting groups. The reaction mixture was evaporated and the raw material washed with acetone, obtaining a white solid (44 mg). Retention time of the final product 275=12.54 minutes; Exact mass=2807.

### Examples 276-277

Following the same procedure to obtain 275, but using only 17 mg of (ε,N)-diBocLysCOOSu, Boc-protected-277 was isolated by preparative HPLC as a major compound (40 mg). Retention time 17.34 min (HPLC conditions as above in Example 275). Boc-protected 276 was obtained as a minor compound (4 mg). Retention time 16.56 minutes (HPLC conditions as above in Example 275). Retention time of deprotected compounds: (HPLC conditions as above in Example 275) 277=13.46 min, 276=13.66 min.

### Examples 278-282

Following the same procedure for preparing 275, but using a different succinimidyl ester, the following compounds were obtained: 278 (Exact mass=2665), 279 (Exact Mass=2693), 280 (Exact mass=2721), 281 (Exact mass=2749), 282 (Exact mass=2777).

### Examples 283-288

Following the same procedure for preparing 275, but substituting OR1 (10-Boc protected ramoplanin) for ramoplanin dihydrochloride, and substituting the suitable succinimidyl ester, the following compounds were obtained. 283 (retention time 11.44 min); 284 (retention time 11.52 min); 285 (retention time 11.47 min); 286 (retention time 11.53 min); 287 (retention time 11.59 min); 288 (retention time 11.1 min). HPLC Conditions: Instrument LC Shimadzu LC 6; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: time 0 minutes % B=30; time 35% B=50; time 40% B=70.

### Example 289

To a solution of OR1 (10-Boc protected ramoplanin) (20 mg, 0.0075 mmol) in dry DMF (1 mL), succinic anhydride (0.8 mg, 0.075 mmol), TEA (2.4 μL, 0.017 mmol) and a catalytic amount of DMAP were added while stirring at room temperature. An additional amount of TEA (4.8 μL) was added after 2 h, and the reaction was reacted for an additional 2 h. The reaction was monitored by HPLC analysis (Instrument LC Shimadzu 2010; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: time 0 minutes % B=30; time 35% B=50; time 40% B=70; retention time desired product: 12.6 min).

The reaction mixture was evaporated under reduced pressure and TFA:DCM 1:1 (1 mL) was then added and reacted for 1 h at room temperature to remove the Boc protection. The reaction mixture was evaporated and the raw material washed with acetone, obtaining a white solid (44 mg). Using the same HPLC conditions, the retention time of the final product was 8.4 min.

### Example 290

To a solution of OR1 (10-Boc protected ramoplanin) (20 mg, 0.075 mmol) in dry DMF (1 mL), propionaldehyde (6.5 μL, 0.090 mmol) and NaBH₃CN (2 mL, 0.3 mmol) were added while stirring at room temperature, and the reaction was reacted for 5 h. The reaction was monitored by HPLC analysis (Instrument LC Shimadzu 2010; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: time 0 minutes % B=30; time 35% B=50; time 40% B=70 retention time desired product: 18.6 min). The reaction mixture was used directly for the purification by HPLC purification. The desired compound was obtained as a white solid by lyophilization.

The compound obtained in the first step was treated with TFA:DCM 1:1 (0.5 mL) at room temperature for 45 minutes, and then evaporated under reduced pressure to obtain the final product 290. HPLC analysis: (Instrument LC Shimadzu 2010; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: time 0 minutes % B=30; time 35% B=50; time 40% B=70 retention time desired product: 12.3 min).

### Example 291

Following the same procedure for preparing 290, but substituting aqueous formaldehyde for propionaldehyde (36%) (12 equivalents) and THF:water 1:1 as a solvent (1 mL), 291 was obtained. Retention time 11.3 minutes (same HPLC conditions as in Example 290).

### Example 292

Following the same procedure for preparing 290, but substituting D-mannose (12 equivalents) for propionaldehyde, DMF:water 1:0.1 as a solvent (1.1 mL), and reacting at room temperature for 27 days, 292 was obtained. Retention time 10.2 minutes (same HPLC conditions as in Example 290).

### Example 293

To a solution of OR1 (10-Boc protected ramoplanin) (20 mg, 0.0075 mmol) in dry DMF (1 mL), DBU (3.4 μL, 0.023 mmol) and Bromoacetic acid (1 mg, 0.0075 mmol) were added while stirring at room temperature, and the reaction was reacted overnight. The reaction was monitored by HPLC analysis (Instrument LC Shimadzu 2010; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: time 0 minutes % B=30; time 35% B=50; time 40% B=70 retention time desired product: 14.8 min). The reaction mixture was directly used for the purification by HPLC purification. The desired compound was obtained as a white solid by lyophilization.

The compound obtained in the first step was treated with TFA:DCM 1:1 (0.5 mL) at room temperature for 1 h, and then evaporated under reduced pressure to obtain the final product OR25. HPLC analysis (Instrument LC Shimadzu 2010; column: Merck Lichrocart 125-4 Lichrospher 100 RP 8 (5 μm); flow: 1 ml/min; detector UV λ=270 nm; inj. vol. 10 μl; phase A: HCOONH₄ 0.05M; phase B: CH₃CN; Gradient: time 0 minutes % B=30; time 35% B=50; time 40% B=70 retention time desired product: 9.7 min).

### FORMULA 1H EXAMPLES

### Example 294

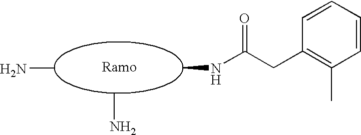

Example 294 was synthesized from 4,10-diFmoc-deacylramoplanin amine and o-tolylacetic acid pentafluorophenyl ester according to Method V. HPLC: Rₜ=4.85 min (Condition 1); Rₜ=3.77 min (Condition 2). ESMS: m/z 1218.2 [(M+2H)/2].

### Example 295

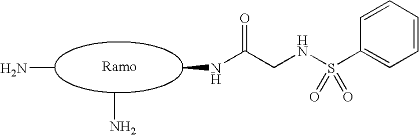

Example 295 was prepared from 4,10-diFmoc-deacylramoplanin amine and benzenesulfonylamino-acetic acid pentafluorophenyl ester according to Method. V. HPLC: Rₜ=4.68 min (Condition 1); 3.68 min (Condition 2). ESMS: m/z 1251.2 [(M+2H)/2].

### Example 296

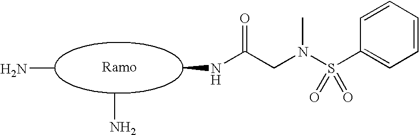

Example 296 was prepared from 4,10-diFmoc-deacylramoplanin amine and (benzenesulfonylmethylamino)acetic acid pentafluorophenyl ester according to Method V. HPLC: Rₜ=4.84 min (Condition 1); Rₜ=3.82 min (Condition 2). ESMS: m/z 1258.4 [(M+2H)/2].

### Example 297

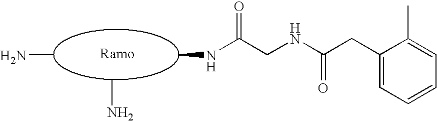

Example 297 was prepared from 4,10-diFmoc-deacylramoplanin amine and (2-o-tolyl-acetylamino)acetic acid pentafluorophenyl ester according to Method V. HPLC: Rₜ=Rₜ=5.07 min (Condition 1); 4.00 min (Condition 2). ESMS: m/z 1247.2 [(M+2H)/2].

The following Examples may be used to test compounds of this invention.

### EXAMPLE A

### Susceptibility Testing

Susceptibility Testing

Compounds were evaluated for antimicrobial activity against a panel of bacterial strains using a broth microdilution assay performed as recommended by the NCCLS (National Committee for Clinical Laboratory Standards (NCCLS). 2003. Methods for Dilution of Antimicrobial Susceptibility Tests for Bacteria That Grow Aerobically—Sixth Edition; Approved Standard. NCCLS Document M7-A6, Vol 23 No. 2.). The minimum inhibitory concentration (MIC) was defined as the lowest concentration of drug which prevented the growth of the bacteria. Prior to addition of test compounds, microtiter plate wells were preincubated with 50 μl of 0.04% w/v solution of bovine serum albumine (BSA) prepared in sterile water. Plates were incubated for 30 minutes at room temperature.

The following 18 organisms constituted the primary panel of evaluation:

| Bacteria | Strain | Characteristic |
|---|---|---|
| Staphylococcus aureus | ATCC 19636 | Wild type |
| Staphylococcus aureus | 613 | MRSA |
| Staphylococcus aureus | HIP-5827 | GISA |
| Staphylococcus aureus | HIP-5836 | GISA |
| Enterococcus faecalis | L559 | VSE |
| Enterococcus faecalis | L560 | VanA |
| Enterococcus faecalis | 2727 | VanB |
| Enterococcus faecalis | J1 | VanB |
| Enterococcus faecalis | 2728 | VanB |
| Enterococcus faecium | A509 | Wild type |
| Enterococcus faecium | D20 | Wild type |
| Enterococcus faecium | F541 | Wild type |

For these organisms of the panel, the assay was performed in cation-adjusted Mueller-Hinton Broth (CAMHB) with a final bacterial inoculum of 5×10⁵ CFU/ml and a final volume of 100 μl. Two controls were also tested, native ramoplanin and BI 603 (Formula IA, wherein R²=R³=R⁴=—NH₂, R⁵=2-O-α-D-mannopyranosyl-α-D-mannopyranosyl, R^{y}=Asn, and R^{x}=2-methyl-benzyl). Compounds were prepared at a concentration of 2 mg/ml in sterile water. A 1:64 dilution was performed in 0.04% BSA to obtain the desired final concentration (8 μg/ml). Dilution of compounds were prepared directly in the plates by serial 2-fold dilution in 0.04% BSA using a multichannel pipette. Positive growth control was included in each plate.

The bacterial inocula were prepared as follows: Bacterial strains were grown overnight at 37° C. on tryptic soy agar (TSA). Bacterial suspensions equivalent to a 0.5 McFarland standard were prepared in sterile saline for each of the strains. Suspensions were used within 15 minutes. The inocula were prepared by diluting the 0.5 McFarland standard 1:100 with fresh cation-adjusted double-concentrated Mueller Hinton (2×CAMHB). A volume of 50 μl of the inocula was added to each well.

Microtiter plates were incubated for 24 h at 37° C. and were read using a microtiterplate reader (Molecular Devices) at 600 nm as well as by visual observation using a microtiterplate reading mirror. The MIC is defined as the lowest concentration of compound at which the visible growth of the organism is completely inhibited.

In addition, compounds were also tested for antimicrobial activity against a strain of Streptococcus pyogenes.

| Bacteria | Strain | Characteristic |
|---|---|---|
| Streptococcus pyogenes | C203, SKF13400 | Wild type |

For this organism, the assay was also performed using a broth microdilution assay as described above but Todd-Hewitt broth was used to addresss the specific growth requirement. S. pyogenes was grown overnight at 37° C. on sheep blood agar. 0.5 McFarland standard was prepared and the suspension was used within 15 minutes. The inoculum was prepared by diluting the 0.5 McFarland standard 1:100 with fresh double-concentrated Todd-Hewitt broth (2×). A volume of 50 μl of the inoculum was added to each well. Endpoints for MIC were also determined as described above.

The anti-fungal activity of these compounds was also studied as a negative control. Activity was assessed against Candida albicans (see table below) using the broth microdilution method as recommended by the NCCLS (National Committee for Clinical Laboratory Standards (NCCLS). 2002. Reference Method for Broth Dilution Antifungal Susceptibility Testing of Yeasts—Second Edition; Approved Standard. NCCLS Document M27-A, Vol 17 No. 9.).

| Bacteria | Strain | Characteristic |
|---|---|---|
| Candida albicans | ATCC14053 | Wild type |

To promote growth, C. albicans were first streaked onto YPD agar and incubated at 37° C. overnight. To prepare inocula, the 0.5 McFarland standard suspension prepared from the overnight culture was diluted 1:2500 using double-concentrated RPMI with MOPS (2×) yielding inocula of 2×10⁴ CFU/ml. Microtiter plates were incubated for 24 h at 37° C. and MIC endpoints were read as described previously.

Serum Effect

The staphylococcal strains ATCC19636 and HIP-5836 and enterococcal isolates 560 and 569 were also tested in 30% bovine serum to obtain a preliminary estimate of the bioactivity of test compounds in serum. The assay was performed in Bovine Serum and cation-adjusted Mueller-Hinton Broth (BSCAMHB) with a final bacterial inoculum of 5×10⁵ CFU/ml and a final volume of 10 μl. Control drugs, native-ramoplanin and BI 603, and new compounds were prepared in 0.04% BSA (see above). Dilution of compounds was performed directly in the plates by serial 2-fold dilution in 0.04% BSA solution using a multichannel pipette. Positive growth control was included in each plate. Strains were grown overnight on TSA. 0.5 McFarland standard were prepared for each of the strains, and suspensions were used within 15 minutes. The inocula were prepared by diluting the 0.5 McFarland standard 1:100 with fresh cation-adjusted double-concentrated Mueller Hinton (2×CAMHB) containing 60% bovin serum. A volume of 50 μl of the inocula was added to each well. This procedure resulted in an inoculum of approximately 5×10⁵ CFU/ml.

Microtiter plates were incubated during 24 h at 37° C. and were read using a microtiterplate reader (Molecular Devices) at 600 nm as well as by visual observation using a microtiterplate reading mirror. The MIC is defined as the lowest concentration of compound at which the visible growth of the organism is completely inhibited. Ratio of MICs in the presence and absence of bovine serum was calculated and provided a measurement of the effect of serum on the bioactivity of test compounds.

Compounds 1-147, 157 and 294-297 were tested according to the methods in Example A. All compounds showed a MIC of 128 μg/mL or less against at least one of the following organisms: Staphylococcus aureus, Staphylococcus aureus, Staphylococcus aureus, Staphylococcus aureus, Enterococcus faecalis, Enterococcus faecalis, Enterococcus faecalis, Enterococcus faecalis, Enterococcus faecalis, Enterococcus faecium, Enterococcus faecium, Enterococcus faecium and Streptococcus pyogenes.

### EXAMPLE B

Compounds 148-156, and 158-293 were tested according to the methods in Example B.

MIC Testing

The reaction solutions (resulting from the addition of 1M HCl as described in the synthetic examples) were diluted to 6000 μg/mL by adding 0.1% peptone (Difco Laboratories, Detroit, Mich.), plus 0.9% NaCl (PBS). The resulting solutions were diluted with water to the desired concentration to perform the following MIC test.

MICs were performed using the broth microdilution methodology following the NCCLS procedure (NCCLS Document M7-A4 Vol. 17 No. 2 January 1997) in the presence of 0.02% albumin bovine serum with inocula of approximately 5×10⁵ cfu/mL. The media employed included cation-adjusted Mueller-Hinton (MH) broth (Difco Laboratories, Detroit, Mich.) supplemented or not with 30% (v/v) bovine serum. Tests were read after 24 hours incubation at 37° C.

Compounds 148-156, and 158-293 showed a MIC of 128 μg/mL or less against at least one of the following organisms: Staphylococcus aureus, Staphylococcus aureus, Staphylococcus aureus, Staphylococcus aureus, Enterococcus faecalis, Enterococcus faecalis, Enterococcus faecalis, Enterococcus faecalis, Enterococcus faecalis, Enterococcus faecium, Enterococcus faecium, Enterococcus faecium and Streptococcus pyogenes.

### Hemolysis

The reaction solutions (resulting from the addition of 1M HCl as described in the synthetic examples) were diluted to 1200 μg/mL by adding 0.1% peptone (Difco Laboratories, Detroit, Mich.), plus 0.9% NaCl (PBS). The resulting solutions were diluted with water to the desired concentration'to perform the following hemolysis test.

The tolerability of the novel ramoplanin derivatives of Formula I in comparison with ramoplanin has been studied by measuring the hemolytic potential on blood cells according to the method in D. Salauze and D. Decouvelare “In vitro assessment of the haemolytic potential of candidate drugs”, Comp. Haematology Intern. 1994; G. Dal Negro and P. Cristofori, “A new approach for evaluation of the in vitro haemolytic potential of solution of a new medicine”, Comp. Hematology Intern. 1996).

Whole blood samples were obtained from the dorsal aorta of rats and diluted 1:100 in PBS before the test. Test groups included:
Group 1: Ramoplanin and ramoplanin derivatives at concentrations ranging from 7.8 μg/mL to 1600 μg/mL.
Group 2: PBS as physiologic haemolysis control
Group 3: Saponin (Sigma), solution at 3% in distilled water, as 100% haemolysis control.

The test groups were diluted 1:5 in blood cells and incubated in a water bath at 37° C. for 45 minutes. After the incubation time, the samples were centrifuged at 2500-3000 g for 10 minutes, and 0.1 mL of each supernatant was diluted in 900 μL of Drabkin's reagent (Sigma). The optical density (OD) of the samples was measured at 540 nm versus a blank preparation of Drabkin's reagent plus 0.1 mL of PBS. The test was performed in triplicate.

The percentage of haemolysis was calculated using the formula:
Δx/Δt×100=% of haemolysis
Δx=mean value of OD₅₄₀ for each concentration tested (Groups 1 and 2).
Δt=mean value of OD₅₄₀ for 100% haemolysis control (Group 3).

The haemolysis was considered significant when it exceeded the haemolytic value of the blank control (Group 2) by at least 3 fold.

Numerous variations of such details can be implied as will be appreciated by those skilled in the art.

What is claimed is:

1. A compound of Formula (I): 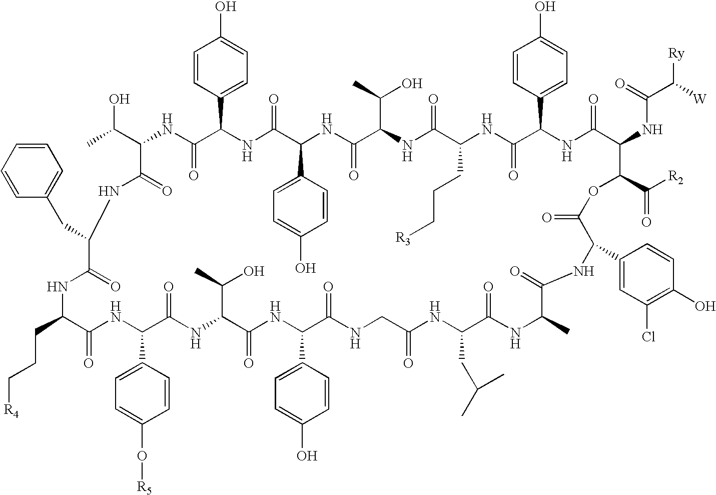
wherein:
R² is selected from the group consisting of: —NH₂, —NHR¹⁸, —OH, and —OR¹⁶ wherein R¹⁸ is alkyl, aminoalkyl, alkylaminoalkyl, or alkoxycarbonylaminoalkyl, and wherein R¹⁶ is alkyl or aminoalkyl;
R³ and R⁴ are independently selected from the group consisting of: —NH₂, —NHCO(CH₂)ₙNH₂, —NHCO(CH₂)ₙCO₂H, —NH(CH₂)ₙCO₂H, —NHCOC(R¹⁷)NH₂, —NH—C(═NH)—NH₂, —NH-alkyl, —NH(CH₂)ₙNH₂, —N(CH₃)₂, —NHCO—C₆H₄-p-CH₂NH₂, and 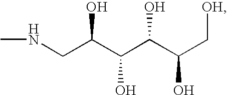
wherein n is 1-5, and wherein R¹⁷ is a natural or synthetic amino acid side chain;
R⁵ is selected from the group consisting of: H, α-D-mannopyranosyl, and 2-O-α-D-mannopyranosyl-α-D-mannopyranosyl;
R^{y} is selected from the group consisting of: —H, —CH₂CONH₂, —CH₂CONHR¹⁸, —CH₂CO₂H, and —CH₂CO₂R¹⁶, wherein R¹⁸ is alkyl, aminoalkyl, alkylaminoalkyl, or alkoxycarbonylaminoalkyl, and wherein R¹⁶ is alkyl or aminoalkyl;
W is selected from the group consisting of: —NH—C(O)—R^{x}, —NH—C(S)—NH—R^{z}, —NH—C(O)—NH—R^{z}, —NH—C(O)O—R^{z}, —NH—R′, —NH—S(O₂)—R″, —N(CH₃)—S(O₂)—R″, —NH—C(O)—CH═N—NH—R²⁰, and substituted aryl;
R^{x} is selected from the group consisting of: alkyl, substituted alkyl, alkenyl, substituted cycloalkyl, cycloalkenyl, aryl, substituted aryl, heteroaryl, substituted heteroaryl, heterocyclic, substituted heterocyclic, substituted carbonyl-amino acid residue (—C(O)—CH(R¹¹)—NH—C(O)—R¹²), wherein R¹¹ is a natural or synthetic amino acid side chain, and R¹² is alkyl, substituted alkyl;
R^{z} is selected from the group consisting of: alkyl, substituted alkyl, cycloalkyl, substituted cycloalkyl, alkenyl, alkynyl, aryl, substituted aryl,
R′ is -alkylene-R¹⁰, wherein R¹⁰ is selected from the group consisting of: H, aryl, substituted aryl, cycloalkyl, substituted cycloalkyl;
R″ is selected from the group consisting of: aryl and haloaryl;
R²⁰ is selected from the group consisting of:
carbonyl substituted with aryl, substituted aryl, arylalkyl, heteroaryl, heterocyclic, and substituted amino, and
thiocarbonyl substituted with substituted amino;
and prodrugs, tautomers or pharmaceutically acceptable salts thereof;
with the following provisos:
(1) when R^{y} is —CH₂CONH₂, R² is —NH₂, R³ and R⁴ are —NH₂ or —NH(protecting group), R⁵ is H, α-D-mannopyranosyl, or 2-O-α-D-mannopyranosyl-α-D-mannopyranosyl, and W is —NH—C(O)—R^{x}, then R^{x} is not alkyl, alkenyl, —R²¹, -(C1-C4 alkylene)-R²¹, or -(C2-C4 alkenylene)-R²¹;
wherein R²¹ is alkoxy; alkenyloxy; alkoxy substituted with halo; alkenyloxy substituted with halo; phenyl; phenyl substituted with R²², halo, cyano, nitro, alkyl, haloalkyl, alkoxy, haloalkoxy, alkylsulfanyl, and/or haloalkylsulfanyl; napthyl; napthyl substituted with halo, alkyl, haloalkyl, alkoxy, and/or haloalkoxy, phenoxy; phenoxy substituted with halo, cyano, nitro, alkyl, haloalkyl, alkoxy, haloalkoxy, alkylsulfanyl, and/or haloalkylsulfanyl; napthoxy, napthoxy substituted with halo, alkyl, haloalkyl, alkoxy, and/or haloalkoxy,
wherein R²² is phenyl, phenylalkyl, phenoxy, or phenoxyalkyl, and wherein the phenyl portion of said phenyl, phenylalkyl, phenoxy, and phenoxyalkyl may be substituted with halo, cyano, nitro, alkyl, haloalkyl, alkoxy, haloalkoxy, alkylsulfanyl, and/or haloalkylsulfanyl; and
(2) when R^{y} is —CH₂CONH₂, R² is —NH₂, R³ and R⁴ are —NH₂ or —NHCOCH(CH₃)NH₂, R⁵ is 2-O-α-D-mannopyranosyl-α-D-mannopyranosyl, and W is —NH—C(O)—R^{x}, then R^{x} is not alkyl or alkenyl.

2. A compound according to claim 1, wherein the compound of Formula I has a minimum inhibition concentration of 128 μg/mL or less against at least one of the organisms selected from the group consisting of Actinomyces spp, Bacillus spp, Bacillus anthracis, Bacillus cereus, Clostridium spp, Clostridium difficile, Clostridium perfringens, Clostridium botulinum, Clostridium tetani, Clostridium ramosum, Clostridium, Corynebacterium spp, Corynebacterium dihpteriae, Enterococcus spp, Enterococcus faecalis, Enterococcus faecium, Enterococcus gallinarum, Enterococcus casseliflavus, Enterococcus avium, Enterococcus durans, Enterococcus raffinosus, Entrerococcus hirae, Enterococcus pseudoavium, Enterococcus malodoratus, Enterococcus mundtii, Erysipelothrix rhusiopathiae, Eubacterium, Gemella haemolysans, Gemella morbillorum, Lactobacillus spp, Lactobacillus rhamnosus, Lactobacillus paracasei, Leuconostoc spp, Leuconostoc mesenteroides, Listeria monocytogenes, Peptostreptococcus magnus, Peptostreptococcus asaccharolyticus, Peptostreptococcus anaerobius, Peptostreptococcus prevotii, Peptostreptococcus micros, Peptostreptococcus hydrogenalis, Propionibacterium acne, Staphylococcus spp, Staphylococcus aureus, Staphylococcus epidermidis, Staphylococcus hominis, Staphylococcus haemolyticus, Staphylococcus saprophyticus, Streptococcus spp, Streptococcus pneumoniae, Streptococcus pyogenes, Streptococcus agalactiae, Streptococcus mutans, Streptococcus sanguis, Streptococcus mitis, Streptococcus bovis, Streptococcus salivarius, Steptococcus anginosus, Streptococcus constellatus, and Streptococcus intermedius.

3. A compound according to claim 1, wherein R² is selected from the group consisting of: —NH₂, —OH, —OCH₃, —NH—CH₂CH(CH₃)₂, —NH—CH₂CH₂NHBoc, —NH—CH₂CH₂NH₂, —NHCH₂CH₂CH₂NH₂, —NHCH₂CH₂CH₂CH₂NH₂, —NHCH₂CH₂NHCH₃, —NHCH₂CH₂N(CH₃)₂, and OCH₂CH₂NH₂.

4. A compound according to claim 1, wherein R² is —NH—CH₂CH₂NH₂.

5. A compound according to claim 1, wherein R² is —NH₂.

6. A compound according to claim 1, wherein R³ and R⁴ are independently selected from the group consisting of: —NH₂, —N-(aminomethyl-carbonyl)-amino, —N-(2-amino-ethyl-carbonyl)amino, —N-(3-amino-propyl-carbonyl)amino, —N-(4-amino-butyl-carbonyl)amino, —N-(5-amino-pentyl-carbonyl)amino, —N-(1,5-diamino-pentyl-carbonyl)amino, —NHCOCH₂CH₂COOH, —NHCH₂CH₂CH₃, —N(CH₃)₂, —NHCH₂COOH, —NH—C(═NH)—NH₂,

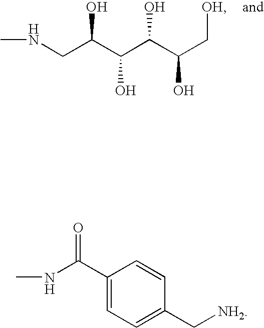

7. A compound according to claim 1, wherein R³ and R⁴ are independently selected from the group consisting of: —N-(1,5-diamino-pentyl-carbonyl)amino and

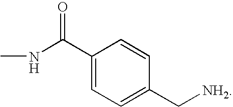

8. A compound according to claim 1, wherein R³ and R⁴ are —NH₂.

9. A compound according to claim 1, wherein R⁵ is 2-O-α-D-mannopyranosyl-α-D-mannopyranosyl.

10. A compound according to claim 1, wherein R⁵ is H.

11. A compound according to claim 1, wherein R^{y} is selected from the group consisting of: —H, —CH₂COOH, —CH₂CONH₂, —CH₂COOCH₃, —CH₂CONHCH₂CH(CH₃)₂, —CH₂CONHCH₂CH₂NHBoc, and —CH₂CONHCH₂CH₂NH₂.

12. A compound according to claim 1, wherein R^{y} is selected from the group consisting of: —CH₂COOH, —CH₂CONH₂, —CH₂COOCH₃, —CH₂CONHCH₂CH(CH₃)₂, —CH₂CONHCH₂CH₂NHBoc, and —CH₂CONHCH₂CH₂NH₂.

13. A compound according to claim 1, wherein R^{y} is —CH₂CONHCH₂CH₂NH₂.

14. A compound according to claim 1, wherein R^{y} is —CH₂CONH₂.

15. A compound according to claim 1, wherein W is —NH—C(O)—R^{x}.

16. A compound according to claim 15, wherein R^{x} is selected from the group consisting of: thiophen-2-yl-methyl; 3-methyl-benzo[b]thiophen-2-yl-methyl; benzo[b]thiophen-3-yl-methyl; 5-chloro-benzo[b]thiophen-3-yl-methyl; thiophen-3-yl-methyl; benzo[1,3]dioxol-5-yl-methyl; (±)-2,3-dihydro-benzo[1,4]dioxin-2-yl; 2-benzyloxy-benzyl; 2-phenylsulfanyl-benzyl; 4-thiophen-2-yl-phenyl; benzo[d]isoxazol-3-yl-methyl; benzothiazol-5-yl; 5-phenyl-thiophen-2-yl; 3-methyl-thiophen-2-yl-methyl; 2-E-(3-methyl-thiophen-2-yl)-ethenyl; 2-(3-methyl-thiophen-2-yl)-ethyl; 3-phenyl-isoxazol-5-yl; 5-methyl-isoxazol-3-yl; 5-methyl-2-phenyl-2H-[1,2,3]-triazol-4-yl; 5-tert-butyl-2-methyl-2H-pyrazol-3-yl; 3-pyridin-2-yl-isoxazol-5-yl; 3-ethyl-isoxazol-5-yl; 3-propyl-isoxazol-5-yl; 3-isopropyl-isoxazol-5-yl; 3-isobutyl-isoxazol-5-yl; 3-butyl-isoxazol-5-yl; 3-tert-butyl-isoxazol-5-yl; 3-(1-methylpropyl)-isoxazol-5-yl; indol-1-yl-methyl; 2-E-(5-methyl-thiophen-2-yl)-ethenyl; 2-(5-methyl-thiophen-2-yl)-ethyl; methyl-sulfonyl-N-phenyl-amino-methyl; phenyl-sulfonyl-N-phenyl-amino-methyl; 5-methyl-thiophen-2-yl; 4-methyl-thiophen-2-yl; 3-methyl-thiophen-2-yl; 5-methyl-thiophen-2-yl-methyl; 4-methyl-thiophen-2-yl-methyl; 2-E-(4-methyl-thiophen-2-yl)-ethenyl; 2-(4-methyl-thiophen-2-yl)-ethyl; 5-phenyl-isoxazol-3-yl; 3-phenyl-isoxazol-5-yl-methyl; 3-isobutyl-isoxazol-5-yl-methyl; (5-phenylimidazol-1-yl)methyl; (benzimidazol-1-yl)methyl; (2-phenylimidazol-1-yl)methyl; biphenyl-2-yl-oxy-methyl; biphenyl-3-yl-oxy-methyl; biphenyl-4-yl-oxy-methyl; 3-methyl-isoxazol-5-yl-methyl; benzofuran-2-yl; 1H-indol-3-yl-methyl; 1H-indol-2-yl; 5-ethyl-8-oxo-5,8-dihydro-[1,3]dioxolo-[4,5-g]quinolin-7-yl; 7-chloro-1-cyclopropyl-6-fluoro-4-oxo-1,4-dihydro-quinolin-3-yl; 8-Fluoro-3-methyl-9-(4-methyl-piperazin-1-yl)-2,3-dihydro-1-oxa-3a-aza-phenalen-6-one-5-yl; 1-Ethyl-7-methyl-4-oxo-1,4-dihydro-[1,8]naphthyridin-3-yl; quinolin-4-yl; quinolin-8-yl; quinolin-6-yl; 2,2-difluoro-benzo[1,3]dioxol-5-yl; 2,2-Difluoro-benzo[1,3]dioxol-4-yl; quinolin-2-yl; quinolin-5-yl; quinolin-3-yl; (1-oxo-1,3-dihydroisoindol-2-yl)methyl; (2-oxo-2,3-dihydroindol-1-yl)methyl; (2-oxo-benzoxazol-3-yl)methyl; (benzotriazol-1-yl)methyl; (indazol-1-yl)methyl; 2,2-difluoro-benzo[1,3]dioxol-4-yl-methyl; 1-methyl-1H-indol-3-yl-methyl; 5-phenyl-isoxazol-3-yl-methyl; 3-isopropyl-isoxazol-5-yl-methyl; benzo[1,3]dioxol-yl; 2,2-difluoro-benzo[1,3]dioxol-5-yl-methyl; (3-methyl-2-oxo-2,3-dihydrobenzimidazol-1-yl)methyl; (2-oxo-2,3-dihydrobenzimidazol-1-yl)methyl; (3-ethyl-2-oxo-2,3-dihydro-benzimidazol-1-yl)methyl; (4-methyl-2-oxo-benzooxazol-3-yl)methyl; (5-methyl-2-oxo-benzooxazol-3-yl)methyl; (6-methyl-2-oxo-benzooxazol-3-yl)methyl; 4-(4-methoxy-phenyl)-thiophen-2-yl; 2-phenyl-thiazol-4-yl-methyl; 2-phenyl-thiazol-4-yl; 2-phenyl-oxazol-4-yl-methyl; 1-methyl-1H-indol-2-yl; 2-phenyl-oxazol-4-yl; 2-methyl-thiazolyl-methyl; 2-methyl-oxazol-4-yl-methyl; (5-methyl-2-phenyl-2H-[1,2,3]triazol-4-yl)methyl; (5-phenyltetrazol-1-yl)methyl; (4R,5S)-(+)-4-methyl-5-phenyl-oxazolidin-2-one-3-yl-methyl; (4S,5R)-(−)-4-methyl-5-phenyl-oxazolidin-2-one-3-yl-methyl; pyrrolidin-2-one-1-yl-methyl; 2-cyclohexyl-oxazol-4-yl-methyl; (4R)-4-phenyl-oxazolidin-2-one-3-yl-methyl; (4S)-4-phenyl-oxazolidin-2-one-3-yl-methyl; (2-cyclohexylthiazol-4-yl)methyl; 5-(4-methyl-phenyl)-tetrazol-1-yl-methyl; 5-(4-methoxy-phenyl)-tetrazol-1-yl-methyl; 2-ethenyl-benzyl; 4-difluoromethoxy-phenyl; 4-trifluoromethoxy-phenyl; 2-ethynyl-benzyl; 1-aceto-piperidin-4-yl; 1-(4-chloro-benzyl)-pyrrolidin-2-one-4-yl; bicyclo[4.2.0]octa-1(6),2,4-trien-7-yl; 5-methyl-1-phenyl-1H-pyrazolyl; 1-methyl-5-phenyl-1H-pyrazol-3-yl-methyl; (2-methyl-5-phenyl-2H-pyrazol-3-yl)methyl; 1-ethyl-5-phenyl-1H-pyrazol-3-yl-methyl; (2-ethyl-5-phenyl-2H-pyrazol-3-yl)methyl; (2,5-diphenyl-2H-pyrazol-3-yl)methyl; (2-tert-butyl-5-phenyl-2H-pyrazol-3-yl)methyl; (2-cyclohexyl-5-phenyl-2H-pyrazol-3-yl)methyl; (5-methyl-2-phenyl-2H-pyrazol-3-yl)methyl; 2-methyl-5-phenyl-2H-pyrazol-3-yl; 1-methyl-5-phenyl-1H-pyrazol-3-yl; (5-phenyl-1-propyl-1H-pyrazol-3-yl)methyl; 1-butyl-5-phenyl-1H-pyrazol-3-yl-methyl; 1-isobutyl-5-phenyl-1H-pyrazol-3-yl-methyl; (5-phenyl-pyrazol-1-yl)methyl; (3-methyl-5-phenyl-pyrazol-1-yl)methyl; (5-methyl-3-phenylpyrazol-1-yl)methyl; (3-phenylpyrazol-1-yl)methyl; 2-phenyl-2H-pyrazol-3-yl; 2-(bis-methylsulfonylamino)-benzyl; L-phenyl-sulfonyl-amino-phenylmethyl; L-phenyl-sulfonyl-N-methyl-amino-phenylmethyl; phenyl-sulfonyl-amino-methyl; phenyl-sulfonyl-N-methyl-amino-methyl; phenyl-sulfonyl-N-ethyl-amino-methyl; phenyl-sulfonyl-N-isopropyl-amino-methyl; phenyl-sulfonyl-N-propyl-amino-methyl; phenyl-sulfonyl-N-benzyl-amino-methyl; benzyl-sulfonyl-amino-methyl; benzyl-sulfonyl-N-methyl-amino-methyl; benzyl-sulfonyl-N-propyl-amino-methyl; benzyl-sulfonyl-N-benzyl-amino-methyl; benzyl-sulfonyl-N-ethyl-amino-methyl; benzyl-sulfonyl-N-isopropyl-amino-methyl; (4-phenyl-[1,2,3]triazol-1-yl)methyl; (5-phenyl-[1,2,3]triazol-1-yl)methyl; (5-phenyltetrazol-2-yl)methyl; 5-phenyl-oxazol-4-yl; 5-phenyl-oxazol-4-yl-methyl; N-(n-butyl-carbonyl)amino-methyl; N-n-butyl-carbonyl)amino-benzylmethyl; N-(1-ethyl-n-pentyl-carbonyl)amino-methyl; N-(2-methyl-benzyl-carbonyl)amino-benzylmethyl; 1-N-(n-butyl-carbonyl)amino-ethyl; 1-N-(2-methyl-benzyl-carbonyl)amino-ethyl; N-(2-methyl-benzyl-carbonyl)amino-methyl; 1-N-(1-ethyl-n-pentyl-carbonyl)amino-ethyl; N-(1-ethyl-n-pentyl-carbonyl)amino-benzylmethyl; 1-N-(2-methyl-benzyl-carbonyl)amino-2-methyl-butyl; cyclopentyl; cyclopentyl-methyl; 2-cyclopentyl-ethyl; 1-phenyl-cyclopentyl; bicyclo[2.2.1]heptylmethyl; cyclohexylmethyl; 4-methyl-cyclohexyl-methyl; 2-methyl-cyclohexyl-methyl; 4-pentyl-cyclohexyl-methyl; cycloheptyl; cyclopropyl; 2-methylcyclopropyl; 1-methylcyclopropyl; 2,2,3,3-tetramethyl-cyclopropyl; 2-(2-methyl-prop-1-enyl)-3,3-dimethyl-cyclopropyl; 2-phenyl-cyclopropyl; 1-phenyl-cyclopropyl; cyclobutyl; cyclohexen-3-yl; and 2-methyl-benzyl.

17. A compound according to claim 15, wherein R^{x} is selected from the group consisting of: thiophen-2-yl-methyl; 3-methyl-benzo[b]thiophen-2-yl-methyl; benzo[b]thiophen-3-yl-methyl; 5-chloro-benzo[b]thiophen-3-yl-methyl; thiophen-3-yl-methyl; benzo[1,3]dioxol-5-yl-methyl; (±)-2,3-dihydro-benzo[1,4]dioxin-2-yl; 2-benzyloxy-benzyl; 2-phenylsulfanyl-benzyl; 4-thiophen-2-yl-phenyl; benzo[d]isoxazol-3-yl-methyl; benzothiazol-5-yl; 5-phenyl-thiophen-2-yl; 3-methyl-thiophen-2-yl-methyl; 2-E-(3-methyl-thiophen-2-yl)-ethenyl; 2-(3-methyl-thiophen-2-yl)-ethyl; 3-phenyl-isoxazol-5-yl; 5-methyl-isoxazol-3-yl; 5-methyl-2-phenyl-2H-[1,2,3]-triazol-4-yl; 5-tert-butyl-2-methyl-2H-pyrazol-3-yl; 3-pyridin-2-yl-isoxazol-5-yl; 3-ethyl-isoxazol-5-yl; 3-propyl-isoxazol-5-yl; 3-isopropyl-isoxazol-5-yl; 3-isobutyl-isoxazol-5-yl; 3-butyl-isoxazol-5-yl; 3-tert-butyl-isoxazol-5-yl; 3-(1-methylpropyl)-isoxazol-5-yl; indol-1-yl-methyl; 2-E-(5-methyl-thiophen-2-yl)-ethenyl; 2-(5-methyl-thiophen-2-yl)-ethyl; methyl-sulfonyl-N-phenyl-amino-methyl; phenyl-sulfonyl-N-phenyl-amino-methyl; 5-methyl-thiophen-2-yl; 4-methyl-thiophen-2-yl; 3-methyl-thiophen-2-yl; 5-methyl-thiophen-2-yl-methyl; 4-methyl-thiophen-2-yl-methyl; 2-E-(4-methyl-thiophen-2-yl)-ethenyl; 2-(4-methyl-thiophen-2-yl)-ethyl; 5-phenyl-isoxazol-3-yl; 3-phenyl-isoxazol-5-yl-methyl; 3-isobutyl-isoxazol-5-yl-methyl; (5-phenylimidazol-1-yl)methyl; (benzimidazol-1-yl)methyl; (2-phenylimidazol-1-yl)methyl; biphenyl-2-yl-oxy-methyl; biphenyl-3-yl-oxy-methyl; biphenyl-4-yl-oxy-methyl; 3-methyl-isoxazol-5-yl-methyl; benzofuran-2-yl; 1H-indol-3-yl-methyl; 1H-indol-2-yl; 5-ethyl-8-oxo-5,8-dihydro-[1,3]dioxolo-[4,5-g]quinolin-7-yl; 7-chloro-1-cyclopropyl-6-fluoro-4-oxo-1,4-dihydro-quinolin-3-yl; 8-Fluoro-3-methyl-9-(4-methyl-piperazin-1-yl)-2,3-dihydro-1-oxa-3a-aza-phenalen-6-one-5-yl; 1-Ethyl-7-methyl-4-oxo-1,4-dihydro-[1,8]naphthyridin-3-yl; quinolin-4-yl; quinolin-8-yl; quinolin-6-yl; 2,2-difluoro-benzo[1,3]dioxol-5-yl; 2,2-Difluoro-benzo[1,3]dioxol-4-yl; quinolin-2-yl; quinolin-5-yl; quinolin-3-yl; (1-oxo-1,3-dihydroisoindol-2-yl)methyl; (2-oxo-2,3-dihydroindol-1-yl)methyl; (2-oxo-benzoxazol-3-yl)methyl; (benzotriazol-1-yl)methyl; (indazol-1-yl)methyl; 2,2-difluoro-benzo[1,3]dioxol-4-yl-methyl; 1-methyl-1H-indol-3-yl-methyl; 5-phenyl-isoxazol-3-yl-methyl; 3-isopropyl-isoxazol-5-yl-methyl; benzo[1,3]dioxol-4-yl; 2,2-difluoro-benzo[1,3]dioxol-5-yl-methyl; (3-methyl-2-oxo-2,3-dihydrobenzimidazol-1-yl)methyl; (2-oxo-2,3-dihydrobenzimidazol-1-yl)methyl; (3-ethyl-2-oxo-2,3-dihydro-benzimidazol-1-yl)methyl; (4-methyl-2-oxo-benzooxazol-3-yl)methyl; (5-methyl-2-oxo-benzooxazol-3-yl)methyl; (6-methyl-2-oxo-benzooxazol-3-yl)methyl; 4-(4-methoxy-phenyl)-thiophen-2-yl; 2-phenyl-thiazol-4-yl-methyl; 2-phenyl-thiazol-4-yl; 2-phenyl-oxazolyl-methyl; 1-methyl-1H-indol-2-yl; 2-phenyl-oxazolyl; 2-methyl-thiazol-4-yl-methyl; 2-methyl-oxazol-4-yl-methyl; (5-methyl-2-phenyl-2H-[1,2,3]triazol-4-yl)methyl; (5-phenyltetrazol-1-yl)methyl; (4R,5S)-(+)-4-methyl-5-phenyl-oxazolidin-2-one-3-yl-methyl; (4S,5R)-(−)-4-methyl-5-phenyl-oxazolidin-2-one-3-yl-methyl; pyrrolidin-2-one-1-yl-methyl; 2-cyclohexyl-oxazol-4-yl-methyl; (4R)-4-phenyl-oxazolidin-2-one-3-yl-methyl; (4S)-4-phenyl-oxazolidin-2-one-3-yl-methyl; (2-cyclohexylthiazol-4-yl)methyl; 5-(4-methyl-phenyl)-tetrazol-1-yl-methyl; 5-(4-methoxy-phenyl)-tetrazol-1-yl-methyl; 2-ethenyl-benzyl; 4-difluoromethoxy-phenyl; 4-trifluoromethoxy-phenyl; 2-ethynyl-benzyl; 1-aceto-piperidinyl; 1-(4-chloro-benzyl)-pyrrolidin-2-one-4-yl; bicyclo[4.2.0]octa-1(6),2,4-trien-7-yl; 5-methyl-1-phenyl-1H-pyrazol-4-yl; 1-methyl-5-phenyl-1H-pyrazol-3-yl-methyl; (2-methyl-5-phenyl-2H-pyrazol-3-yl)methyl; 1-ethyl-5-phenyl-1H-pyrazol-3-yl-methyl; (2-ethyl-5-phenyl-2H-pyrazol-3-yl)methyl; (2,5-diphenyl-2H-pyrazol-3-yl)methyl; (2-tert-butyl-5-phenyl-2H-pyrazol-3-yl)methyl; (2-cyclohexyl-5-phenyl-2H-pyrazol-3-yl)methyl; (5-methyl-2-phenyl-2H-pyrazol-3-yl)methyl; 2-methyl-5-phenyl-2H-pyrazol-3-yl; 1-methyl-5-phenyl-1H-pyrazol-3-yl; (5-phenyl-1-propyl-1H-pyrazol-3-yl)methyl; 1-butyl-5-phenyl-1H-pyrazol-3-yl-methyl; 1-isobutyl-5-phenyl-1H-pyrazol-3-yl-methyl; (5-phenyl-pyrazol-1-yl)methyl; (3-methyl-5-phenyl-pyrazol-1-yl)methyl; (5-methyl-3-phenylpyrazol-1-yl)methyl; (3-phenylpyrazol-1-yl)methyl; 2-phenyl-2H-pyrazol-3-yl; 2-(bis-methylsulfonylamino)-benzyl; L-phenyl-sulfonyl-amino-phenylmethyl; L-phenyl-sulfonyl-N-methyl-amino-phenylmethyl; phenyl-sulfonyl-amino-methyl; phenyl-sulfonyl-N-methyl-amino-methyl; phenyl-sulfonyl-N-ethyl-amino-methyl; phenyl-sulfonyl-N-isopropyl-amino-methyl; phenyl-sulfonyl-N-propyl-amino-methyl;
phenyl-sulfonyl-N-benzyl-amino-methyl; benzyl-sulfonyl-amino-methyl; benzyl-sulfonyl-N-methyl-amino-methyl; benzyl-sulfonyl-N-propyl-amino-methyl; benzyl-sulfonyl-N-benzyl-amino-methyl; benzyl-sulfonyl-N-ethyl-amino-methyl; benzyl-sulfonyl-N-isopropyl-amino-methyl; (4-phenyl-[1,2,3]triazol-1-yl)methyl; (5-phenyl-[1,2,3]triazol-1-yl)methyl; (5-phenyltetrazol-2-yl)methyl; 5-phenyl-oxazol-4-yl; 5-phenyl-oxazol-4-yl-methyl; N-(n-butyl-carbonyl)amino-methyl; N-(n-butyl-carbonyl)amino-benzylmethyl; N-(1-ethyl-n-pentyl-carbonyl)amino-methyl; N-2-methyl-benzyl-carbonyl)amino-benzylmethyl; 1-N-(n-butyl-carbonyl)amino-ethyl; 1-N-2-methyl-benzyl-carbonyl)amino-ethyl; N-(2-methyl-benzyl-carbonyl)amino-methyl; 1-N-(1-ethyl-n-pentyl-carbonyl)amino-ethyl; N-(1-ethyl-n-pentyl-carbonyl)amino-benzylmethyl; 1-N-(2-methyl-benzyl-carbonyl)amino-2-methyl-butyl; cyclopentyl; cyclopentyl-methyl; 2-cyclopentyl-ethyl; 1-phenyl-cyclopentyl; bicyclo[2.2.1]heptylmethyl; cyclohexylmethyl; 4-methyl-cyclohexyl-methyl; 2-methyl-cyclohexyl-methyl; 4-pentyl-cyclohexyl-methyl; cycloheptyl; cyclopropyl; 2-methylcyclopropyl; 1-methylcyclopropyl; 2,2,3,3-tetramethyl-cyclopropyl; 2-(2-methyl-prop-1-enyl)-3,3-dimethyl-cyclopropyl; 2-phenyl-cyclopropyl; 1-phenyl-cyclopropyl; cyclobutyl; and cyclohexen-3-yl.

18. A compound according to claim 15, wherein R^{x} is selected from the group consisting of: benzo[d]isoxazol-3-yl-methyl, 3-methyl-thiophen-2-yl-methyl, 1-methyl-5-phenyl-1H-pyrazol-3-yl-methyl, (2-methyl-5-phenyl-2H-pyrazol-3-yl)methyl, (indazol-1-yl)methyl, (2-oxo-benzoxazol-3-yl)methyl, and (5-phenyltetrazol-1-yl)methyl.

19. A compound according to claim 15, wherein R^{x} is —CH═CH—CH═CH—CH₂—CH(CH₃)₂, —(CH₂)₅CH(CH₃)₂, or 2-methyl-benzyl.

20. A compound according to claim 15, wherein R^{x} is phenyl, a 5-membered heteroaryl ring, a 6-membered heteroaryl ring, a 5-membered heterocyclic ring, or a 6-membered heterocyclic ring, wherein the phenyl, 5-membered heteroaryl ring, 6-membered heteroaryl ring, 5-membered heterocyclic ring, or 6-membered heterocyclic ring has a single substituent at the ortho position.

21. A compound according to claim 15, wherein R^{x} is —H₂—R²³, wherein R²³ is phenyl, a 6-membered heterocyclic ring, or a 6-membered heteroaryl ring, wherein the phenyl, 6-membered heterocyclic ring, or 6-membered heteroaryl ring has a single substituent at the ortho or meta position.

22. A compound according to claim 15, wherein R^{x} is CH₂—R²⁴, wherein R²⁴ is a 5-membered heteroaryl ring or 5-membered heterocyclic ring, wherein the 5-membered heteroaryl or heterocyclic ring has a single substituent at the ortho position.

23. A compound according to claim 1, wherein W is —NH—C(S)—NH—R^{z}.

24. A compound according to claim 23, wherein R^{z} is selected from the group consisting of: 2-methyl-phenyl; 3-methyl-phenyl; 4-methyl-phenyl; 2-fluoro-phenyl; 3-fluoro-phenyl; 4-fluoro-phenyl; 2,6-difluoro-phenyl; benzyl; 2-phenyl-ethyl; napth-1-yl; cyclohexyl; 4′-propyl-4-cyclohexyl-phenyl; and phenyl.

25. A compound according to claim 23, wherein R^{z} is selected from the group consisting of: 2-fluoro-phenyl; 3-fluoro-phenyl; and 4-fluoro-phenyl.

26. A compound according to claim 1, wherein W is —NH—C(O)—NH—R^{z}.

27. A compound according to claim 26, wherein R′ is selected from the group consisting of: n-butyl; n-octyl; cyclohexyl; benzyl; phenyl; 2-trifluoromethyl-phenyl; 3-trifluoromethyl-phenyl; 4-trifluoromethyl-phenyl; 2-methoxy-phenyl; 2,6-dimethyl-phenyl; napth-1-yl; 1-napth-1-yl-ethyl; and 2-methyl-phenyl.

28. A compound according to claim 26, wherein R^{z} is selected from the group consisting of: benzyl; phenyl; and 2-methyl-phenyl.

29. A compound according to claim 26, wherein R^{z} is selected from the group consisting of: n-butyl; n-octyl; cyclohexyl; benzyl; phenyl; 2-trifluoromethyl-phenyl; 3-trifluoromethyl-phenyl; 4-trifluoromethyl-phenyl; 2-methoxy-phenyl; 2,6-dimethyl-phenyl; napth-1-yl; and 1-napth-1-yl-ethyl.

30. A compound according to claim 1, wherein W is —NH—C(O)O—R^{z}.

31. A compound according to claim 30, wherein R^{z} is selected from the group consisting of: propyl; butyl; hexyl; octyl; decyl; isopropyl; isobutyl; 2,2-dimethyl-propyl; 2-ethyl-hexyl; (1S,2R,5S)-2-isopropyl-5-methyl-cyclohex-1-yl; (1R,2S,5R)-2-isopropyl-5-methyl-cyclohex-1-yl; ethenyl; prop-2-enyl; but-3-enyl; 1-methyl-ethenyl; but-3-ynyl; but-2-ynyl; 4-fluorophenyl; 4-bromophenyl; 4-nitrophenyl; 4-methoxycarbonyl-phenyl; 2-chloro-phenyl; 4-chloro-phenyl; 2-methoxy-phenyl; 4-methoxy-phenyl; 4-methyl-phenyl; 2-nitro-phenyl; 3-trifluoromethyl-phenyl; 2-nitro-3,4-dimethoxy-phenyl; benzyl; 2-chloro-phenylmethyl; (2-trifluoromethyl-phenyl)-chloro-methyl; and (4-nitro-phenyl)-methyl.

32. A compound according to claim 30, wherein R^{z} is selected from the group consisting of: hexyl; 4-methyl-phenyl; and 4-nitrophenyl.

33. A compound according to claim 1, wherein W is —NH—R′.

34. A compound according to claim 33, wherein R′ is selected from the group consisting of: 3,6-difluoro-benzyl; 3,6-dimethyl-benzyl; 2,3-dihydro-benzo[1,4]dioxin-6-yl-methyl; 2-phenyl-ethyl; cyclohexyl-methyl; n-nonyl; n-heptyl; 2-phenyl-propyl; 4-bromo-benzyl; napth-2-yl-methyl; and 4-phenoxy-benzyl.

35. A compound according to claim 33, wherein R′ is selected from the group consisting of: 4-bromo-benzyl and napth-2-yl-methyl.

36. A compound according to claim 1, wherein W is —NH—S(O₂)—R″.

37. A compound according to claim 36, wherein R″ is selected from the group consisting of: 4-fluoro-phenyl, napth-2-yl, and phenyl.

38. A compound according to claim 36, wherein R″ is napth-2-yl.

39. A compound according to claim 1, wherein W is —N(CH₃)—S(O₂)—R″.

40. A compound according to claim 39, wherein R″ is phenyl-sulfonyl-N-methyl-amino.

41. A compound according to claim 1, wherein W is —NH—C(O)—CH═N—NH—R²⁰.

42. A compound according to claim 41, wherein R²⁰ is selected from the group consisting of: phenylaminothiocarbonyl; N-ethylaminothiocarbonyl; N-prop-2-enylamino-thiocarbonyl; phenylaminocarbonyl; phenylcarbonyl; 3-methoxy-phenylcarbonyl; pyridine-4-yl-carbonyl; thiophen-2-ylcarbonyl; and benzylcarbonyl.

43. A compound according to claim 41, wherein R²⁰ is selected from the group consisting of: phenylaminothiocarbonyl and benzylcarbonyl.

44. A compound according to claim 1, wherein W is substituted aryl.

45. A compound according to claim 44, wherein W is 2-methyl-phenyl.

46. A compound according to claim 1, with the proviso: when R^{y} is CH₂CONH₂, R² is —NH₂, R³ and R⁴ are —NH₂ or —NH(protecting group), R⁵ is H, α-D-mannopyranosyl, or 2-O-α-D-mannopyranosyl-α-D-mannopyranosyl, and W is substituted carbonyl, then W is not —CO-alkyl, —CO-alkenyl, —CO—R²¹, —CO-(C1-C4 alkylene)-R²¹, or —CO-(C2-C4 alkenylene)-R²¹ wherein R²¹ is alkoxy; substituted alkoxy; alkenyloxy; substituted alkenyloxy, phenyl; substituted phenyl; napthyl; substituted napthyl; phenoxy; substituted phenoxy; napthoxy; or substituted napthoxy.

47. A compound according to claim 1, wherein the compound is selected from the group consisting of compounds 1-297 as shown in Tables I-VIII and prodrugs, tautomers and pharmaceutically acceptable salts thereof.

48. A compound according to claim 1, wherein the compound is selected from the group consisting of: compounds 11, 14, 29, 37, 38, 42, 44, 68, 70, 77, 88, 91, 92, 105, 108, 110, 111, 112, 113, 118, 119, 123, 124, 126, 144, 147, and 271, and prodrugs, tautomers and pharmaceutically acceptable salts thereof.

49. A compound according to claim 1, wherein the compound is selected from the group consisting of: compounds 92, 123, 147, and 271, and prodrugs, tautomers and pharmaceutically acceptable salts thereof.

50. A compound according to claim 1, wherein the compound is compound 92.

51. A compound according to claim 1, wherein the compound is compound 123.

52. A compound according to claim 1, wherein the compound is compound 147.

53. A compound according to claim 1, wherein the compound is compound 271.

54. A pharmaceutical composition comprising a pharmaceutically acceptable carrier and a therapeutically effective amount of a compound of claim 1.

55. A method for the treatment of a microbial infection in a mammal comprising administering to the mammal a therapeutically effective amount of a compound of claim 1.

56. The method according to claim 55, wherein the compound is administered to the mammal orally, parenterally, transdermally, topically, rectally, or intranasally in a pharmaceutical composition.

57. The method according to claim 55, wherein the compound is administered in an amount of from about 0.1 to about 100 mg/kg of body weight/day.
